(19) United States
(12) Patent Application Publication
(10) Pub. No.: US 2010/0158858 A1
(43) Pub. Date: Jun. 24, 2010

(54) ADMINISTRATION OF CARBOLINE DERIVATIVES USEFUL IN THE TREATMENT OF CANCER AND OTHER DISEASES

(76) Inventors: Liangxian Cao, Parlin, NJ (US); Samit Hirawat, Chatham, NJ (US); Langdon Miller, Lebanon, NJ (US); Gary Elfring, Lebanon, NJ (US); Thomas Davis, South Orange, NJ (US); Marla L. Weetall, Morristown, NJ (US)

Correspondence Address: JONES DAY, 222 EAST 41ST ST, NEW YORK, NY, 10017, US

(21) Appl. No.: 12/595,134

(22) PCT Filed: Apr. 12, 2008
(86) PCT No.: PCT/US08/04809

§ 371 Date: Mar. 1, 2010

Related U.S. Application Data
(60) Provisional application No. 60/911,612, filed on Apr. 13, 2007.

(51) Int. Cl. A61K 38/20; A61K 38/21; A61K 39/395; A61K 31/66; A61K 31/551; A61K 31/5377; A61K 31/497; A61K 31/506; C07D 413/14; C07D 471/04; A61P 35/00

(52) U.S. Cl. 424/85.2; 424/85.7; 424/133.1; 514/130; 514/218; 514/232.8; 514/253.03; 514/255.05; 514/275; 540/575; 544/126; 544/331; 544/361; 544/405

(57) ABSTRACT

In accordance with the present invention, compounds are provided which are useful in a method or in the manufacture of a medicament for post-transcriptionally inhibiting the expression of VEGF in a subject in need thereof comprising inhibiting VEGF mRNA translation by orally administering said medicament once, twice or thrice daily to the subject.

### CROSS REFERENCE TO RELATED APPLICATIONS

This application claims the benefit of U.S. Provisional Application No. 60/911,612, filed Apr. 13, 2007.

### STATEMENT REGARDING FEDERALLY SPONSORED RESEARCH OR DEVELOPMENT

Not Applicable

### FIELD OF THE INVENTION

The present invention relates to the use of compounds and methods for post-transcriptionally inhibiting the expression of VEGF.

### BACKGROUND OF THE INVENTION

Aberrant angiogenesis plays a critical role in the pathogenesis of numerous diseases, including malignant, ischemic, inflammatory and immune disorders (Carmeliet, Nat. Med., 9(6):653-60 (2003), Ferrara, Semin. Oncol., 29 (6 Suppl 16):10-4 (2002)). The best-known of these disorders are cancer, exudative macular degeneration and diabetic retinopathy (DR), the last two of which are leading causes of blindness in the United States (Witmer et al., Prog. Retinal and Eye Res., 22(1):1-29 (2003), Clark et al., Nat. Rev. Drug Discovery, 2:448-459 (2003)). During the last decade, an understanding of the molecular basis of angiogenesis has grown considerably. Numerous cytokines and growth factors that stimulate angiogenesis, such as VEGF, FGF-2, PDGF, IGF-1, TGF, TNF-α and G-CSF have been identified (Ferrara et al., Nat. Med., 5(12):1359-64 (1999), Kerbel et al., Nat. Rev. Cancer, 2(10):727-39 (2002), Rofstad et al., Cancer Res., 60(17):4932-8 (2000)). Among these growth factors, Vascular Endothelial Growth Factor (VEGF) plays a central role in angiogenesis (Ferrara, Semin. Oncol., 29(6 Suppl 16):10-4 (2002)).

VEGF, also known as VEGF-A, was initially identified for its ability to induce vascular permeability and to promote vascular endothelial cell proliferation (Leung et al., Science, 246:1306-1309 (1989), Plouet et al., EMBO J., 8:3801-3806 (1989), Connolly et al., J. Biol. Chem., 264:20017-20024 (1989)). VEGF is encoded by a single gene that gives rise to four isoforms by alternative splicing (Tischer et al., J. Biol. Chem., 266:11947-11954 (1991)). All four isoforms share the same unusually long and GC rich 5′-UTR, as well as a 3′-UTR that includes multiple RNA stability determinants. The receptors VEGFR-2 (also known as KDR or Flk-1) and VEGFR-1 (previously known as Flt1) recognize the dimeric form of VEGF (Ortega et al., Front. Biosci., 4:D141-52 (1999), Sato et al., Annals of New York Academy of Science, 902:201-207, (2000)). The highly specific VEGFR-2 receptor is expressed on endothelial cells. VEGF binding to the VEGFR-2 receptor activates the receptor's tyrosine kinase activity, leading to endothelial cell proliferation, differentiation and primitive vessel formation (Shalaby et al., Nature, 376:62-66, (1995)). VEGFR-1 inhibits endothelial cell growth either by acting as a decoy or by suppressing signaling pathways through VEGFR-2 (Fong et al., Nature, 376:66-70 (1995)).

Over 30 years ago, it was proposed that inhibition of tumor angiogenesis could be an effective approach for the treatment of cancer (Folkman, N. Engl. J. Med., 285(21):1182-6 (1971)). VEGF and its receptor have been demonstrated to have a central role in tumor angiogenesis, especially in the early stages of tumor growth (Hanahan et al., Cell, 86:353-364, 1996)). Indeed, increased levels of VEGF expression have been correlated with microvessel density in primary tumor tissues (Gasparini et al., J. Natl. Cancer Inst., 89:139-147 (1997)). Moreover, increased levels of the VEGF transcript are found in virtually all of the common solid tumors (Ferrara et al., Endocr. Rev., 18:4-25, 1997)). In general, tumor-bearing patients have higher levels of VEGF compared to those in tumor-free individuals, and high VEGF levels in serum/plasma are associated with poor prognosis (Dirix et al., Br. J. Cancer, 76:238-243 (1997)). Tumor volume has also been correlated with the levels of VEGF, where tumor volume tended to be larger in the high VEGF group than in the low VEGF group (Uesaka et al., J Neurooncol., 83(3):259-66 (2007 July). Epub 2007 Feb. 14).

Consistent with the role of VEGF in tumor angiogenesis, VEGF null embryonic stem cells showed a dramatically reduced ability to form tumors in nude mice (Carmeliet et al., Nature, 380:435-439 (1996)). Direct evidence for the involvement of VEGF in tumorgenesis was demonstrated by using specific antibodies against VEGF in human xenografts implanted in nude mice (Kim et al., Nature, 362:841-844 (1993), Hichlin et al., Drug Discovery Today, 6:517-528 (2001)). In these studies, the inhibition of tumor growth correlated positively with decreased vessel formation in the antibody-treated tumors. Subsequent experiments using the soluble receptors substantiated the importance of VEGF activity in tumor growth (Lin et al., Cell Growth Differ., 9(1):49-58 (1998)), and demonstrated that inactivation of VEGF by specific antibody treatment directly resulted in a nearly complete suppression of tumor-associated neovascularization (Borgstrom et al., Prostate, 35:1-10 (1998), Yuan et al. Proc. Natl. Acad. Sci. USA, 93:14765-14770 (1996)).

In exudative macular degeneration and diabetic retinopathy, pre-clinical experiments and clinical trials have demonstrated that over production of VEGF is critical for aberrant retinal or choroidal neovascularization (reviewed in Witmer et al., Prog. Retin Eye Res., 22(1):1-29 (2003)). Evidence has been obtained that intra-ocular VEGF levels are strongly correlated with active retinal/choroidal neovascularization (CNV) in patients with diseases such as diabetic retinopathy and wet form macular degeneration (Funatsu et al., Am. J. Opthalmol., 133(4):537-43 (2002), Lip et al., Opthalmology, 108(4):705-10 (2001)). In addition, studies using transgenic mice demonstrated that overexpression of VEGF in retinal pigment epithelial cells or photoreceptor cells results in choroidal or retinal neovascularization (Schwesinger et al., Am. J. Pathol., 158(3):1161-72 (2001), Ohno-Matsui et al., Am. J. Pathol., 160(2):711-9 (2002)). In recent studies neutralizing antibodies, soluble receptor, receptor antagonists, or siRNA have proven efficacious in reducing VEGF-mediated blood vessel formation in animal models and in the clinic. (Eyetech Study Group, 22(2):143-52 (2002), Krzystolik et al., Arch. Opthalmol., 120(3):338-46 (2002), Shen et al., Lab Invest., 82(2):167-82 (2002), Honda et al., Gene Ther., 7(11):978-85 (2000), Saishin et al., J Cell Physiol., 195(2):241-8 (2003)).

VEGF expression is regulated by a number of factors and agents including cytokines, growth factors, steroid hormones and chemicals, and mutations that modulate the activity of oncogenes such as Ras or the tumor suppressor gene VHL (Maxwell et al., Nature, 399:271-275 (1999), Rak et al., Cancer Res., 60:490-498 (2000)). Nevertheless, hypoxia is the most significant physiologic signal for regulating VEGF expression. Hypoxia results in enhanced VEGF expression by increasing both the transcription rate and stability of the VEGF transcript (Ikeda et al., J. Biol. Chem. 270:19761-19766 (1995), Stein et al., Mol. Cell. Biol. 18:3112-3119 (1998), Levy et al., J. Biol. Chem. 271:2746-2753 (1996)). Hypoxia-inducible factor 1α (HIF-1α) is a transcription factor that increases VEGF gene expression in cells undergoing hypoxia by binding to the hypoxia response element (HRE) located in the VEGF promoter (Liu et al., Circ. Res., 77:638-643 (1995), Semenza, Annu. Rev. Cell. Dev. Biol., 5:551-578 (1999)).

Both the stability and translation efficiency of the VEGF transcript is influenced by sequences in the 5′- and 3′-untranslated regions (UTRs). The 5′-UTR contains an internal ribosomal entry site (IRES) and mediates cap-independent translation initiation while the 3′-UTR harbors multiple AU-rich (AUR) stability determinants that have been previously shown to regulate turnover of VEGF mRNA. In addition, the translation initiation of the VEGF transcript is uniquely regulated. Under hypoxic conditions, translation of most cellular transcripts mediated by cap-dependent translation initiation process is greatly impaired (Kraggerud et al., Anticancer Res., 15:683-686 (1995)). Initiation of translation of the VEGF mRNA, however, is unique under hypoxic conditions in that it is mediated via an internal ribosome entry site (IRES) within the VEGF 5′-UTR (Stein et al., Mol. Cell. Biol. 18:3112-3119 (1998), Levy et al., J. Biol. Chem. 271:2746-2753 (1996), Huez et al., Mol. Cell. Biol., 18:6178-6190 (1998), Akin et al., Oncogene, 17:227-236 (1998)). Thus, this form of post-transcriptional regulation permits cells to produce large amounts of VEGF protein to support either further tumor growth or aberrant neovascularization in ocular diseases under hypoxic conditions. The stability of VEGF mRNA is also greatly enhanced as a consequence of the binding of factors to elements in the 3′-UTR (Goldberg et al., J. Biol. Cell. J. Biol. Chem., 277(16):13635-40 (2002)).

There is a large body of experimental evidence indicating that tumor growth can be inhibited by the prevention of neovascularization (Lin et al., Cell Growth Differ., 9(1):49-58 (1998), Zhu et al., Invest. New Drugs, 17:195-212 (1999)). Tumor vessels are generally immature and constantly undergo remodeling (Carmeliet, Nat. Med., 9(6):653-60 (2003), Carmeliet et al., Nature, 407:249-257 (2000)). Active and aberrant angiogenesis is the result of a disruption in the normal balance of proangiogenic and anti-angiogenic factors, including various cytokines, growth factors and steroid hormones. Despite the complexity of the regulation of tumor angiogenesis, accumulated evidence indicates that targeting a single proangiogenic factor might be sufficient to inhibit tumor angiogenesis and suppress tumor growth (Kim et al., Nature, 362:841-844 (1993), Millauer et al., Nature, 367:576-579 (1994), Fong et al., Cancer Res., 59:99-106 (1999)). Among many angiogenesis targets, VEGF and its receptor are most attractive (Carmeliet, Nat. Med., 9(6):653-60 (2003), Ortega et al., Front. Biosci., 4:D141-52 (1999)). As noted above, treatment with a monoclonal antibody specifically targeting VEGF inhibited the growth of tumors in human xenografts implanted in nude mice. While oncogenic mutations resulting in activated Ras Guanosine Triphosphate (GTP) are prevalent in 30% of all human cancers, inhibition of Ras by genetic or pharmacological strategies leads to decreased astrocytoma tumorgenic growth in vitro and decreased expression of VEGF (Guha, Can J Neurol Sci., 25(4):267-81 (1998 November)). Subsequently, various approaches designed to inactivate VEGF signaling have been tested in tumor models and have proven to be highly effective in a broad range of tumor cell lines including carcinomas, sarcomas and gliomas (Ferrara et al., Endocr. Rev., 18:4-25, 1997), Kim et al., Nature, 362:841-844 (1993), Millauer et al., Nature, 367:576-579 (1994), Fong et al., Cancer Res., 59:99-106 (1999), Geng et al., Cancer Res., 61:2413-2419 (2001)). Since reduction in tumor growth may be due to decreased tumor angiogenesis, leading to reduction of tumor cell proliferation and increased apoptosis, inhibiting VEGF function may therefore be a useful adjuvant therapy for neurogenic sarcomas (Angelov, et al., Cancer Res., 59(21):5536-41 (1999 Nov. 1)) and human neurofibromas (Arbiser, et al., J Am Acad Dermatol., 38(6 Pt 1):950-4 (1998 June)). In addition, inhibition of VEGF by an anti-VEGF antibody did not result in significant side effects in fully developed rodents or primates (Ryan et al, Toxicol. Pathol., 27:78-86 (1999), Ferrara et al., Nat. Med., 4:336-340 (1998)). Taken together, these results indicate that VEGF is a valid target for use in tumor therapy. Indeed, a number of clinical trials are underway using VEGF inhibitors (Matter, Drug Discovery Today, 6:1005-1024 (2001), Hichlin et al., Drug Discovery Today, 6:517-528 (2001)).

Although several pro-angiogenic factors are implicated in the pathology of exudative age-related macular degeneration, VEGF appears to be the most critical in the pathogenesis and development of this disease (Witmer et al., Prog. Retin Eye Res., 22(1):1-29 (2003), Holash et al., Science, 284:1994-1998 (1999)). Data from preclinical experiments and clinical trials have demonstrated that blockade of VEGF alone is sufficient to alleviate or stabilize disease progression (Eyetech Study Group, 22(2):143-52 (2002), Krzystolik et al., Arch. Opthalmol., 120(3):338-46 (2002), Shen et al., Lab Invest., 82(2):167-82 (2002), Honda et al., Gene Ther., 7(11):978-85 (2000), Saishin et al., J. Cell Physiol., 195(2):241-8 (2003)). For example, inhibition of VEGFR signaling by a specific tyrosine kinase inhibitor is sufficient to completely prevent retinal neovascularization in a murine retinopathy of prematurity model (Ozaki H, Seo M S, Ozaki et al., Am. J. Pathol., 156(2):697-707 (2000)). Furthermore, it has recently been demonstrated that small interfering RNAs (siRNA) directed against murine VEGF significantly inhibited ocular neovascularization after laser photocoagulation in a mouse model (Reich et al., Mol. Vis. 30; 9:210-6 (2003)). These results indicate that selective inhibition of VEGF expression is achievable and offers validation of this approach for the treatment of ocular neovascular diseases such as exudative macular degeneration and diabetic retinopathy.

Three approaches have been used to inhibit VEGF activity, including (1) neutralization of VEGF activity by using a specific antibody, soluble VEGF receptor or aptamer oligos against the VEGF/VEGFR interaction (Kim et al., Nature, 362:841-844 (1993), Lin et al., Cell Growth Differ., 9(1):49-58 (1998), Borgstrom et al., Prostate, 35:1-10 (1998), Zhu et al., Invest. New Drugs, 17:195-212 (1999), Millauer et al., Nature, 367:576-579 (1994), Asano et al., Jpn. J. Cancer Res., 90(1):93-100 (1999), Brekken et al., Cancer Res., 60(18):5117-24 (2000)); (2) inhibition of VEGFR mediated signal transduction by specific small molecule tyrosine kinase inhibitors (Fong et al., Cancer Res., 59:99-106 (1999), Wedge et al., Cancer Res., 60(4):970-5 (2000), Laird et al., Cancer Res., 60(15):4152-60 (2000)); and (3) inhibition of VEGF/VEGFR expression by using antisense, siRNA or ribozyme (Reich et al., Mol. Vis. 30; 9:210-6 (2003), Parry et al., Nucleic Acids Res., 27:2569-2577 (1999), Ellis et al., Surgery, 120:871-878 (1996), Filleur et al., Cancer Res., 63(14):3919-22 (2003)). Although all of these approaches show significant inhibition of angiogenesis in vivo, they all possess significant limitations. For example, therapeutic proteins (antibody and soluble receptors) or oligos (antisense, siRNA and ribozyme) are large molecules with poor permeability that usually require parenteral administration and are costly to produce. For treatment of chronic ocular neovascularization, multiple injections may be impractical due to potential complications such as retinal detachment and procedure related infection. Moreover, tyrosine kinase inhibitors have the potential for limited specificity. VEGF is constitutively expressed at a low level in normal eyes and other tissues and thus it may be harmful to completely suppress VEGF function by administration of antibody or tyrosine kinase inhibitors systemically, especially for patients with AMD and RD many of whom are also hypertensive (Giles et al., Cancer, 97(8):1920-8 (2003), Sugimoto et al., J. Biol. Chem., 278(15):12605-8 (2003), Bergsland et al., American Society of Clinical Oncology 36^{th} Annual Meeting, 20-23 May, 2000, New Orleans, La., USA, Abstract 939), DeVore et al., American Society of Clinical Oncology 36^{th} Annual Meeting, 20-23 May, 2000, New Orleans, La., USA, Abstract 1896).

Carboline derivatives have been disclosed in European Patent Publication 0549916, International Publication WO97/37658, International Publication WO03/033496, International Publication WO03/099821, United States Patent Publication 2003/0040527 and U.S. Pat. No. 7,122,554.

Carboline derivatives of the present invention have been disclosed in U.S. patent application Ser. No. 11/735,069, filed Apr. 13, 2007, U.S. patent application Ser. No. 11/107,783, filed Apr. 18, 2005 (having corresponding International Application No. PCT/US2006/014547, filed on Apr. 17, 2006) and U.S. patent application Ser. No. 11/079,420, filed Mar. 15, 2005 (having corresponding International Application No. PCT/US2005/008481, filed Mar. 15, 2005), each of which are incorporated herein by reference in their entirety and for all purposes.

However, there remains a need to develop, characterize and optimize the use of compounds and methods for post-transcriptionally inhibiting the expression of VEGF. Accordingly, it is an object of the present invention to provide for such use and methods.

All documents referred to herein are incorporated by reference into the present application as though fully set forth herein.

### SUMMARY OF THE INVENTION

The present invention relates to use of compounds and methods for post-transcriptionally inhibiting the expression of VEGF.

In accordance with the present invention, compounds that inhibit the expression of VEGF post-transcriptionally have been identified, and methods for their use provided.

In one aspect of the invention, compounds of Formula (V) are provided which are useful in a method for post-transcriptionally inhibiting the expression of VEGF in a subject in need thereof comprising inhibiting VEGF mRNA translation by orally administering once, twice or thrice daily to the subject either (i) a therapeutically effective amount of one or more compounds of Formula (V) or one or more pharmaceutically acceptable salts, hydrates, solvates, clathrates, polymorphs, racemates or stereoisomers thereof, or (ii) a pharmaceutical composition comprising one or more pharmaceutically acceptable excipients and a therapeutically effective amount of one or more compounds of Formula (V) or one or more pharmaceutically acceptable salts, hydrates, solvates, clathrates, polymorphs, racemates or stereoisomers thereof.

In this aspect of the invention, inhibiting VEGF mRNA translation treats a VEGF mediated disorder or a solid tumor cancer by reducing plasma and solid tumor VEGF levels, reducing perivascularly sequestered VEGF, reducing aberrant vascular permeability, or inhibiting angiogenesis.

In another aspect of the invention, the compounds of Formula (V) or said pharmaceutical composition thereof, are selected from compounds of Formulas (I), (II), (III) and (IV), including Formulas (I-a) to (I-m) or pharmaceutically acceptable salts, hydrates, solvates, clathrates, polymorphs, racemates or stereoisomers thereof.

In another aspect of the invention, said compounds are useful for post-transcriptionally inhibiting the expression of VEGF in a method for treating a VEGF mediated disorder selected from cancer, diabetic retinopathy, exudative macular degeneration, rheumatoid arthritis, psoriasis, atherosclerosis, chronic inflammation, other chronic inflammation-related diseases and disorders or obesity.

In another aspect of the invention, compounds of Formulas (I), (II), (III), (IV) and (V), including Formulas (I-a) to (I-m) or pharmaceutically acceptable salts, hydrates, solvates, clathrates, polymorphs, racemates or stereoisomers thereof, are useful in the manufacture of a medicament for post-transcriptionally inhibiting the expression of VEGF in a subject in need thereof, wherein inhibiting VEGF mRNA translation results in treating a VEGF mediated disorder selected from a solid tumor cancer, diabetic retinopathy, exudative macular degeneration, rheumatoid arthritis, psoriasis, atherosclerosis, chronic inflammation, other chronic inflammation-related diseases and disorders or obesity in the subject by reducing plasma and solid tumor VEGF levels.

In another aspect of the invention, compounds of Formulas (I), (II), (III), (IV) and (V), including Formulas (I-a) to (I-m), are useful in the manufacture of a medicament or in a method for post-transcriptionally inhibiting the expression of VEGF in a subject in need thereof, wherein inhibiting VEGF mRNA translation results in treating a VEGF mediated solid tumor cancer by reducing plasma and solid tumor VEGF levels, thus slowing tumorigenesis of a solid tumor, reducing perivascularly sequestered VEGF, reducing aberrant vascular permeability and inhibiting angiogenesis.

In one embodiment, the invention is directed to methods for inhibiting VEGF production in a subject in need thereof comprising administering a therapeutically effective amount of at least one compound of the invention to the subject.

In another embodiment, methods for inhibiting angiogenesis in a subject in need thereof are provided comprising administering a therapeutically effective amount of at least one compound of the invention to the subject.

The present invention also provides methods for treating a solid tumor cancer in a subject in need thereof comprising administering a therapeutically effective amount of a compound of Formula (V), or a pharmaceutically acceptable salt, hydrate, solvate, clathrate, polymorph, racemate or stereoisomer of said compound to the subject.

The present invention also provides methods for treating a solid tumor cancer selected from a pediatric solid tumor, an Ewing's sarcoma, a Wilms tumor, a neuroblastoma, a neurofibroma, a carcinoma of the epidermis, a malignant melanoma, a cervical carcinoma, a colon carcinoma, a lung carcinoma, a renal carcinoma, a breast cancinoma or a breast sarcoma in a subject in need thereof, comprising administering a therapeutically effective amount of a compound of Formula (V), or a pharmaceutically acceptable salt, hydrate, solvate, clathrate, polymorph, racemate or stereoisomer thereof, to a subject in need thereof.

The present invention further provides methods for treating a solid tumor cancer by post-transcriptionally inhibiting VEGF expression comprising administering a therapeutically effective amount of a compound of Formula (V), or a pharmaceutically acceptable salt, hydrate, solvate, clathrate, polymorph, racemate or stereoisomer of said compound, to a subject in need thereof.

The present invention yet further provides methods for treating a solid tumor cancer by slowing tumorigenesis of a solid tumor comprising administering a therapeutically effective amount of a compound of Formula (V), or a pharmaceutically acceptable salt, hydrate, solvate, clathrate, polymorph, racemate or stereoisomer of said compound, to a subject in need thereof.

The present invention also provides methods for treating a solid tumor cancer by reducing solid tumor VEGF levels comprising administering a therapeutically effective amount of a compound of Formula (V), or a pharmaceutically acceptable salt, hydrate, solvate, clathrate, polymorph, racemate or stereoisomer of said compound, to a subject in need thereof.

The present invention further provides methods for treating a solid tumor cancer by reducing perivascularly sequestered VEGF comprising administering a therapeutically effective amount of a compound of Formula (V), or a pharmaceutically acceptable salt, hydrate, solvate, clathrate, polymorph, racemate or stereoisomer of said compound, to a subject in need thereof.

The present invention also provides methods for treating a VEGF mediated disorder by post-transcriptionally inhibiting VEGF mRNA translation comprising administering a therapeutically effective amount of a compound of Formula (V), or a pharmaceutically acceptable salt, hydrate, solvate, clathrate, polymorph, racemate or stereoisomer of said compound, to a subject in need thereof.

The present invention yet further provides methods for treating a solid tumor cancer by reducing aberrant vascular permeability comprising administering a therapeutically effective amount of a compound of Formula (V), or a pharmaceutically acceptable salt, hydrate, solvate, clathrate, polymorph, racemate or stereoisomer of said compound, to a subject in need thereof.

The present invention also provides methods for treating a VEGF mediated disorder by reducing plasma VEGF levels comprising administering a therapeutically effective amount of a compound of Formula (V), or a pharmaceutically acceptable salt, hydrate, solvate, clathrate, polymorph, racemate or stereoisomer of said compound, to a subject in need thereof.

The present invention yet further provides methods of treating a VEGF mediated disorder or a solid tumor cancer comprising measuring serum or plasma levels of VEGF, tumor levels of VEGF, or both, and administering a therapeutically effective amount of a compound of Formula (V), or a pharmaceutically acceptable salt, hydrate, solvate, clathrate, polymorph, racemate or stereoisomer of said compound, to a subject in need thereof.

The present invention also provides methods of diagnosing solid tumor cancers comprising measuring serum or plasma levels of VEGF.

The present invention further provides methods of diagnosing solid tumor cancers comprising measuring tumor levels of VEGF.

The present invention yet further provides methods of treating a solid tumor cancer comprising administering a therapeutically effective amount of a compound of Formula (V), or a pharmaceutically acceptable salt, hydrate, solvate, clathrate, polymorph, racemate or stereoisomer thereof, optionally administered in combination with one or more additional agents useful for treating cancer to a subject in need thereof.

These and other aspects of the invention will be more clearly understood with reference to the following preferred embodiments and detailed description.

### BRIEF DESCRIPTION OF THE DRAWINGS

FIG. 1 illustrates the mean (average) plasma concentrations of a compound of the invention at several times following administration of a single dose of a compound to normal healthy subjects. The error bars show the standard deviation.

FIG. 2 illustrates the mean plasma concentrations of a compound of the invention at several times after administration of a single dose of 1 mg/kg of a compound to normal healthy subjects without or with (fed and fasted) a high fat high calorie meal fed to the subjects immediately before administration of the compound

FIG. 3 illustrates the mean plasma concentrations of a compound of the invention at day one and seven of a seven day dosing study of normal healthy subjects at three different doses administered twice per day (0.3, 0.6 or 1.2 mg/kg). The error bars show the standard deviation.

FIG. 4 illustrates the mean plasma concentrations of a compound of the invention at day one and seven of a seven day dosing study of normal healthy subjects at a dose 1.6 mg/kg (a total of 4.8 mg/kg/day) administered three times per day. The error bars show the standard deviation.

FIG. 5 illustrates the effect on mean tumor volume over a time period by oral administration (QID) of a certain compound of the invention in comparison to letrozole.

### DETAILED DESCRIPTION OF THE INVENTION

Aberrant up-regulation of Vascular Endothelial Growth Factor (VEGF), a key factor for angiogenesis, is an important contributor to the pathogenesis of VEGF mediated disorders such as cancer, diabetic retinopathy, rheumatoid arthritis, psoriasis, atherosclerosis, chronic inflammation, other chronic inflammation-related diseases and disorders, obesity, or exudative macular degeneration. In accordance with the present invention, compounds that post-transcriptionally inhibit the expression of VEGF have been identified, and methods for their use provided. The compounds of the invention have nanomolar to sub-nanomolar activity for the inhibition of VEGF expression.

### Compounds of the Invention

In one aspect of the invention, compounds of Formula (V) are demonstrated to be useful in the inhibition of VEGF production, in the inhibition of angiogenesis, and/or in the treatment of VEGF mediated disorders. In certain embodiments, the compounds of the invention specifically inhibit VEGF production, while in other embodiments, the compounds of the invention inhibit VEGF expression as well as that of other angiogenesis factors such as FGF-2. In this regard, a pan-angiogenic inhibitor may be preferred in methods of inhibiting tumor growth, while VEGF specific inhibitors may be preferred for the treatment of ocular VEGF mediated neovascular disorders (Eyetech Study Group, 22(2):143-52 (2002)).

The compounds of the invention may include one or more chiral centers, and as such may exist as racemic mixtures (R/S) or as substantially pure enantiomers. The compounds may also exist as substantially pure (R) or (5) enantiomers (when one chiral center is present). In one embodiment, the compounds of the invention are (S) isomers and may exist as enantiomerically pure compositions substantially comprising only the (S) isomer. As one of skill in the art will recognize, when more than one chiral center is present, the compounds of the invention may also exist as (R,R), (R,S), (S,R), (S,S), etc. isomers.

As used herein, the term “substantially pure” refers to compounds consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of a single isomer.

In one aspect of the invention, a compound of Formula (V) is a substantially pure (S) enantiomer present in an amount greater than or equal to 90%, in an amount greater than or equal to 92%, in an amount greater than or equal to 95%, in an amount greater than or equal to 98%, in an amount greater than or equal to 99%, or in an amount equal to 100%.

More particularly, the compounds of the invention are present as the substantially pure (S) enantiomer at the chiral carbon on position 1 of the compound of Formula (V).

As used herein, a “racemic mixture” is any mixture of isometric forms that are not “substantially pure,” including, without limitation, about 50/50, about 60/40, and about 70/30 mixtures.

Compounds of Formula (V) useful for post-transcriptionally inhibiting the expression of VEGF include those of Formula (I), as shown below, as previously disclosed in U.S. patent application Ser. No. 11/735,069, filed Apr. 13, 2007, U.S. patent application Ser. No. 11/107,783, filed Apr. 18, 2005 (having corresponding International Application No. PCT/US2006/014547, filed on Apr. 17, 2006) and U.S. patent application Ser. No. 11/079,420, filed Mar. 15, 2005 (having corresponding International Application No. PCT/US2005/008481, filed Mar. 15, 2005), each of which are incorporated herein by reference in their entirety and for all purposes:

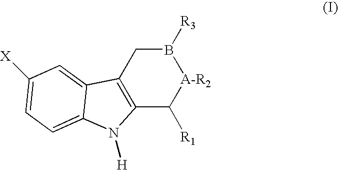

wherein,

X is hydrogen; a C₁ to C₆ alkyl, optionally substituted with one or more halogens; a hydroxyl group; a halogen; a C₁ to C₅ alkoxy, optionally substituted with a C₆ to C₁₀ aryl group;

A is CH or N;

B is CH or N, with the proviso that at least one of A or B is N, and that when A is N, B is CH;

R₁ is a hydroxyl group; a C₁ to C₈ alkyl group, optionally substituted with an alkylthio group, a 5 to 10 membered heteroaryl, a C₆ to C₁₀ aryl group optionally substituted with at least one independently selected Rₒ group; a C₂ to C₈ alkenyl group; a C₂ to C₈ alkynyl group; a 3 to 12 membered heterocycle group, wherein the heterocycle group is optionally substituted with at least one independently selected halogen, oxo, amino, alkylamino, acetamino, thio, or alkylthio group; a 5 to 12 membered heteroaryl group, wherein the heteroaryl group is optionally substituted with at least one independently selected halogen, oxo, amino, alkylamino, acetamino, thio, or alkylthio group; or a C₆ to C₁₀ aryl group, optionally substituted with at least one independently selected Rₒ group;

Rₒ is a halogen; a cyano; a nitro; a sulfonyl, wherein the sulfonyl is optionally substituted with a C₁ to C₆ alkyl or a 3 to 10 membered heterocycle; an amino group, wherein the amino group is optionally substituted with a C₁ to C₆ alkyl, —C(O)—R_{b}, —C(O)O—R_{b}, a sulfonyl, an alkylsulfonyl, a 3 to 10 membered heterocycle group optionally substituted with a —C(O)O—Rₙ; —C(O)—NH—R_{b}; a 5 to 6 membered heterocycle; a 5 to 6 membered heteroaryl; a C₁ to C₆ alkyl group, wherein the alkyl group is optionally substituted with at least one independently selected hydroxyl, halogen, amino, or 3 to 12 membered heterocycle group, wherein the amino group and heterocycle group are optionally substituted with at least one independently selected C₁ to C₄ alkyl group, which C₁ to C₄ alkyl group is optionally substituted with at least one independently selected C₁ to C₄ alkoxy group, amino group, alkylamino group, or 5 to 10 membered heterocycle group; a —C(O)—Rₙ group; or an —ORₐ group;

Rₐ is hydrogen; C₂ to C₈ alkenyl; a —C(O)O—R_{b} group; a —C(O)—NH—R_{b}; a C₁ to C₈ alkyl, wherein the alkyl group is optionally substituted with at least one independently selected hydroxyl, halogen, C₁ to C₄ alkoxy, amino, alkylamino, acetamide, —C(O)—R_{b}, —C(O)O—R_{b}, C₆ to C₁₀ aryl, 3 to 12 membered heterocycle, or 5 to 12 heteroaryl group, further wherein the alkylamino is optionally substituted with a hydroxyl, a C₁ to C₄ alkoxy, or a 5 to 12 membered heteroaryl optionally substituted with a C₁ to C₄ alkyl, further wherein the acetamide is optionally substituted with a C₁ to C₄ alkoxy, sulfonyl, or alkylsulfonyl, further wherein and the heterocycle group is optionally substituted with a C₁ to C₄ alkyl optionally substituted with a hydroxyl group, —C(O)—Rₙ, —C(O)O—Rₙ, or an oxo group;

R_{b} is hydroxyl; an amino; an alkylamino, wherein the alkylamino is optionally substituted with a hydroxyl, an amino, an alkylamino, a C₁ to C₄ alkoxy, a 3 to 12 membered heterocycle optionally substituted with at least one independently selected C₁ to C₆ alkyl, oxo, —C(O)O—Rₙ, or a 5 to 12 membered heteroaryl optionally substituted with a C₁ to C₄ alkyl; a C₁ to C₄ alkoxy; a C₂ to C₈ alkenyl; a C₂ to C₈ alkynyl; a C₆ to C₁₀ aryl, wherein the aryl is optionally substituted with at least one independently selected halogen or C₁ to C₄ alkoxy; a 5 to 12 membered heteroaryl; 3 to 12 membered heterocycle group, wherein the heterocycle is optionally substituted with at least one independently selected acetamide, —C(O)O—Rₙ, 5 to 6 membered heterocycle, or C₁ to C₆ alkyl optionally substituted with a hydroxyl, C₁ to C₄ alkoxy, amino group, or alkylamino group; or a C₁ to C₈ alkyl, wherein the alkyl is optionally substituted with at least one independently selected C₁ to C₄ alkoxy, C₆ to C₁₀ aryl, amino, or 3 to 12 membered heterocycle group, wherein the amino and heterocycle groups are optionally substituted with at least one independently selected C₁ to C₆ alkyl, oxo, or —C(O)O—Rₙ group;

R₂ is a hydrogen; a hydroxyl; a 5 to 10 membered heteroaryl group; a C₁ to C₈ alkyl group, wherein the alkyl group is optionally substituted with a hydroxyl, a C₁ to C₄ alkoxy, a 3 to 10 membered heterocycle, a 5 to 10 membered heteroaryl, or C₆ to C₁₀ aryl group; a —C(O)—R_{c} group; a —C(O)O—R_{d} group; a —C(O)—N(R_{d}R_{d}) group; a —C(S)—N(R_{d}R_{d}) group; a —C(S)—O—Rₑ group; a —S(O₂)—Rₑ group; a —C(NRₑ)—S—Rₑ group; or a —C(S)—S—R_{f} group;

R_{c} is hydrogen; an amino, wherein the amino is optionally substituted with at least one independently selected C₁ to C₆ alkyl or C₆ to C₁₀ aryl group; a C₆ to C₁₀ aryl, wherein the aryl is optionally substituted with at least one independently selected halogen, haloalkyl, hydroxyl, C₁ to C₄ alkoxy, or C₁ to C₆ alkyl group; —C(O)—Rₙ; a 5 to 6 membered heterocycle, wherein the heterocycle is optionally substituted with a —C(O)—Rₙ group; a 5 to 6 membered heteroaryl; a thiazoleamino group; a C₁ to C₈ alkyl group, wherein the alkyl group is optionally substituted with at least one independently selected halogen, a C₁ to C₄ alkoxy, a phenyloxy, a C₆ to C₁₀ aryl, —C(O)—Rₙ, —O—C(O)—Rₙ, hydroxyl, or amino group, optionally substituted with a —C(O)O—Rₙ group;

R_{d} is independently hydrogen; a C₂ to C₈ alkenyl group; a C₂ to C₈ alkynyl group; a C₆ to C₁₀ aryl group, wherein the aryl is optionally substituted with at least one independently selected halogen, nitro, C₁ to C₆ alkyl, —C(O)O—Rₑ, or —ORₑ; or a C₁ to C₈ alkyl group, wherein the alkyl group is optionally substituted with at least one independently selected halogen, C₁ to C₄ alkyl, C₁ to C₄ alkoxy, phenyloxy, C₆ to C₁₀ aryl, 5 to 6 membered heteroaryl, —C(O)—Rₙ, —O—C(O)—Rₙ, or hydroxyl group, wherein the C₆ to C₁₀ aryl group is optionally substituted with at least one independently selected halogen or haloalkyl group;

Rₑ is a hydrogen; a C₁ to C₆ alkyl group, wherein the alkyl group is optionally substituted with at least one independently selected halogen or alkoxy group; or a C₆ to C₁₀ aryl group, wherein the aryl group is optionally substituted with at least one independently selected halogen or alkoxy group;

R_{f} is a C₁ to C₆ alkyl group, optionally substituted with at least one independently selected halogen, hydroxyl, C₁ to C₄ alkoxy, cyano, C₆ to C₁₀ aryl, or —C(O)—Rₙ group, wherein the alkoxy group may be optionally substituted with at least one C₁ to C₄ alkoxy group and the aryl group may be optionally substituted with at least one independently selected halogen, hydroxyl, C₁ to C₄ alkoxy, cyano, or C₁ to C₆ alkyl group;

Rₙ is a hydroxyl, C₁ to C₄ alkoxy, amino, or C₁ to C₆ alkyl group;

R₃ is hydrogen or —C(O)—R_{g}; and

R_{g} is a hydroxyl group; an amino group, wherein the amino is optionally substituted with a C₆ to C₁₀ cycloalkyl group or a 5 to 10 membered heteroaryl group; or a 5 to 10 membered heterocycle group, wherein the heterocycle group is optionally substituted with a —C(O)—Rₙ group.

Embodiments of compounds of the present invention useful for post-transcriptionally inhibiting the expression of VEGF include those of Formula (I), wherein

X is hydrogen; a C₁ to C₆ alkyl, optionally substituted with one or more halogens; a hydroxyl group; a halogen; a C₁ to C₅ alkoxy, optionally substituted with a C₆ to C₁₀ aryl group,

with the proviso that, when X is C₁ to C₅ alkoxy and R₂ is —C(O)O—R_{d}, wherein R_{d} is C₁ to C₄ alkyl, then R₁ is other than unsubstituted C₁ to C₈ alkyl;

A is CH or N;

B is CH or N, with the proviso that at least one of A or B is N, and that when A is N, B is CH;

R₁ is a hydroxyl group; a C₁ to C₈ alkyl group, optionally substituted with an alkylthio group, a 5 to 10 membered heteroaryl, a C₆ to C₁₀ aryl group optionally substituted with at least one independently selected Rₒ group; a C₂ to C₈ alkenyl group; a C₂ to C₈ alkynyl group; a 3 to 12 membered heterocycle group, wherein the heterocycle group is optionally substituted with at least one independently selected halogen, oxo, amino, alkylamino, acetamino, thio, or alkylthio group; a 5 to 12 membered heteroaryl group, wherein the heteroaryl group is optionally substituted with at least one independently selected halogen, oxo, amino, alkylamino, acetamino, thio, or alkylthio group; or a C₆ to C₁₀ aryl group, optionally substituted with at least one independently selected Rₒ group,

with the proviso that, when R₁ is unsubstituted phenyl, then X is other than hydrogen;

Rₒ is a halogen; a cyano; a nitro; a sulfonyl, wherein the sulfonyl is optionally substituted with a C₁ to C₆ alkyl or a 3 to 10 membered heterocycle; an amino group, wherein the amino group is optionally substituted with a C₁ to C₆ alkyl, —C(O)—R_{b}, —C(O)O—R_{b}, a sulfonyl, an alkylsulfonyl, a 3 to 10 membered heterocycle group optionally substituted with a —C(O)O—Rₙ; —C(O)—NH—R_{b}; a 5 to 6 membered heterocycle; a 5 to 6 membered heteroaryl; a C₁ to C₆ alkyl group, wherein the alkyl group is optionally substituted with at least one independently selected hydroxyl, halogen, amino, or 3 to 12 membered heterocycle group, wherein the amino group and heterocycle group are optionally substituted with at least one independently selected C₁ to C₄ alkyl group, which C₁ to C₄ alkyl group is optionally substituted with at least one independently selected C₁ to C₄ alkoxy group, amino group, alkylamino group, or 5 to 10 membered heterocycle group; a —C(O)—Rₙ group; or an —ORₐ group;

Rₐ is hydrogen; C₂ to C₈ alkenyl; a —C(O)O—R_{b} group; a —C(O)—NH—R_{b}; a C₁ to C₈ alkyl, wherein the alkyl group is optionally substituted with at least one independently selected hydroxyl, halogen, C₁ to C₄ alkoxy, amino, alkylamino, acetamide, —C(O)—R_{b}, —C(O)O—R_{b}, C₆ to C₁₀ aryl, 3 to 12 membered heterocycle, or 5 to 12 heteroaryl group, further wherein the alkylamino is optionally substituted with a hydroxyl, a C₁ to C₄ alkoxy, or a 5 to 12 membered heteroaryl optionally substituted with a C₁ to C₄ alkyl, further wherein the acetamide is optionally substituted with a C₁ to C₄ alkoxy, sulfonyl, or alkylsulfonyl, further wherein and the heterocycle group is optionally substituted with a C₁ to C₄ alkyl optionally substituted with a hydroxyl group, —C(O)—Rₙ, —C(O)O—Rₙ, or an oxo group;

R_{b} is hydroxyl; an amino; an alkylamino, wherein the alkylamino is optionally substituted with a hydroxyl, an amino, an alkylamino, a C₁ to C₄ alkoxy, a 3 to 12 membered heterocycle optionally substituted with at least one independently selected C₁ to C₆ alkyl, oxo, —C(O)O—Rₙ, or a 5 to 12 membered heteroaryl optionally substituted with a C₁ to C₄ alkyl; a C₁ to C₄ alkoxy; a C₂ to C₈ alkenyl; a C₂ to C₈ alkynyl; a C₆ to C₁₀ aryl, wherein the aryl is optionally substituted with at least one independently selected halogen or C₁ to C₄ alkoxy; a 5 to 12 membered heteroaryl; 3 to 12 membered heterocycle group, wherein the heterocycle is optionally substituted with at least one independently selected acetamide, —C(O)O—Rₙ, 5 to 6 membered heterocycle, or C₁ to C₆ alkyl optionally substituted with a hydroxyl, C₁ to C₄ alkoxy, amino group, or alkylamino group; or a C₁ to C₈ alkyl, wherein the alkyl is optionally substituted with at least one independently selected C₁ to C₄ alkoxy, C₆ to C₁₀ aryl, amino, or 3 to 12 membered heterocycle group, wherein the amino and heterocycle groups are optionally substituted with at least one independently selected C₁ to C₆ alkyl, oxo, or —C(O)O—Rₙ group;

R₂ is a hydrogen; a hydroxyl; a 5 to 10 membered heteroaryl group; a C₁ to C₈ alkyl group, wherein the alkyl group is optionally substituted with a hydroxyl, a C₁ to C₄ alkoxy, a 3 to 10 membered heterocycle, a 5 to 10 membered heteroaryl, or C₆ to C₁₀ aryl group; a —C(O)—R_{c} group; a —C(O)O—R_{d} group; a —C(O)—N(R_{d}R_{d}) group; a —C(S)—N(R_{d}R_{d}) group; a —C(S)—O—Rₑ group; a —S(O₂)—Rₑ group; a —C(NRₑ)—S—Rₑ group; or a —C(S)—S—R_{f} group,

with the proviso that, when R₂, R₃ and X are hydrogen, then R₁ is other than fluorenyl, substituted carbazolyl or phenyl, wherein phenyl is optionally monosubstituted with halogen, nitro or substituted amino, or di- and tri-substituted with C₁ to C₄ alkoxy;

with the proviso that, when R₂ is —C(O)—R_{c}, —C(O)O—R_{d}, —C(O)—NH(R_{d}) or —C(S)—NH(R_{d}), wherein R_{c} is C₁ to C₈ alkyl substituted with optionally substituted phenyl, wherein R_{d} is optionally substituted phenyl, cyclohexyl or C₁ to C₈ alkyl optionally substituted with optionally substituted phenyl or —C(O)O—Rₙ, and R₃ and X are hydrogen, then R₁ is other than unsubstituted benzo[1,3]dioxolyl or optionally substituted phenyl, wherein phenyl is optionally disubstituted with chloro and methoxy;

Rₑ is hydrogen; an amino, wherein the amino is optionally substituted with at least one independently selected C₁ to C₆ alkyl or C₆ to C₁₀ aryl group; a C₆ to C₁₀ aryl, wherein the aryl is optionally substituted with at least one independently selected halogen, haloalkyl, hydroxyl, C₁ to C₄ alkoxy, or C₁ to C₆ alkyl group; —C(O)—Rₙ; a 5 to 6 membered heterocycle, wherein the heterocycle is optionally substituted with a —C(O)—Rₙ group; a 5 to 6 membered heteroaryl; a thiazoleamino group; a C₁ to C₈ alkyl group, wherein the alkyl group is optionally substituted with at least one independently selected halogen, a C₁ to C₄ alkoxy, a phenyloxy, a C₆ to C₁₀ aryl, —C(O)—Rₙ, —O—C(O)—Rₙ, hydroxyl, or amino group, optionally substituted with a —C(O)O—Rₙ group;

R_{d} is independently hydrogen; a C₂ to C₈ alkenyl group; a C₂ to C₈ alkynyl group; a C₆ to C₁₀ aryl group, wherein the aryl is optionally substituted with at least one independently selected halogen, nitro, C₁ to C₆ alkyl, —C(O)O—Rₑ, or —ORₑ; or a C₁ to C₈ alkyl group, wherein the alkyl group is optionally substituted with at least one independently selected halogen, C₁ to C₄ alkyl, C₁ to C₄ alkoxy, phenyloxy, C₆ to C₁₀ aryl, 5 to 6 membered heteroaryl, —C(O)—Rₙ, —O—C(O)—Rₙ, or hydroxyl group, wherein the C₆ to C₁₀ aryl group is optionally substituted with at least one independently selected halogen or haloalkyl group;

Rₑ is a hydrogen; a C₁ to C₆ alkyl group, wherein the alkyl group is optionally substituted with at least one independently selected halogen or alkoxy group; or a C₆ to C₁₀ aryl group, wherein the aryl group is optionally substituted with at least one independently selected halogen or alkoxy group;

R_{f} is a C₁ to C₆ alkyl group, optionally substituted with at least one independently selected halogen, hydroxyl, C₁ to C₄ alkoxy, cyano, C₆ to C₁₀ aryl, or —C(O)—Rₙ group, wherein the alkoxy group may be optionally substituted with at least one C₁ to C₄ alkoxy group and the aryl group may be optionally substituted with at least one independently selected halogen, hydroxyl, C₁ to C₄ alkoxy, cyano, or C₁ to C₆ alkyl group;

Rₙ is a hydroxyl, C₁ to C₄ alkoxy, amino, or C₁ to C₆ alkyl group;

R₃ is hydrogen or —C(O)—R_{g}; and,

R_{g} is a hydroxyl group; an amino group, wherein the amino is optionally substituted with a C₆ to C₁₀ cycloalkyl group or a 5 to 10 membered heteroaryl group; or a 5 to 10 membered heterocycle group, wherein the heterocycle group is optionally substituted with a —C(O)—Rₙ group,

with the proviso that, when R₃ is —C(O)—R_{g} and R_{g} is hydroxyl and R₂ and X are hydrogen, then R₁ is other than unsubstituted C₁ to C_{g} alkyl, unsubstituted phenyl or (4-methoxy)phenyl,

with the proviso that, when R₃ is —C(O)—R_{g} and R_{g} is hydroxyl and R₂ is tert-butoxycarbonyl, then R₁ is other than indole optionally substituted with C₁ to C₈ alkyl or benzyl, and

with the proviso that, when R₃ is —C(O)—R_{g} and R_{g} is amino substituted with benzothiazolyl and R₂ is hydrogen or tert-butoxycarbonyl, then R₁ is other than cyclohexyl.

Embodiments of compounds of the present invention useful for post-transcriptionally inhibiting the expression of VEGF include those of Formula (I), wherein

X is C₁ to C₆ alkyl substituted with one or more halogens; a hydroxyl group; a halogen; a C₁ to C₅ alkoxy substituted with a C₆ to C₁₀ aryl group,

A is CH or N;

B is CH or N, with the proviso that at least one of A or B is N, and that when A is N, B is CH;

R₁ is a hydroxyl group; a C₁ to C₈ alkyl group, optionally substituted with an alkylthio group, a 5 to 10 membered heteroaryl, a C₆ to C₁₀ aryl group optionally substituted with at least one independently selected Rₒ group; a C₂ to C₈ alkenyl group; a C₂ to C₈ alkynyl group; a 3 to 12 membered heterocycle group, wherein the heterocycle group is optionally substituted with at least one independently selected halogen, oxo, amino, alkylamino, acetamino, thio, or alkylthio group; a 5 to 12 membered heteroaryl group, wherein the heteroaryl group is optionally substituted with at least one independently selected halogen, oxo, amino, alkylamino, acetamino, thio, or alkylthio group; or a C₆ to C₁₀ aryl group, optionally substituted with at least one independently selected Rₒ group,

Rₒ is a halogen; a cyano; a nitro; a sulfonyl, wherein the sulfonyl is optionally substituted with a C₁ to C₆ alkyl or a 3 to 10 membered heterocycle; an amino group, wherein the amino group is optionally substituted with a C₁ to C₆ alkyl, —C(O)—R_{b}, —C(O)O—R_{b}, a sulfonyl, an alkylsulfonyl, a 3 to 10 membered heterocycle group optionally substituted with a —C(O)O—Rₙ; —C(O)—NH—R_{b}; a 5 to 6 membered heterocycle; a 5 to 6 membered heteroaryl; a C₁ to C₆ alkyl group, wherein the alkyl group is optionally substituted with at least one independently selected hydroxyl, halogen, amino, or 3 to 12 membered heterocycle group, wherein the amino group and heterocycle group are optionally substituted with at least one independently selected C₁ to C₄ alkyl group, which C₁ to C₄ alkyl group is optionally substituted with at least one independently selected C₁ to C₄ alkoxy group, amino group, alkylamino group, or 5 to 10 membered heterocycle group; a —C(O)—Rₙ group; or an —ORₐ group;

Rₐ is hydrogen; C₂ to C₈ alkenyl; a —C(O)O—R_{b} group; a —C(O)—NH—R_{b}; a C₁ to C₈ alkyl, wherein the alkyl group is optionally substituted with at least one independently selected hydroxyl, halogen, C₁ to C₄ alkoxy, amino, alkylamino, acetamide, —C(O)—R_{b}, —C(O)O—R_{b}, C₆ to C₁₀ aryl, 3 to 12 membered heterocycle, or 5 to 12 heteroaryl group, further wherein the alkylamino is optionally substituted with a hydroxyl, a C₁ to C₄ alkoxy, or a 5 to 12 membered heteroaryl optionally substituted with a C₁ to C₄ alkyl, further wherein the acetamide is optionally substituted with a C₁ to C₄ alkoxy, sulfonyl, or alkylsulfonyl, further wherein and the heterocycle group is optionally substituted with a C₁ to C₄ alkyl optionally substituted with a hydroxyl group, —C(O)—Rₙ, —C(O)O—Rₙ, or an oxo group;

R_{b} is hydroxyl; an amino; an alkylamino, wherein the alkylamino is optionally substituted with a hydroxyl, an amino, an alkylamino, a C₁ to C₄ alkoxy, a 3 to 12 membered heterocycle optionally substituted with at least one independently selected C₁ to C₆ alkyl, oxo, —C(O)O—Rₙ, or a 5 to 12 membered heteroaryl optionally substituted with a C₁ to C₄ alkyl; a C₁ to C₄ alkoxy; a C₂ to C₈ alkenyl; a C₂ to C₈ alkynyl; a C₆ to C₁₀ aryl, wherein the aryl is optionally substituted with at least one independently selected halogen or C₁ to C₄ alkoxy; a 5 to 12 membered heteroaryl; 3 to 12 membered heterocycle group, wherein the heterocycle is optionally substituted with at least one independently selected acetamide, —C(O)O—Rₙ, 5 to 6 membered heterocycle, or C₁ to C₆ alkyl optionally substituted with a hydroxyl, C₁ to C₄ alkoxy, amino group, or alkylamino group; or a C₁ to C₈ alkyl, wherein the alkyl is optionally substituted with at least one independently selected C₁ to C₄ alkoxy, C₆ to C₁₀ aryl, amino, or 3 to 12 membered heterocycle group, wherein the amino and heterocycle groups are optionally substituted with at least one independently selected C₁ to C₆ alkyl, oxo, or —C(O)O—Rₙ group;

R₂ is a hydroxyl; a 5 to 10 membered heteroaryl group; a C₁ to C₈ alkyl group, wherein the alkyl group is optionally substituted with a hydroxyl, a C₁ to C₄ alkoxy, a 3 to 10 membered heterocycle, a 5 to 10 membered heteroaryl, or C₆ to C₁₀ aryl group; a —C(O)—R_{c}, group; a —C(O)O—R_{d} group; a —C(O)—N(R_{d}R_{d}) group; a —C(S)—N(R_{d}R_{d}) group; a —C(S)—O—Rₑ, group; a —S(O₂)—Rₑ group; a —C(NRₑ)—S—Rₑ, group; or a —C(S)—S—R_{f} group,

R_{c} is hydrogen; an amino, wherein the amino is optionally substituted with at least one independently selected C₁ to C₆ alkyl or C₆ to C₁₀ aryl group; a C₆ to C₁₀ aryl, wherein the aryl is optionally substituted with at least one independently selected halogen, haloalkyl, hydroxyl, C₁ to C₄ alkoxy, or C₁ to C₆ alkyl group; —C(O)—Rₙ; a 5 to 6 membered heterocycle, wherein the heterocycle is optionally substituted with a —C(O)—Rₙ group; a 5 to 6 membered heteroaryl; a thiazoleamino group; a C₁ to C₈ alkyl group, wherein the alkyl group is optionally substituted with at least one independently selected halogen, a C₁ to C₄ alkoxy, a phenyloxy, a C₆ to C₁₀ aryl, —C(O)—Rₙ, —O—C(O)—Rₙ, hydroxyl, or amino group, optionally substituted with a —C(O)O—Rₙ group;

R_{d} is independently hydrogen; a C₂ to C₈ alkenyl group; a C₂ to C₈ alkynyl group; a C₆ to C₁₀ aryl group, wherein the aryl is optionally substituted with at least one independently selected halogen, nitro, C₁ to C₆ alkyl, —C(O)O—Rₑ, or —ORₑ; or a C₁ to C₈ alkyl group, wherein the alkyl group is optionally substituted with at least one independently selected halogen, C₁ to C₄ alkyl, C₁ to C₄ alkoxy, phenyloxy, C₆ to C₁₀ aryl, 5 to 6 membered heteroaryl, —C(O)—Rₙ, —O—C(O)—Rₙ, or hydroxyl group, wherein the C₆ to C₁₀ aryl group is optionally substituted with at least one independently selected halogen or haloalkyl group;

Rₑ is a hydrogen; a C₁ to C₆ alkyl group, wherein the alkyl group is optionally substituted with at least one independently selected halogen or alkoxy group; or a C₆ to C₁₀ aryl group, wherein the aryl group is optionally substituted with at least one independently selected halogen or alkoxy group;

R_{f} is a C₁ to C₆ alkyl group, optionally substituted with at least one independently selected halogen, hydroxyl, C₁ to C₄ alkoxy, cyano, C₆ to C₁₀ aryl, or —C(O)—Rₙ group, wherein the alkoxy group may be optionally substituted with at least one C₁ to C₄ alkoxy group and the aryl group may be optionally substituted with at least one independently selected halogen, hydroxyl, C₁ to C₄ alkoxy, cyano, or C₁ to C₆ alkyl group;

Rₙ is a hydroxyl, C₁ to C₄ alkoxy, amino, or C₁ to C₆ alkyl group;

R₃ is hydrogen or —C(O)—R_{g}; and,

R_{g} is a 5 to 10 membered heterocycle group, wherein the heterocycle group is optionally substituted with a —C(O)—Rₙ group.

Embodiments of compounds of the present invention useful for post-transcriptionally inhibiting the expression of VEGF include those of Formula (V):

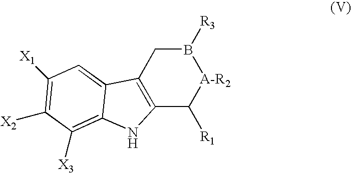

wherein,

X₁ is hydrogen; C₁ to C₆ alkyl optionally substituted with one or more halogen substituents; hydroxyl; halogen; or, C₁ to C₅ alkoxy optionally substituted with aryl;

X₂ is hydrogen or C₁ to C₆ alkoxy;

X₃ is hydrogen or C₁ to C₆ alkyl;

A is CH or N;

B is CH or N, with the proviso that at least one of A or B is N, and the other is CH;

R₁ is one substituent selected from hydroxyl; C₁ to C₈ alkyl, wherein C₁ to C₈ alkyl is optionally substituted with C₁ to C₄ alkylthio, 5 to 10 membered heteroaryl, or aryl, wherein aryl is optionally substituted with one or more substituents independently selected from Rₒ; C₂ to C₈ alkenyl; C₂ to C₈ alkynyl; C₃₋₁₄cycloalkyl; 3 to 12 membered heterocycle, wherein heterocycle is optionally substituted with one or more substituents independently selected from halogen, oxo, amino, C₁ to C₄ alkylamino, acetamino, thio, or C₁ to C₄ alkylthio; 5 to 12 membered heteroaryl, wherein heteroaryl is optionally substituted with one or more substituents independently selected from halogen, oxo, amino, C₁ to C₄ alkylamino, acetamino or C₁ to C₄ alkylthio; or aryl, wherein aryl is optionally substituted with one or more substituents independently selected from Rₒ;

Rₒ is one, two, three, four or five substituents selected from halogen; cyano; nitro; sulfonyl substituted with C₁ to C₆ alkyl or 3 to 10 membered heterocycle; amino, wherein amino is optionally mono- or disubstituted with C₁ to C₆ alkyl, —C(O)—R_{b}, —C(O)O—R_{b}, C₁ to C₄ alkylsulfonyl, or 3 to 10 membered heterocycle, wherein heterocycle is optionally substituted with oxo or —C(O)O—R_{f}; 5 to 6 membered heterocycle; 5 to 6 membered heteroaryl; C₁ to C₆ alkyl, wherein C₁ to C₆ alkyl is optionally substituted with one or more substituents independently selected from hydroxyl, halogen, amino or 3 to 12 membered heterocycle, wherein amino and heterocycle are optionally substituted with one or more substituents independently selected from C₁ to C₄ alkyl or C₁ to C₄ acetyl, wherein C₁ to C₄ alkyl is optionally substituted with one or more substituents independently selected from C₁ to C₄ alkoxy, amino, C₁ to C₄ alkylamino, or 5 to 10 membered heterocycle; —C(O)—R_{b}; —C(O)O—Rₑ; or —ORₐ;

Rₐ is hydrogen; C₂ to C₈ alkenyl; —C(O)—R_{b}; —C(O)O—R_{b}; —C(O)—NH—R_{b}; or C₁ to C₈ alkyl, wherein C₁ to C₈ alkyl is optionally substituted with one or more substituents independently selected from hydroxyl, halogen, C₁ to C₄ alkoxy, amino, C₁ to C₄ alkylamino, acetamino, —OC(O)—R_{b}, —C(O)—R_{b}, —C(O)O—R_{b}, aryl, 3 to 12 membered heterocycle, or 5 to 12 heteroaryl; further wherein C₁ to C₄ alkoxy is optionally substituted with —C(O)—R_{b}, —C(O)O—R_{b} or is optionally further substituted with C₁ to C₄ alkoxy; further wherein amino is optionally substituted with —C(O)—R_{b}, —C(O)O—R_{b}, C₁ to C₄ alkylsulfonyl or 5 to 12 membered heteroaryl, wherein heteroaryl is optionally substituted with C₁ to C₄ alkyl; further wherein C₁ to C₄ alkylamino is optionally substituted on C₁ to C₄ alkyl with hydroxyl, C₁ to C₄ alkoxy, or 5 to 12 membered heteroaryl, wherein heteroaryl is optionally substituted with C₁ to C₄ alkyl; further wherein acetamide is optionally substituted with C₁ to C₄ alkoxy or C₁ to C₄ alkylsulfonyl; further wherein aryl is optionally substituted with 5 to 12 membered heteroaryl optionally substituted with C₁ to C₄ alkyl; and, further wherein heterocycle is optionally substituted with oxo or C₁ to C₄ alkyl optionally substituted with hydroxyl, amino, C₁ to C₄ alkylamino, —C(O)—R_{f}, —C(O)O—R_{f}, or oxo;

R_{b} is hydroxyl; amino optionally substituted with 3 to 12 membered heterocycle optionally substituted with one or more substituents selected from C₁ to C₆ alkyl, C₁ to C₄ alkoxy, oxo or —C(O)O—R_{f}; C₁ to C₄ alkylamino, wherein C₁ to C₄ alkylamino is optionally substituted on C₁ to C₄ alkyl with hydroxyl, amino, C₁ to C₄ alkylamino, C₁ to C₄ alkoxy, 5 to 12 membered heteroaryl, 3 to 12 membered heterocycle optionally substituted with one or more substituents independently selected from C₁ to C₆ alkyl, C₁ to C₄ alkoxy, oxo, —C(O)O—Rₙ; or 5 to 12 membered heteroaryl optionally substituted with a C₁ to C₄ alkyl; C₂ to C₈ alkenyl; C₂ to C₈ alkynyl; aryl, wherein the aryl is optionally substituted with one or more substituents selected from halogen or C₁ to C₄ alkoxy; 5 to 12 membered heteroaryl; 3 to 12 membered heterocycle, wherein heterocycle is optionally substituted with one or more substituents independently selected from acetamino, —C(O)O—Rₙ, 5 to 6 membered heterocycle, C₃₋₁₄cycloalkyl or C₁ to C₆ alkyl, wherein C₁ to C₆ alkyl is optionally further substituted with one or more substituents independently selected from hydroxyl, C₁ to C₄ alkoxy, amino or C₁ to C₄ alkylamino; or C₁ to C₈ alkyl, wherein C₁ to C₈ alkyl is optionally substituted with one or more substituents independently selected from C₁ to C₄ alkoxy, aryl, amino, C₁ to C₄ alkylamino, —C(O)O—Rₙ, —NH—C(O)O—R_{f}, or 3 to 12 membered heterocycle, wherein heterocycle is optionally substituted with one or more substituents independently selected from C₁ to C₆ alkyl, oxo, or —C(O)O—Rₙ;

R₂ is hydrogen, hydroxyl, 5 to 10 membered heteroaryl, C₁ to C₈ alkyl, wherein C₁ to C₈ alkyl is optionally substituted with hydroxyl, C₁ to C₄ alkoxy, 3 to 10 membered heterocycle, 5 to 10 membered heteroaryl or aryl, —C(O)—R_{c}, —C(O)O—R_{d}, —C(O)—N(R_{d}R_{d}), —C(S)—N(R_{d}R_{d}), —C(S)—O—Rₑ, —SO₂—Rₑ, —C(NRₑ)—S—Rₑ, —C(S)—S—R_{f}, or —C(O)—C(O)O—R_{f};

R_{c} is hydrogen; aryl, wherein aryl is optionally substituted with one or more substituents independently selected from halogen, haloalkyl, hydroxyl, C₁ to C₄ alkoxy, C₁ to C₆ alkyl, aryl or —C(O)—Rₙ; 5 to 6 membered heterocycle, wherein heterocycle is optionally substituted with —C(O)—Rₙ; 5 to 6 membered heteroaryl; thiazole-amino; C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)—Rₙ, —C(O)O—Rₙ, —OC(O)—Rₙ, hydroxyl or amino, wherein C₁ to C₄ alkoxy is optionally further substituted with C₁ to C₄ alkoxy, and wherein amino is optionally further substituted with —C(O)O—Rₙ;

R_{d} is independently hydrogen; C₂ to C₈ alkenyl; C₂ to C₈ alkynyl; aryl, wherein aryl is optionally substituted with one or more substituents independently selected from halogen, nitro, C₁ to C₆ alkyl, haloalkyl, —C(O)O—Rₑ or —ORₑ; 5 to 6 membered heteroaryl, wherein heteroaryl is optionally substituted with C₁ to C₆ alkyl or haloalkyl; C₃₋₁₄cycloalkyl, wherein C₃₋₁₄cycloalkyl is optionally substituted with one or more substituents independently selected from halogen, C₁ to C₄ alkyl or C₁ to C₄ alkoxy; or, C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)—Rₙ, —O—C(O)—Rₙ, or hydroxyl, wherein aryl is optionally substituted with one or more substituents independently selected from halogen or haloalkyl;

Rₑ is hydrogen; C₁ to C₆ alkyl, C₃₋₁₄cycloalkyl or aryl, wherein C₁ to C₆ alkyl is optionally substituted with one or more substituents independently selected from halogen, C₁ to C₄ alkoxy or aryl, wherein each instance of aryl is optionally substituted with one or more substituents independently selected from halogen or C₁ to C₄ alkoxy;

R_{f} is C₁ to C₆ alkyl optionally substituted with one or more substituents independently selected from halogen, hydroxyl, C₁ to C₄ alkoxy, cyano, aryl or —C(O)—Rₙ, wherein C₁ to C₄ alkoxy may be optionally substituted with C₁ to C₄ alkoxy and wherein aryl may be optionally substituted with one or more substituents independently selected from halogen, hydroxyl, C₁ to C₄ alkoxy, cyano, or C₁ to C₆ alkyl;

Rₙ, is hydroxyl, C₁ to C₄ alkoxy, amino, or C₁ to C₆ alkyl optionally substituted with C₁ to C₄ alkoxy optionally further substituted with C₁ to C₄ alkoxy which is optionally further substituted with C₁ to C₄ alkoxy;

R₃ is hydrogen; C₁ to C₆ alkyl optionally substituted with hydroxy; aryl optionally substituted with C₁ to C₄ alkoxy; or —C(O)—R_{g}; and

R_{g} is hydroxyl or amino, wherein amino is optionally substituted with C₃₋₁₄cycloalkyl or 5 to 10 membered heteroaryl, wherein heteroaryl is optionally substituted with C₁ to C₄ alkyl; or 5 to 10 membered heterocycle, wherein heterocycle is optionally substituted with —C(O)—Rₙ.

Embodiments of compounds of the present invention useful for post-transcriptionally inhibiting the expression of VEGF include those of Formula (V) wherein,

X₁ is hydrogen; C₁ to C₆ alkyl optionally substituted with one or more halogen substituents; hydroxyl; halogen; or, C₁ to C₅ alkoxy optionally substituted with aryl,

with the proviso that, when X₁ is C₁ to C₅ alkoxy and R₂ is —C(O)O—R_{d}, wherein R_{d} is C₁ to C₄ alkyl, then R₁ is other than unsubstituted C₁ to C₈ alkyl;

X₂ is hydrogen or C₁ to C₆ alkoxy;

X₃ is hydrogen or C₁ to C₆ alkyl;

A is CH or N;

B is CH or N, with the proviso that at least one of A or B is N, and the other is CH;

R₁ is one substituent selected from hydroxyl; C₁ to C₈ alkyl, wherein C₁ to C₈ alkyl is optionally substituted with C₁ to C₄ alkylthio, 5 to 10 membered heteroaryl, or aryl, wherein aryl is optionally substituted with one or more substituents independently selected from Rₒ; C₂ to C₈ alkenyl; C₂ to C₈ alkynyl; C₃₋₁₄cycloalkyl; 3 to 12 membered heterocycle, wherein heterocycle is optionally substituted with one or more substituents independently selected from halogen, oxo, amino, C₁ to C₄ alkylamino, acetamino, thio, or C₁ to C₄ alkylthio; 5 to 12 membered heteroaryl, wherein heteroaryl is optionally substituted with one or more substituents independently selected from halogen, oxo, amino, C₁ to C₄ alkylamino, acetamino or C₁ to C₄ alkylthio; or aryl, wherein aryl is optionally substituted with one or more substituents independently selected from Rₒ,

with the proviso that, when R₁ is unsubstituted phenyl, then X₁ is other than hydrogen;

Rₒ is one, two, three, four or five substituents selected from halogen; cyano; nitro; sulfonyl substituted with C₁ to C₆ alkyl or 3 to 10 membered heterocycle; amino, wherein amino is optionally mono- or disubstituted with C₁ to C₆ alkyl, —C(O)—R_{b}, —C(O)O—R_{b}, C₁ to C₄ alkylsulfonyl, or 3 to 10 membered heterocycle, wherein heterocycle is optionally substituted with oxo or —C(O)O—R_{f}; 5 to 6 membered heterocycle; 5 to 6 membered heteroaryl; C₁ to C₆ alkyl, wherein C₁ to C₆ alkyl is optionally substituted with one or more substituents independently selected from hydroxyl, halogen, amino or 3 to 12 membered heterocycle, wherein amino and heterocycle are optionally substituted with one or more substituents independently selected from C₁ to C₄ alkyl or C₁ to C₄ acetyl, wherein C₁ to C₄ alkyl is optionally substituted with one or more substituents independently selected from C₁ to C₄ alkoxy, amino, C₁ to C₄ alkylamino, or 5 to 10 membered heterocycle; —C(O)—R_{b}; —C(O)O—Rₑ; or —ORₐ;

Rₐ is hydrogen; C₂ to C₈ alkenyl; —C(O)—R_{b}; —C(O)O—R_{b}; —C(O)—NH—R_{b}; or C₁ to C₈ alkyl, wherein C₁ to C₈ alkyl is optionally substituted with one or more substituents independently selected from hydroxyl, halogen, C₁ to C₄ alkoxy, amino, C₁ to C₄ alkylamino, acetamino, —OC(O)—R_{b}, —C(O)—R_{b}, —C(O)O—R_{b}, aryl, 3 to 12 membered heterocycle, or 5 to 12 heteroaryl; further wherein C₁ to C₄ alkoxy is optionally substituted with —C(O)—R_{b}, —C(O)O—R_{b} or is optionally further substituted with C₁ to C₄ alkoxy; further wherein amino is optionally substituted with —C(O)—R_{b}, —C(O)O—R_{b}, C₁ to C₄ alkylsulfonyl or 5 to 12 membered heteroaryl, wherein heteroaryl is optionally substituted with C₁ to C₄ alkyl; further wherein C₁ to C₄ alkylamino is optionally substituted on C₁ to C₄ alkyl with hydroxyl, C₁ to C₄ alkoxy, or 5 to 12 membered heteroaryl, wherein heteroaryl is optionally substituted with C₁ to C₄ alkyl; further wherein acetamide is optionally substituted with C₁ to C₄ alkoxy or C₁ to C₄ alkylsulfonyl; further wherein aryl is optionally substituted with 5 to 12 membered heteroaryl optionally substituted with C₁ to C₄ alkyl; and, further wherein heterocycle is optionally substituted with oxo or C₁ to C₄ alkyl optionally substituted with hydroxyl, amino, C₁ to C₄ alkylamino, —C(O)—R_{f}, —C(O)O—R_{f}, or oxo;

R_{b} is hydroxyl; amino optionally substituted with 3 to 12 membered heterocycle optionally substituted with one or more substituents selected from C₁ to C₆ alkyl, C₁ to C₄ alkoxy, oxo or —C(O)O—R_{f}; C₁ to C₄ alkylamino, wherein C₁ to C₄ alkylamino is optionally substituted on C₁ to C₄ alkyl with hydroxyl, amino, C₁ to C₄ alkylamino, C₁ to C₄ alkoxy, 5 to 12 membered heteroaryl, 3 to 12 membered heterocycle optionally substituted with one or more substituents independently selected from C₁ to C₆ alkyl, C₁ to C₄ alkoxy, oxo, —C(O)O—Rₙ, or 5 to 12 membered heteroaryl optionally substituted with a C₁ to C₄ alkyl; C₂ to C₈ alkenyl; C₂ to C₈ alkynyl; aryl, wherein the aryl is optionally substituted with one or more substituents selected from halogen or C₁ to C₄ alkoxy; 5 to 12 membered heteroaryl; 3 to 12 membered heterocycle, wherein heterocycle is optionally substituted with one or more substituents independently selected from acetamino, —C(O)O—Rₙ, 5 to 6 membered heterocycle, C₃₋₁₄cycloalkyl or C₁ to C₆ alkyl, wherein C₁ to C₆ alkyl is optionally further substituted with one or more substituents independently selected from hydroxyl, C₁ to C₄ alkoxy, amino or C₁ to C₄ alkylamino; or C₁ to C₈ alkyl, wherein C₁ to C₈ alkyl is optionally substituted with one or more substituents independently selected from C₁ to C₄ alkoxy, aryl, amino, C₁ to C₄ alkylamino, —C(O)O—Rₙ, —NH—C(O)O—R_{f}, or 3 to 12 membered heterocycle, wherein heterocycle is optionally substituted with one or more substituents independently selected from C₁ to C₆ alkyl, oxo, or —C(O)O—Rₙ;

R₂ is hydrogen, hydroxyl, 5 to 10 membered heteroaryl, C₁ to C₈ alkyl, wherein C₁ to C₈ alkyl is optionally substituted with hydroxyl, C₁ to C₄ alkoxy, 3 to 10 membered heterocycle, 5 to 10 membered heteroaryl or aryl, —C(O)—R_{c}, —C(O)O—R_{d}, —C(O)—N(R_{d}R_{d}), —C(S)—N(R_{d}R_{d}), —C(S)—O—Rₑ, —SO₂—Rₑ, —C(NRₑ)—S—Rₑ, —C(S)—S—R_{f}, or —C(O)—C(O)O—R_{f},

with the proviso that, when R₂, R₃, X₁, X₂ and X₃ are hydrogen, then R₁ is other than fluorenyl, substituted carbazolyl or phenyl, wherein phenyl is optionally monosubstituted with halogen, nitro or substituted amino, or di- and tri-substituted with C₁ to C₄ alkoxy;

with the proviso that, when R₂ is —C(O)—R_{c}, —C(O)O—R_{d}, —C(O)—NH(R_{d}) or —C(S)—NH(R_{d}), wherein R_{c} is C₁ to C₈ alkyl substituted with optionally substituted phenyl, wherein R_{d} is optionally substituted phenyl, cyclohexyl or C₁ to C₈ alkyl optionally substituted with optionally substituted phenyl or —C(O)O—Rₙ, and R₃, X₁, X₂ and X₃ are hydrogen, then R₁ is other than unsubstituted benzo[1,3]dioxolyl or optionally substituted phenyl, wherein phenyl is optionally disubstituted with chloro and methoxy;

R_{c} is hydrogen; aryl, wherein aryl is optionally substituted with one or more substituents independently selected from halogen, haloalkyl, hydroxyl, C₁ to C₄ alkoxy, C₁ to C₆ alkyl, aryl or —C(O)—Rₙ; 5 to 6 membered heterocycle, wherein heterocycle is optionally substituted with —C(O)—Rₙ; 5 to 6 membered heteroaryl; thiazole-amino; C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)—Rₙ, —C(O)O—Rₙ, —OC(O)—Rₙ, hydroxyl or amino, wherein C₁ to C₄ alkoxy is optionally further substituted with C₁ to C₄ alkoxy, and wherein amino is optionally further substituted with —C(O)O—Rₙ;

R_{d} is independently hydrogen; C₂ to C₈ alkenyl; C₂ to C₈ alkynyl; aryl, wherein aryl is optionally substituted with one or more substituents independently selected from halogen, nitro, C₁ to C₆ alkyl, haloalkyl, —C(O)O—Rₑ or —ORₑ; 5 to 6 membered heteroaryl, wherein heteroaryl is optionally substituted with C₁ to C₆ alkyl or haloalkyl; C₃₋₁₄cycloalkyl, wherein C₃₋₁₄cycloalkyl is optionally substituted with one or more substituents independently selected from halogen, C₁ to C₄ alkyl or C₁ to C₄ alkoxy; or, C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)—Rₙ, —O—C(O)—Rₙ, or hydroxyl, wherein aryl is optionally substituted with one or more substituents independently selected from halogen or haloalkyl;

Rₑ is hydrogen; C₁ to C₆ alkyl, C₃₋₁₄cycloalkyl or aryl, wherein C₁ to C₆ alkyl is optionally substituted with one or more substituents independently selected from halogen, C₁ to C₄ alkoxy or aryl, wherein each instance of aryl is optionally substituted with one or more substituents independently selected from halogen or C₁ to C₄ alkoxy;

R_{f} is C₁ to C₆ alkyl optionally substituted with one or more substituents independently selected from halogen, hydroxyl, C₁ to C₄ alkoxy, cyano, aryl or —C(O)—Rₙ, wherein C₁ to C₄ alkoxy may be optionally substituted with C₁ to C₄ alkoxy and wherein aryl may be optionally substituted with one or more substituents independently selected from halogen, hydroxyl, C₁ to C₄ alkoxy, cyano, or C₁ to C₆ alkyl;

Rₙ is hydroxyl, C₁ to C₄ alkoxy, amino, or C₁ to C₆ alkyl optionally substituted with C₁ to C₄ alkoxy optionally further substituted with C₁ to C₄ alkoxy which is optionally further substituted with C₁ to C₄ alkoxy;

R₃ is hydrogen; C₁ to C₆ alkyl optionally substituted with hydroxy; aryl optionally substituted with C₁ to C₄ alkoxy; or —C(O)—R_{g}; and

R_{g} is hydroxyl or amino, wherein amino is optionally substituted with C₃₋₁₄cycloalkyl or 5 to 10 membered heteroaryl, wherein heteroaryl is optionally substituted with C₁ to C₄ alkyl; or 5 to 10 membered heterocycle, wherein heterocycle is optionally substituted with —C(O)—Rₙ,

with the proviso that, when R₃ is —C(O)—R_{g} and R_{g} is hydroxyl and R₂, X₁, X₂ and X₃ are hydrogen, then R₁ is other than unsubstituted C₁ to C_{g} alkyl, unsubstituted phenyl or (4-methoxy)phenyl,

with the proviso that, when R₃ is —C(O)—R_{g} and R_{g} is hydroxyl and R₂ is tert-butoxycarbonyl, then R₁ is other than indole optionally substituted with C₁ to C₈ alkyl or benzyl, and

with the proviso that, when R₃ is —C(O)—R_{g} and R_{g} is amino substituted with benzothiazolyl and R₂ is hydrogen or tert-butoxycarbonyl, then R₁ is other than cyclohexyl.

In another embodiment, compounds of Formula (V) include compounds wherein,

X is hydrogen; C₁ to C₆ alkyl; hydroxyl; halogen; or, C₁ to C₅ alkoxy optionally substituted with aryl,

with the proviso that, when X is C₁ to C₅ alkoxy and R₂ is —C(O)O—R_{d}, wherein R_{d} is C₁ to C₄ alkyl, then R₁ is other than unsubstituted C₁ to C₈ alkyl;

R₁ is one substituent selected from hydroxyl; C₁ to C₈ alkyl, wherein C₁ to C₈ alkyl is optionally substituted with C₁ to C₄ alkylthio or aryl, wherein aryl is optionally substituted with one or more substituents independently selected from Rₒ; C₂ to C₈ alkenyl; C₃₋₁₄cycloalkyl; 3 to 12 membered heterocycle, wherein heterocycle is optionally substituted with one or more substituents independently selected from halogen or oxo; 5 to 12 membered heteroaryl, wherein heteroaryl is optionally substituted with one or more substituents independently selected from halogen, oxo, C₁ to C₄ alkylamino, acetamino or C₁ to C₄ alkylthio; or, aryl, wherein aryl is optionally substituted with one or more substituents independently selected from Rₒ,

with the proviso that, when R₁ is unsubstituted phenyl, then X is other than hydrogen;

R₁ is one, two or three substituents selected from halogen; cyano; nitro; sulfonyl substituted with C₁ to C₆ alkyl or 3 to 10 membered heterocycle; amino, wherein amino is optionally mono- or disubstituted with C₁ to C₆ alkyl, —C(O)—R_{b}, —C(O)O—R_{b} or 3 to 10 membered heterocycle, wherein heterocycle is optionally substituted with —C(O)O—R_{f}; C₁ to C₆ alkyl, wherein C₁ to C₆ alkyl is optionally substituted with one or more substituents independently selected from hydroxyl, halogen, amino or 3 to 12 membered heterocycle, wherein amino and heterocycle are optionally substituted with C₁ to C₄ alkyl, wherein C₁ to C₄ alkyl is optionally substituted with C₁ to C₄ alkoxy or 5 to 10 membered heterocycle; —C(O)—R_{b}; —C(O)O—Rₑ; or —ORₐ;

Rₐ is hydrogen; C₂ to C₈ alkenyl; —C(O)—R_{b}; —C(O)O—R_{b} or C₁ to C₈ alkyl, wherein C₁ to C₈ alkyl is optionally substituted with one or more substituents independently selected from hydroxyl, halogen, C₁ to C₄ alkoxy, amino, C₁ to C₄ alkylamino, —OC(O)—R_{b}, aryl, 3 to 12 membered heterocycle, or 5 to 12 heteroaryl; further wherein C₁ to C₄ alkoxy is optionally further substituted with C₁ to C₄ alkoxy; further wherein amino is optionally substituted with —C(O)—R_{b}, —C(O)O—R_{b}, C₁ to C₄ alkylsulfonyl or 5 to 12 membered heteroaryl, wherein heteroaryl is optionally substituted with C₁ to C₄ alkyl; further wherein C₁ to C₄ alkylamino is optionally substituted on C₁ to C₄ alkyl with hydroxyl, C₁ to C₄ alkoxy, or 5 to 12 membered heteroaryl, further wherein heterocycle is optionally substituted with oxo or C₁ to C₄ alkyl optionally substituted with hydroxyl, C₁ to C₄ alkylamino, —C(O)—R_{f} or —C(O)O—R_{f};

R_{b} is amino optionally substituted with 3 to 12 membered heterocycle, optionally substituted on heterocycle with —C(O)O—R_{f}; C₁ to C₄ alkylamino, wherein C₁ to C₄ alkylamino is optionally substituted on C₁ to C₄ alkyl with hydroxyl, C₁ to C₄ alkylamino, C₁ to C₄ alkoxy, 5 to 12 membered heteroaryl, 3 to 12 membered heterocycle optionally substituted with one or more substituents independently selected from C₁ to C₆ alkyl or oxo; C₂ to C₈ alkenyl; aryl, wherein the aryl is optionally substituted with one or more substituents selected from halogen or C₁ to C₄ alkoxy; 5 to 12 membered heteroaryl; 3 to 12 membered heterocycle, wherein heterocycle is optionally substituted with one or more substituents independently selected from acetamino, —C(O)O—Rₙ, 5 to 6 membered heterocycle, C₃₋₁₄cycloalkyl or C₁ to C₆ alkyl, wherein C₁ to C₆ alkyl is optionally further substituted with one or more substituents independently selected from hydroxyl, C₁ to C₄ alkoxy, amino or C₁ to C₄ alkylamino; or C₁ to C₈ alkyl, wherein C₁ to C₈ alkyl is optionally substituted with one or more substituents independently selected from C₁ to C₄ alkoxy, aryl, amino, C₁ to C₄ alkylamino, —C(O)O—Rₙ, —NH—C(O)O—R_{f}, or 3 to 12 membered heterocycle, wherein heterocycle is optionally substituted with one or more oxo substituents;

R₂ is hydrogen, hydroxyl, 5 to 10 membered heteroaryl, C₁ to C₈ alkyl, wherein C₁ to C₈ alkyl is optionally substituted with 3 to 10 membered heterocycle, 5 to 10 membered heteroaryl or aryl, —C(O)—R_{c}, —C(O)O—R_{d}, —C(O)—N(R_{d}R_{d}), —C(S)—N(R_{d}R_{d}), —C(S)—O—Rₑ, —SO₂—Rₑ, —C(NRₑ)—S—Rₑ, —C(S)—S—R_{f}, or —C(O)—C(O)O—R_{f},

with the proviso that, when R₂, R₃, X₁, X₂ and X₃ are hydrogen, then R₁ is other than fluorenyl, substituted carbazolyl or phenyl, wherein phenyl is optionally monosubstituted with halogen, nitro or substituted amino, or di- and tri-substituted with C₁ to C₄ alkoxy;

with the proviso that, when R₂ is —C(O)—R_{c}, —C(O)O—R_{d}, —C(O)—NH(R_{d}) or —C(S)—NH(R_{d}), wherein R_{c} is C₁ to C₈ alkyl substituted with optionally substituted phenyl, wherein R_{d} is optionally substituted phenyl, cyclohexyl or C₁ to C₈ alkyl optionally substituted with optionally substituted phenyl or —C(O)O—Rₙ, and R₃, X₁, X₂ and X₃ are hydrogen, then R₁ is other than unsubstituted benzo[1,3]dioxolyl or optionally substituted phenyl, wherein phenyl is optionally disubstituted with chloro and methoxy;

R_{c} is aryl, wherein aryl is optionally substituted with one or more substituents independently selected from halogen or aryl; 5 to 6 membered heterocycle, wherein heterocycle is optionally substituted with —C(O)—Rₙ; 5 to 6 membered heteroaryl; C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)O—Rₙ, —OC(O)—Rₙ or amino, wherein C₁ to C₄ alkoxy is optionally further substituted with C₁ to C₄ alkoxy, and wherein amino is optionally further substituted with —C(O)O—Rₙ;

R_{d} is independently hydrogen; C₂ to C₈ alkenyl; C₂ to C₈ alkynyl; aryl, wherein aryl is optionally substituted with one or more substituents independently selected from halogen, nitro, C₁ to C₆ alkyl, haloalkyl, —C(O)O—Rₑ, or —ORₑ; 5 to 6 membered heteroaryl, wherein heteroaryl is optionally substituted with C₁ to C₆ alkyl; C₃₋₁₄cycloalkyl, wherein C₃₋₁₄cycloalkyl is optionally substituted with one or more C₁ to C₄ alkyl substituents; or, C₁ to C₈ alkyl, wherein C₁ to C₈ alkyl is optionally substituted with one or more substituents independently selected from halogen, C₁ to C₄ alkoxy, aryl or 5 to 6 membered heteroaryl;

Rₑ is hydrogen; C₁ to C₆ alkyl, C₃₋₁₄cycloalkyl or aryl, wherein C₁ to C₆ alkyl is optionally substituted with aryl, wherein each instance of aryl is optionally substituted with one or more halogen substituents;

R_{f} is C₁ to C₆ alkyl optionally substituted with one or more substituents independently selected from halogen, hydroxyl, C₁ to C₄ alkoxy, cyano, aryl or —C(O)—Rₙ, wherein C₁ to C₄ alkoxy may be optionally substituted with C₁ to C₄ alkoxy and wherein aryl may be optionally substituted with one or more substituents independently selected from halogen, cyano, or C₁ to C₆ alkyl;

Rₙ is C₁ to C₄ alkoxy, amino, or C₁ to C₆ alkyl;

R₃ is hydrogen; C₁ to C₆ alkyl optionally substituted with hydroxy; aryl optionally substituted with C₁ to C₄ alkoxy; or —C(O)—R_{g}; and

R_{g} is hydroxyl or amino, wherein amino is optionally substituted with C₃₋₁₄cycloalkyl or 5 to 10 membered heteroaryl, wherein heteroaryl is optionally substituted with C₁ to C₄ alkyl; or 5 to 10 membered heterocycle, wherein heterocycle is optionally substituted with —C(O)—Rₙ,

with the proviso that, when R₃ is —C(O)—R_{g} and R_{g} is hydroxyl and R₂, X₁, X₂ and X₃ are hydrogen, then R₁ is other than unsubstituted C₁ to C₈ alkyl, unsubstituted phenyl or (4-methoxy)phenyl,

with the proviso that, when R₃ is —C(O)—R_{g} and R_{g} is hydroxyl and R₂ is tert-butoxycarbonyl, then R₁ is other than indole optionally substituted with C₁ to C₈ alkyl or benzyl, and

with the proviso that, when R₃ is —C(O)—R_{g} and R_{g} is amino substituted with benzothiazolyl and R₂ is hydrogen or tert-butoxycarbonyl, then R₁ is other than cyclohexyl.

In another embodiment, compounds of Formula (V) include compounds wherein,

X is hydrogen; C₁ to C₆ alkyl; hydroxyl; halogen; or, C₁ to C₅ alkoxy optionally substituted with phenyl,

with the proviso that, when X is C₁ to C₅ alkoxy and R₂ is —C(O)O—R_{d}, wherein R_{d} is C₁ to C₄ alkyl, then R₁ is other than unsubstituted C₁ to C₈ alkyl;

R₁ is one substituent selected from hydroxyl; C₁ to C₈ alkyl, wherein C₁ to C₈ alkyl is optionally substituted with C₁ to C₄ alkylthio or aryl, wherein aryl is optionally substituted with one or more substituents independently selected from Rₒ; C₂ to C₈ alkenyl; cyclohex-3-enyl; benzo[1,3]dioxolyl optionally substituted with halogen; 4H-chromenyl optionally substituted with oxo; dihydro-benzofuranyl, tetrahydrofuranyl, furanyl, thiazolyl, pyrimidinyl, indolyl, wherein each of furanyl, thiazolyl, pyrimidinyl and indolyl are optionally substituted with one or more substituents independently selected from halogen, oxo, C₁ to C₄ alkylamino, acetamino or C₁ to C₄ alkylthio; or, phenyl, wherein phenyl is optionally substituted with one or more substituents independently selected from Rₒ,

with the proviso that, when R₁ is unsubstituted phenyl, then X is other than hydrogen;

Rₒ is one, two or three substituents selected from halogen; cyano; nitro; sulfonyl substituted with C₁ to C₆ alkyl or morpholinyl; amino, wherein amino is optionally mono- or disubstituted with C₁ to C₆ alkyl, —C(O)—R_{b}, —C(O)O—R_{b}, piperidinyl or tetrahydro-2H-pyranyl, wherein piperidinyl is optionally substituted with —C(O)O—R_{f}; C₁ to C₆ alkyl, wherein C₁ to C₆ alkyl is optionally substituted with one or more substituents independently selected from hydroxyl, halogen, amino or piperazinyl, wherein amino and piperazinyl are optionally substituted with C₁ to C₄ alkyl, wherein C₁ to C₄ alkyl is optionally substituted with C₁ to C₄ alkoxy or morpholinyl; —C(O)—R_{b}; —C(O)O—Rₑ; or —ORₐ;

Rₐ is hydrogen; C₂ to C₈ alkenyl; —C(O)—R_{b}; —C(O)O—R_{b} or C₁ to C₈ alkyl, wherein C₁ to C₈ alkyl is optionally substituted with one or more substituents independently selected from hydroxyl, halogen, C₁ to C₄ alkoxy, amino, C₁ to C₄ alkylamino, —OC(O)—R_{b}, phenyl, oxiranyl, pyrrolidinyl, morpholinyl, thiomorpholinyl, piperidinyl, piperazinyl, dioxolidinyl, imidazolyl, pyrazolyl or triazolyl; further wherein C₁ to C₄ alkoxy is optionally further substituted with C₁ to C₄ alkoxy; further wherein amino is optionally substituted with —C(O)—R_{b}, —C(O)O—R_{b}, C₁ to C₄ alkylsulfonyl, thiazolyl or pyridinyl, wherein thiazolyl is optionally substituted with C₁ to C₄ alkyl; further wherein C₁ to C₄ alkylamino is optionally substituted on C₁ to C₄ alkyl with hydroxyl, C₁ to C₄ alkoxy or imidazolyl, wherein imidazolyl is optionally substituted with C₁ to C₄ alkyl; wherein dioxolidinyl is optionally substituted with oxo; and, wherein each of pyrrolidinyl, piperidinyl and piperazinyl are optionally substituted with C₁ to C₄ alkyl, wherein C₁ to C₄ alkyl is optionally substituted with hydroxyl, C₁ to C₄ alkylamino, —C(O)—R_{f} or —C(O)O—R_{f};

R_{b} is amino optionally substituted with piperidinyl, wherein piperidinyl is optionally substituted with —C(O)O—R_{f}; C₁ to C₄ alkylamino, wherein C₁ to C₄ alkylamino is optionally substituted on C₁ to C₄ alkyl with hydroxyl, C₁ to C₄ alkylamino, C₁ to C₄ alkoxy, imidazolyl; pyridinyl, tetrahydrofuranyl, pyrrolidinyl, dioxolidinyl or morpholinyl, wherein each of pyrrolidinyl and dioxolidinyl are optionally substituted with one or more substituents independently selected from C₁ to C₆ alkyl or oxo; C₂ to C₈ alkenyl; phenyl, wherein phenyl is optionally substituted with one or more halogen substituents; furanyl, pyrrolidinyl, piperidinyl, piperazinyl, oxazolidinyl, 1,4-diazepanyl, wherein each of pyrrolidinyl, piperidinyl, piperazinyl and 1,4-diazepanyl are optionally substituted with one or more substituents independently selected from acetamino, —C(O)O—Rₙ, pyrrolidinyl, piperidinyl, cyclohexyl or C₁ to C₆ alkyl, wherein C₁ to C₆ alkyl is optionally further substituted with one or more substituents independently selected from hydroxyl, C₁ to C₄ alkoxy, amino or C₁ to C₄ alkylamino; or C₁ to C₈ alkyl, wherein C₁ to C₈ alkyl is optionally substituted with one or more substituents independently selected from C₁ to C₄ alkoxy, aryl, amino, C₁ to C₄ alkylamino, —C(O)O—Rₙ, —NH—C(O)O—R_{f}, morpholinyl or hexahydro-1H-thieno[3,4-d]imidazolyl substituted on the imidazolyl portion with oxo;

R₂ is hydrogen, hydroxyl, pyrazinyl, pyrimidinyl, C₁ to C₈ alkyl, wherein C₁ to C₈ alkyl is optionally substituted with 1,3-dioxanyl, furanyl or phenyl, —C(O)—R_{c}, —C(O)O—R_{d}, —C(O)—N(R_{d}R_{d}), —C(S)—N(R_{d}R_{d}), —C(S)—O—Rₑ, —SO₂—Rₑ, —C(NRₑ)—S—Rₑ, —C(S)—S—R_{f}, or —C(O)—C(O)O—R_{f},

with the proviso that, when R₂, R₃ and X are hydrogen, then R₁ is other than fluorenyl, substituted carbazolyl or phenyl, wherein phenyl is optionally monosubstituted with halogen, nitro or substituted amino, or di- and tri-substituted with C₁ to C₄ alkoxy;

with the proviso that, when R₂ is —C(O)—R_{c}, —C(O)O—R_{d}, —C(O)—NH(R_{d}) or —C(S)—NH(R_{d}), wherein R_{c} is C₁ to C₈ alkyl substituted with optionally substituted phenyl, wherein R_{d} is optionally substituted phenyl, cyclohexyl or C₁ to C₈ alkyl optionally substituted with optionally substituted phenyl or —C(O)O—Rₙ, and R₃ and X are hydrogen, then R₁ is other than unsubstituted benzo[1,3]dioxolyl or optionally substituted phenyl, wherein phenyl is optionally disubstituted with chloro and methoxy;

R_{c} is phenyl, wherein phenyl is optionally substituted with one or more substituents independently selected from halogen or phenyl; morpholinyl, pyrrolidinyl or piperazinyl, wherein each of pyrrolidinyl and piperazinyl are optionally substituted with —C(O)—Rₙ; 5 to 6 membered heteroaryl; C₁ to C₈ alkyl, wherein C₁ to C₈ alkyl is optionally substituted with one or more substituents independently selected from halogen, C₁ to C₄ alkoxy, phenyloxy, phenyl, thienyl, —C(O)O—Rₙ, —OC(O)—Rₙ or amino, wherein C₁ to C₄ alkoxy is optionally further substituted with C₁ to C₄ alkoxy, and wherein amino is optionally further substituted with —C(O)O—Rₙ;

R_{d} is independently hydrogen; C₂ to C₈ alkenyl; C₂ to C₈ alkynyl; phenyl, wherein phenyl is optionally substituted with one or more substituents independently selected from halogen, nitro, C₁ to C₆ alkyl, haloalkyl, —C(O)O—Rₑ or —ORₑ; imidazolyl or thiazolyl, wherein thiazolyl is optionally substituted with C₁ to C₆ alkyl; cyclohexyl, wherein cyclohexyl is optionally substituted with one or more C₁ to C₄ alkyl substituents; or, C₁ to C₈ alkyl, wherein C₁ to C₈ alkyl is optionally substituted with one or more substituents independently selected from halogen, C₁ to C₄ alkoxy, phenyl or imidazolyl;

Rₑ is hydrogen; C₁ to C₆ alkyl, cyclohexyl or phenyl, wherein C₁ to C₆ alkyl is optionally substituted with phenyl, wherein each instance of phenyl is optionally substituted with one or more halogen substituents;

R_{f} is C₁ to C₆ alkyl optionally substituted with one or more substituents independently selected from halogen, hydroxyl, C₁ to C₄ alkoxy, cyano, phenyl or —C(O)—Rₙ, wherein C₁ to C₄ alkoxy may be optionally substituted with C₁ to C₄ alkoxy and wherein phenyl may be optionally substituted with one or more substituents independently selected from halogen, cyano, or C₁ to C₆ alkyl;

R₃ is hydrogen; C₁ to C₆ alkyl optionally substituted with hydroxy; phenyl optionally substituted with C₁ to C₄ alkoxy; or —C(O)—R_{g}; and

R_{g} is hydroxyl or amino, wherein amino is optionally substituted with cyclohexyl or thiazolyl, wherein thiazolyl is optionally substituted with C₁ to C₄ alkyl; or piperazinyl, wherein piperazinyl is optionally substituted with —C(O)—Rₙ,

with the proviso that, when R₃ is —C(O)—R_{g} and R_{g} is hydroxyl and R₂ and X are hydrogen, then R₁ is other than unsubstituted C₁ to C₈ alkyl, unsubstituted phenyl or (4-methoxy)phenyl,

with the proviso that, when R₃ is —C(O)—R_{g} and R_{g} is hydroxyl and R₂ is tert-butoxycarbonyl, then R₁ is other than indole optionally substituted with C₁ to C₈ alkyl or benzyl, and

with the proviso that, when R₃ is —C(O)—R_{g} and R_{g} is amino substituted with benzothiazolyl and R₂ is hydrogen or tert-butoxycarbonyl, then R₁ is other than cyclohexyl; and, all other variables are as previously described.

As will be evident to one skilled in the art, the compounds of Formula (V) may exist as a racemic mixture or as substantially pure enantiomers.

As used herein, the term “alkyl” generally refers to saturated hydrocarbyl radicals of straight or branched chain configuration. “C₁₋₈alkyl” refers to an “alkyl” radical having from one to eight carbon atoms, including but not limited to, including methyl, ethyl, n-propyl, isopropyl, n-butyl, isobutyl, sec-butyl, tert-butyl, n-pentyl, n-hexyl, n-heptyl, octyl, n-octyl, and the like. In some embodiments, C₁₋₈alkyl includes C₁₋₆alkyl, C₁₋₄alkyl and the like. A C₁₋₈alkyl radical may be optionally substituted where allowed by available valences.

As used herein, the term “alkenyl” generally refers to partially unsaturated hydrocarbon radicals having a straight or branched chain configuration and one or more carbon-carbon double bonds therein. As used herein, the term “C₂₋₈alkenyl” refers to an “alkenyl” radical having from two to eight carbon atoms, including ethenyl, allyl, propenyl and the like. In some embodiments, C₂₋₈alkenyl includes C₂₋₆alkenyl, C₂₋₄alkenyl and the like. A C₂₋₈alkenyl radical may be optionally substituted where allowed by available valences.

As used herein, the term “alkynyl” refers to partially unsaturated hydrocarbon radicals having one or more carbon-carbon triple bonds therein. As used herein, the term “C₂₋₈alkynyl” generally refers to an “alkynyl” radicals having from two to eight carbon atoms, including acetylenyl, propynyl, butynyl and the like. In some embodiments, C₂₋₈alkynyl includes C₂₋₆alkynyl, C₂₋₄alkynyl and the like. A C₂₋₈alkynyl radical may be optionally substituted where allowed by available valences.

As used herein, the term “alkoxy” generally refers to saturated hydrocarbon radicals having a straight or branched chain configuration of the formula: —O-alkyl. “C₁₋₈alkoxy” generally refers to an “alkoxy” radical having from one to eight carbon atoms of the formula: —O—C₁₋₈alkyl, including methoxy, ethoxy, n-propoxy, isopropoxy, n-butoxy, isobutoxy, sec-butoxy, tert-butoxy, n-pentoxy, n-hexoxy and the like. In some embodiments, C₁₋₈alkoxy includes C₁₋₆alkoxy, C₁₋₅alkoxy, C₁₋₄alkoxy and the like. A C₁₋₈alkoxy radical may be optionally substituted where allowed by available valences.

As used herein, the term “cycloalkyl” generally refers to a saturated or partially unsaturated monocyclic, bicyclic or polycyclic hydrocarbon radical. “C₃₋₁₄cycloalkyl” generally refers to a “cycloalkyl” radical having from 3 to 14 carbon atoms, including cyclopropyl, cyclobutyl, cyclopentyl cyclohexyl, cyclohexenyl, cycloheptyl, cyclooctyl, 1H-indanyl, indenyl, tetrahydro-naphthalenyl and the like. In some embodiments, C₃₋₁₄cycloalkyl includes C₃₋₈cycloalkyl, C₅₋₈cycloalkyl, C₃₋₁₀cycloalkyl, C₆₋₁₀cycloalkyl and the like. A C₃₋₁₄cycloalkyl radical may be optionally substituted where allowed by available valences.

As used herein, the term “aryl” generally refers to a monocyclic, bicyclic or polycyclic aromatic carbon atom ring structure radical, including phenyl, naphthyl, anthracenyl, fluorenyl, azulenyl, phenanthrenyl and the like. In some embodiments, aryl may be C₆₋₁₀aryl, C₆aryl (e.g., phenyl), or C₆₋₁₀aryl (e.g., napthyl). An aryl radical may be optionally substituted where allowed by available valences.

As used herein, the term “heteroaryl” generally refers to a monocyclic, bicyclic or polycyclic aromatic carbon atom ring structure radical in which one or more carbon atom ring members have been replaced, where allowed by structural stability, with one or more heteroatoms, such as an O, S or N atom, including furanyl, thienyl (or thiophenyl), 2H-pyrrolyl, 3H-pyrrolyl, pyrazolyl, imidazolyl, isoxazolyl, isothiazolyl, oxazolyl, thiazolyl, triazolyl (and regioisomers thereof), oxadiazolyl (and regioisomers thereof), thiadiazolyl (and regioisomers thereof), tetrazolyl (and regioisomers thereof), pyranyl, thiopyranyl, pyridinyl, pyrimidinyl, pyrazinyl, pyridazinyl, triazinyl (and regioisomers thereof), indole, indazolyl, isoindolyl, benzofuranyl, benzothienyl, benzimidazolyl, benzoxazolyl, purinyl, quinolinyl, isoquinolinyl, quinazolinyl, quinoxalinyl, 1,3-diazinyl, 1,2-diazinyl, 1,2-diazolyl, 1,4-diazanaphthalenyl, acridinyl and the like. In some embodiments, heteroaryl may be a monocyclic, bicyclic or polycyclic ring structure having 5 to 6, 5 to 10, or 5 to 12, atoms (members) in the ring structure. A heteroaryl radical may be optionally substituted where allowed by available valences.

As used herein, the term “heterocycle” generally refers to a saturated or partially unsaturated monocyclic, bicyclic or polycyclic carbon atom ring structure radical in which one or more carbon atom ring members have been replaced, where allowed by structural stability, with a heteroatom, such as an O, S or N atom, including oxiranyl, oxetanyl, azetidinyl, dihydrofuranyl, tetrahydrofuranyl, dioxolidinyl, dihydrothienyl, tetrahydrothienyl, pyrrolinyl, pyrrolidinyl, dihydropyrazolyl, pyrazolinyl, pyrazolidinyl, dihydroimidazolyl, imidazolinyl, imidazolidinyl, isoxazolinyl, isoxazolidinyl, isothiazolinyl, isothiazolidinyl, oxazolinyl, oxazolidinyl, thiazolinyl, thiazolidinyl, triazolinyl (and regioisomers thereof), triazolidinyl (and regioisomers thereof), oxadiazolinyl (and regioisomers thereof), oxadiazolidinyl (and regioisomers thereof), thiadiazolinyl (and regioisomers thereof), thiadiazolidinyl (and regioisomers thereof), tetrazolinyl (and regioisomers thereof), tetrazolidinyl (and regioisomers thereof), dihydro-2H-pyranyl, tetrahydro-2H-pyranyl, 1,3-dioxanyl, 1,4-dioxanyl, tetrahydro-thiopyranyl, dihydro-pyridinyl, tetrahydro-pyridinyl, hexahydro-pyridinyl, dihydro-pyrimidinyl, tetrahydro-pyrimidinyl, dihydro-pyrazinyl, tetrahydro-pyrazinyl, dihydro-pyridazinyl, tetrahydro-pyridazinyl, piperazinyl, piperidinyl, morpholinyl, thiomorpholinyl, dihydro-triazinyl (and regioisomers thereof), tetrahydro-triazinyl (and regioisomers thereof), hexahydro-triazinyl (and regioisomers thereof), 1,4-diazepanyl, hexahydro-1H-thieno[3,4-d]imidazolyl, dihydro-indole, tetrahydro-indole, dihydro-indazolyl, tetrahydro-indazolyl, dihydro-isoindolyl, tetrahydro-isoindolyl, dihydro-benzofuranyl, tetrahydro-benzofuranyl, dihydro-benzothienyl, tetrahydro-benzothienyl, dihydro-benzimidazolyl, tetrahydro-benzimidazolyl, dihydro-benzoxazolyl, tetrahydro-benzoxazolyl, chromanyl, 2H-chromenyl, 4H-chromenyl, benzo[1,3]dioxolyl, benzo[1,4]dioxanyl, dihydro-purinyl, tetrahydro-purinyl, dihydro-quinolinyl, tetrahydro-quinolinyl, dihydro-isoquinolinyl, tetrahydro-isoquinolinyl, dihydro-quinazolinyl, tetrahydro-quinazolinyl, dihydro-quinoxalinyl, tetrahydro-quinoxalinyl and the like. In some embodiments, heterocycle may be a monocyclic, bicyclic or polycyclic ring structure having 3 to 12, 3 to 10, or 5 to 6, atoms (members) in the ring structure. A heterocycle radical may be optionally substituted where allowed by available valences.

As used herein, the term “halo” or “halogen” generally refers to a halogen atom radical selected from fluoro, chloro, bromo or iodo.

As used herein, the term “haloalkyl” generally refers to a C₁ to C₈ alkyl radical substituted where allowed by available valences with one or more halogen atoms selected from fluoro, chloro, bromo or iodo.

As used herein, the term “acetamino” generally refers to a methyl substituted amido radical of the formula: —NH—C(O)—CH₃.

As used herein, the term “acetyl” generally refers to a methyl substituted carbonyl radical of the formula: —C(O)—CH₃.

As used herein, the term “C₁ to C₄ alkylamino” or “alkylamino” generally refers to a mono- or dialkyl substituted amino radical having from one to four carbon atoms of the formula: —NH—C₁ to C₄ alkyl or —N(C₁ to C₄ alkyl)₂.

As used herein, the term “C₁ to C₄ alkylthio” or “alkylthio” generally refers to a C₁ to C₄ alkyl substituted sulfur atom radical of the formula: —S—C₁ to C₄ alkyl. As used herein, the term “C₁ to C₄ alkylsulfonyl” generally refers to a C₁ to C₄ alkyl substituted sulfonyl atom radical of the formula: —SO₂—C₁ to C₄ alkyl.

As used herein, the term “amino” generally refers to an amino radical of the formula: —NH₂.

Preferred R₁ substituents also include the following, where the * indicates the bond of attachment to the carboline scaffold molecule of Formula (V).

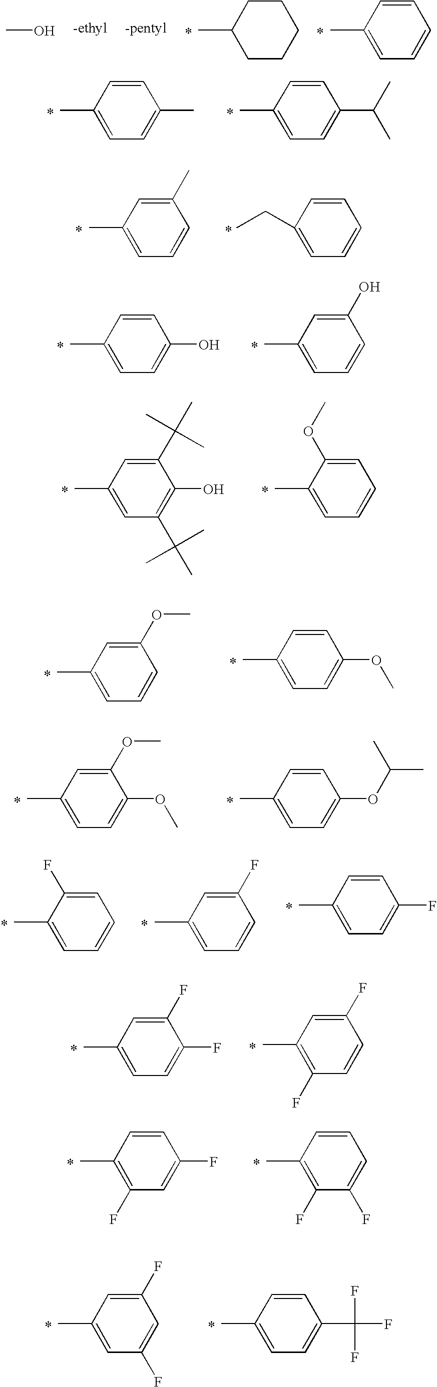

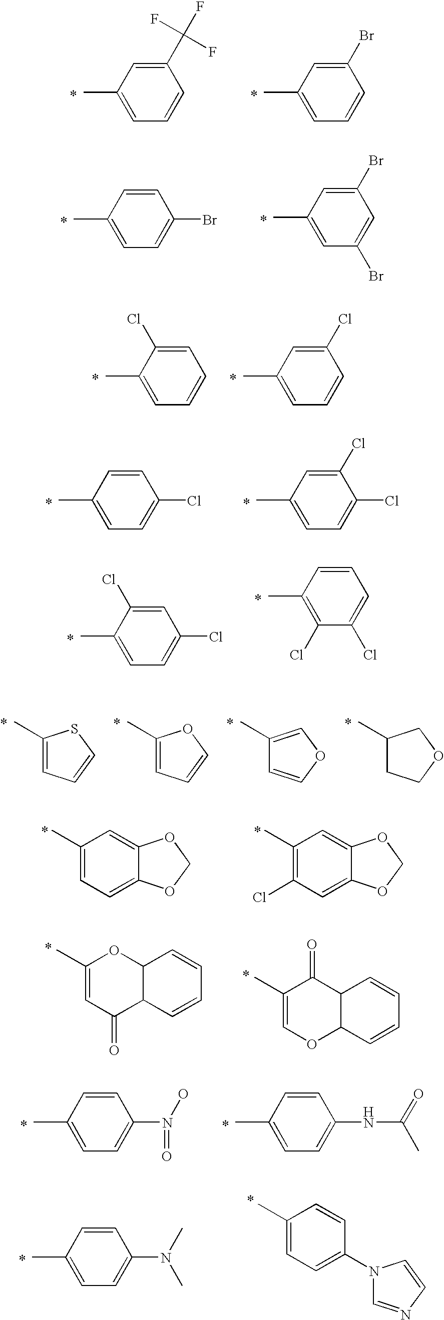

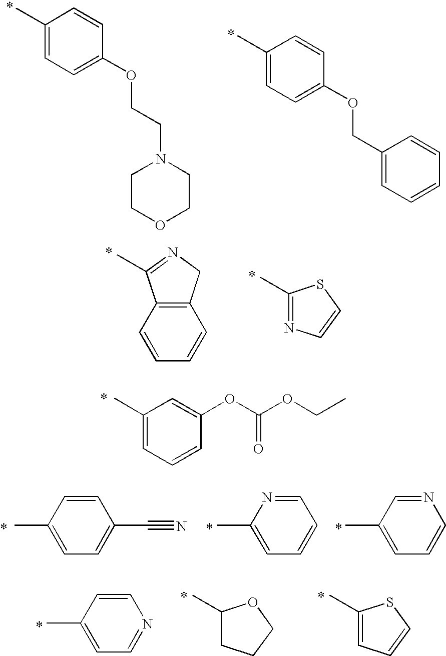

Other preferred R₁ substituents include the following, where the * indicates the bond of attachment to the carboline scaffold molecule.

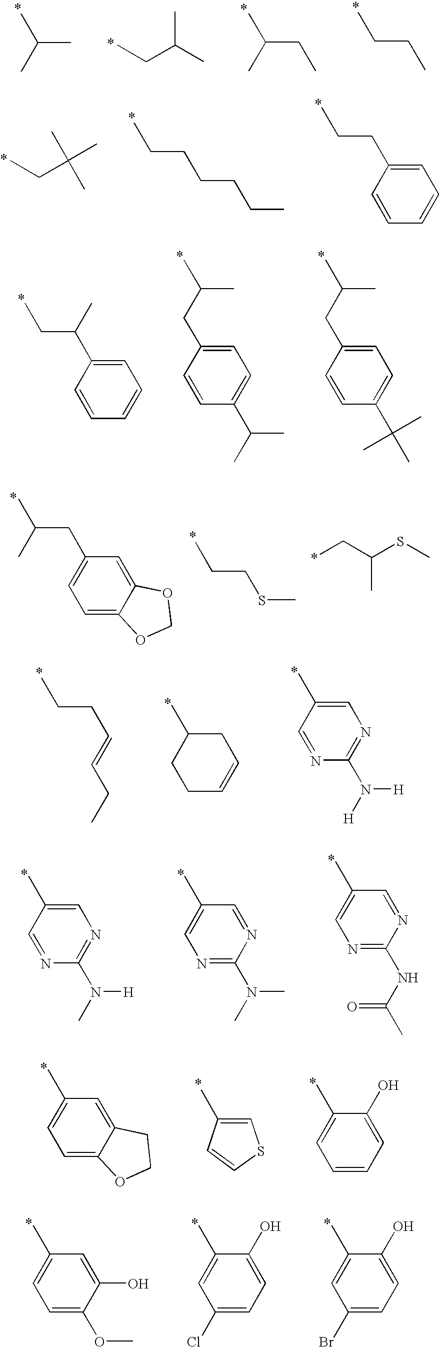

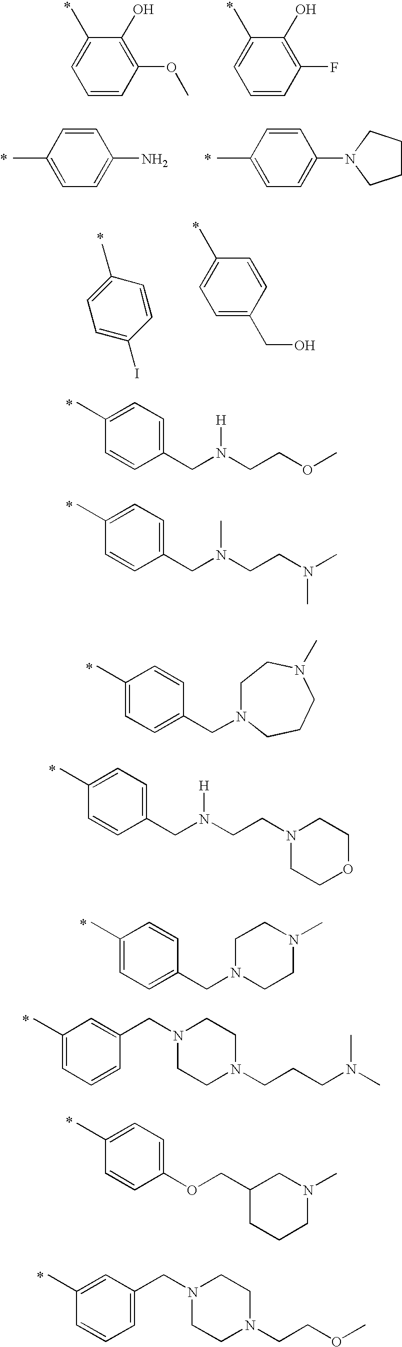

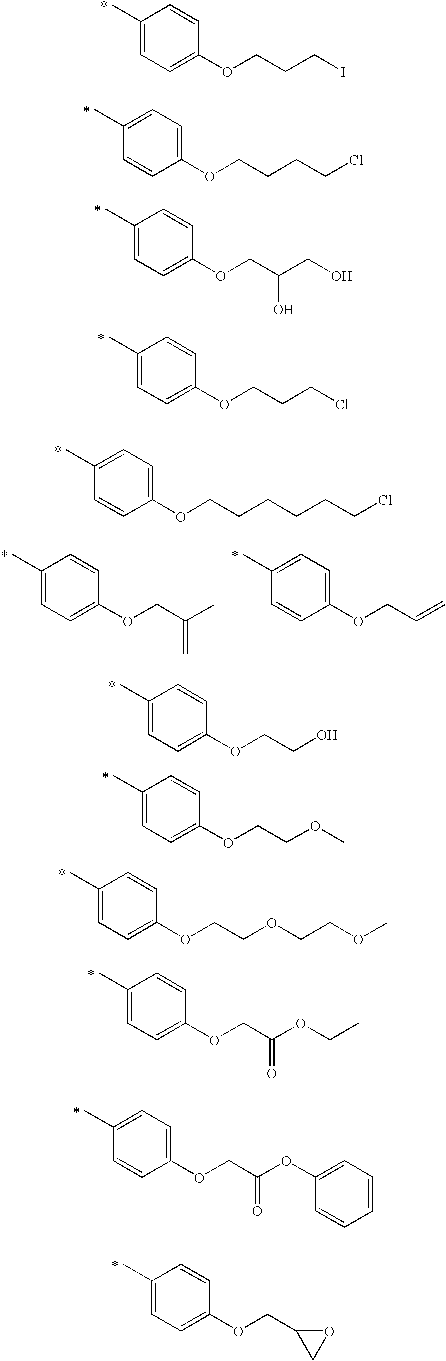

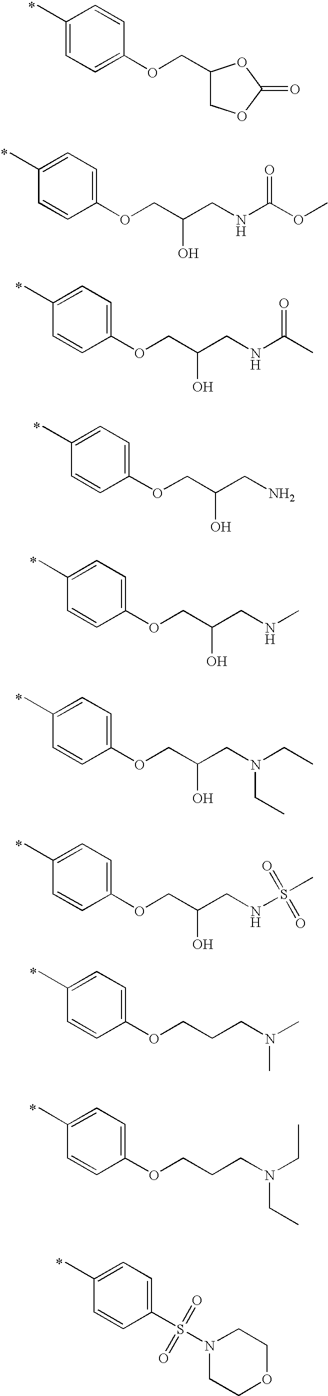

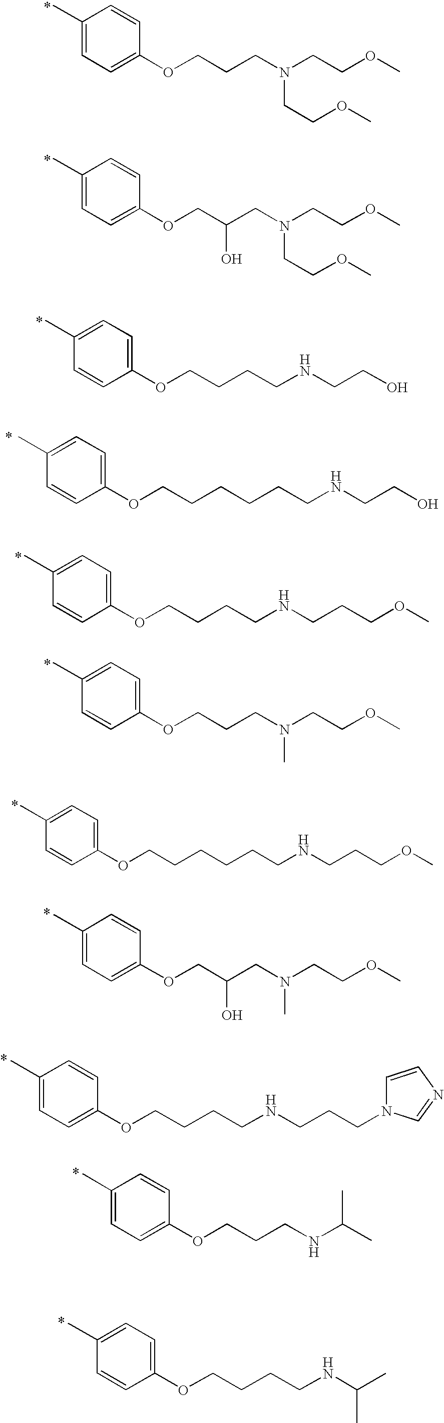

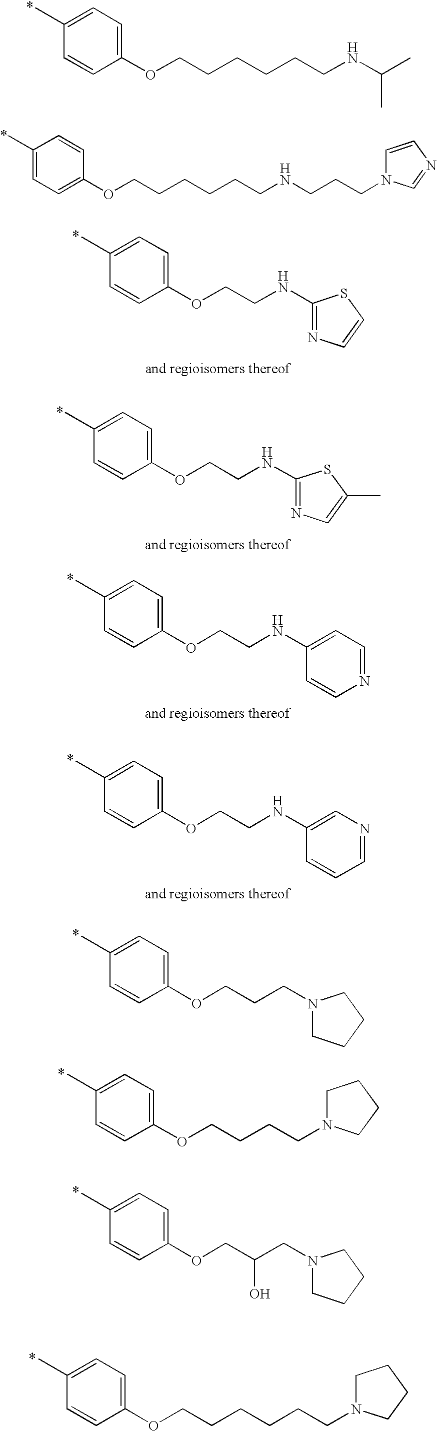

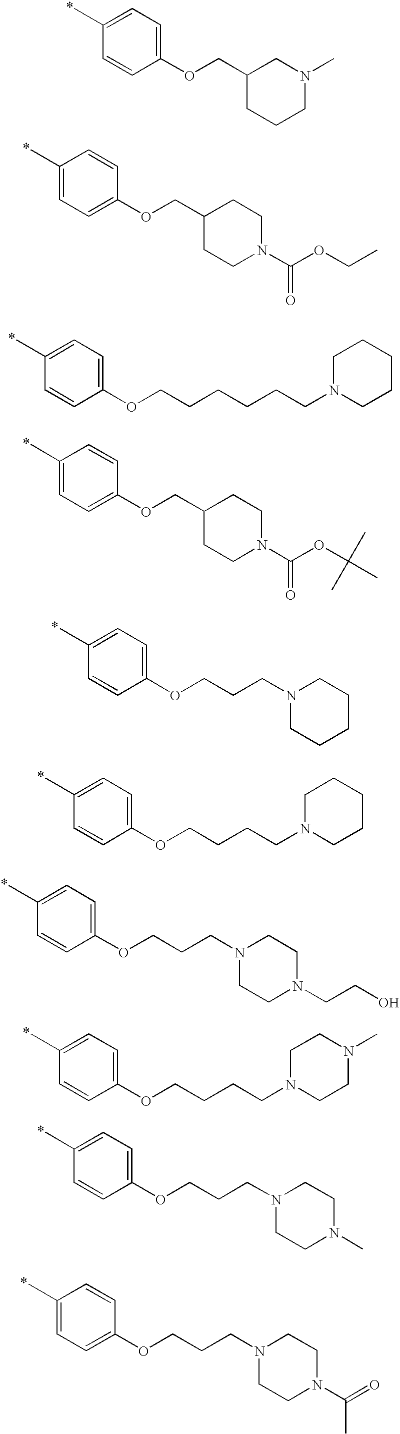

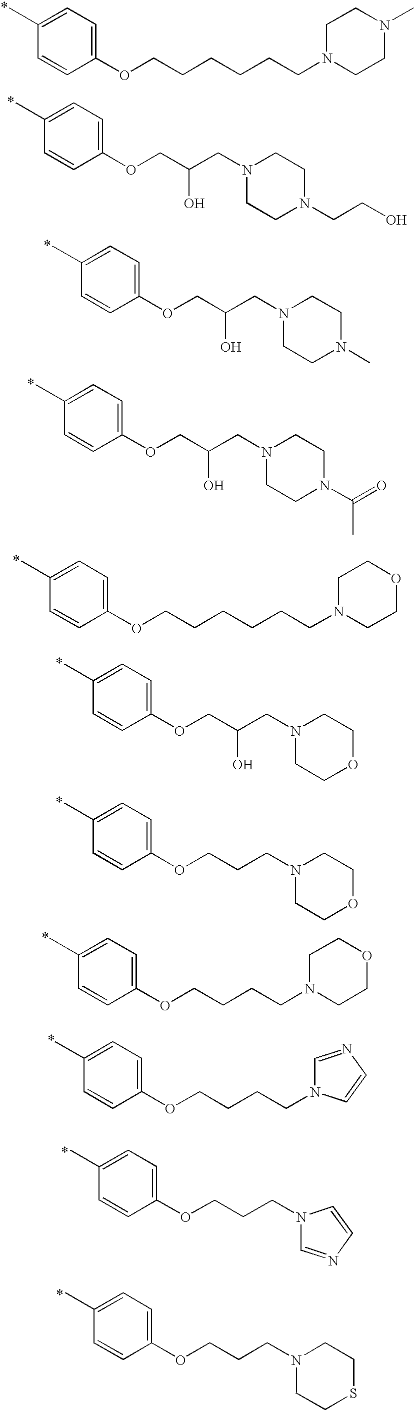

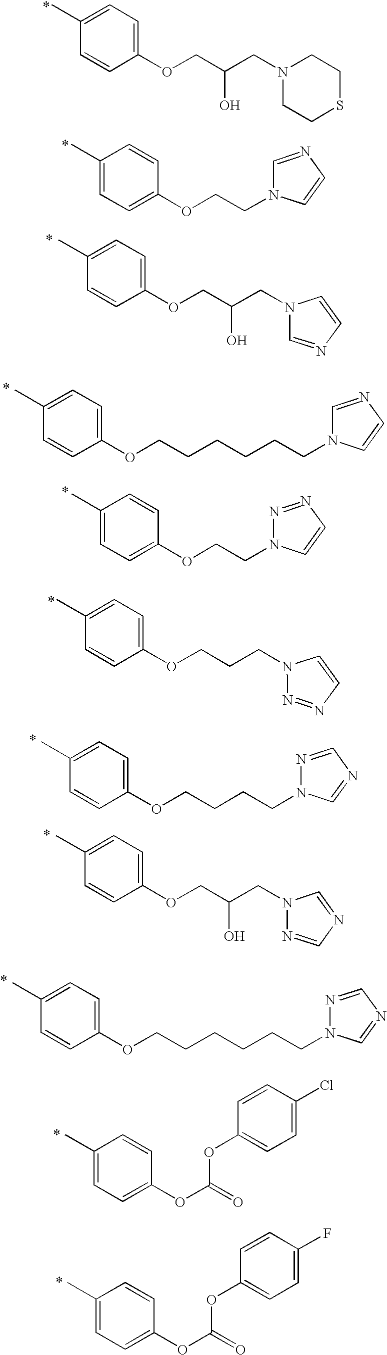

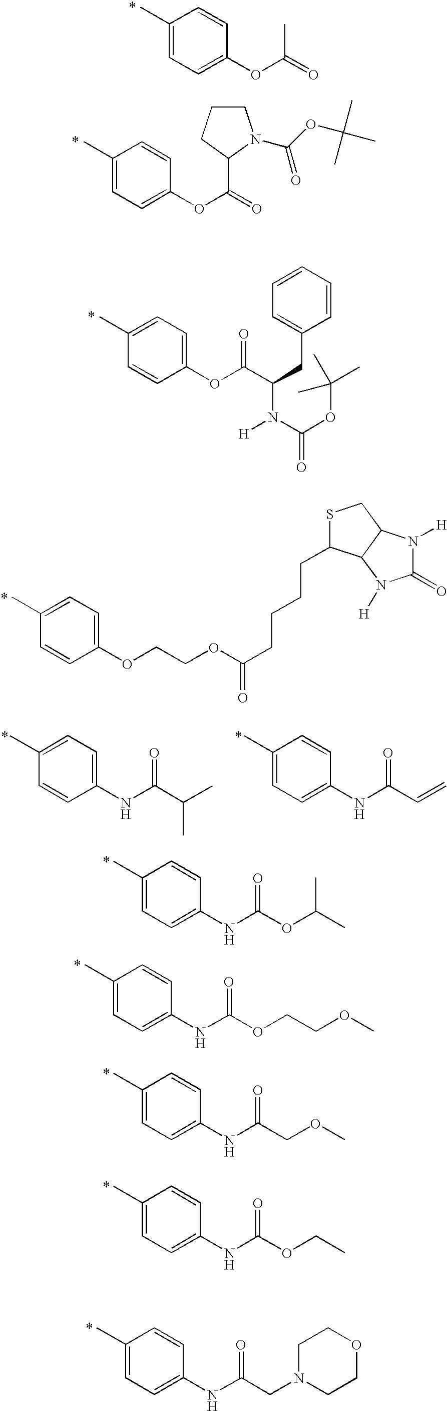

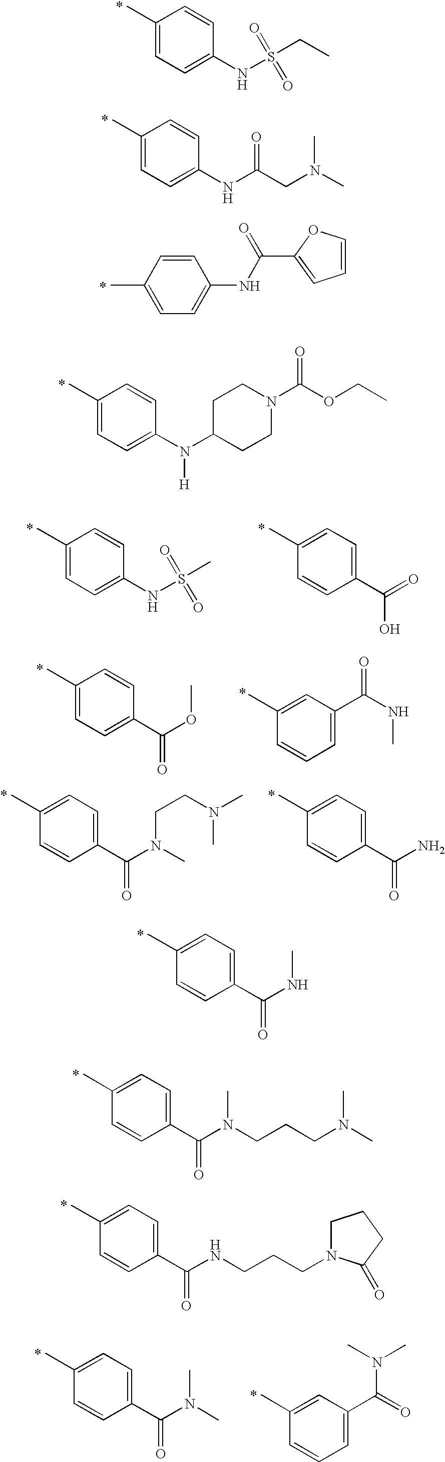

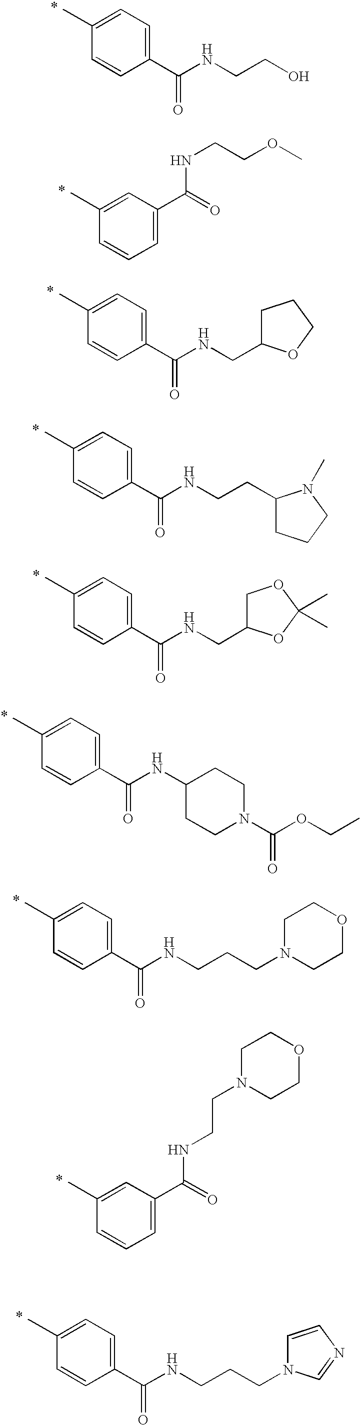

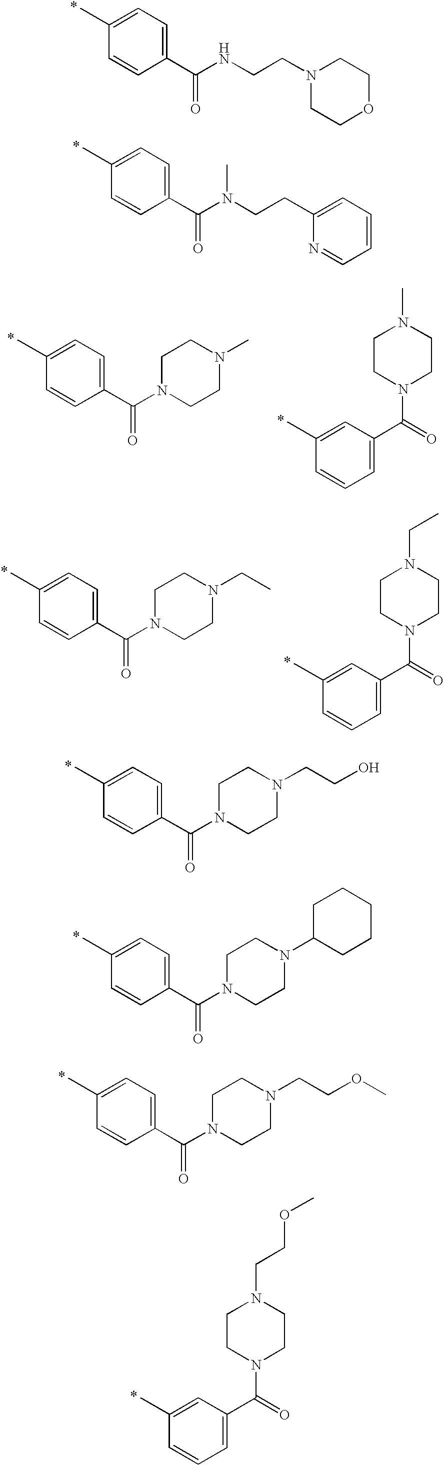

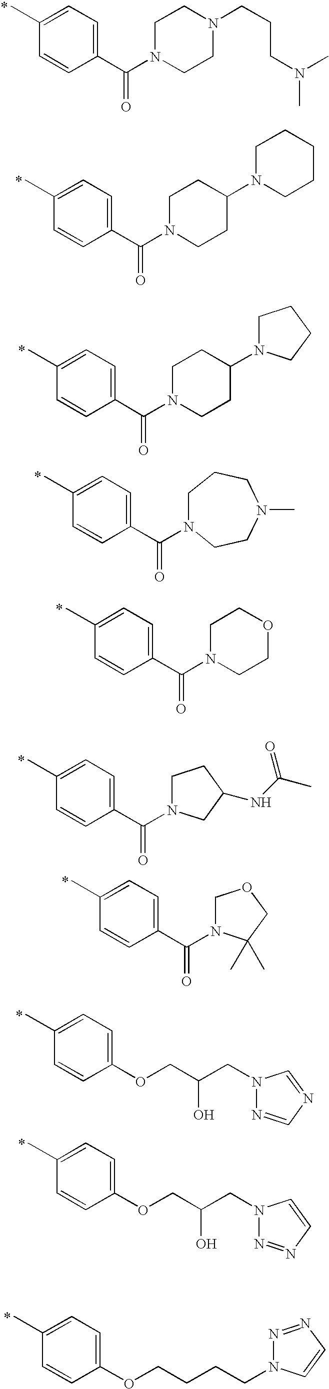

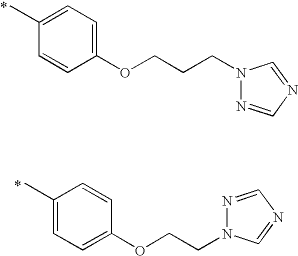

Preferred R₂ substituents also include the following, where the * indicates the bond of attachment to the carboline scaffold molecule.

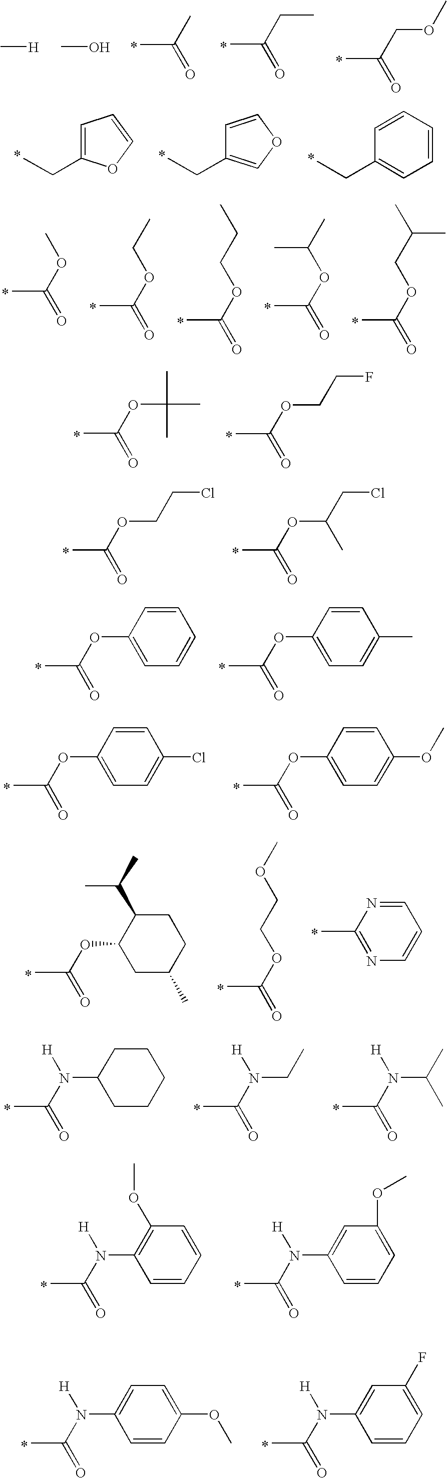

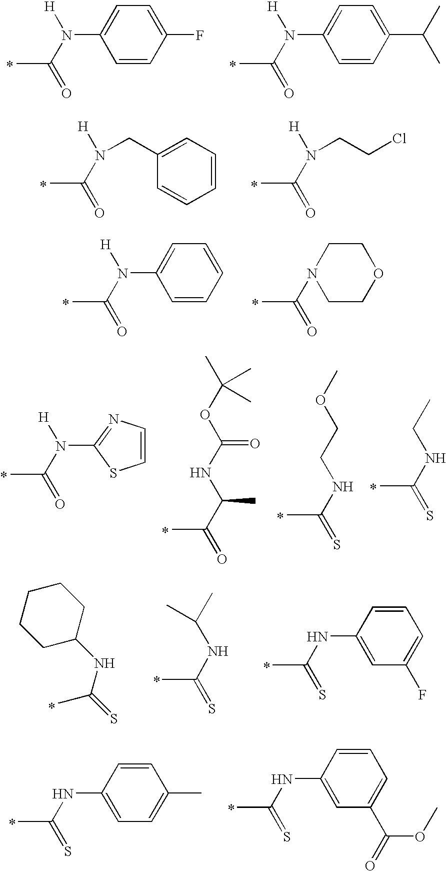

Other preferred R₂ substituents include the following, where the * indicates the bond of attachment to the carboline scaffold molecule.

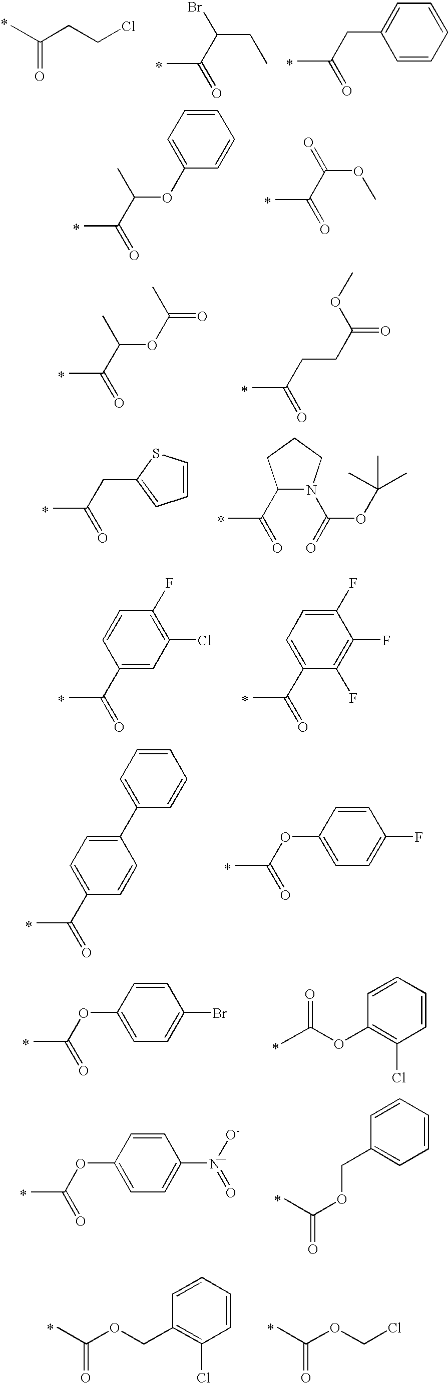

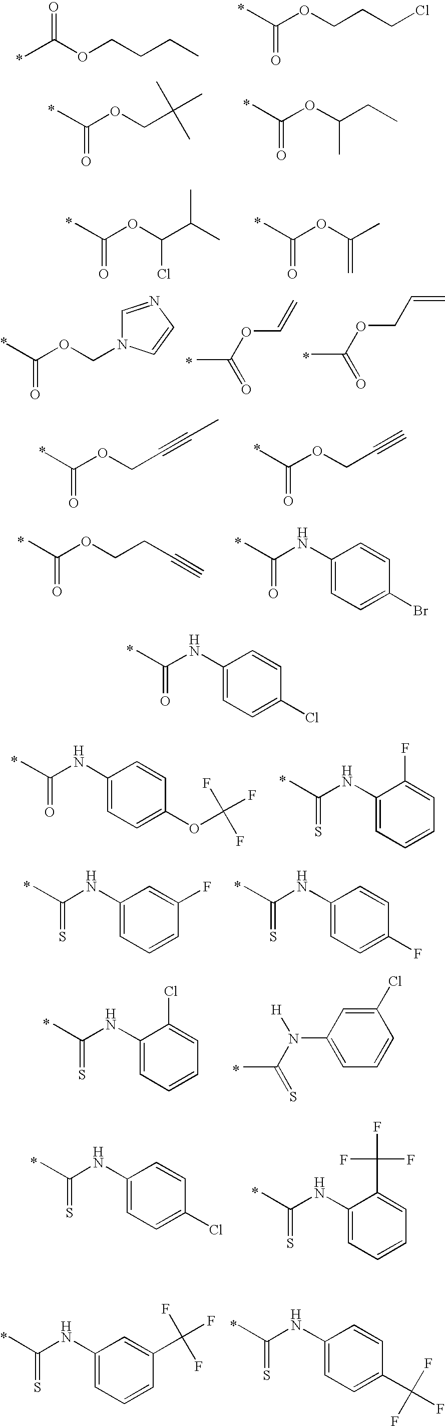

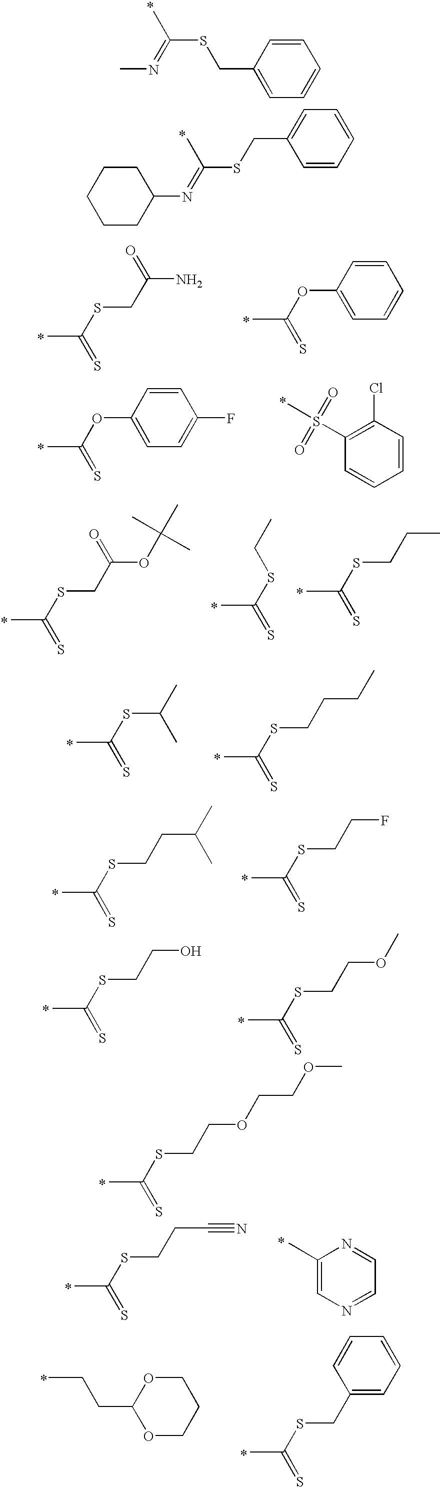

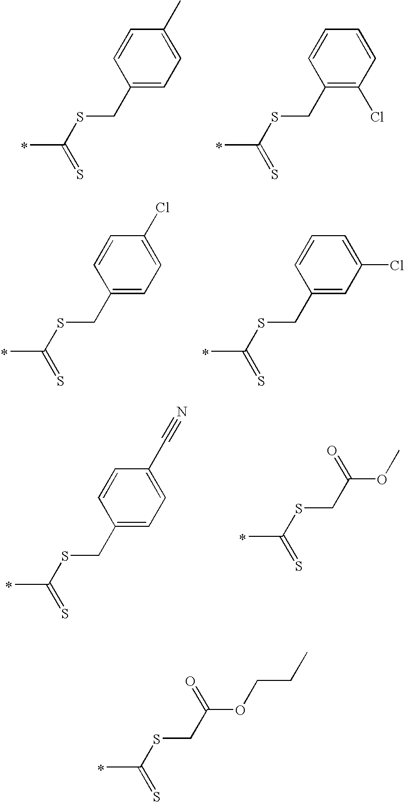

Preferred R₃ substituents include the following, where the * indicates the bond of attachment to the carboline scaffold molecule.

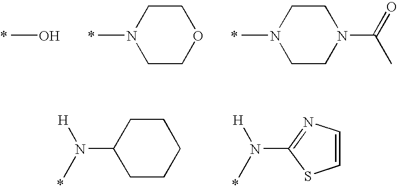

A class of compounds within the scope of Formula (V) include those compounds of Formula (I-a) as shown below.

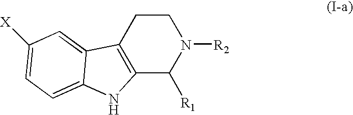

wherein X, R₁ and R₂ are defined as described with regard to Formula (V) and the embodiments described above.

Another class of compounds within Formula (V) include those compounds of Formula (I-b) as shown below.

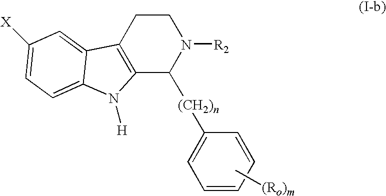

wherein:

X is as described above with regard to Formula (V);

R₂ is as described above with regard to Formula (V);

Rₒ is as described above with regard to Formula (V);

m is 1, 2, or 3; and

n is 0, 1, 2, or 3.

Other compounds within the scope of Formula (V) include the following:

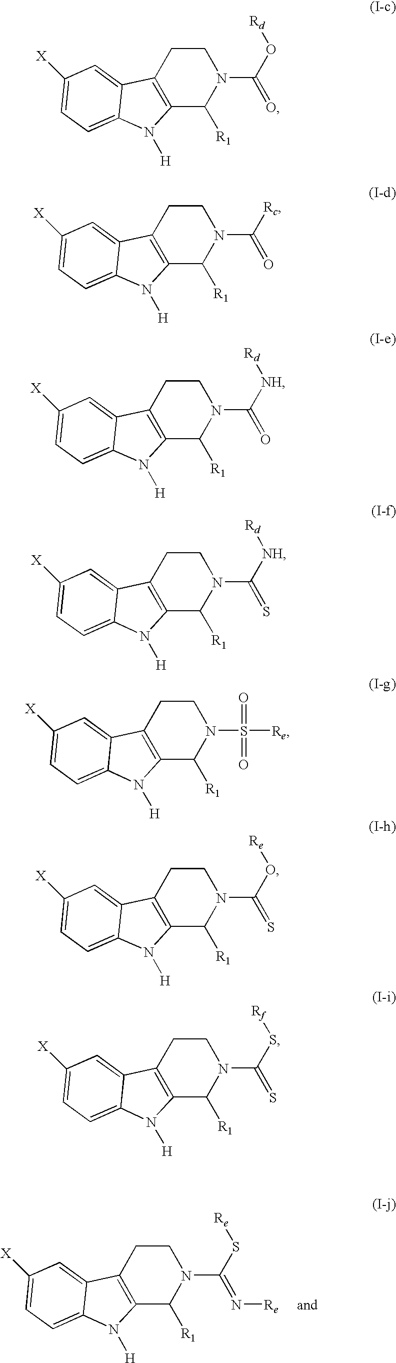

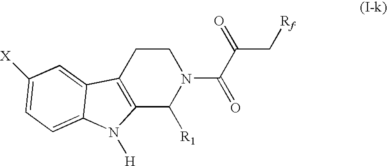

It is understood that substituents X, R₁, R_{c}, R_{d}, Rₑ and R_{f} of the compounds of Formulas (I-c) to (I-k) are defined as in Formula (V).

In other embodiments, preferred compounds of the present invention useful for post-transcriptionally inhibiting the expression of VEGF include those of Formulas (I-l) through (I-m), as shown below. In the embodiments of Formulas (I-l) through (I-m), substituents X, R₁, R₂, and R₃ are defined as in Formula (V).

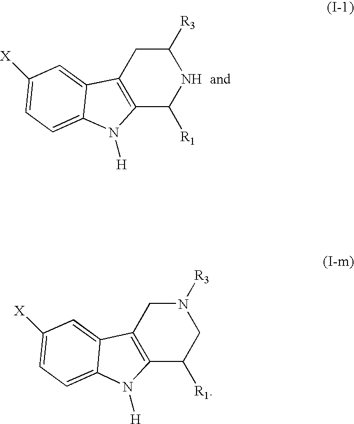

Also included within the scope of the invention are pharmaceutically acceptable salts, hydrates, solvates, calthrates, polymorphs, racemates and stereoisomers of the compounds described herein.

In another aspect of the invention, preferred compounds of the present invention useful for post-transcriptionally inhibiting the expression of VEGF include those of Formula (I-a):

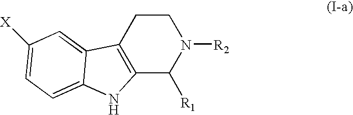

wherein,

X is hydrogen; a hydroxyl group; a halogen; a C₁-C₄ alkyl; a C₁ to C₅ alkoxy, optionally substituted with an aryl group,

with the proviso that, when X is C₁ to C₅ alkoxy and R₂ is —C(O)O—R_{d}, wherein R_{d} is C₁ to C₄ alkyl, then R₁ is other than unsubstituted C₁ to C₈ alkyl;

R₁ is a hydroxyl group; a C₁ to C₈ alkyl group, optionally substituted with an aryl group, wherein the aryl group is optionally substituted with at least one R₀ group; a heterocycle group; a heteroaryl group; and an aryl group, optionally substituted with at least one R₀ group,

with the proviso that, when R₁ is unsubstituted phenyl, then X is other than hydrogen;

Rₒ is a halogen; a C₁ to C₆ alkyl, optionally substituted with one or more halogen groups; a cyano group; a nitro group; an amino group; an aminoalkyl group; an acetamide group; an imidazole group; or ORₐ;

Rₐ is hydrogen; a C₁ to C₆ alkyl, optionally substituted with a heterocycle group or an aryl group; or a —C(O)O—R_{b};

R_{b} is C₁ to C₄ alkyl group;

R₂ is a hydrogen; a hydroxyl; a heteroaryl group; a C₁ to C₈ alkyl group, optionally substituted with an alkoxy, hydroxyl, heteroaryl, or aryl group; a —C(O)—R_{c} group; a —C(O)O—R_{d} group; a —C(O)NH—R_{d} group; a —C(S)NH—R_{d} group; a —S(O₂)—Rₑ group; or (1S)-isopropyl-carbamic acid tert-butyl ester,

with the proviso that, when R₂ and X are hydrogen, then R₁ is other than fluorenyl, substituted carbazolyl or phenyl, wherein phenyl is optionally monosubstituted with halogen, nitro or substituted amino, or di- and tri-substituted with C₁ to C₄ alkoxy;

with the proviso that, when R₂ is —C(O)—R_{c}, —C(O)O—R_{d}, —C(O)—NH(R_{d}) or —C(S)—NH(R_{d}), wherein R_{c} is C₁ to C₈ alkyl substituted with optionally substituted phenyl, wherein R_{d} is optionally substituted phenyl, cyclohexyl or C₁ to C₈ alkyl optionally substituted with optionally substituted phenyl or —C(O)O—Rₙ, and X is hydrogen, then R₁ is other than unsubstituted benzo[1,3]dioxolyl or optionally substituted phenyl, wherein phenyl is optionally disubstituted with chloro and methoxy;

R_{c} is hydrogen; a 4-morpholinyl group; a thiazoleamino group; a piperazinyl group, optionally substituted with a —C(O)CH₃ group; a C₁ to C₆ alkyl group, optionally substituted with a halogen, an alkoxy, or hydroxyl group;

R_{d} is hydrogen; a benzyl group; a C₁ to C₈ alkyl group, optionally substituted with a halogen or an alkoxy group; a aryl group, optionally substituted with at least one halogen, C₁ to C₅ alkyl, —C(O)ORₑ, or ORₑ; and,

Rₑ is a hydrogen; a C₁ to C₆ alkyl group, optionally substituted with at least one halogen or alkoxy group; or an aryl group.

In another embodiment, compounds of Formulas (II), (III), (IV) and (V) are provided, which are useful for post-transcriptionally inhibiting VEGF production, of the formula:

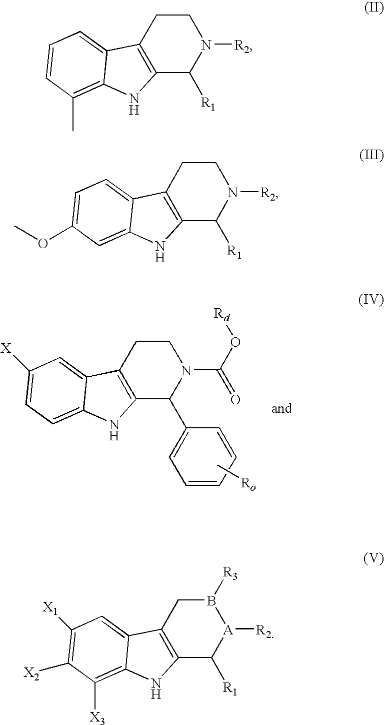

wherein A, B, X, X₁, X₂, X₃, R₁, R₂, R₃, Rₒ and R_{d} are defined as described above with regard to Formula (V).

For the purposes of this invention, wherein one or more of the functionalities encompassing A, B, X, X₁, X₂, X₃, R₁, R₂, R₃, Rₐ, R_{b}, Rₑ, R_{d}, Rₑ, R_{f}, R_{g}, Rₒ and Rₙ, are incorporated into a molecule of Formulas (I), (II), (III), (IV) and (V), including Formulas (I-a) to (I-m), each of the functionalities appearing at any location within the disclosed may be independently selected, and as appropriate, independently substituted. Further, where a more generic substituent is set forth for any position in the molecules of the present invention, it is understood that the generic substituent may be replaced with more specific substituents, and the resulting molecules are within the scope of the molecules of the present invention.

Compounds of the present invention include the following:

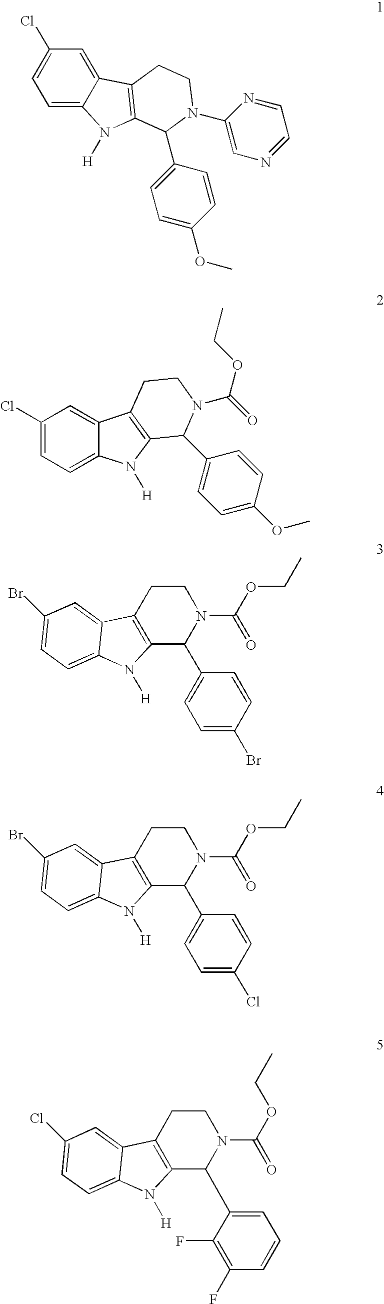

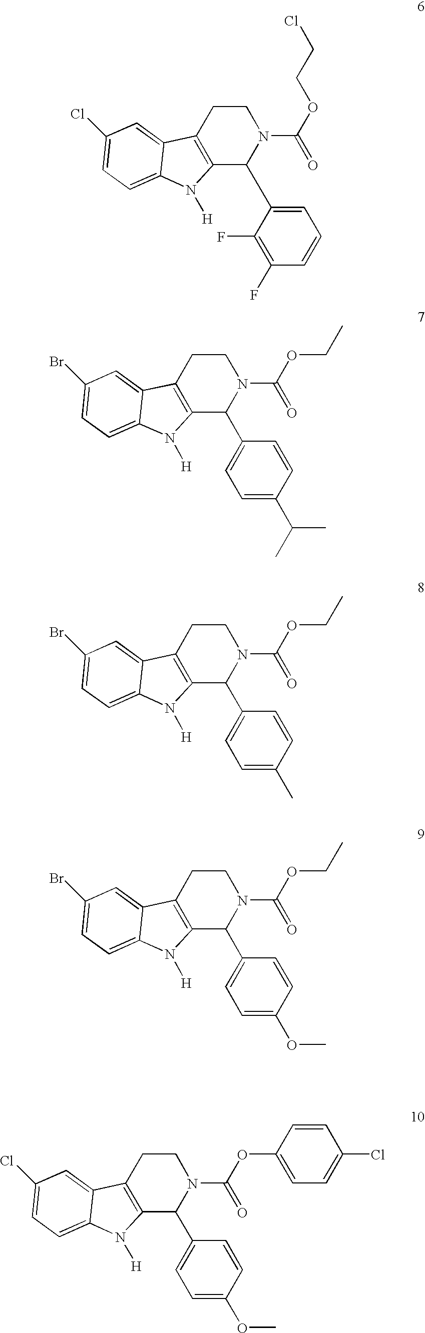

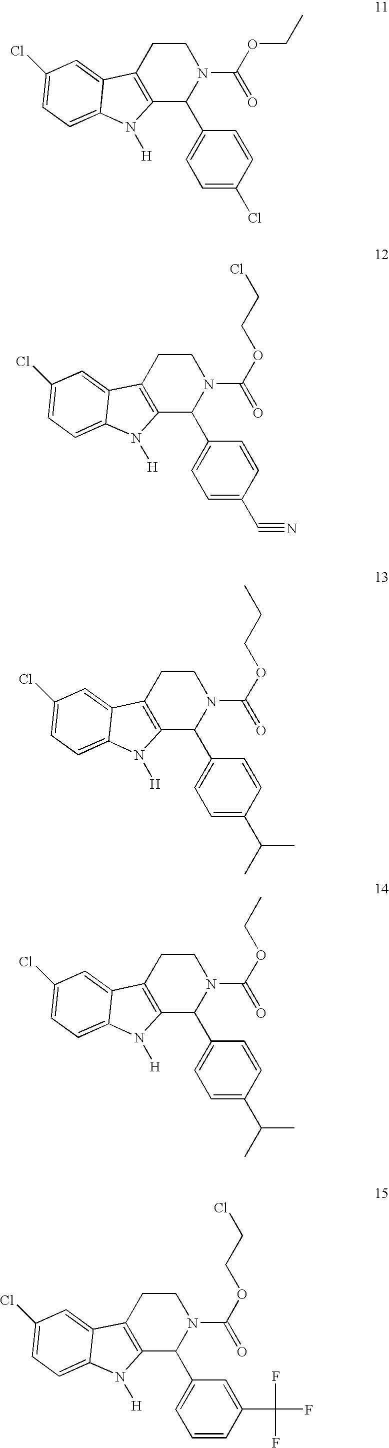

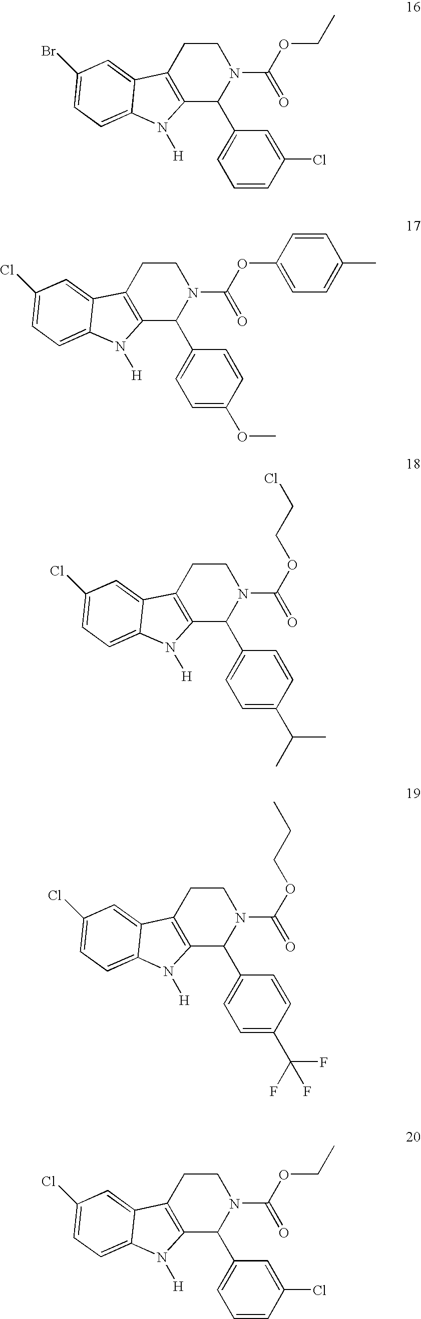

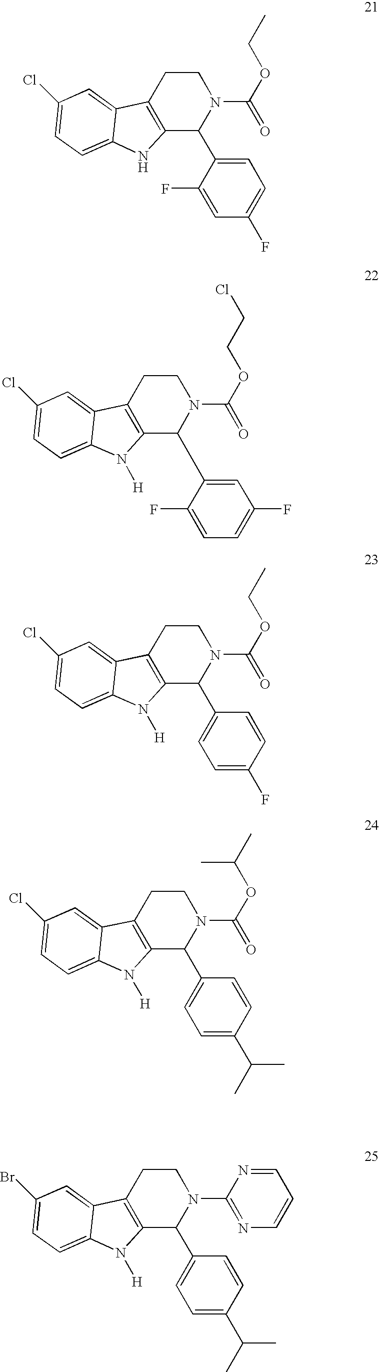

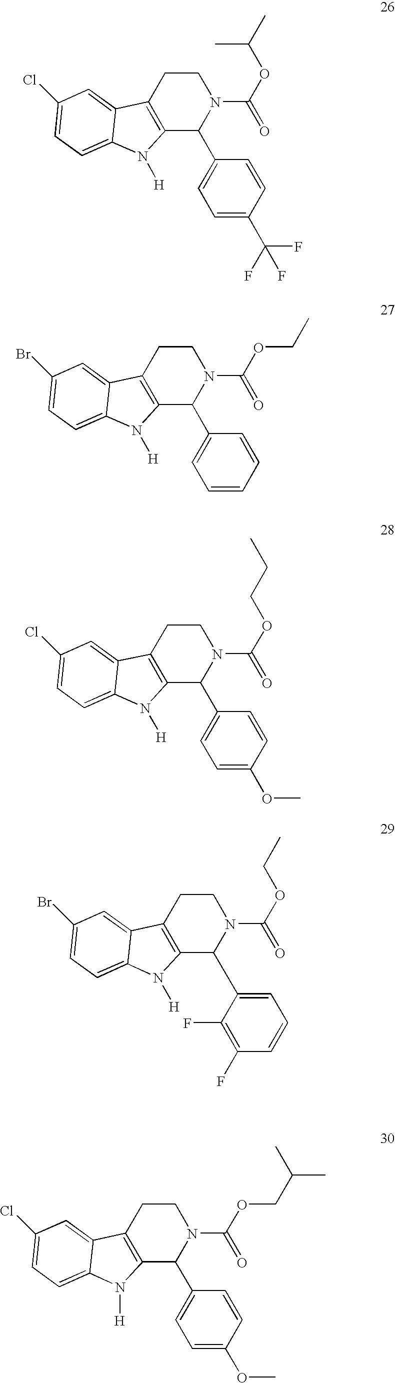

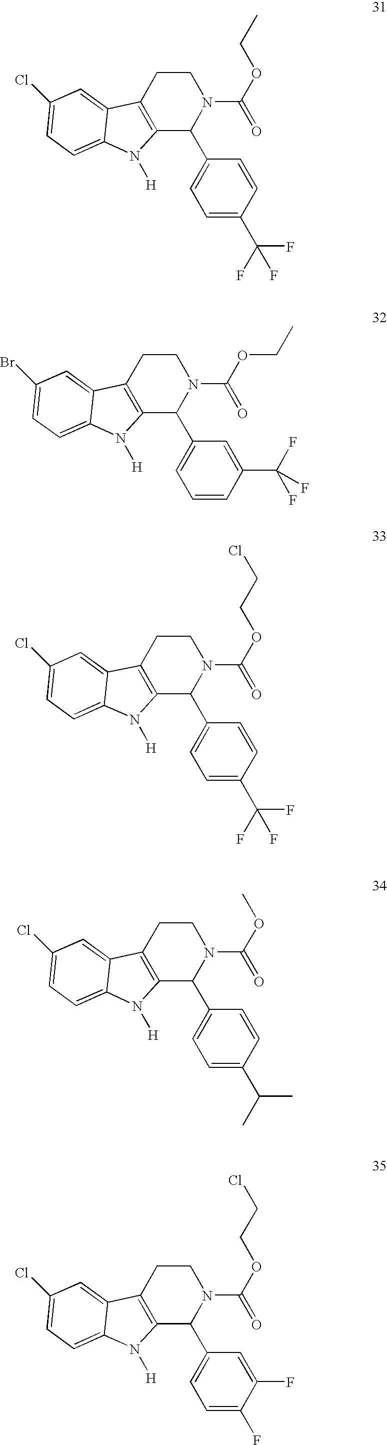

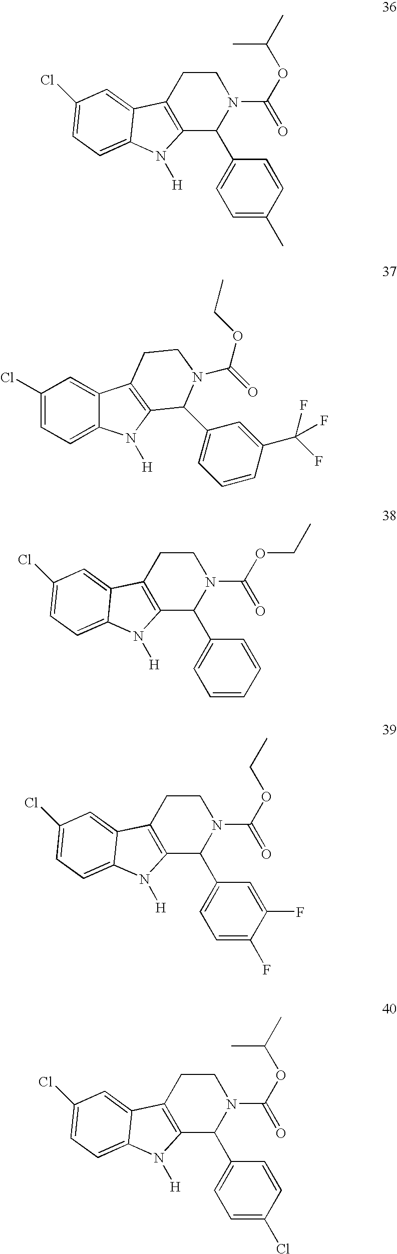

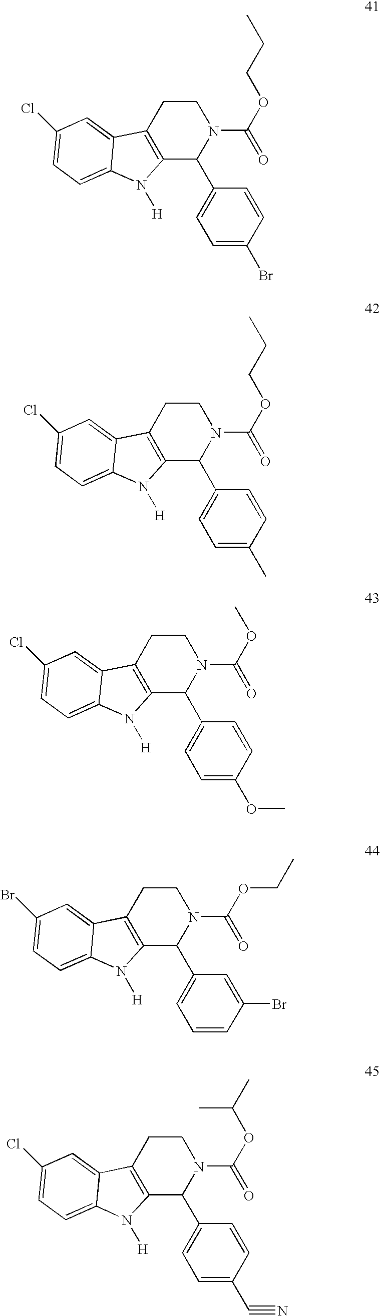

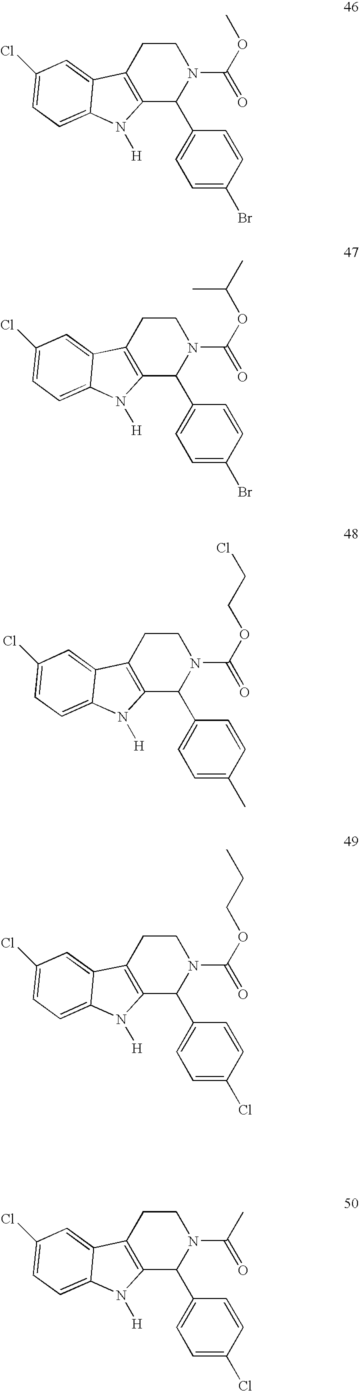

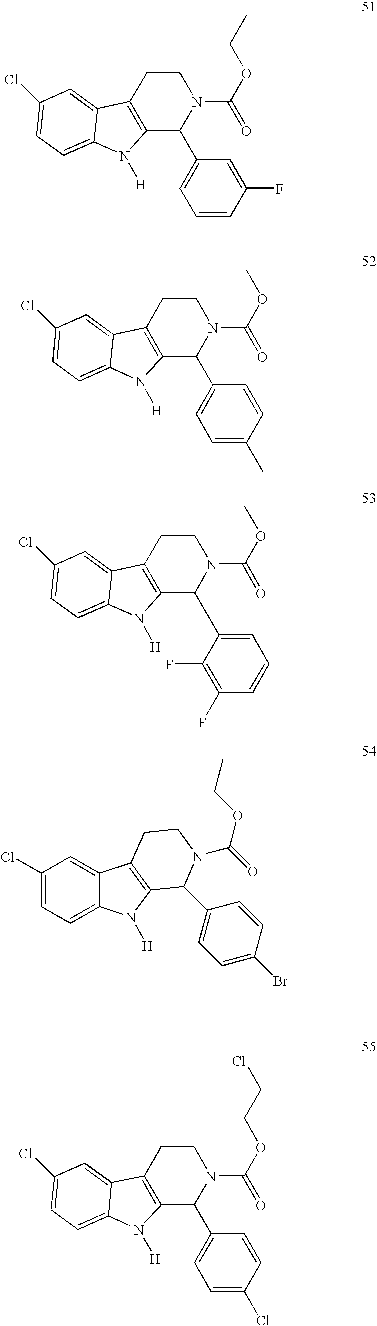

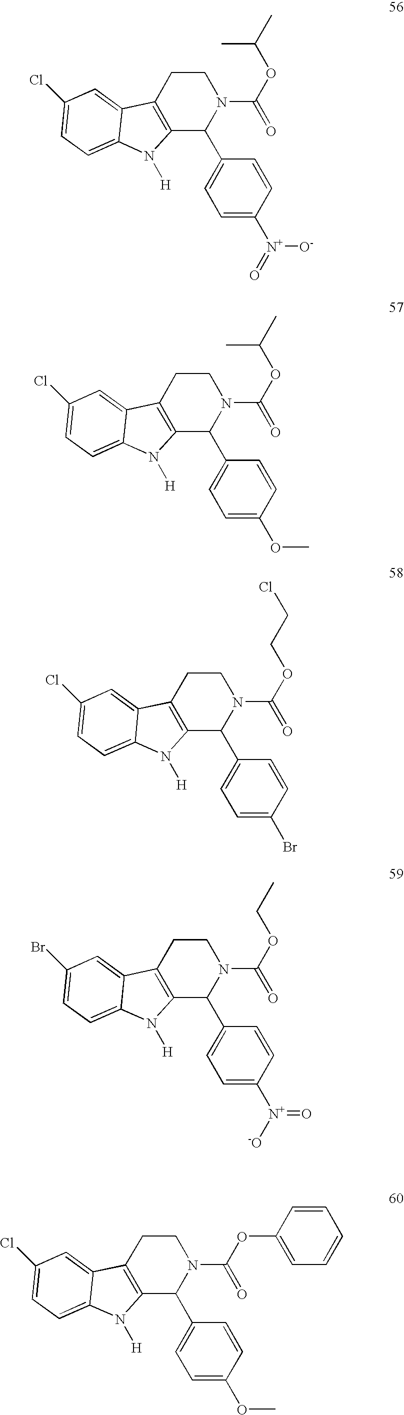

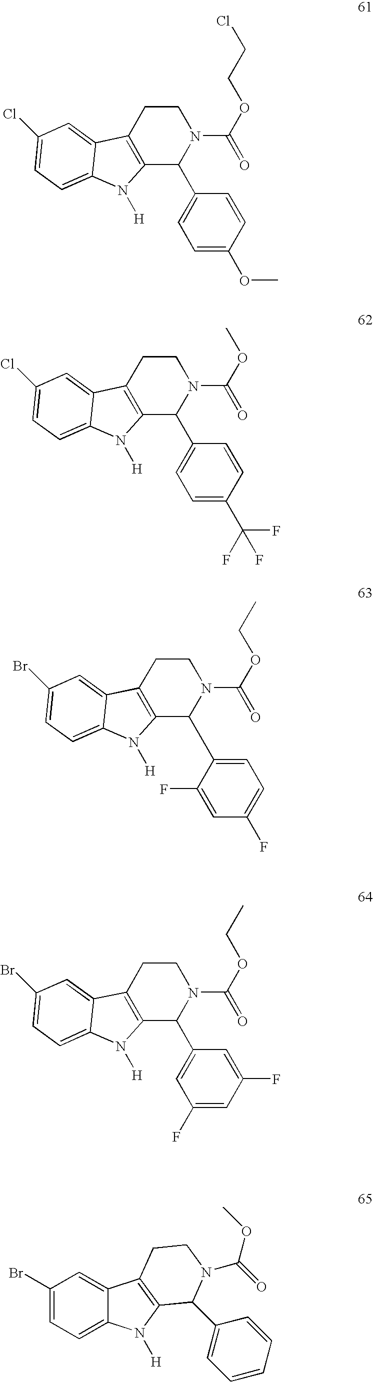

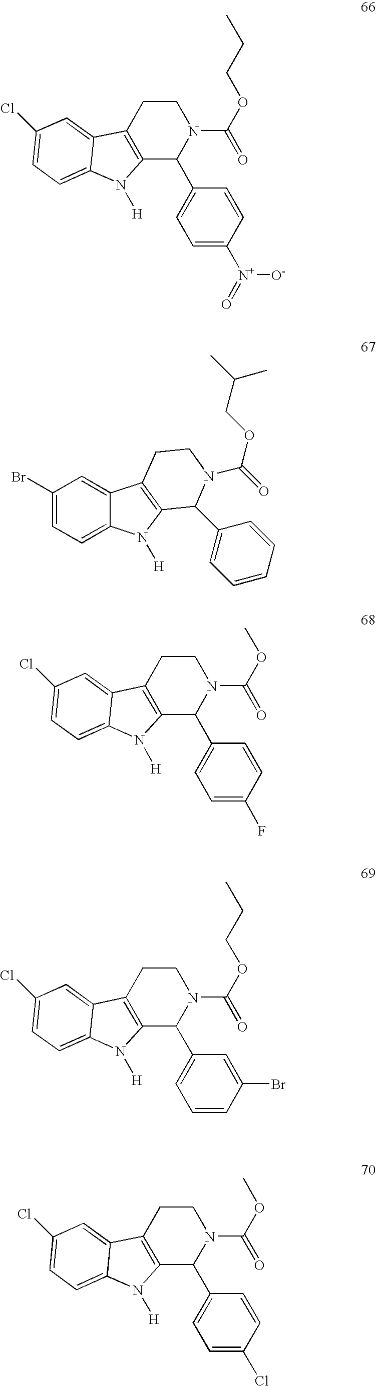

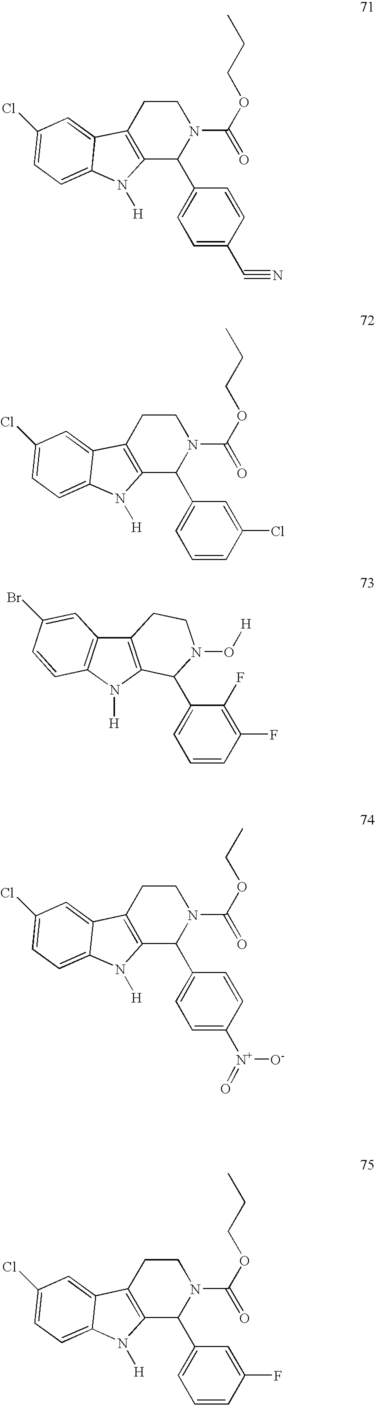

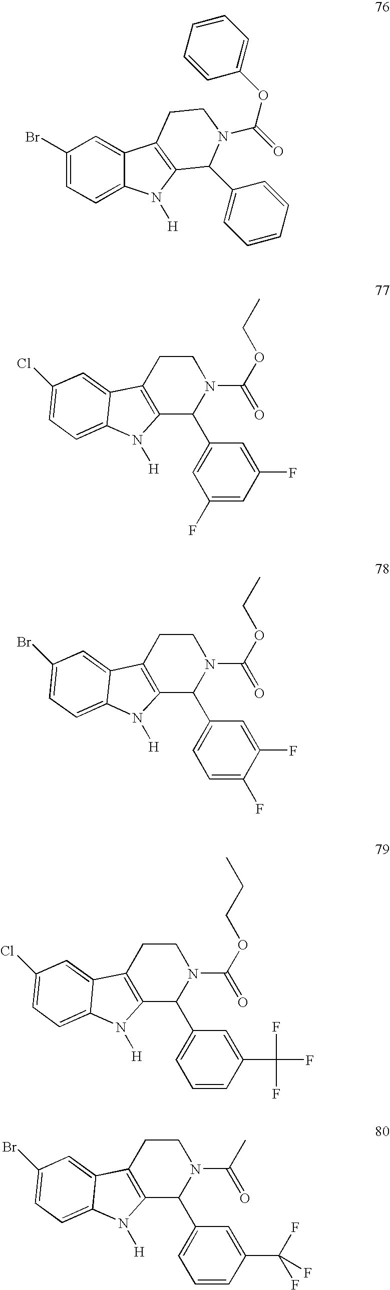

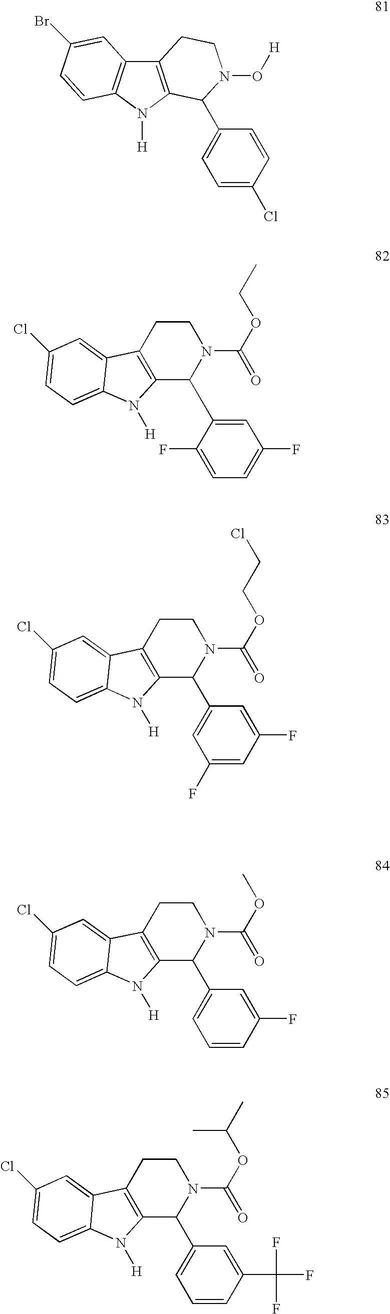

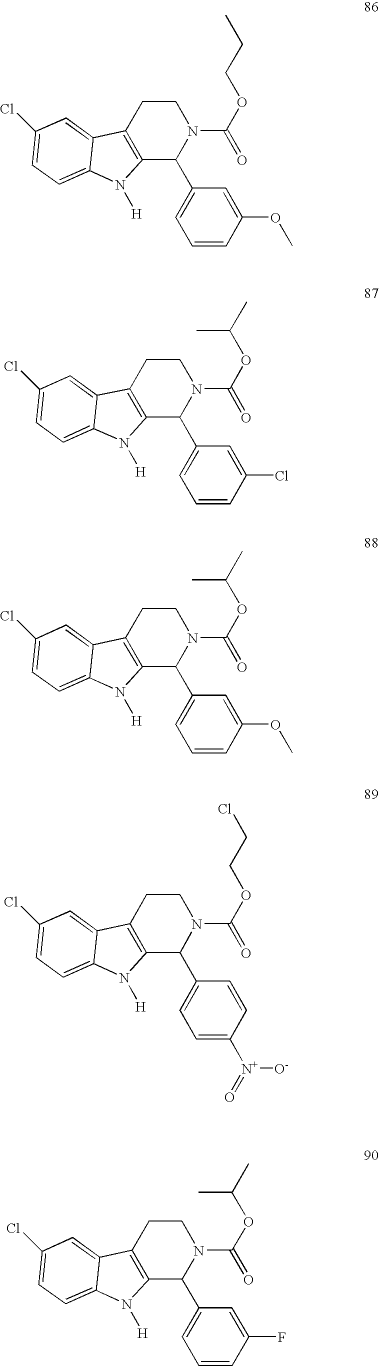

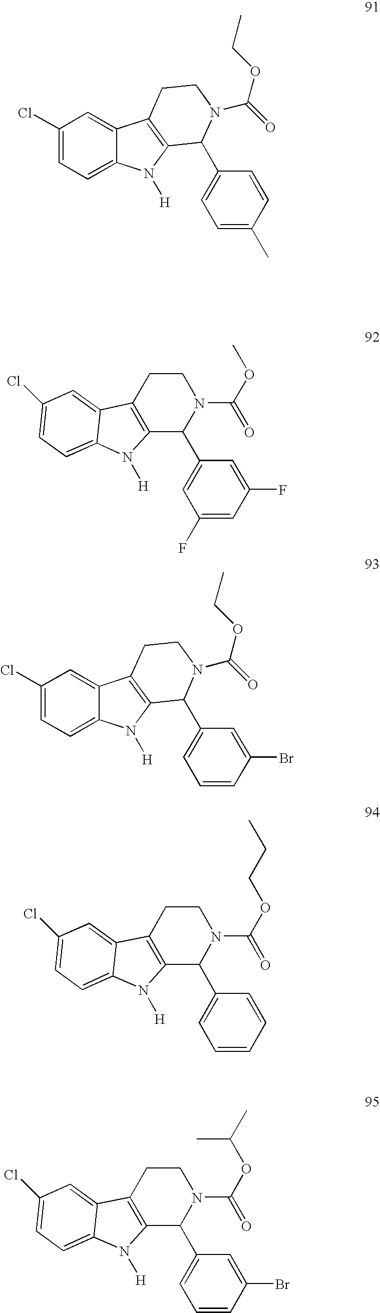

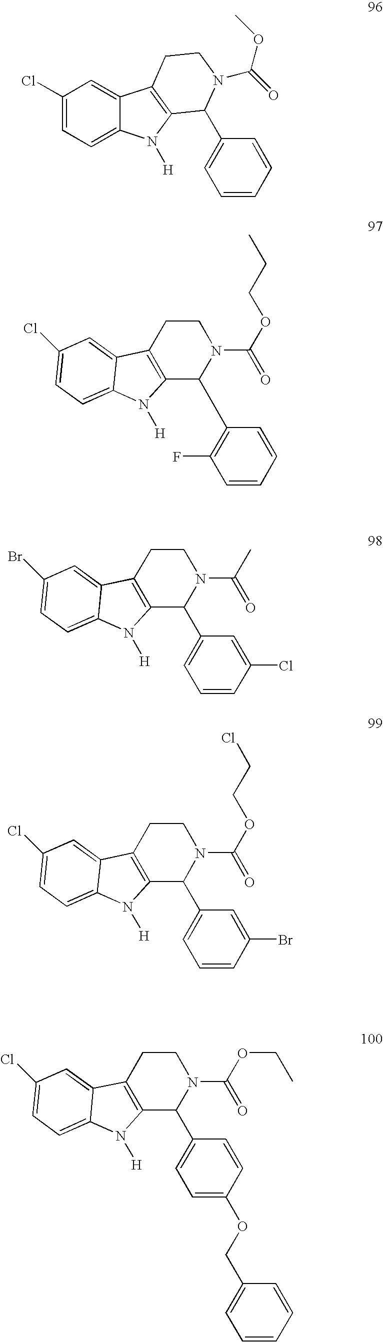

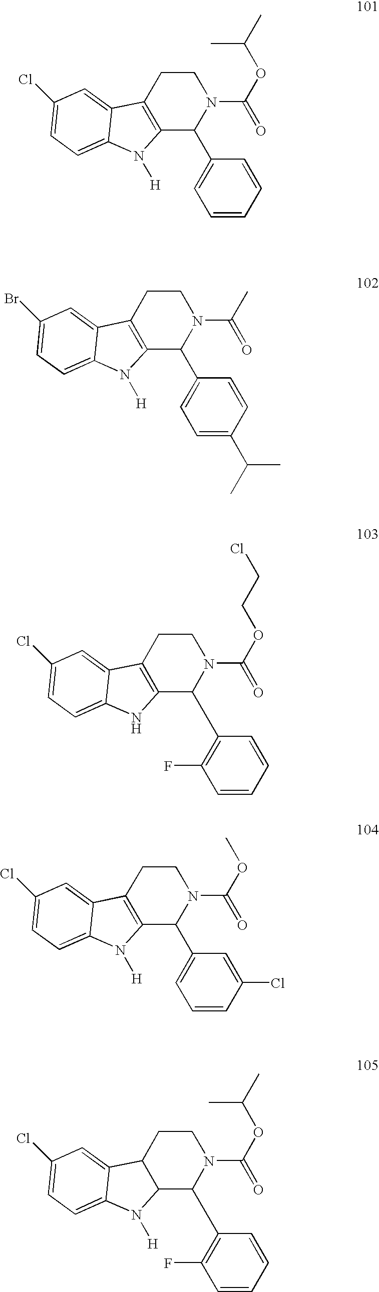

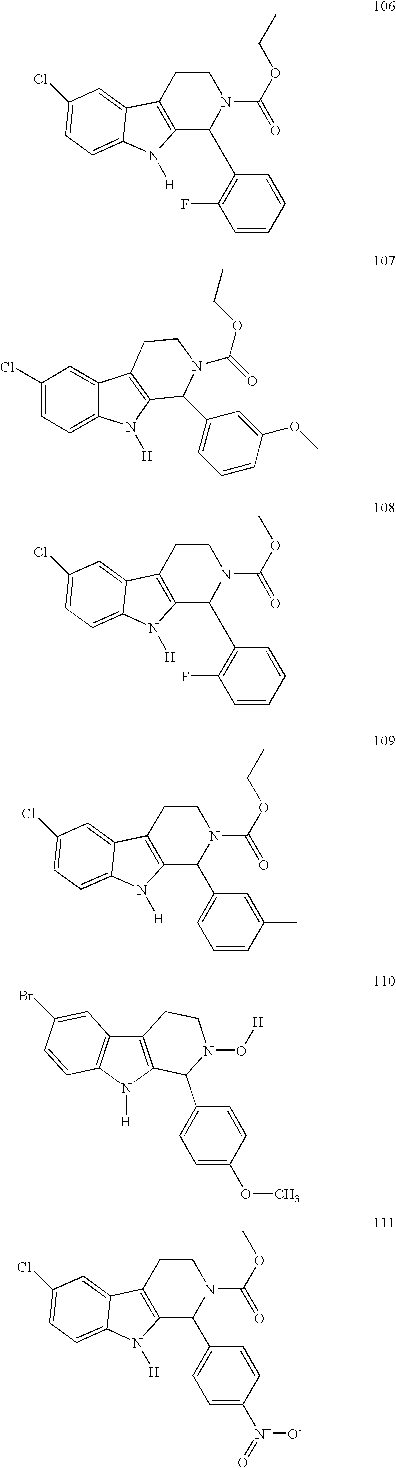

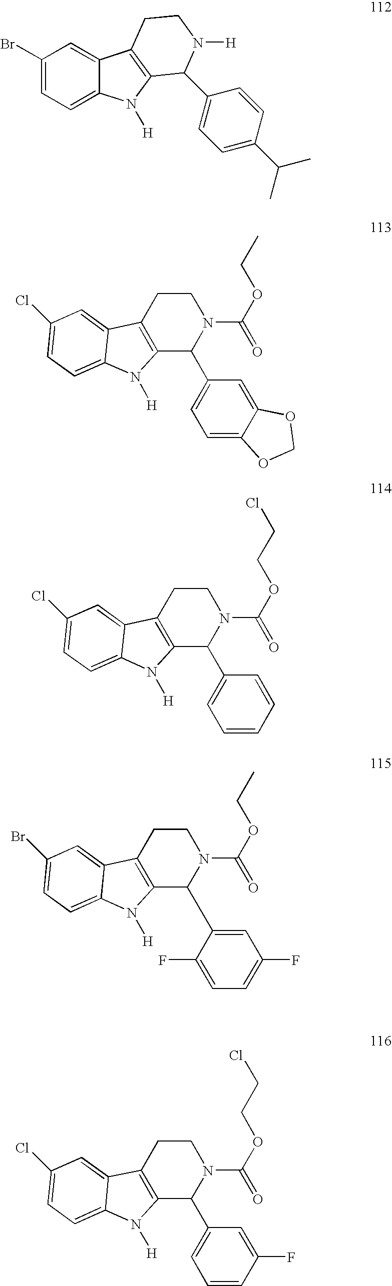

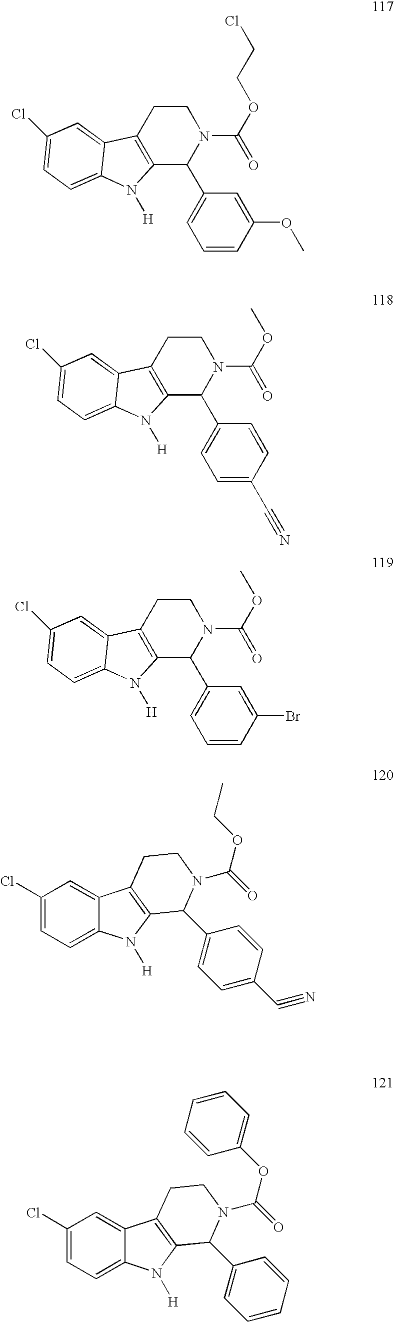

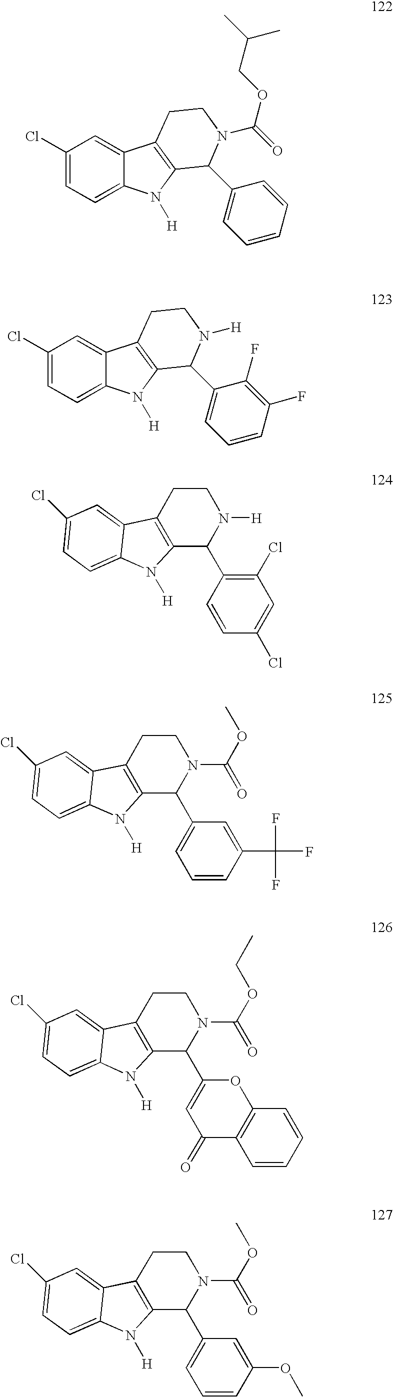

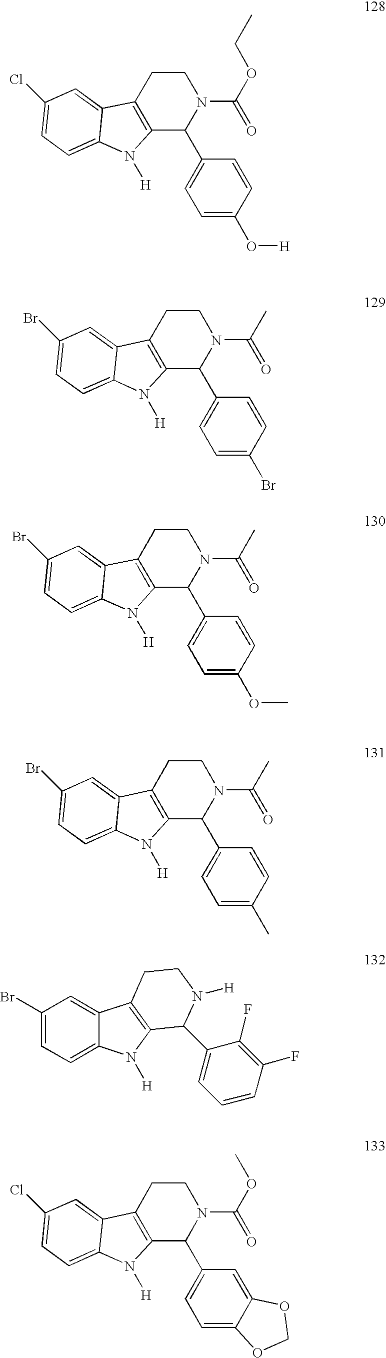

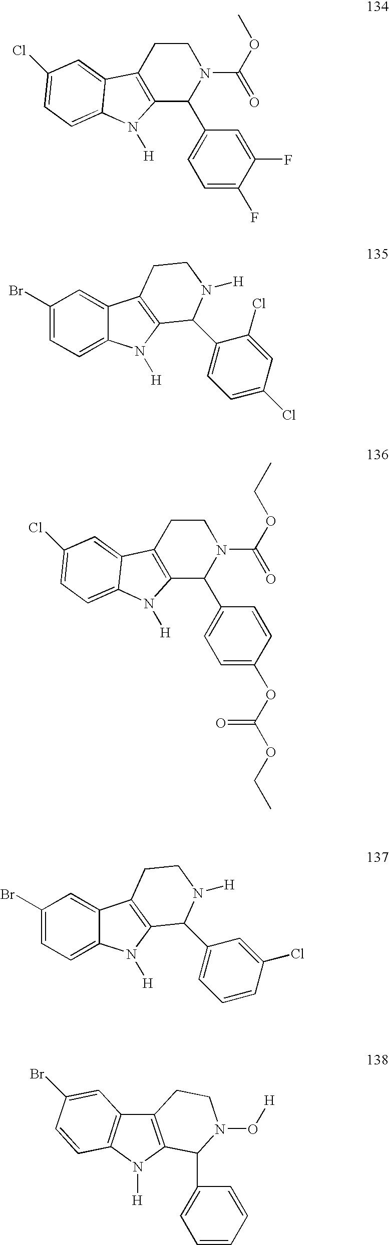

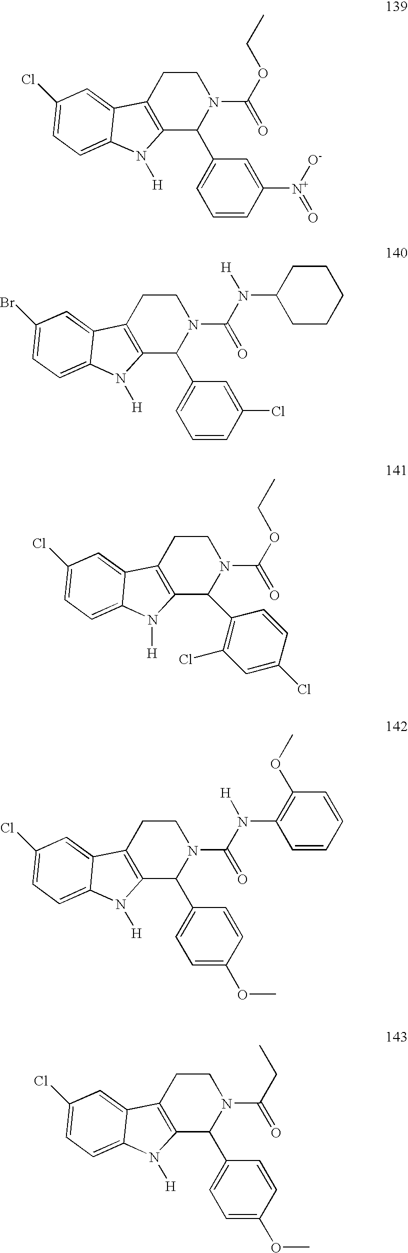

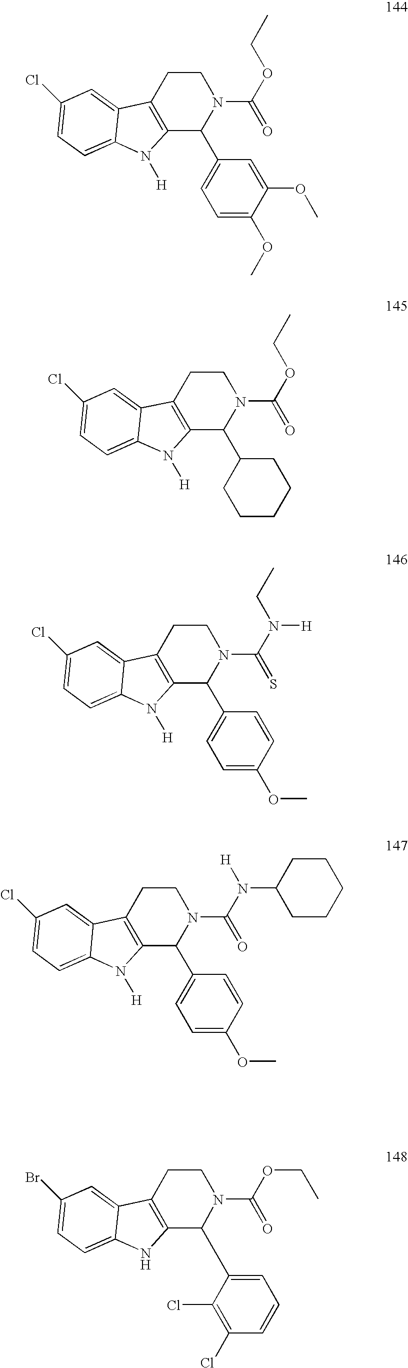

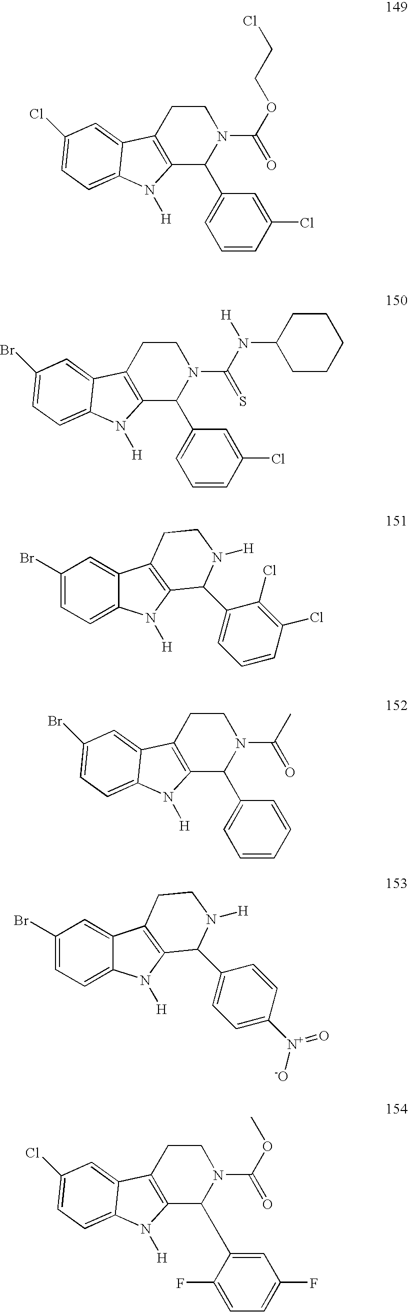

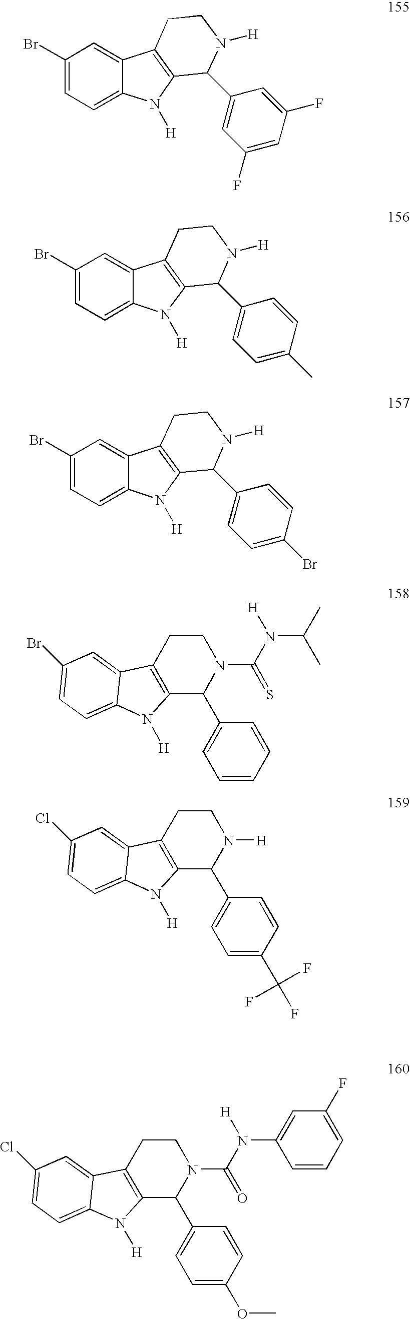

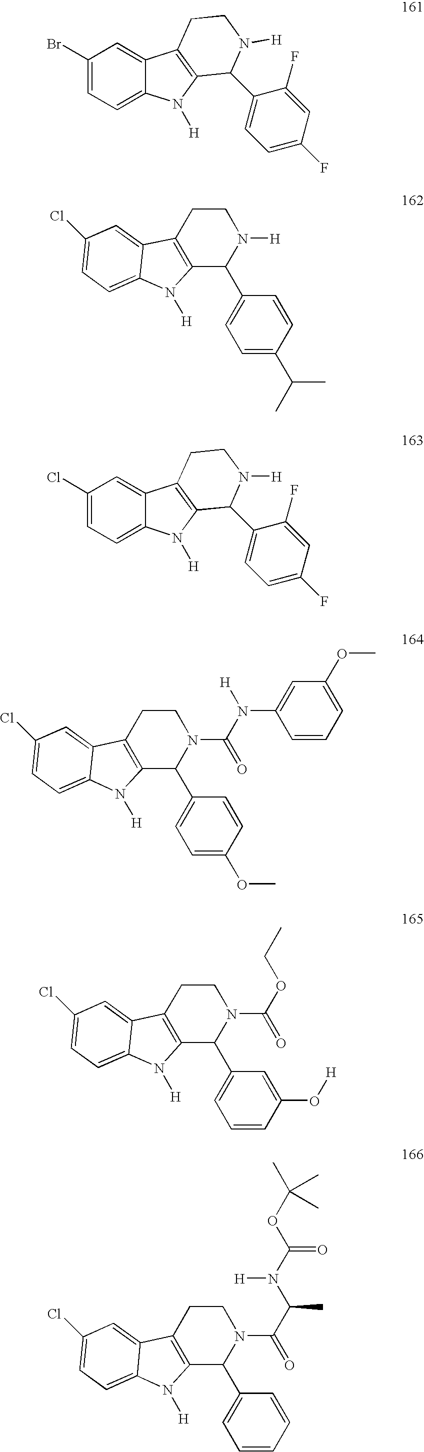

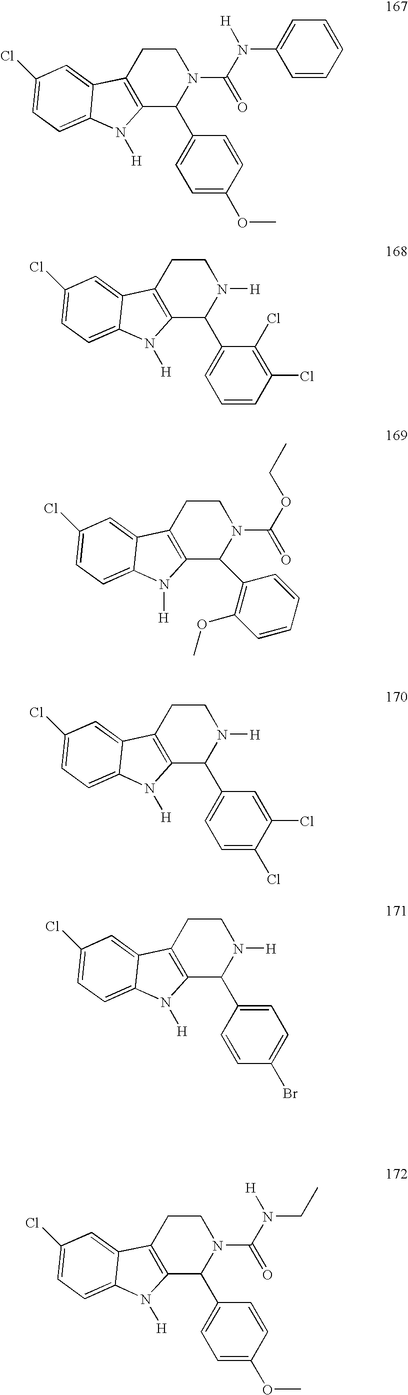

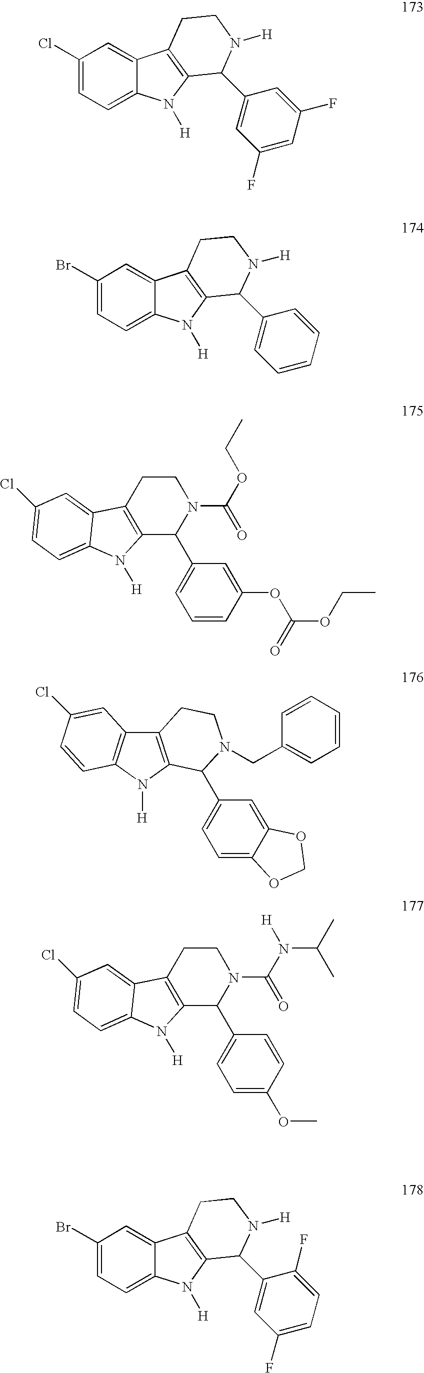

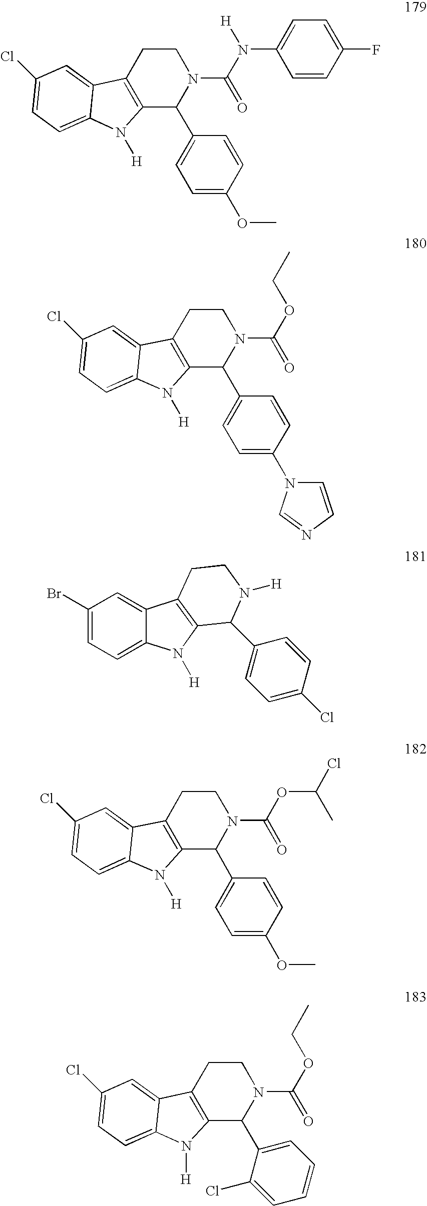

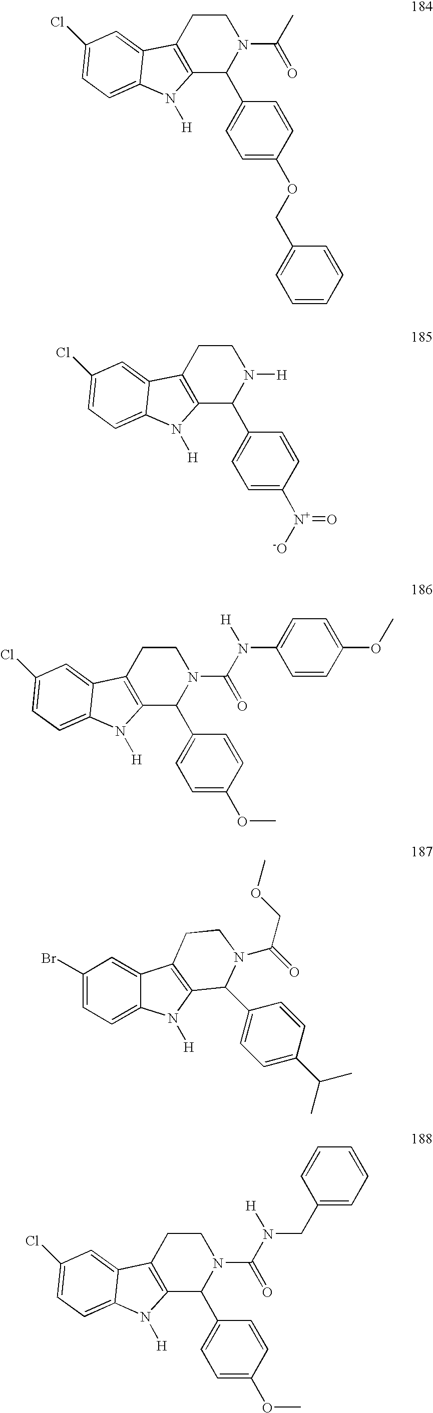

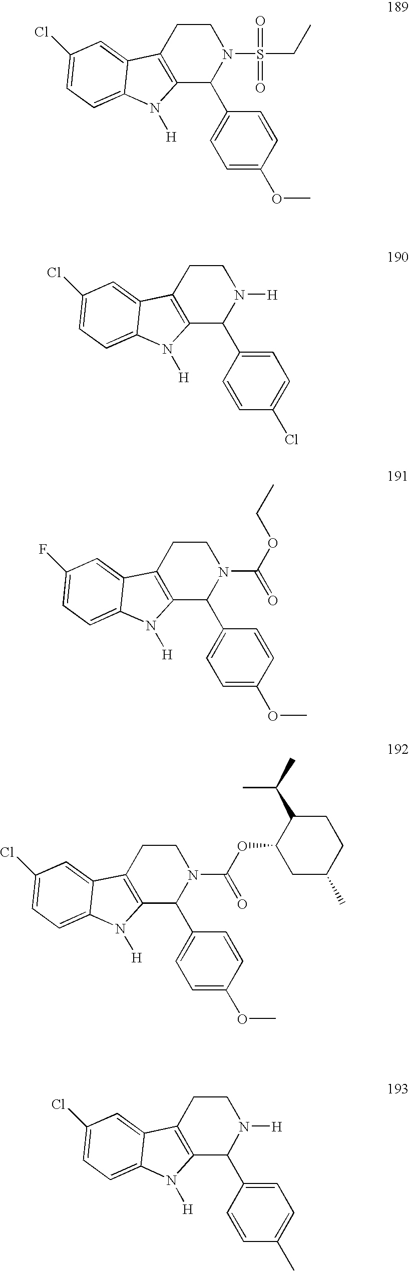

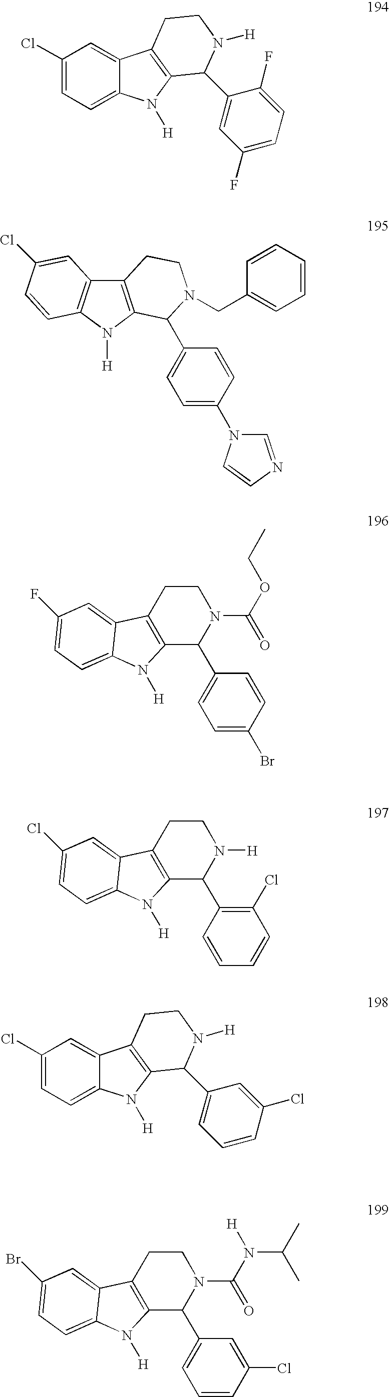

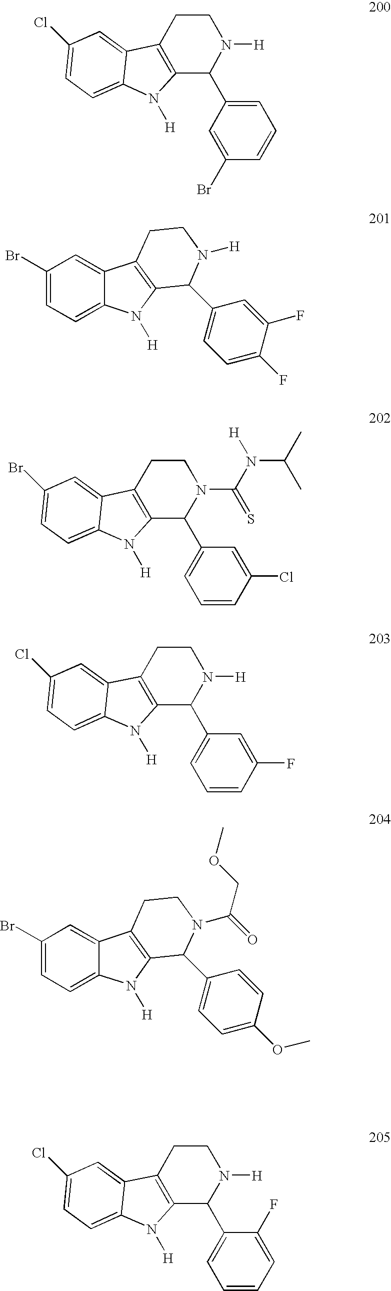

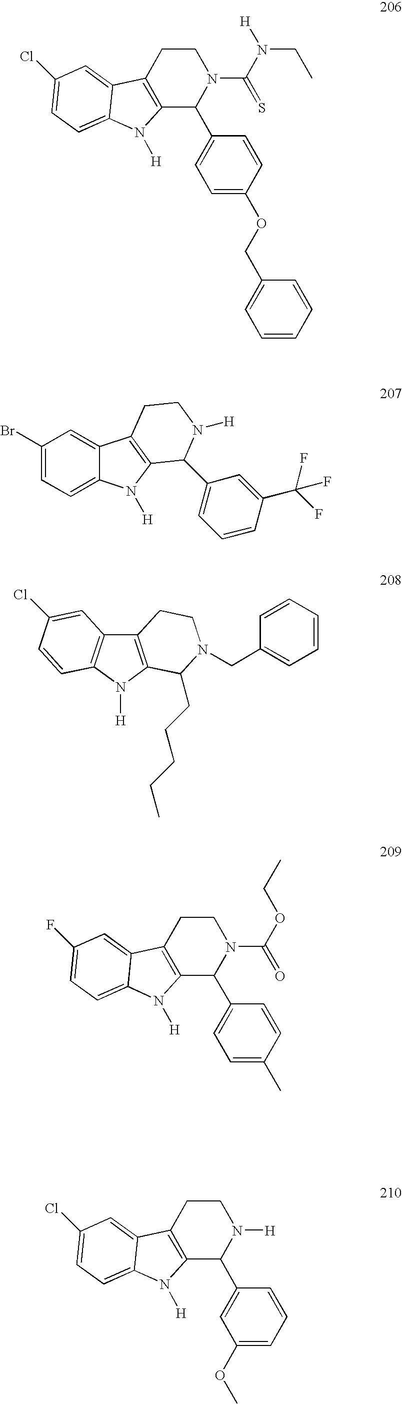

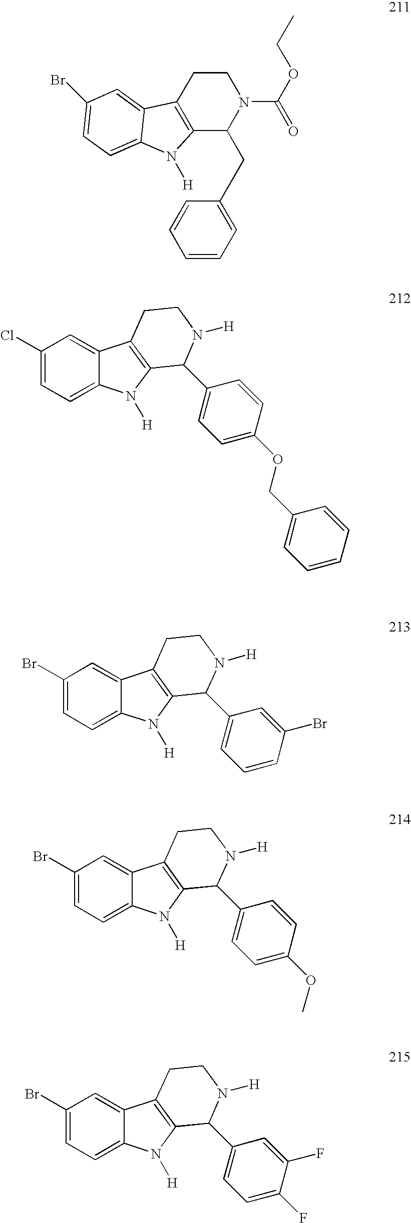

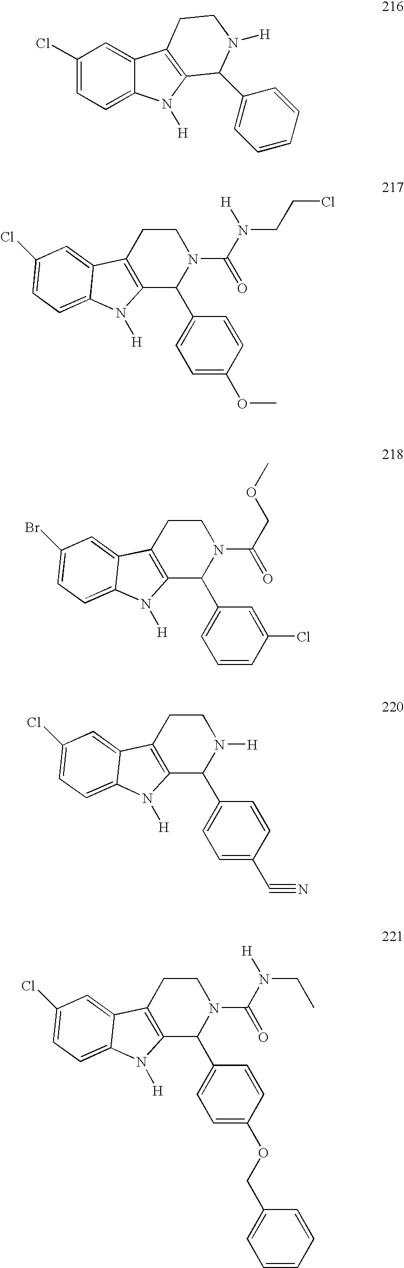

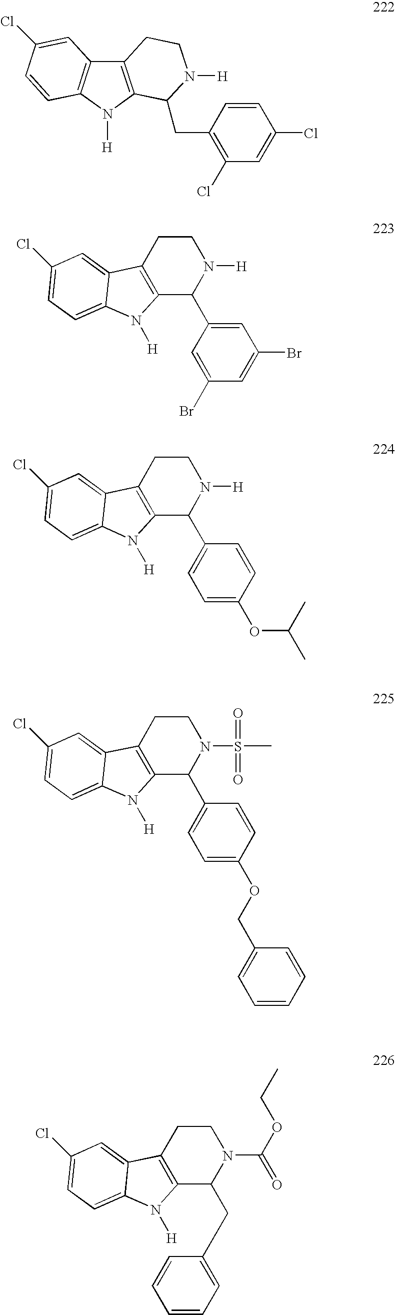

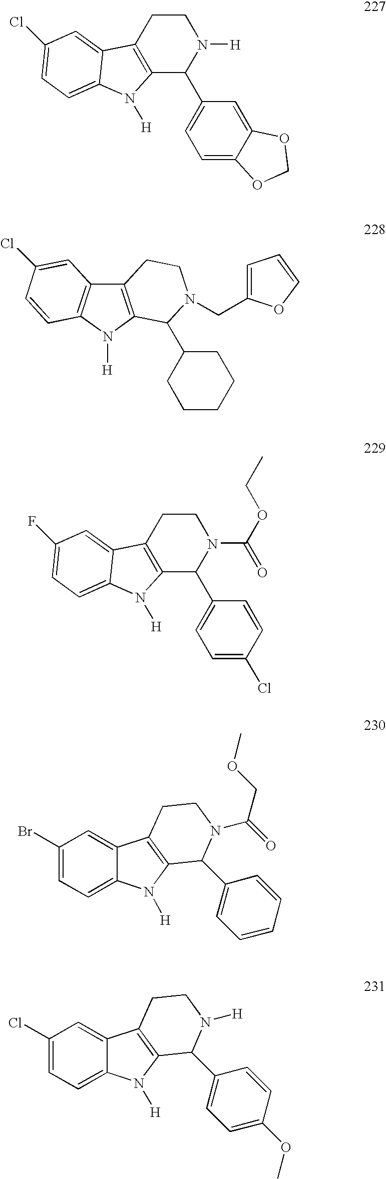

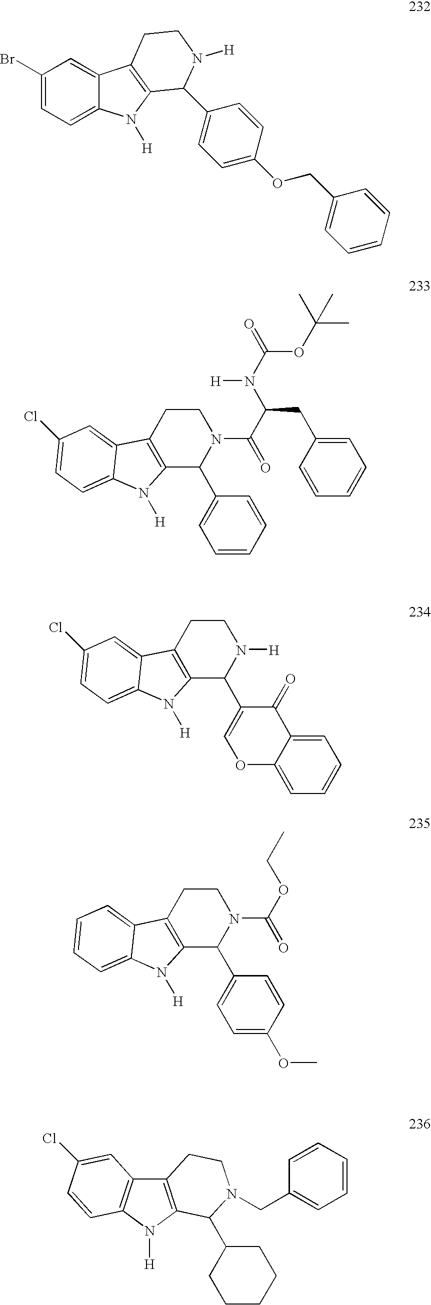

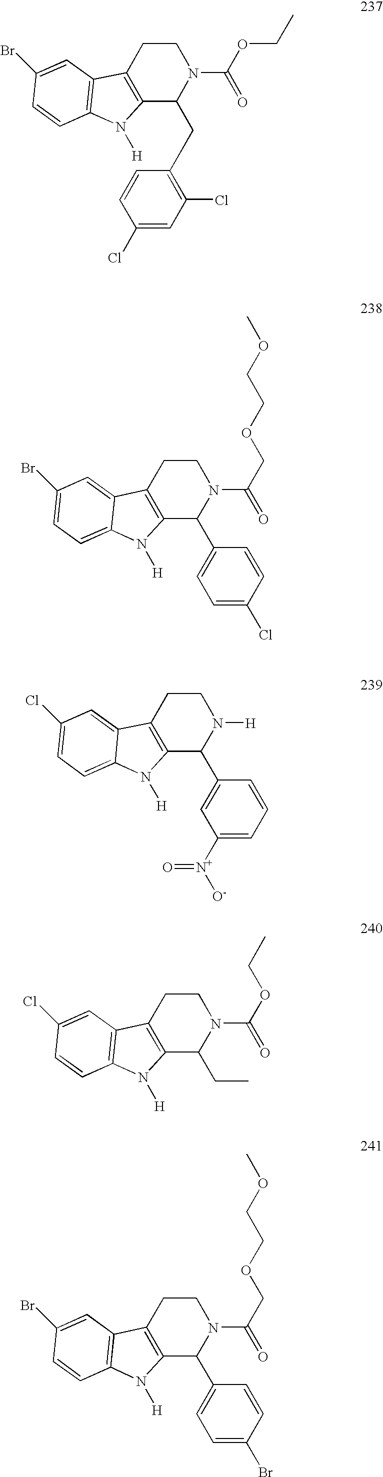

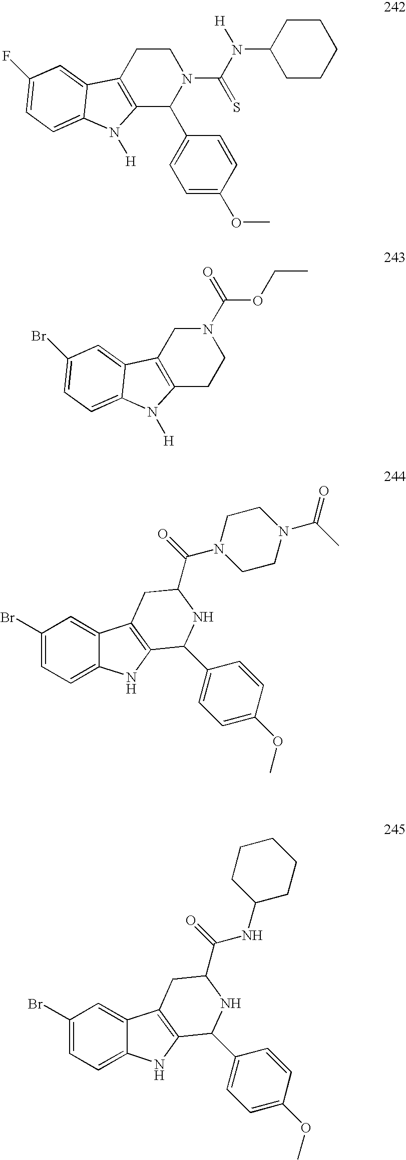

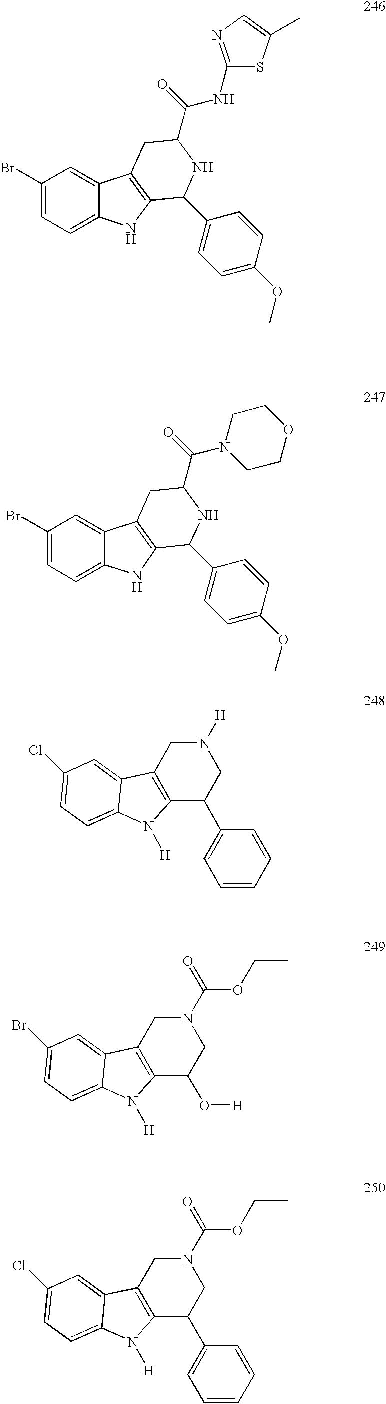

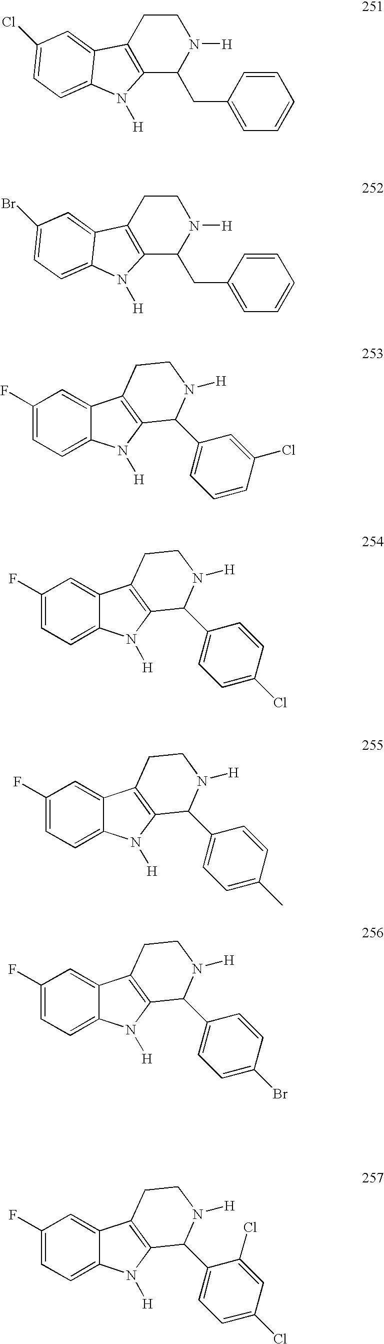

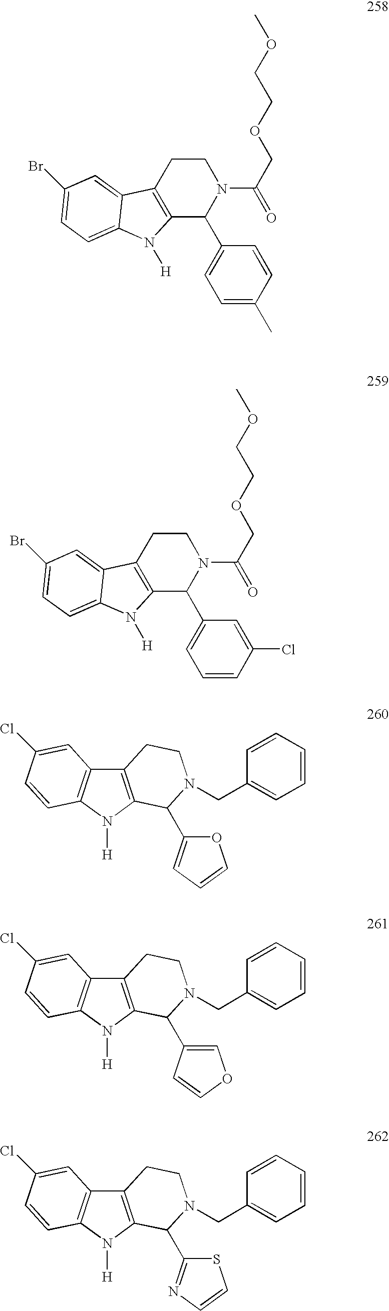

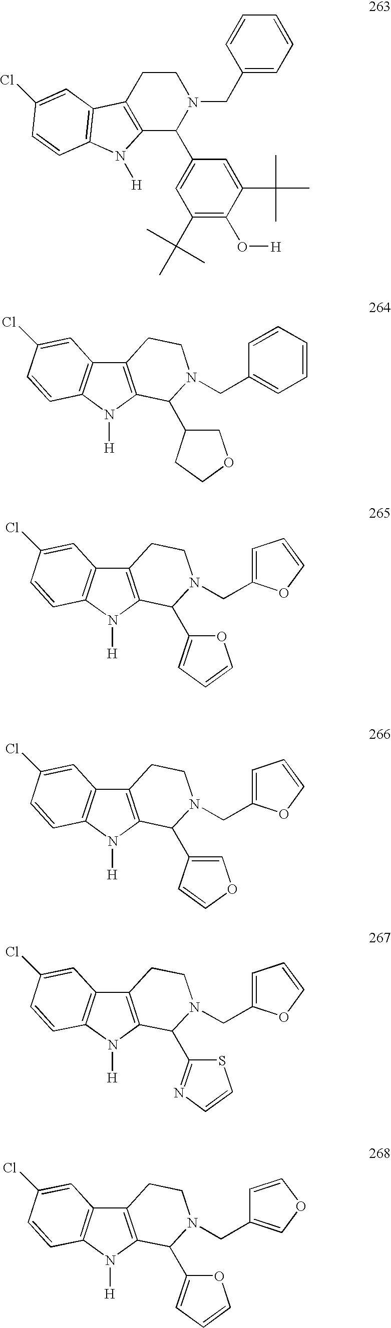

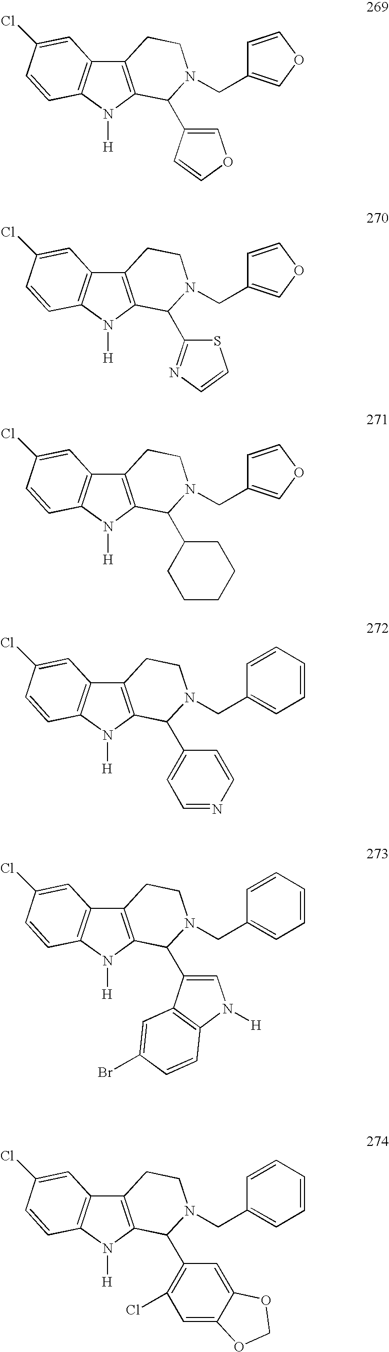

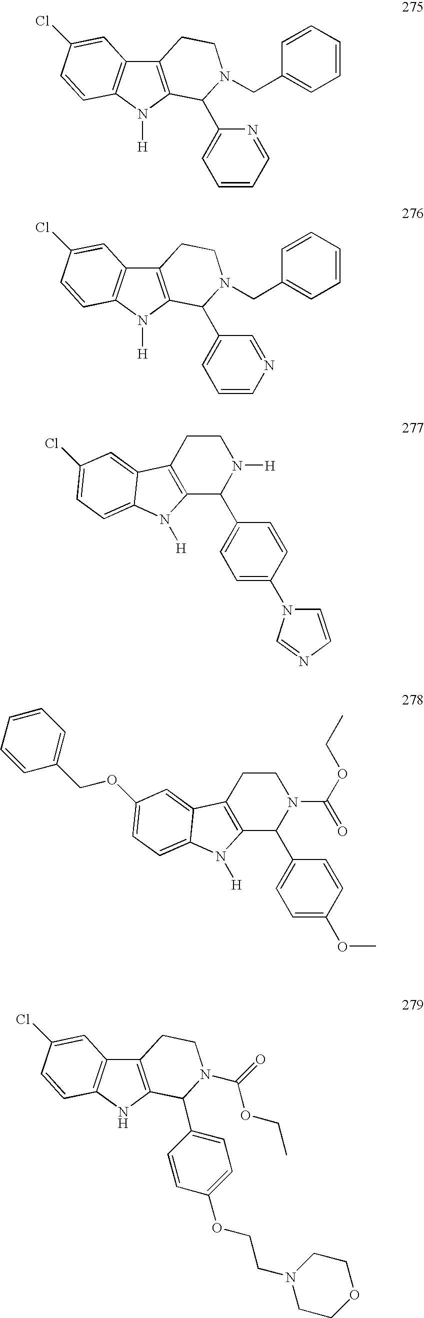

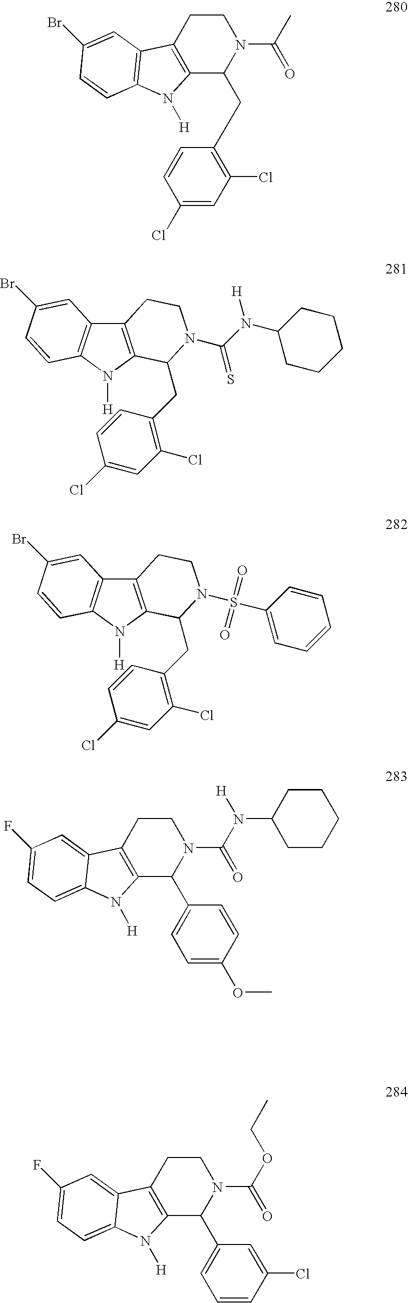

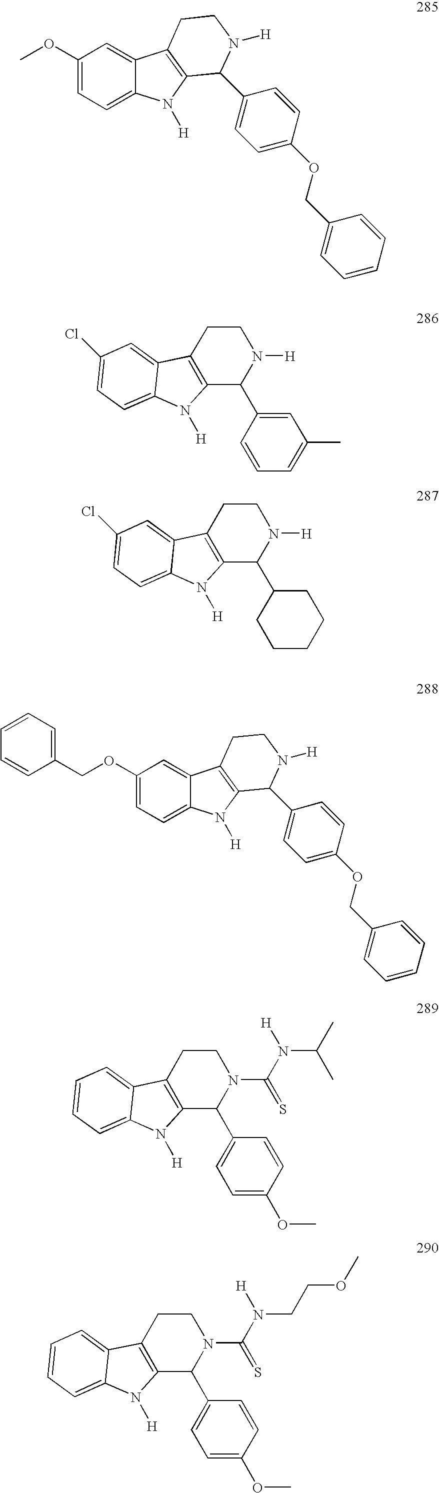

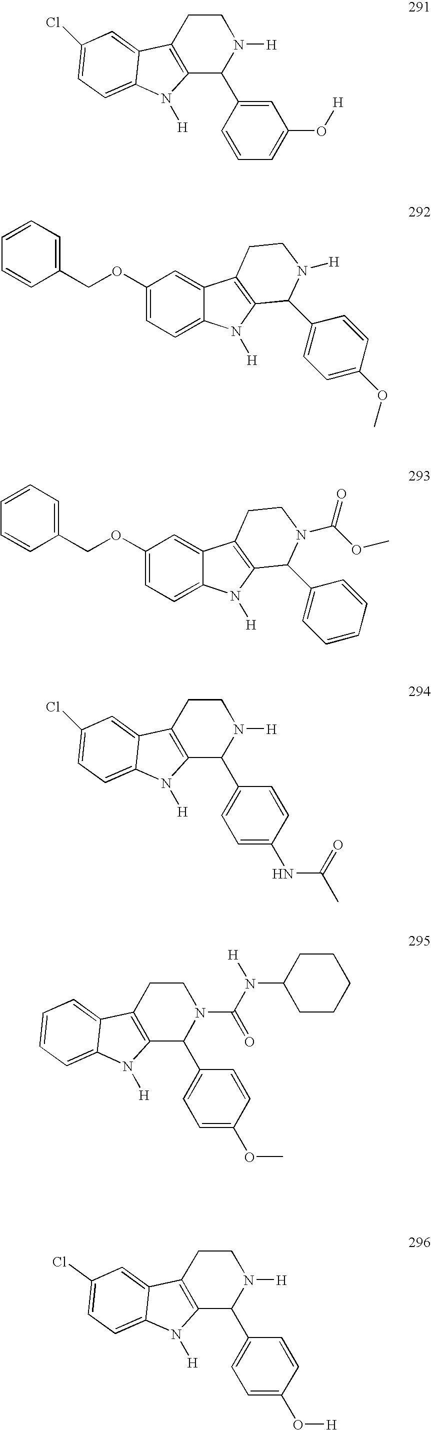

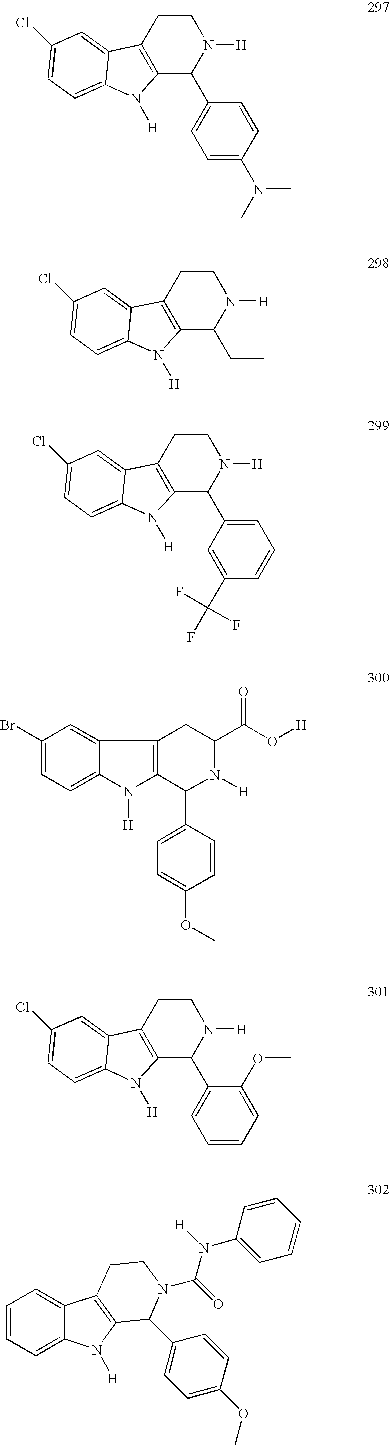

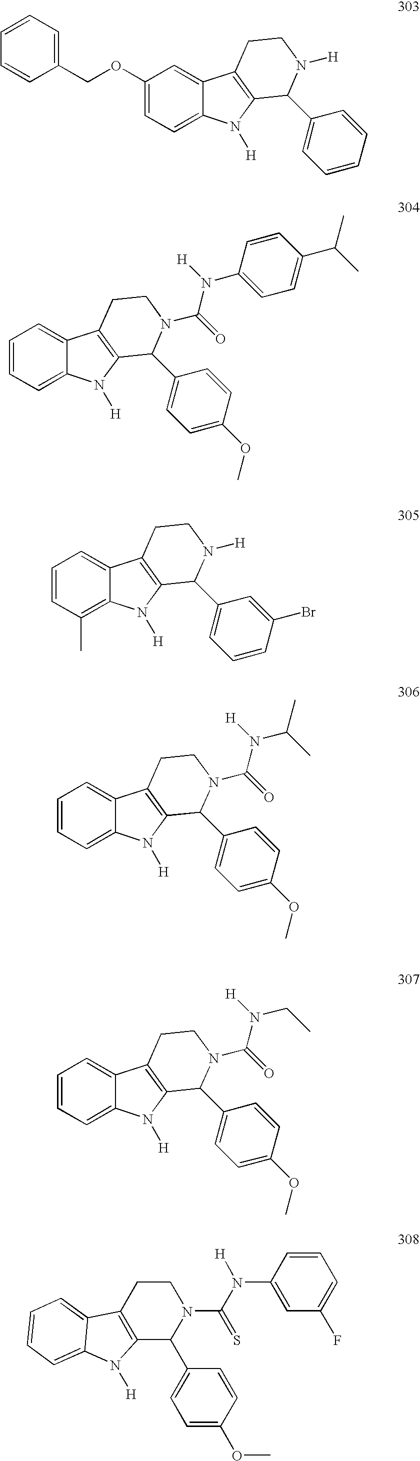

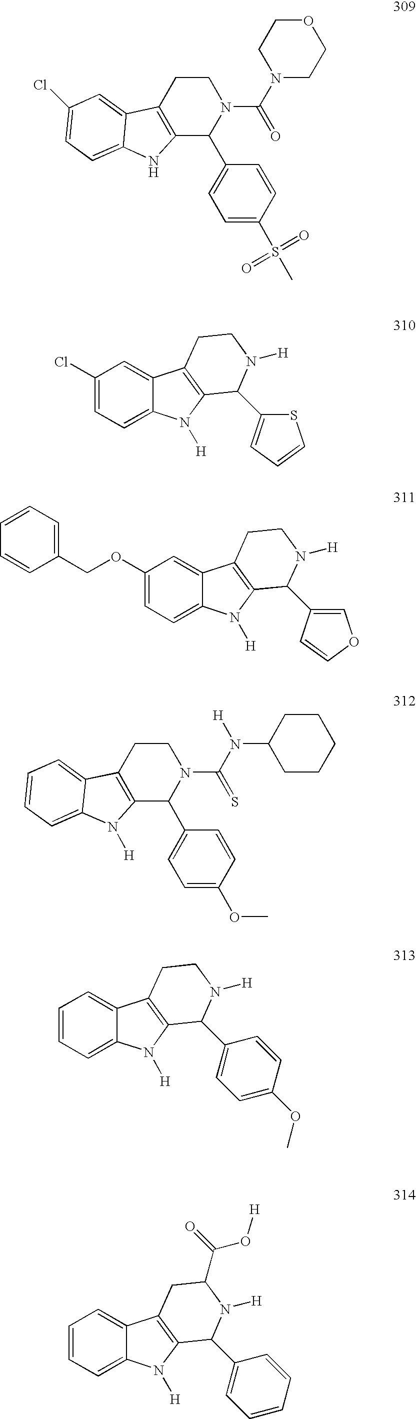

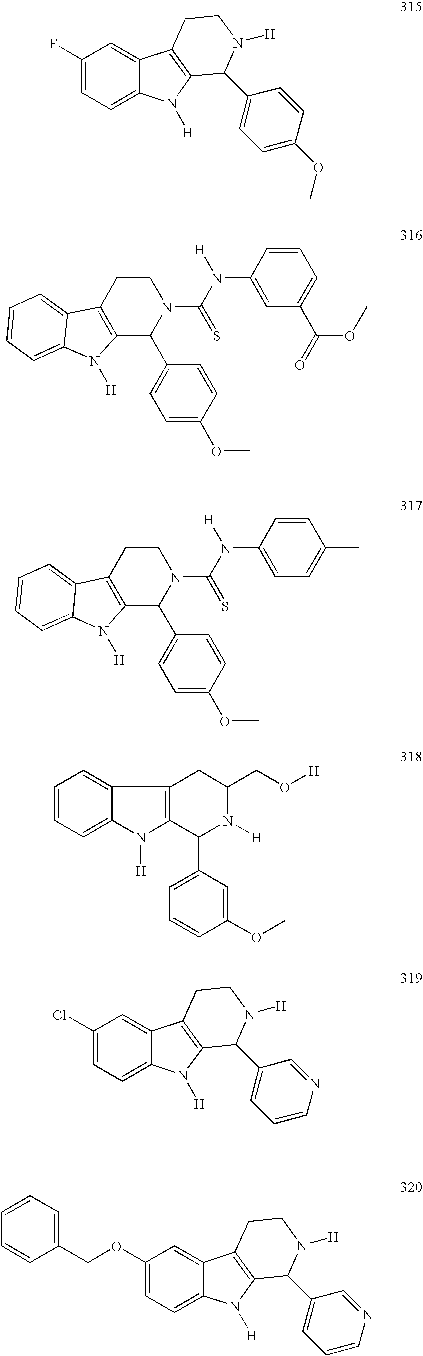

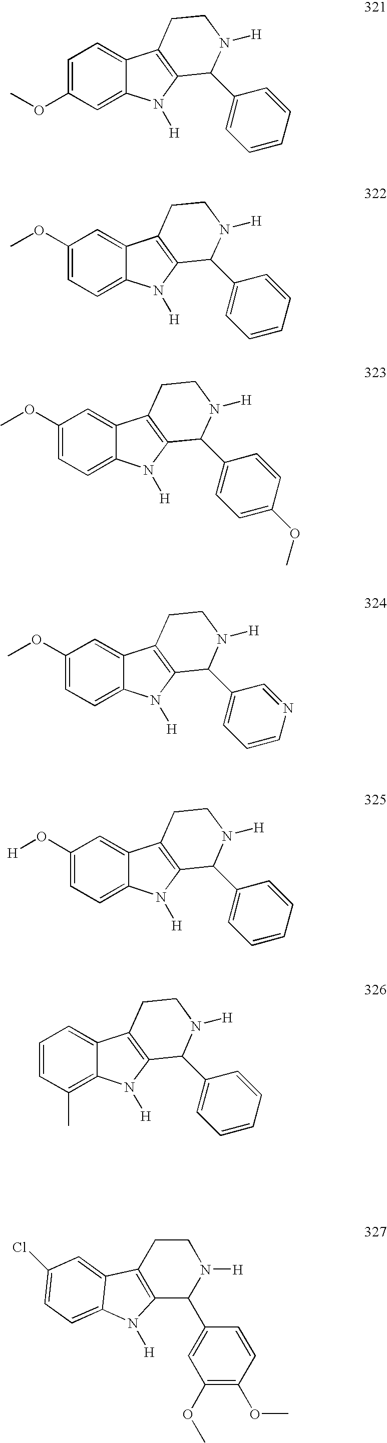

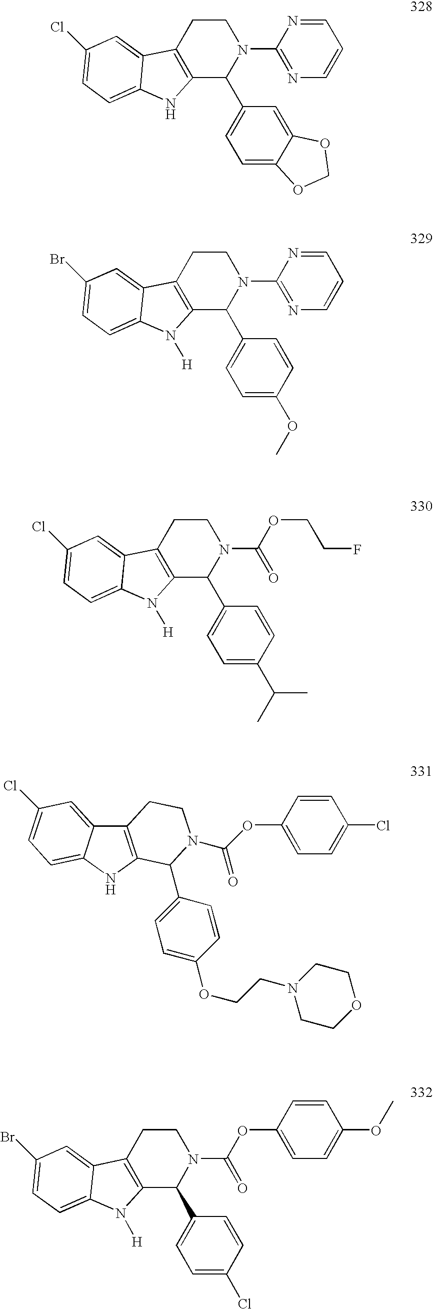

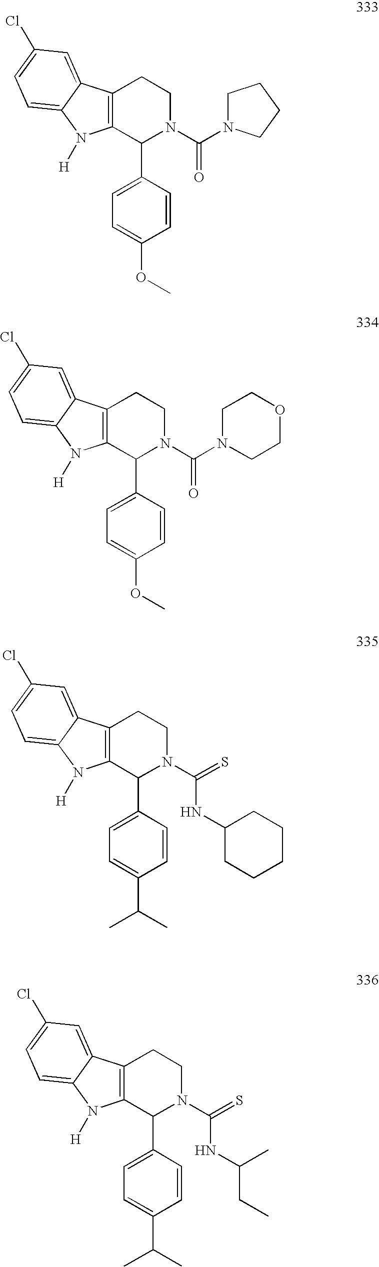

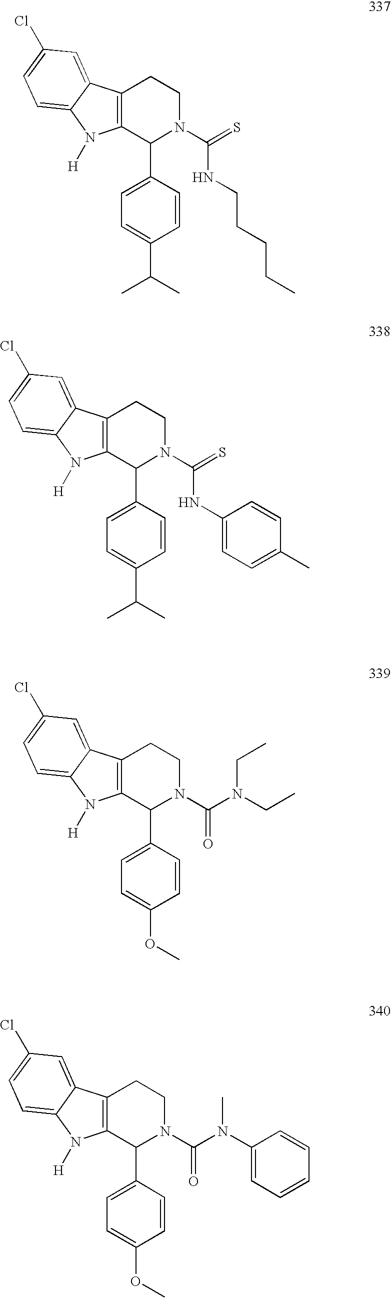

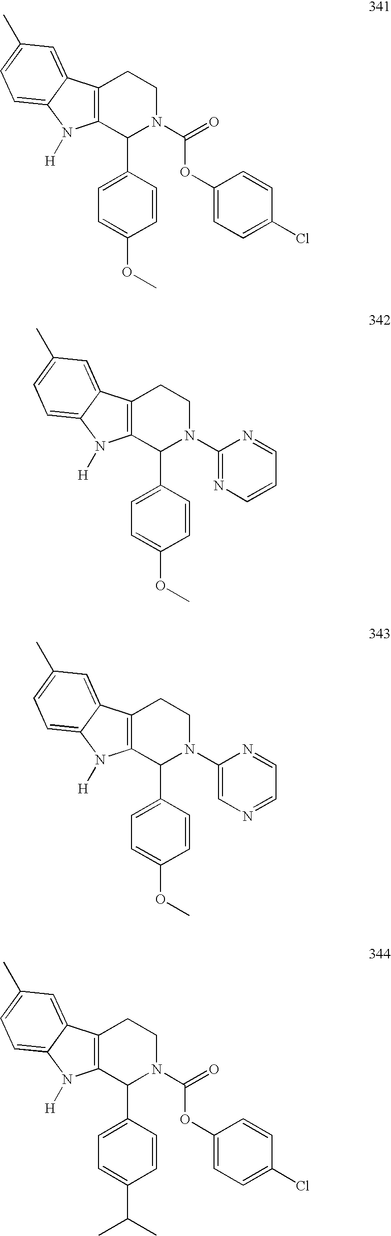

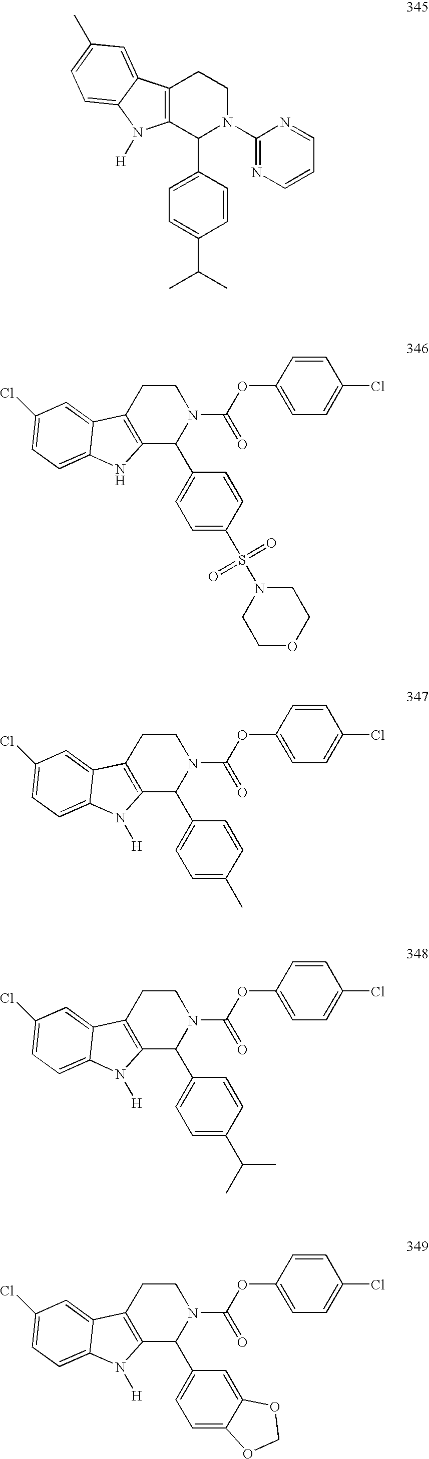

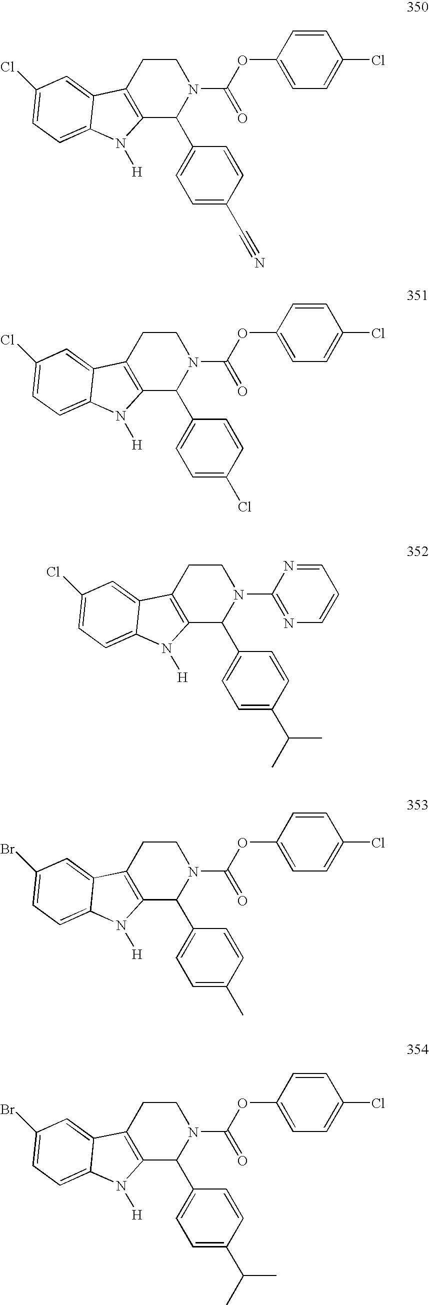

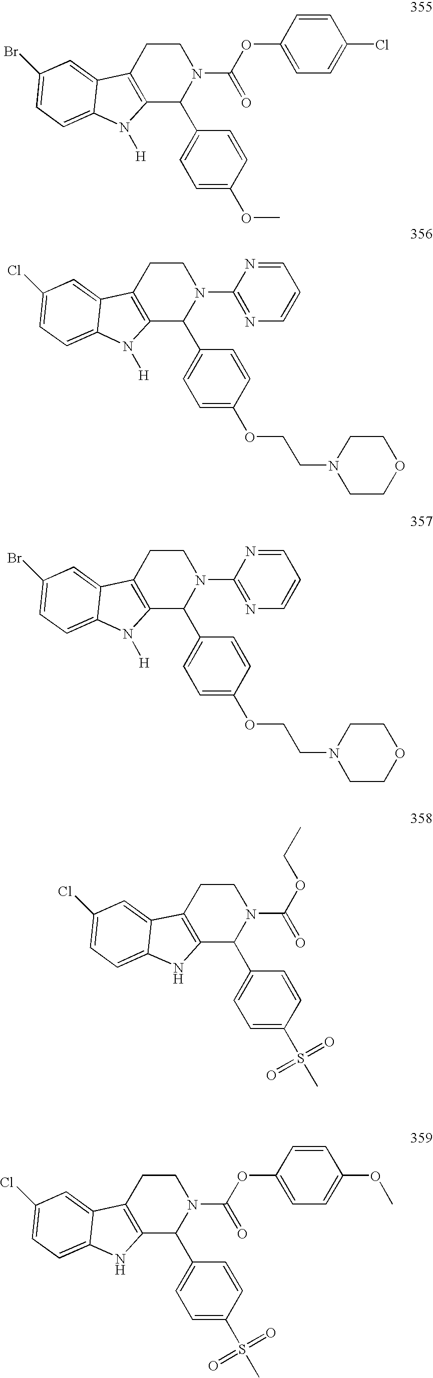

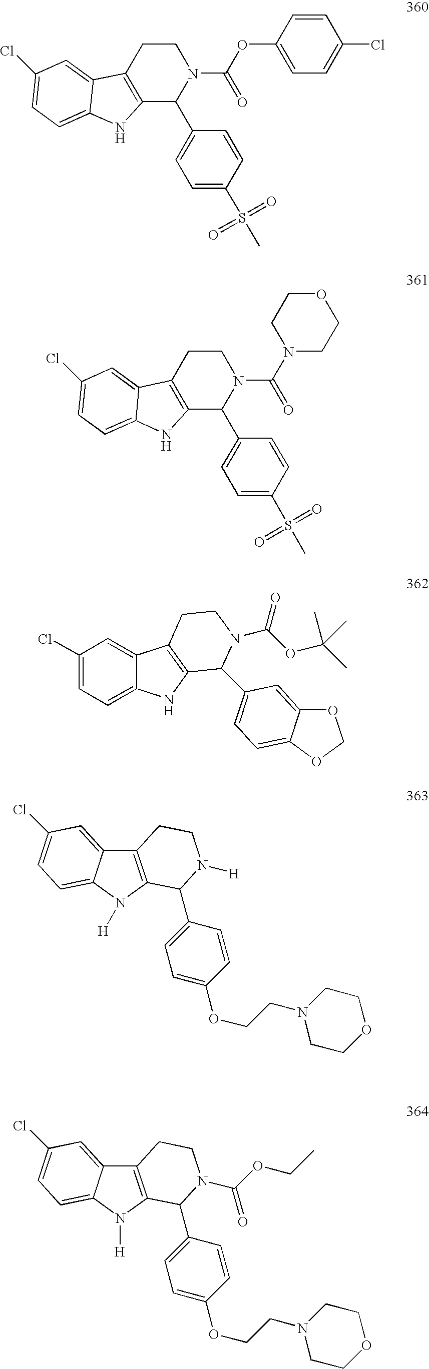

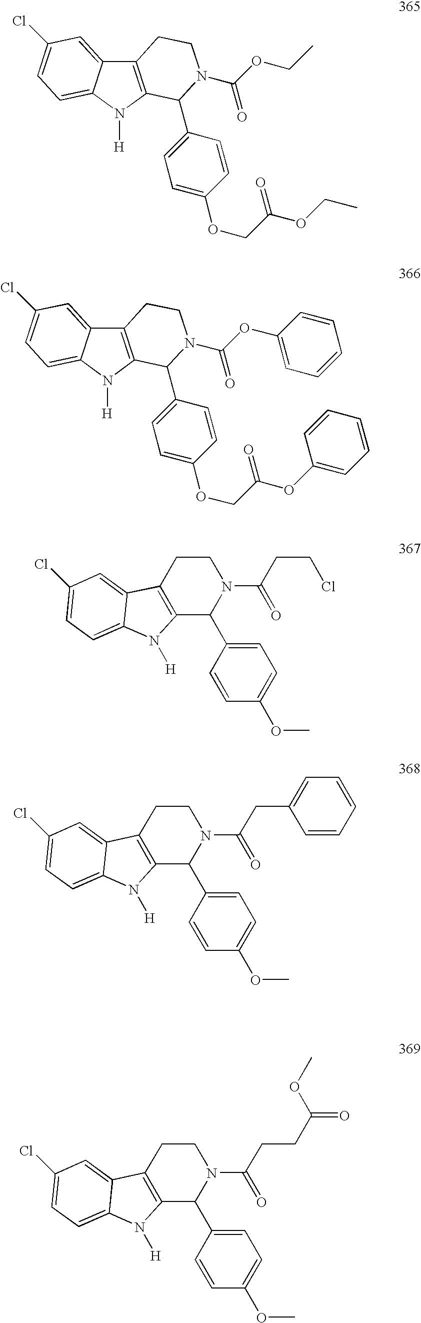

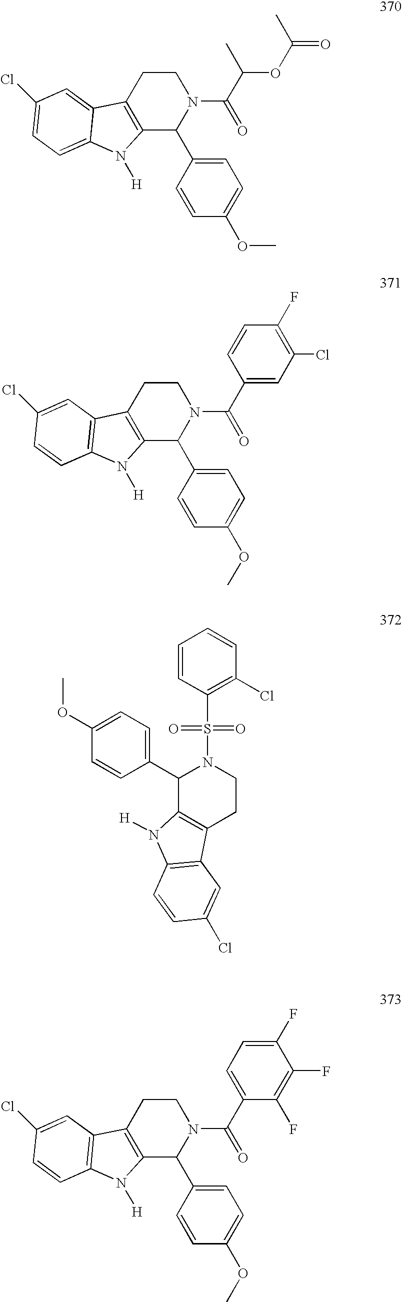

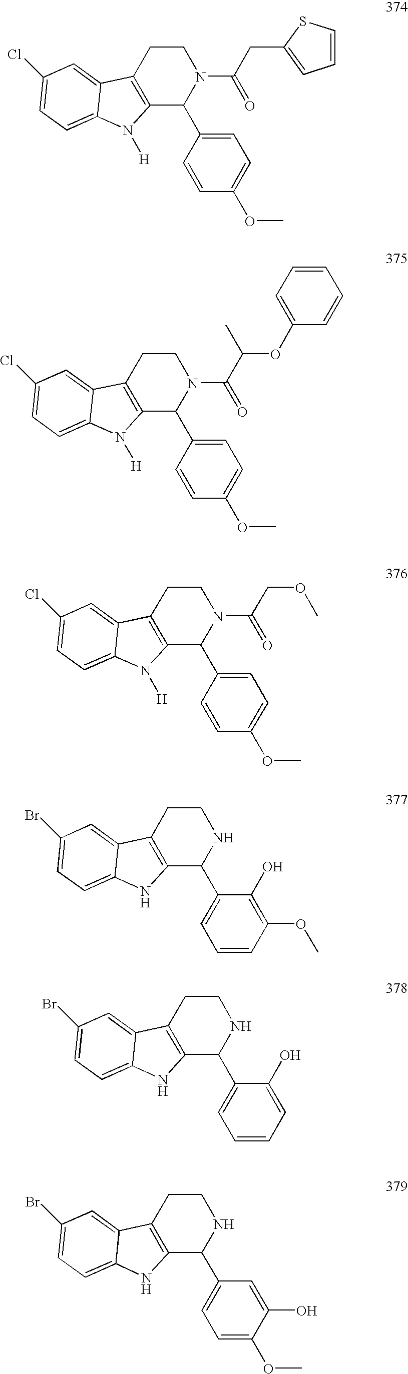

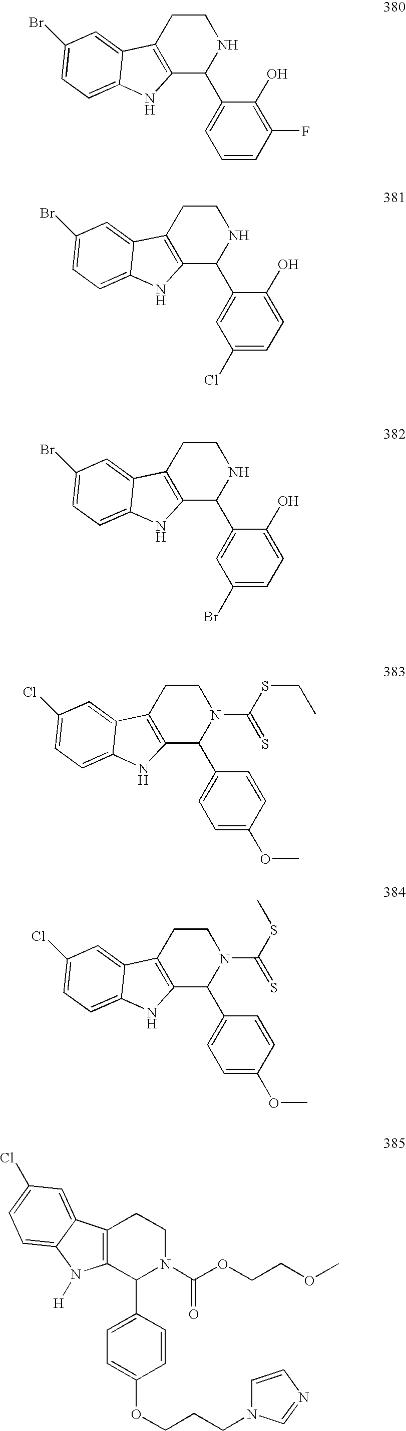

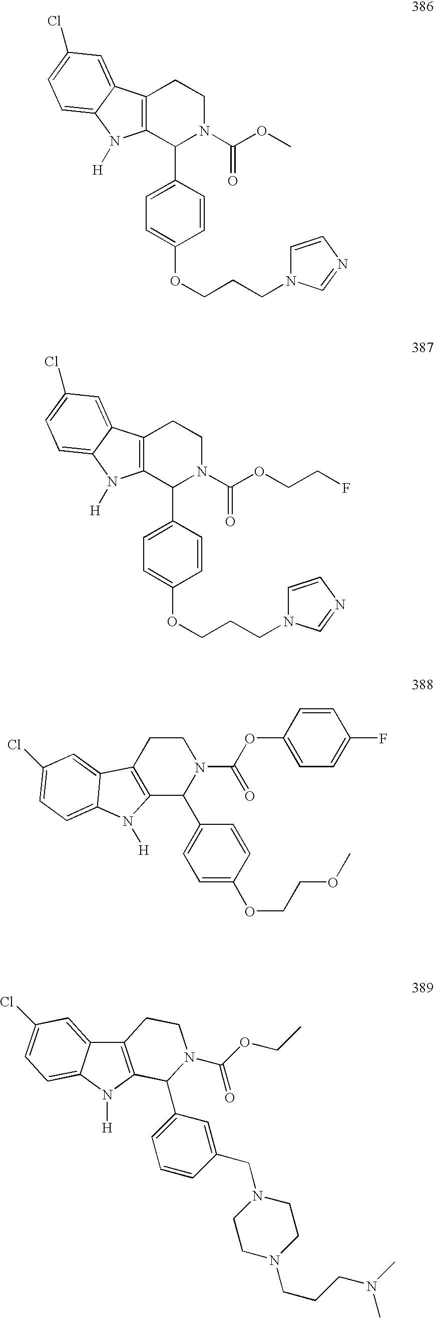

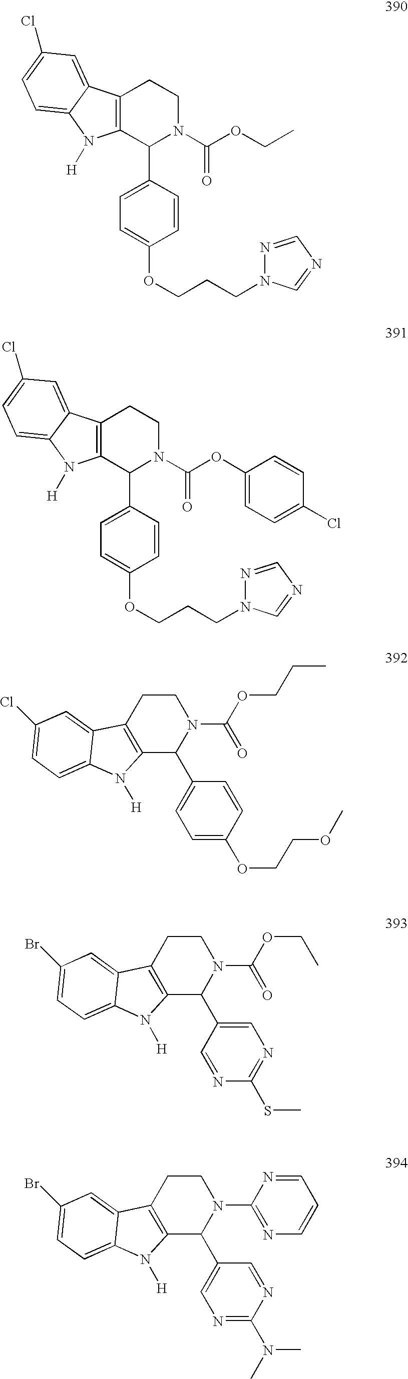

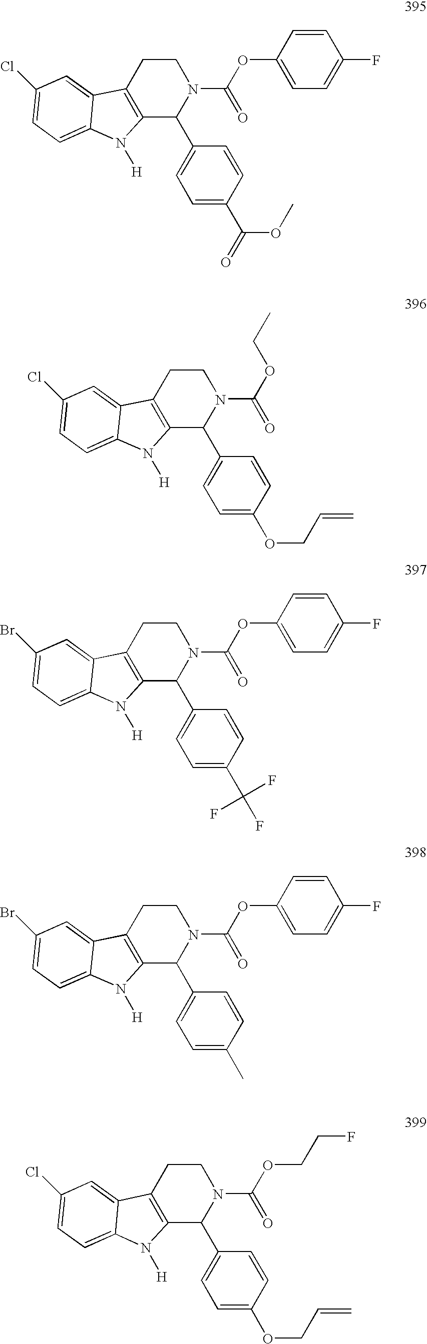

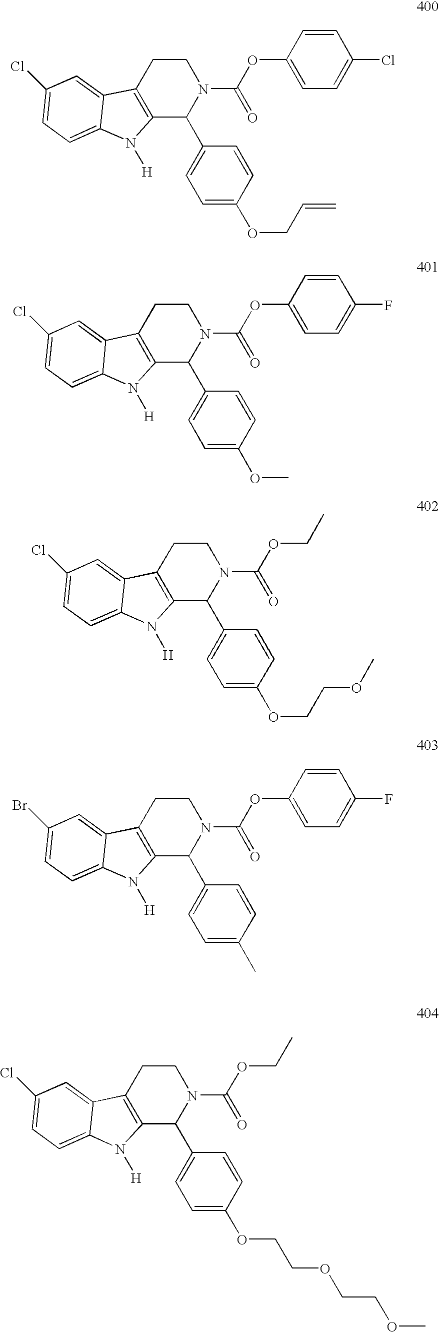

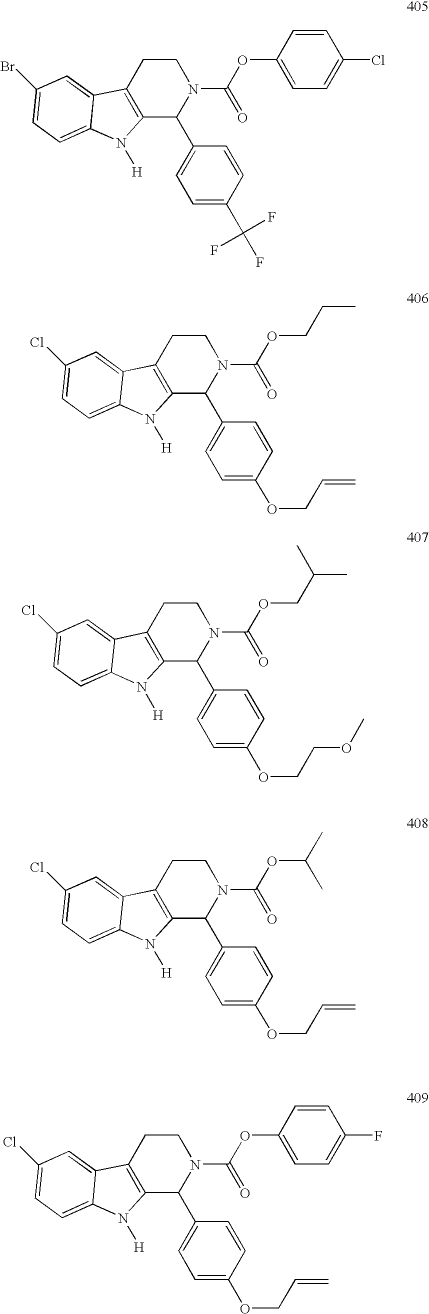

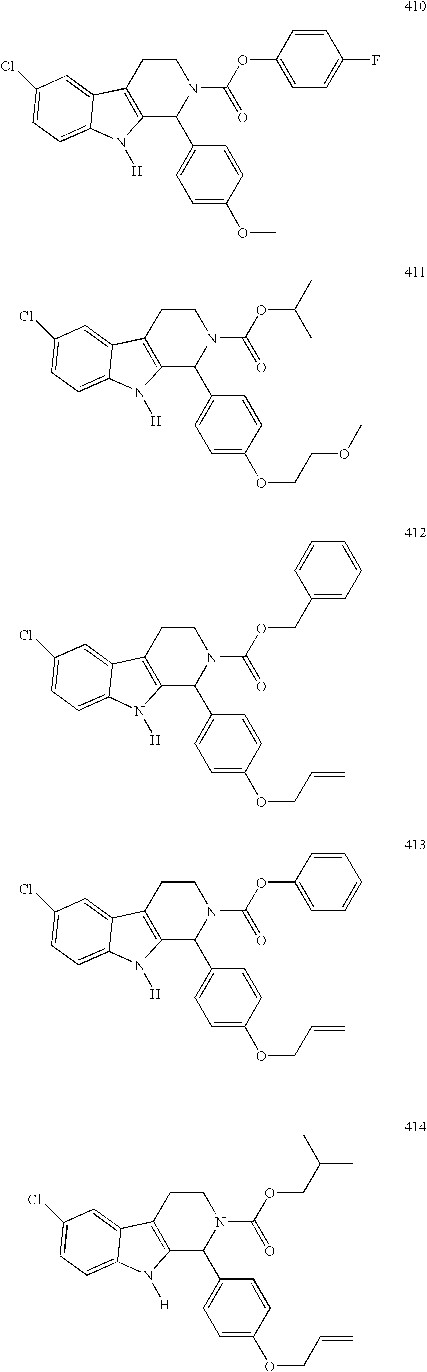

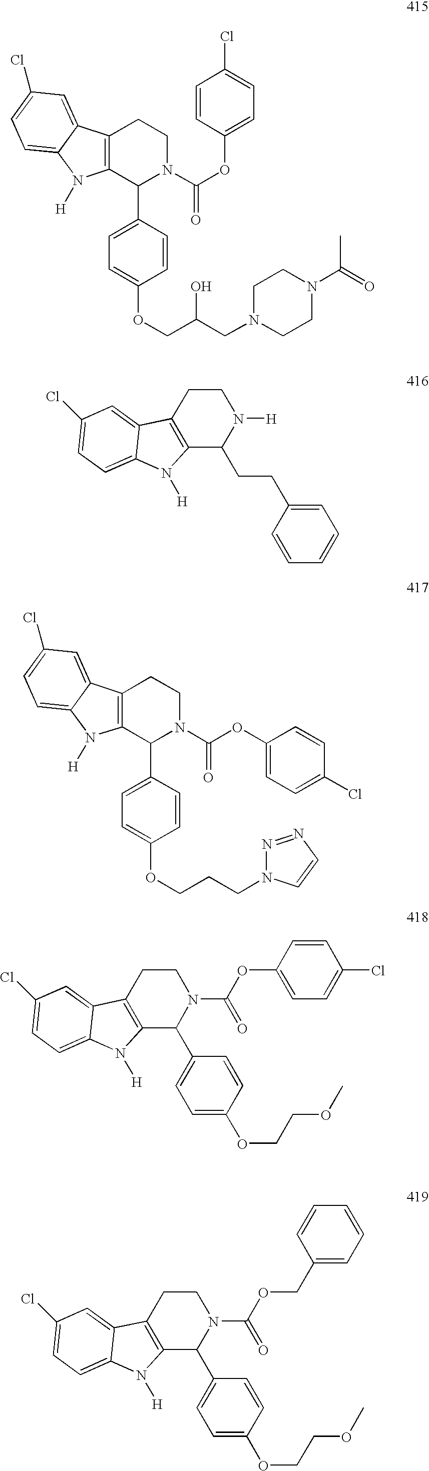

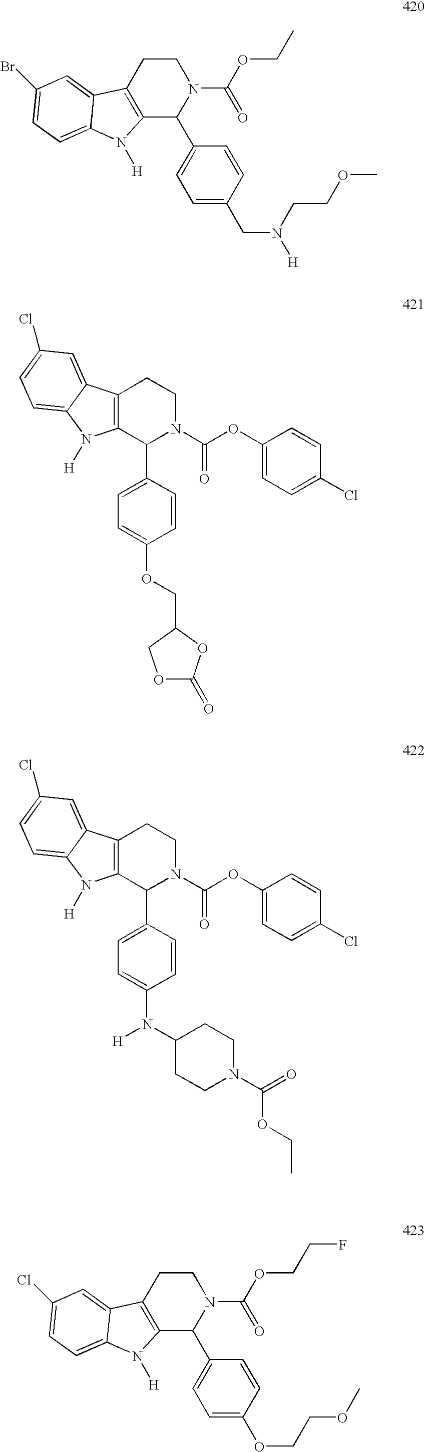

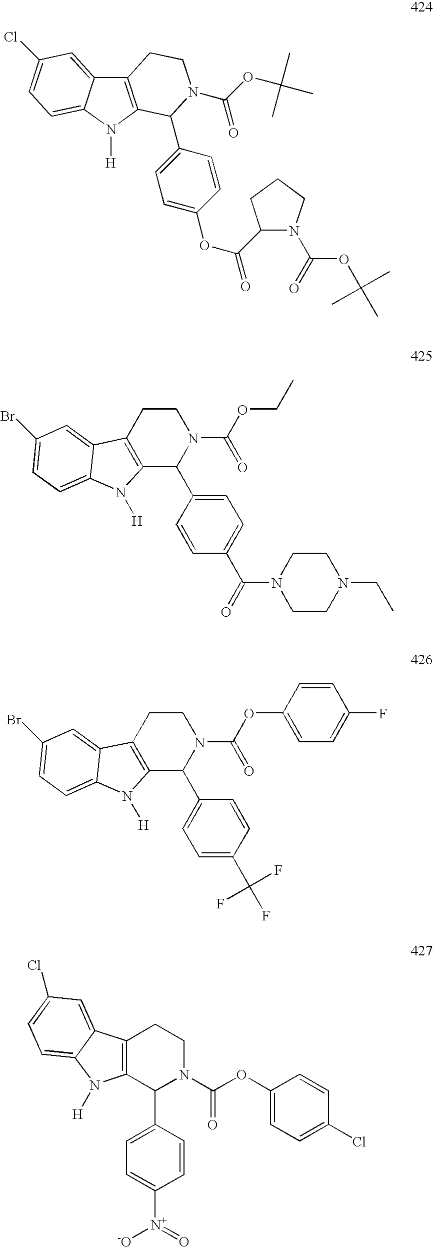

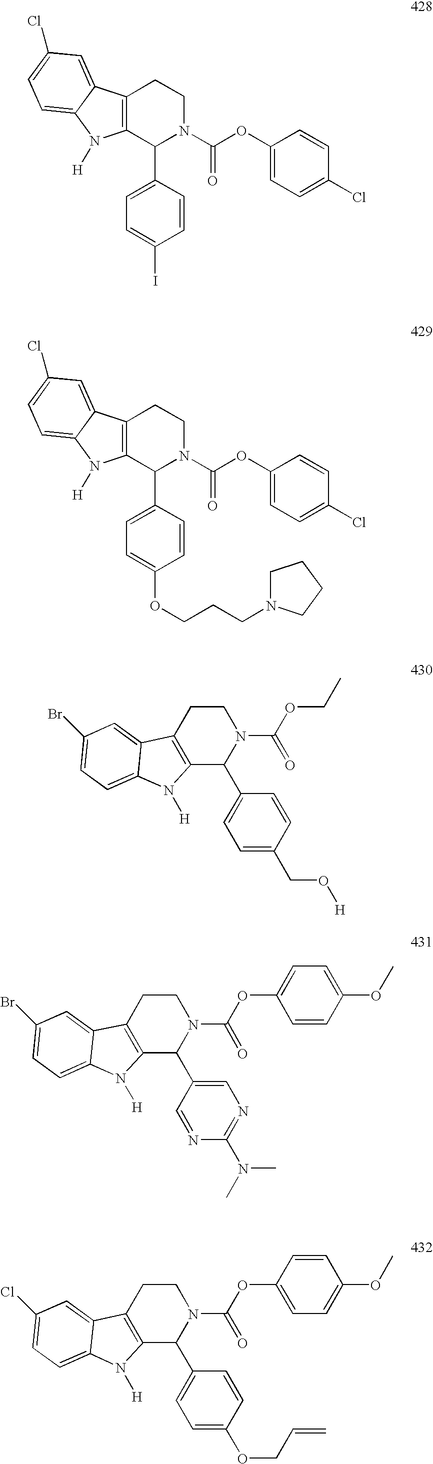

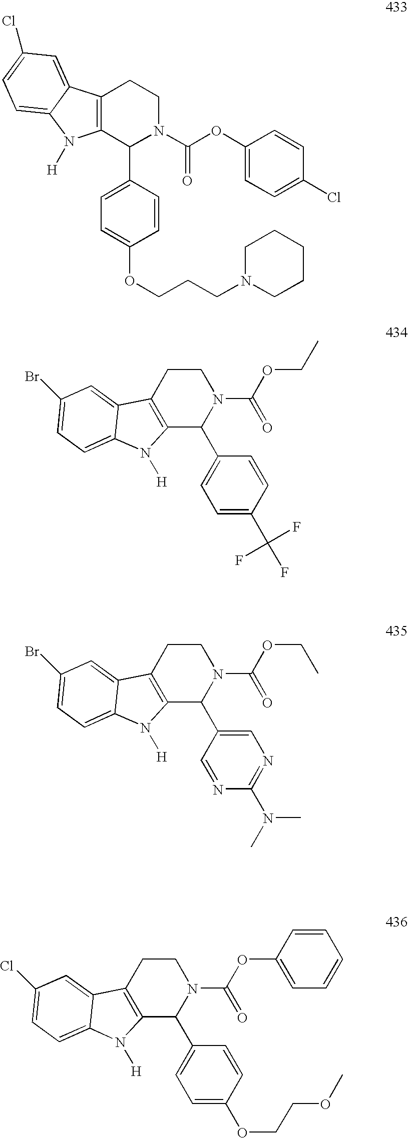

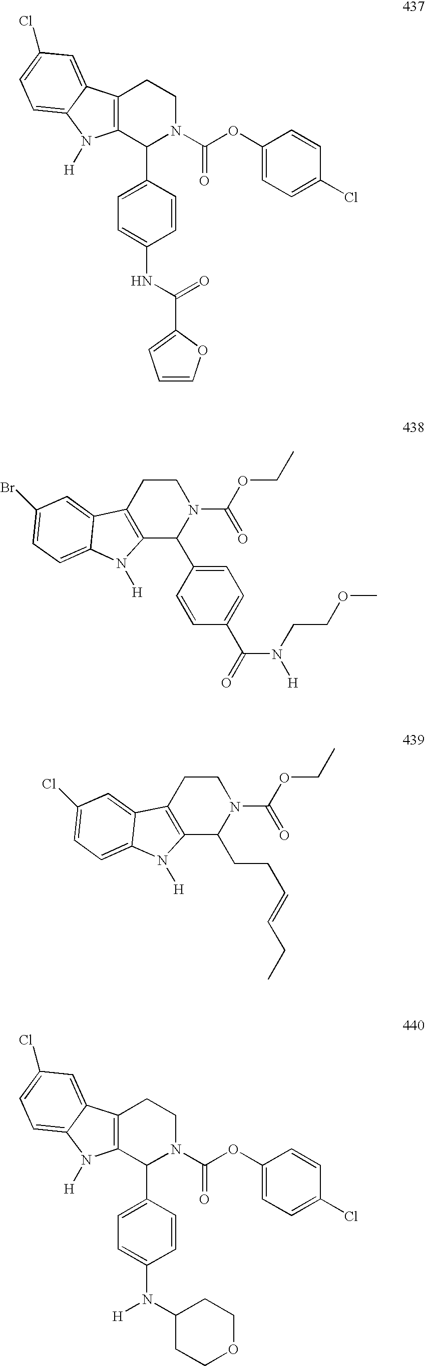

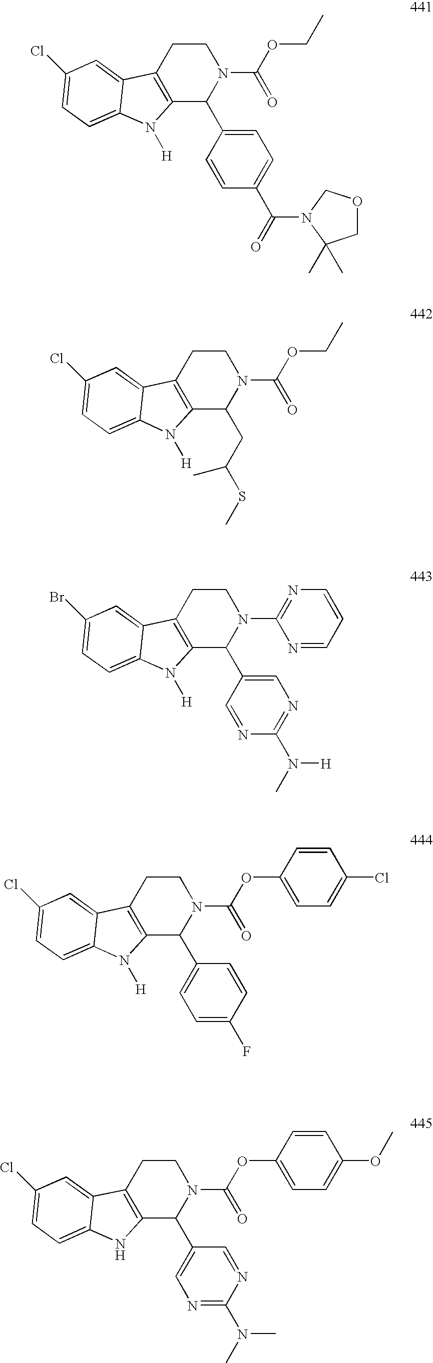

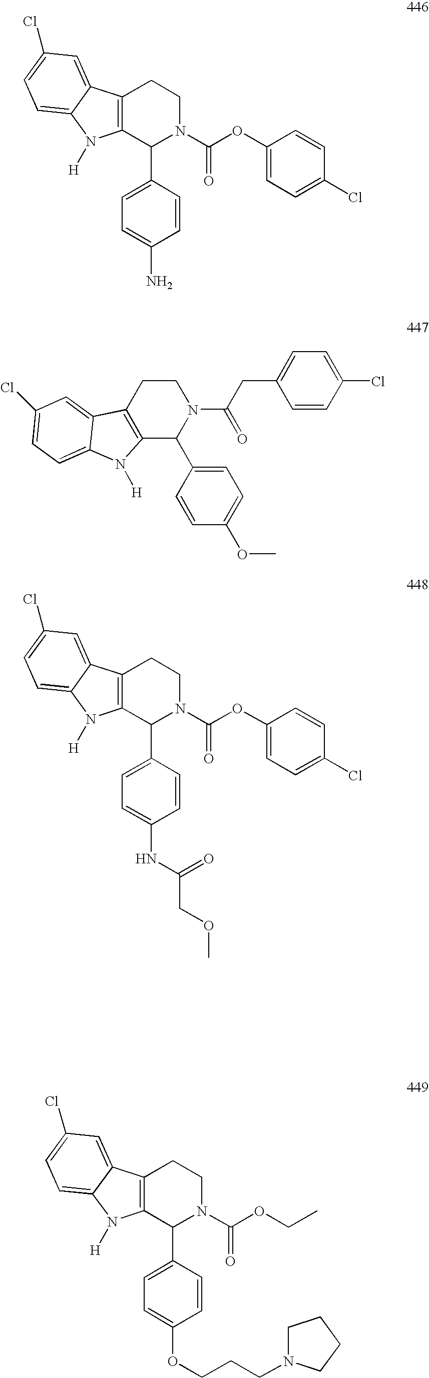

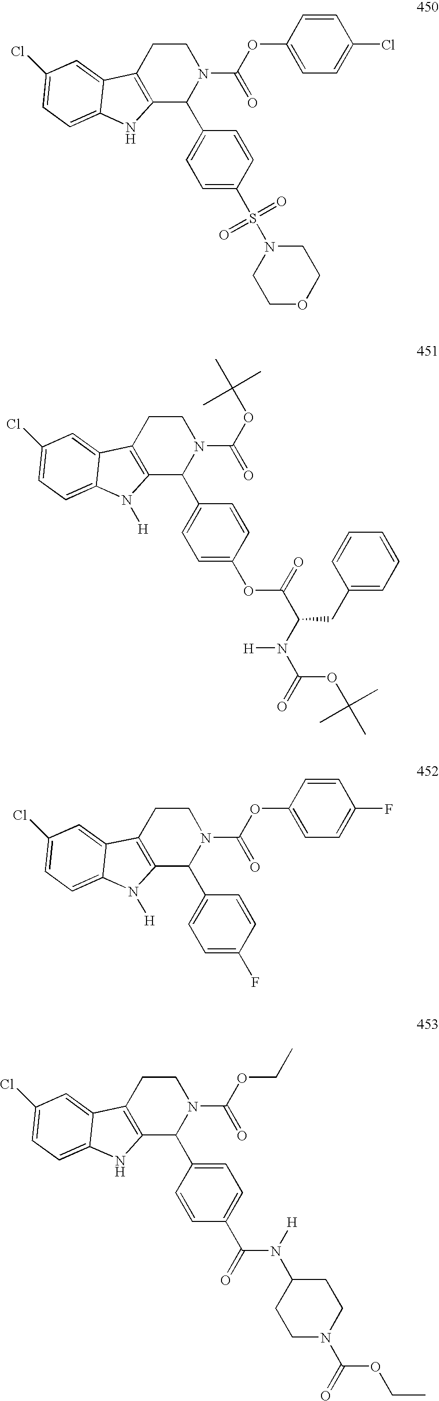

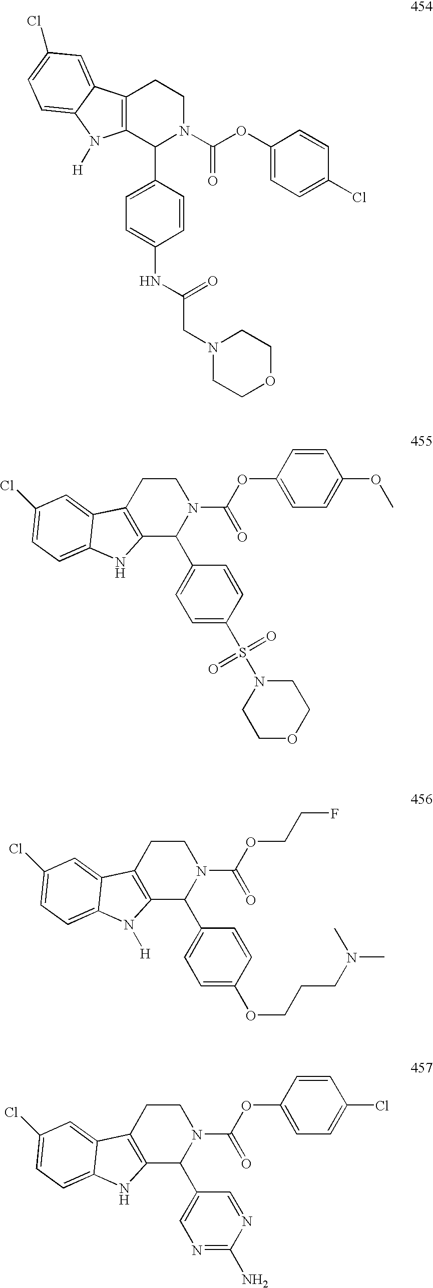

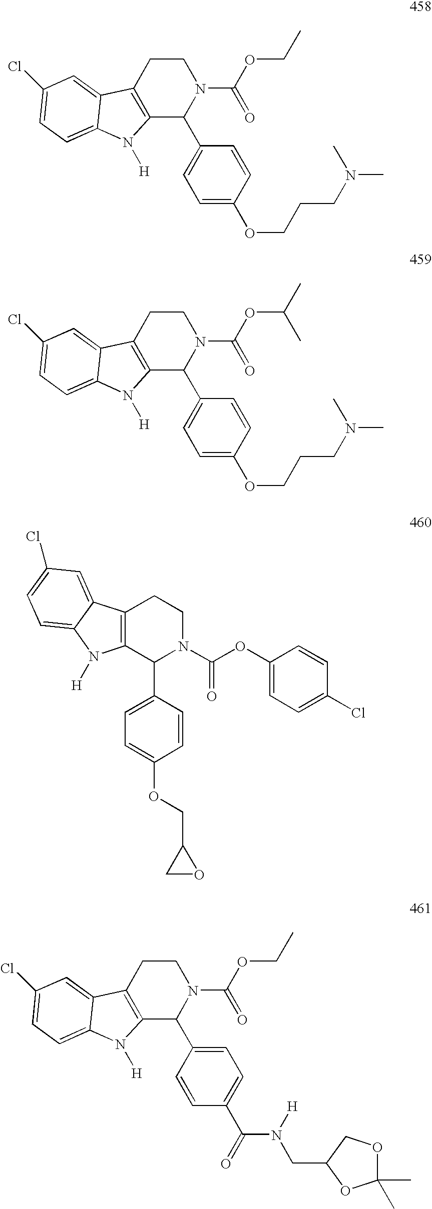

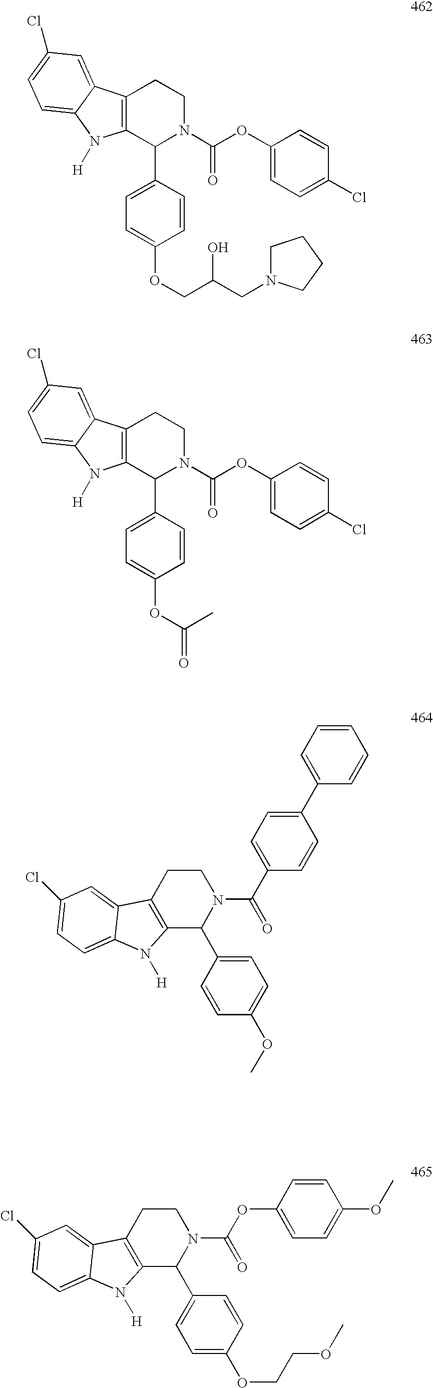

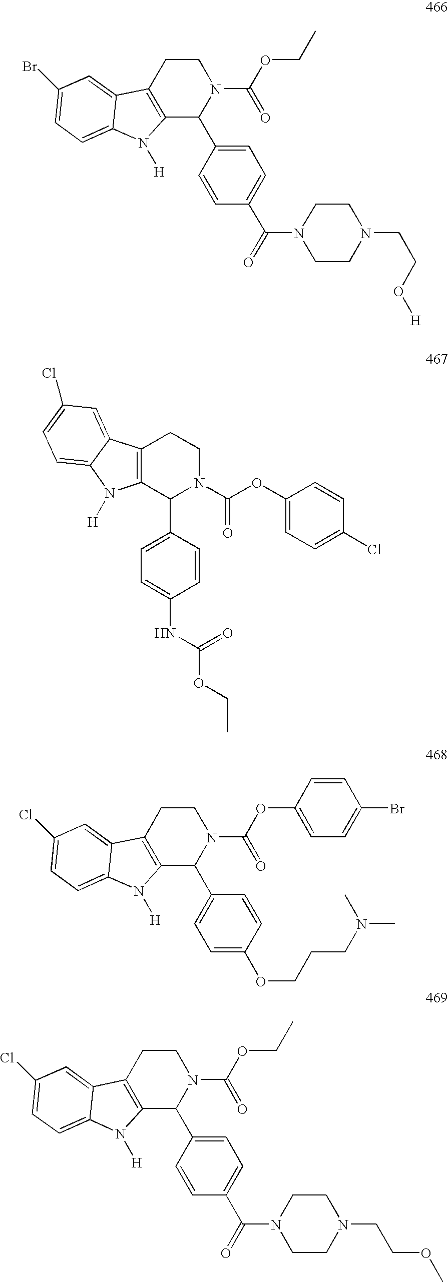

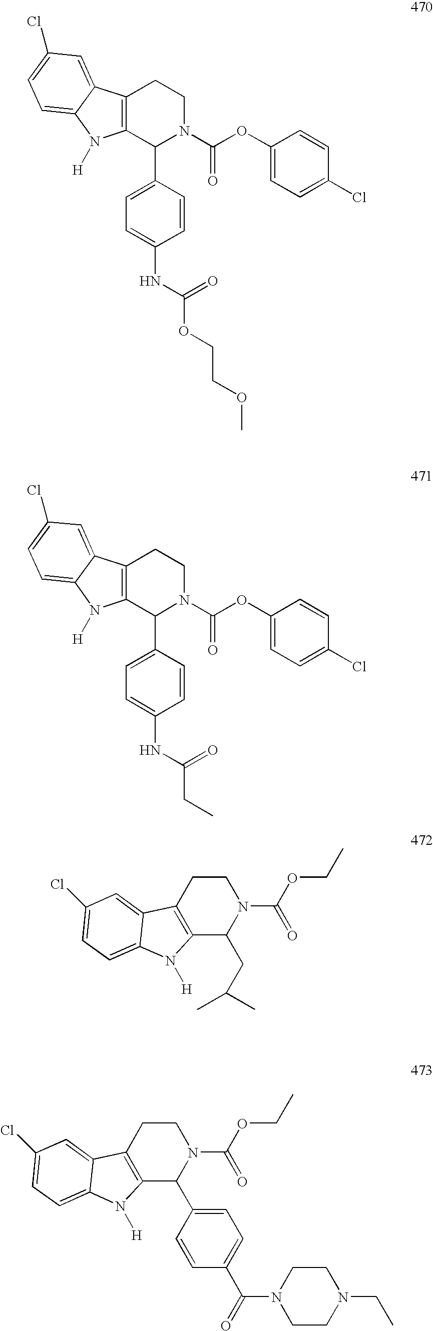

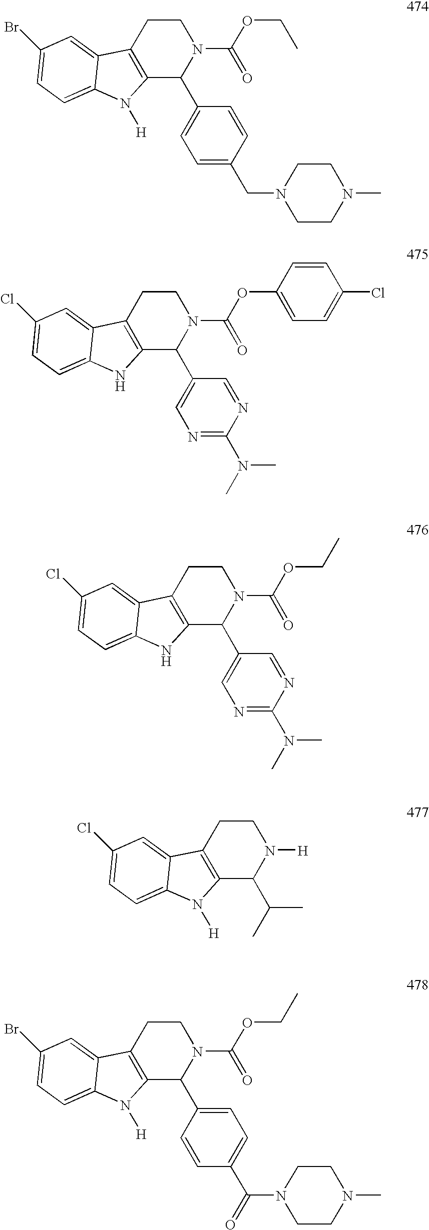

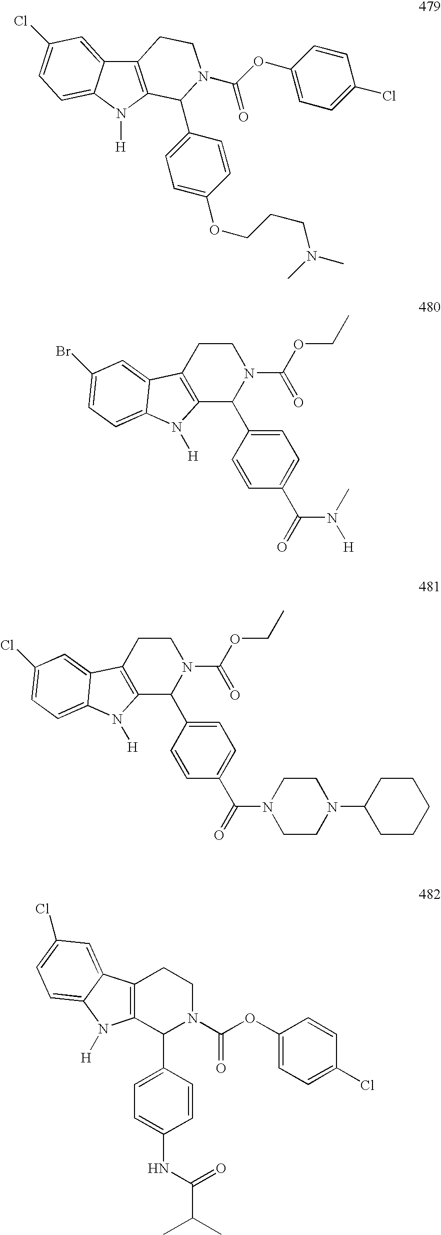

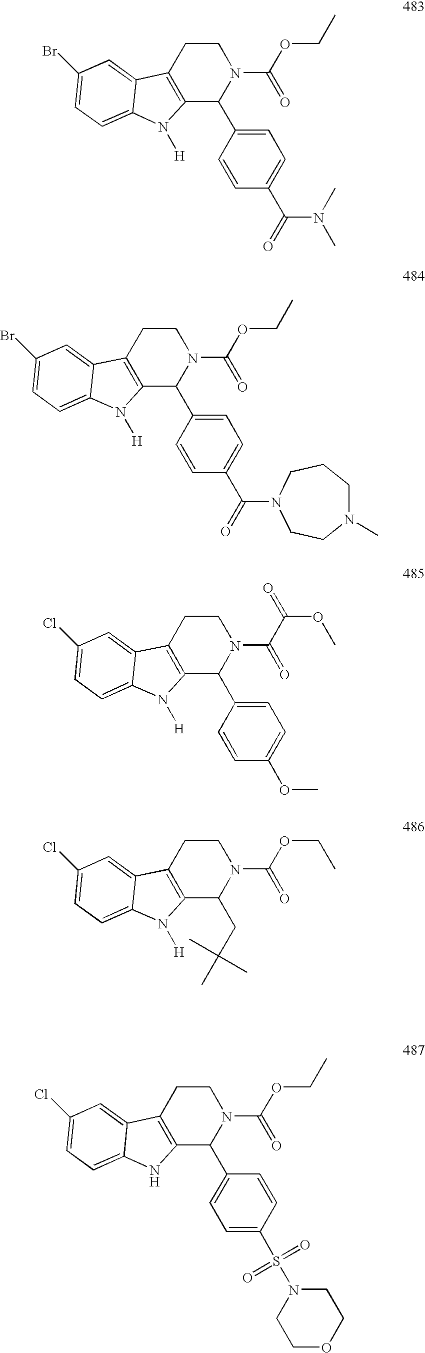

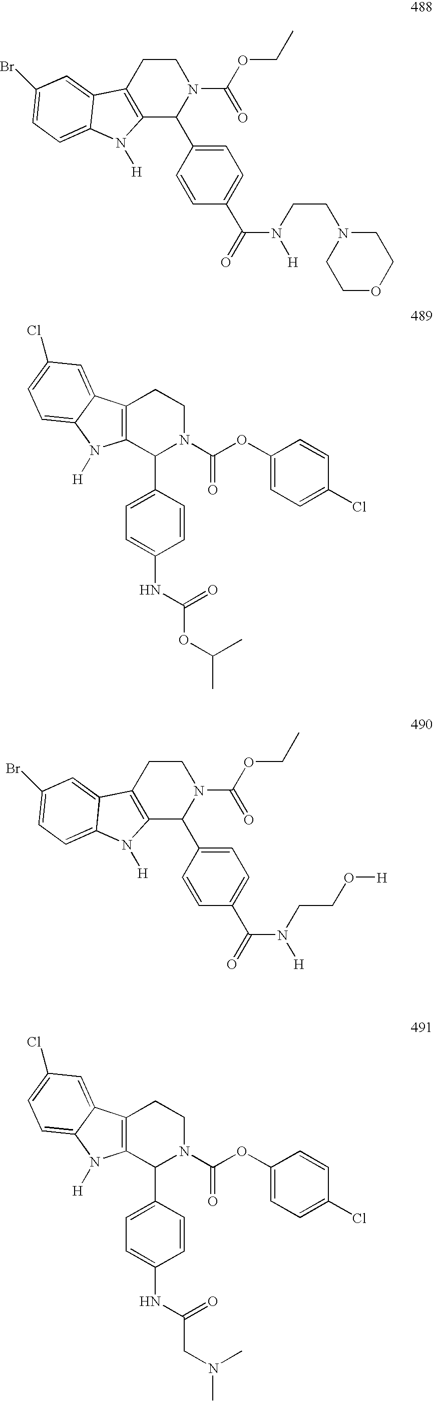

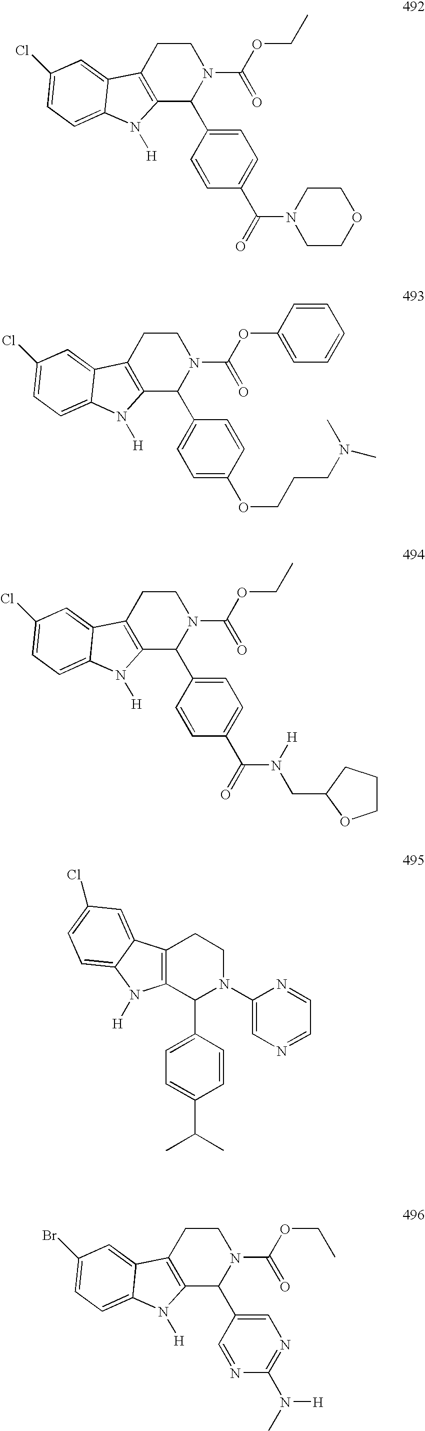

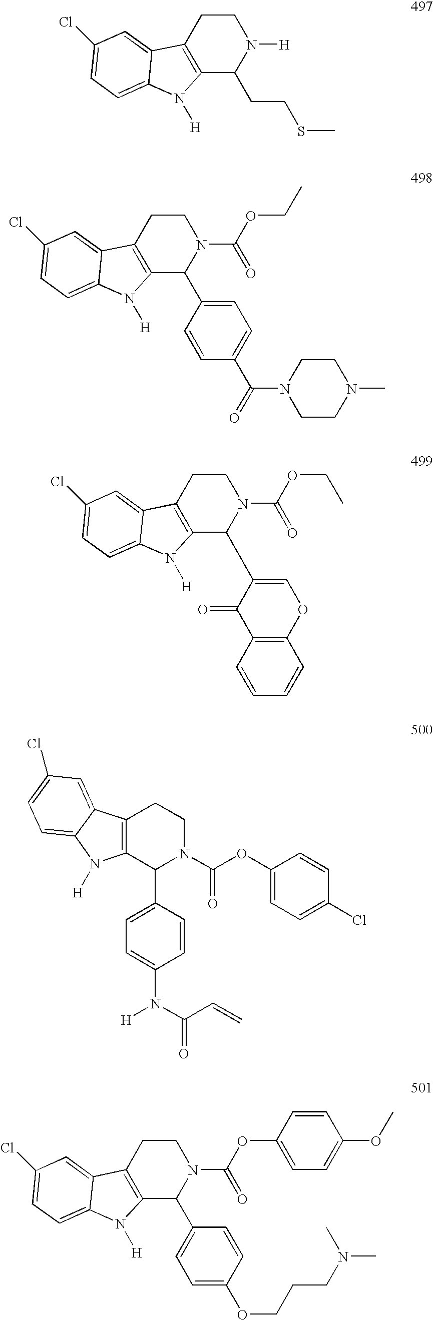

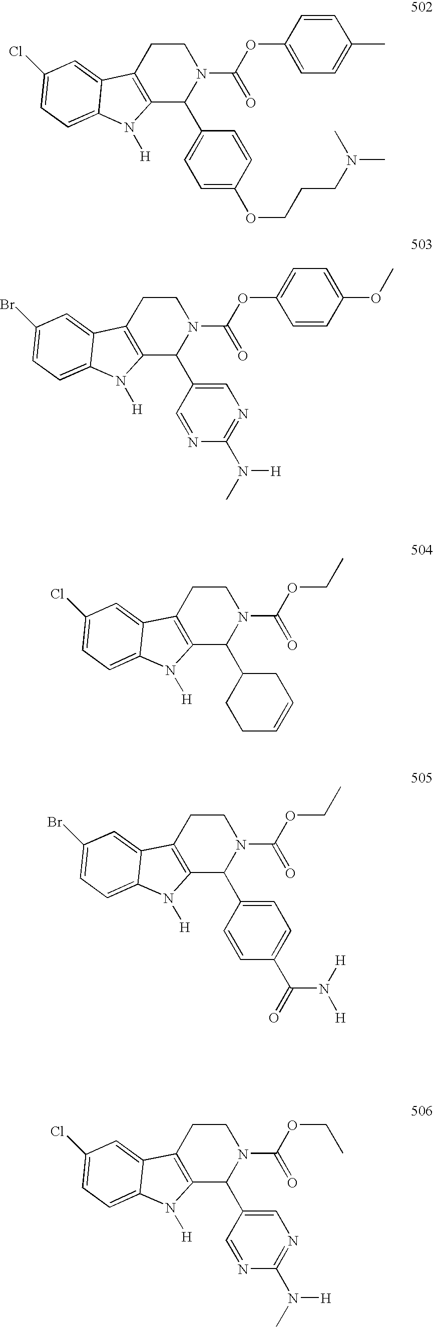

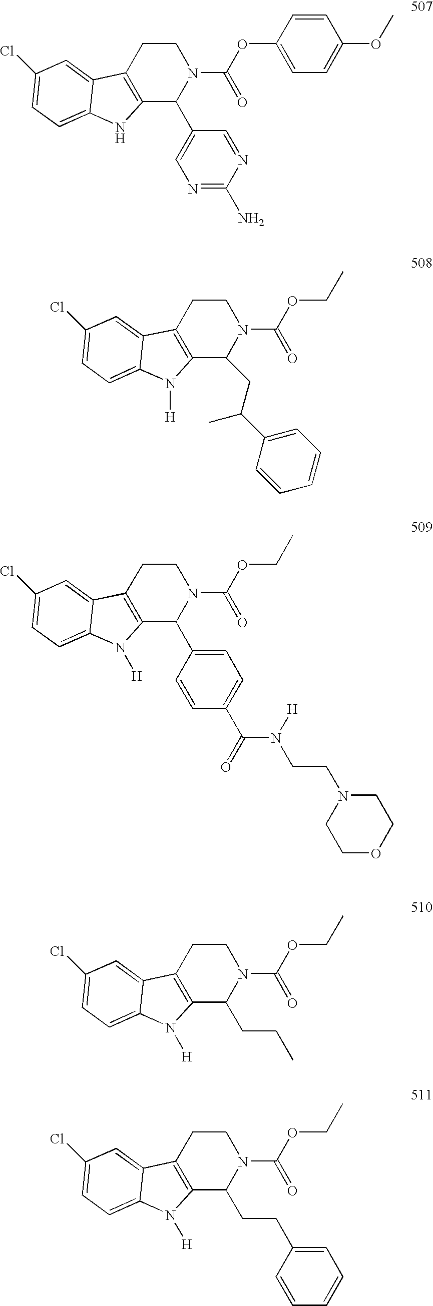

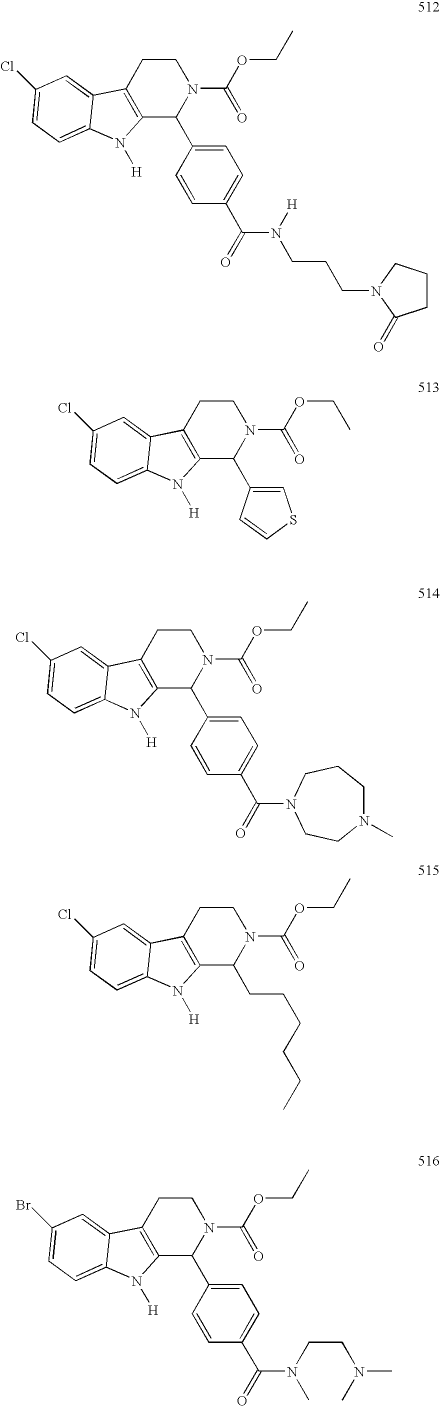

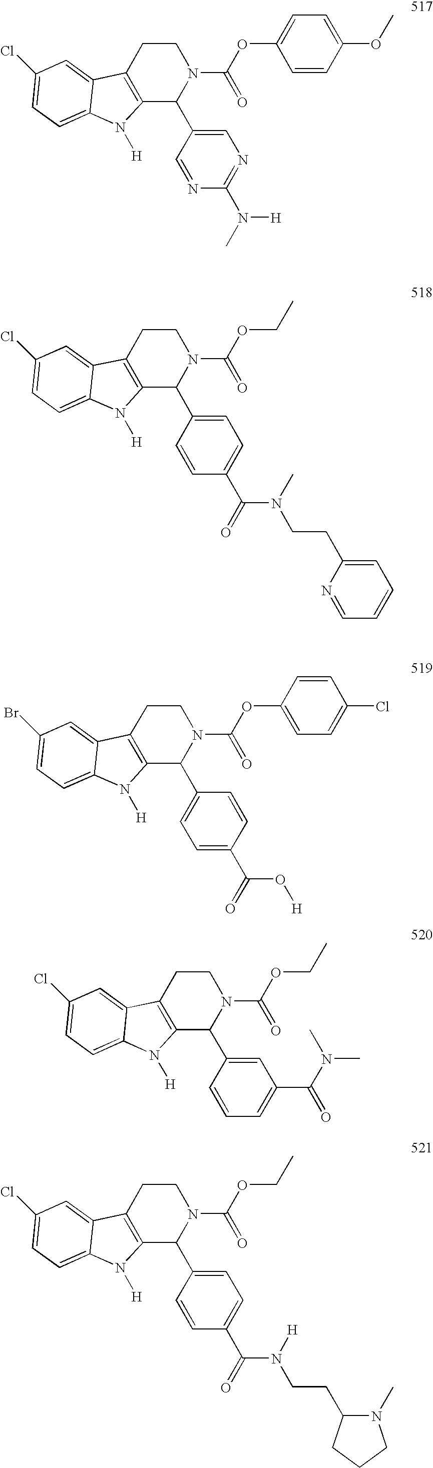

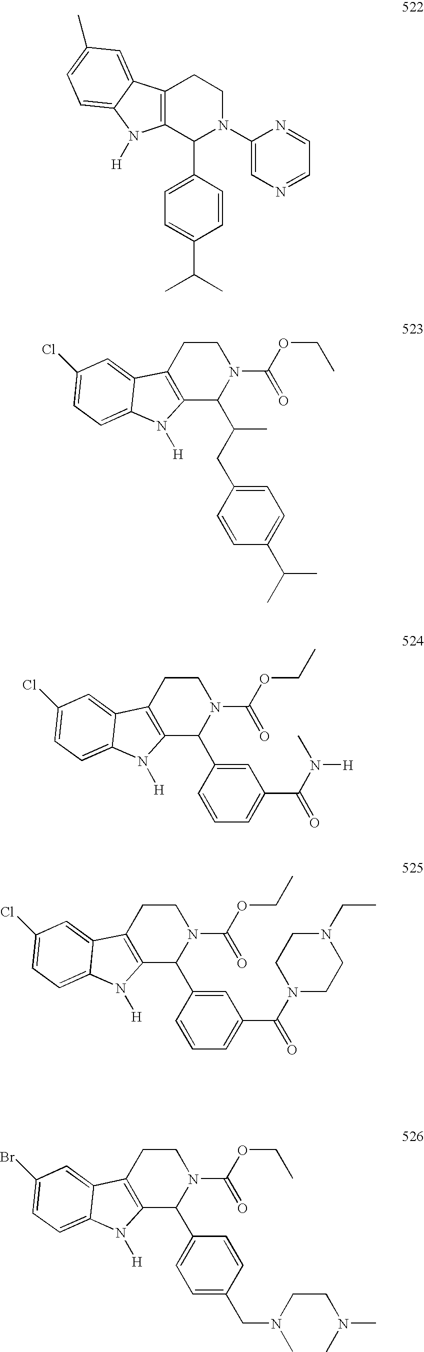

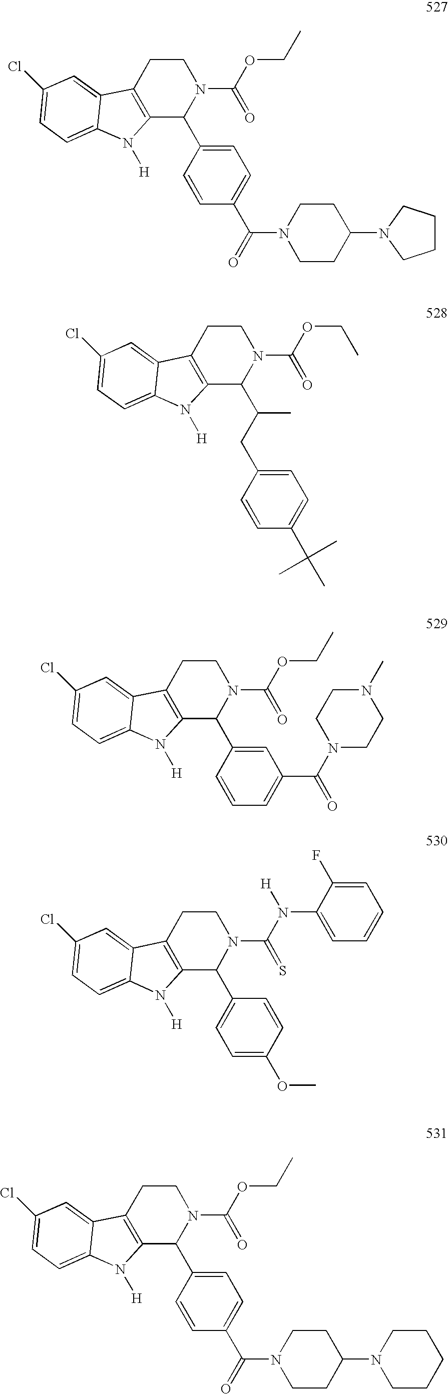

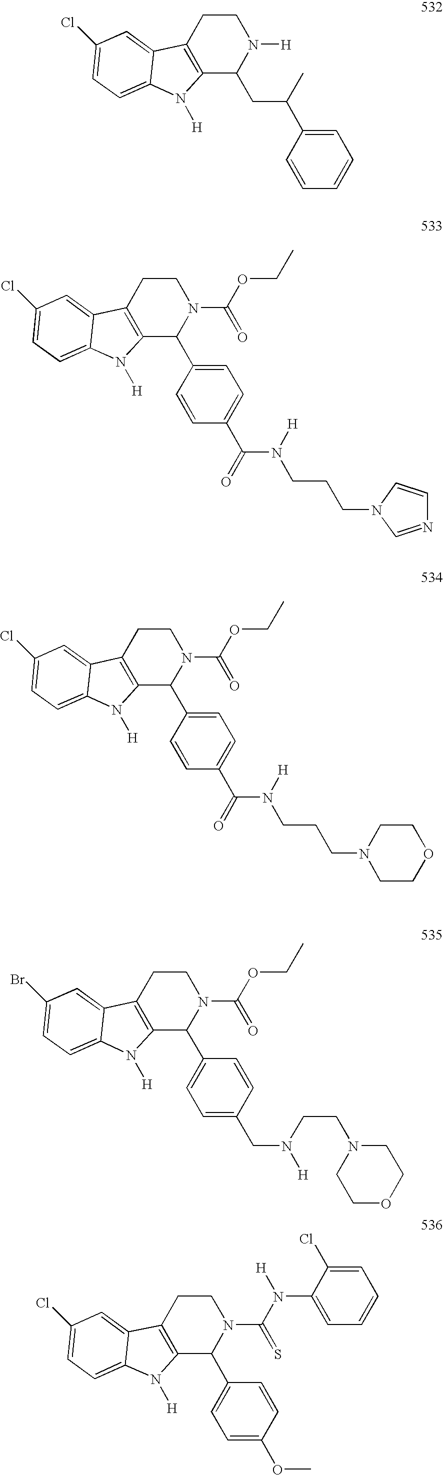

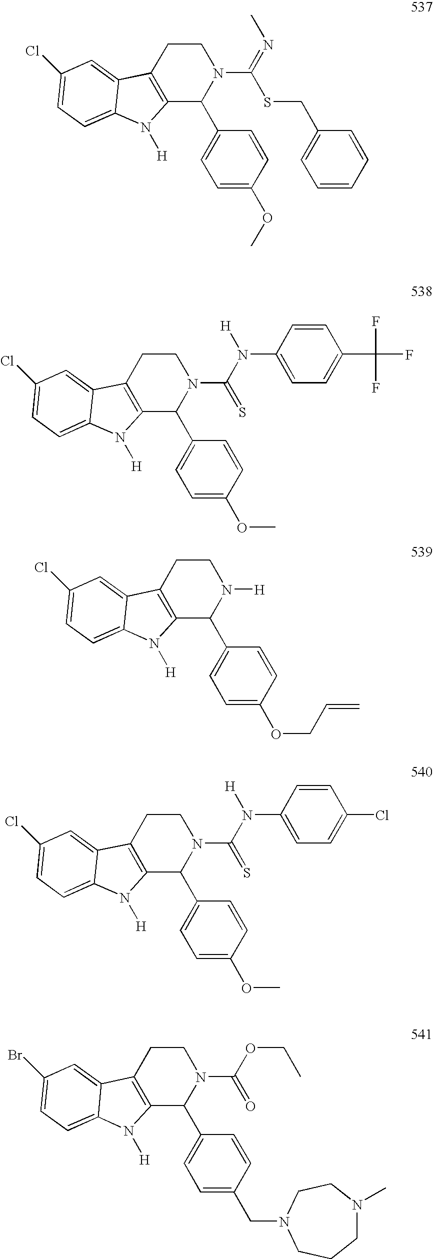

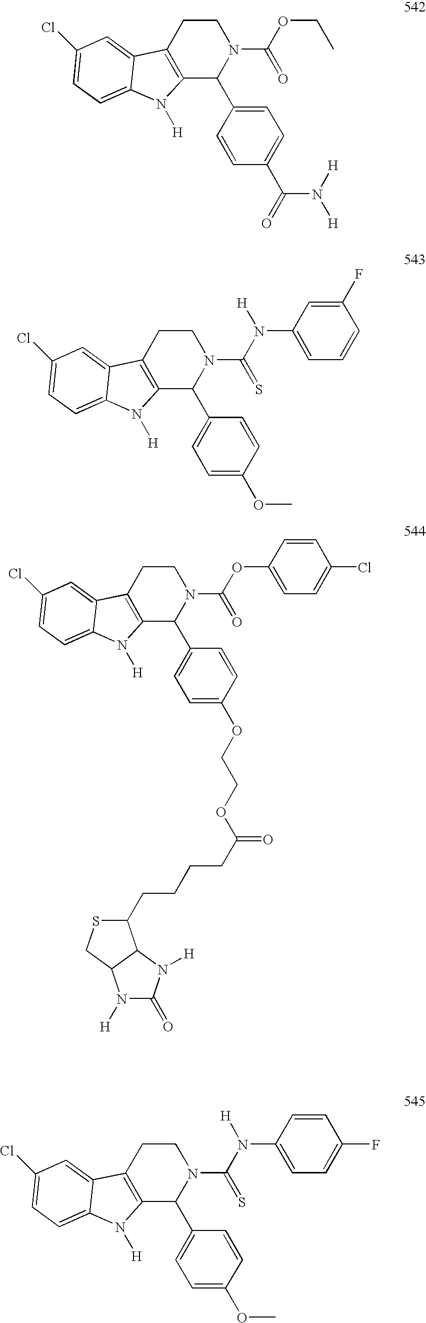

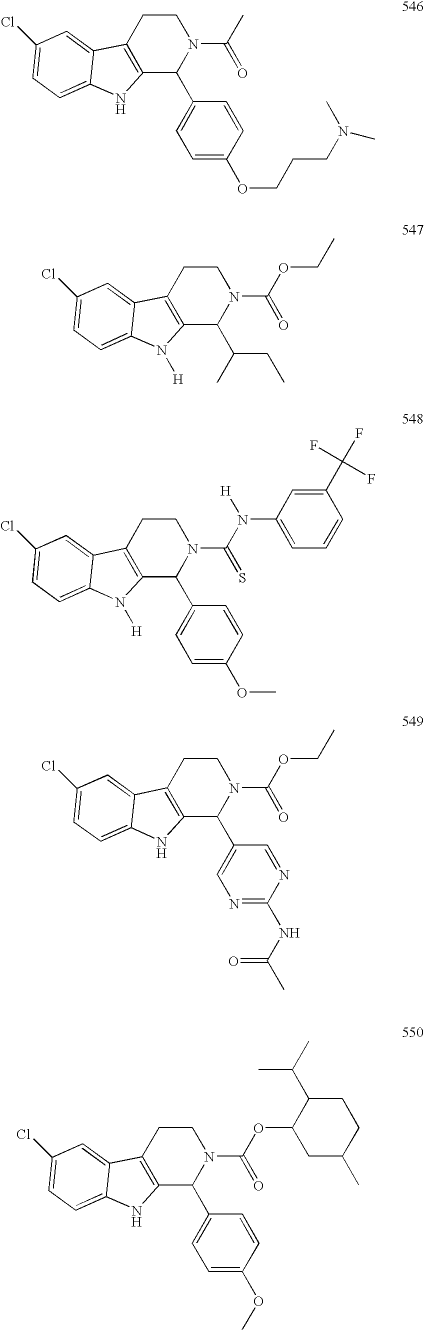

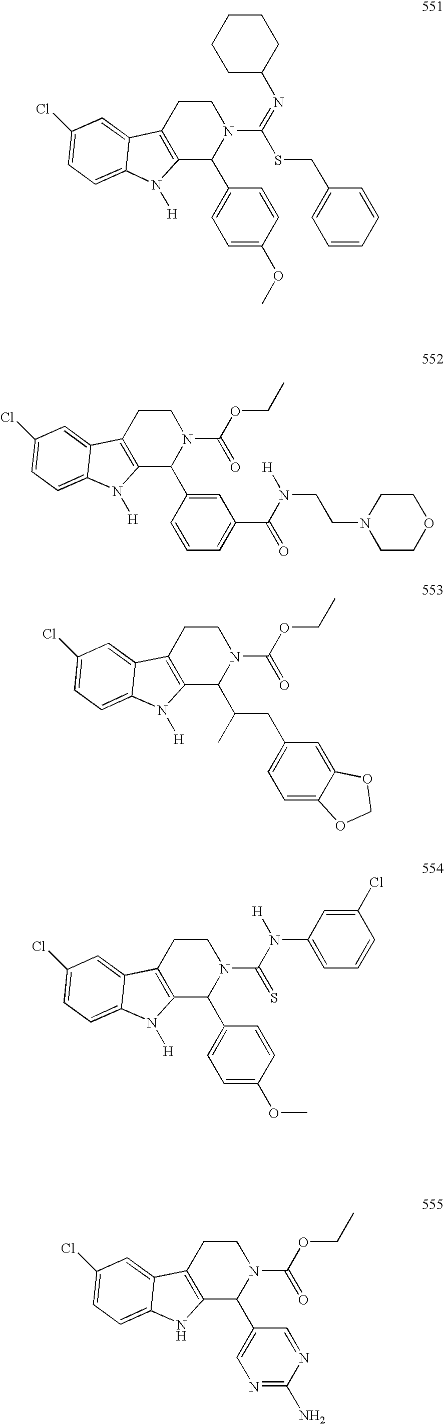

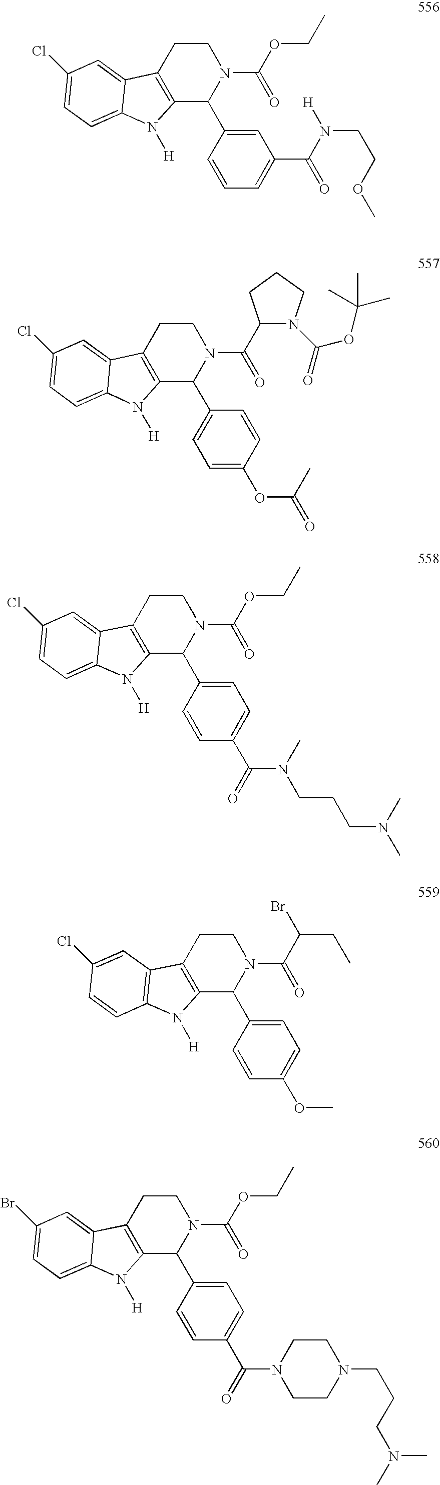

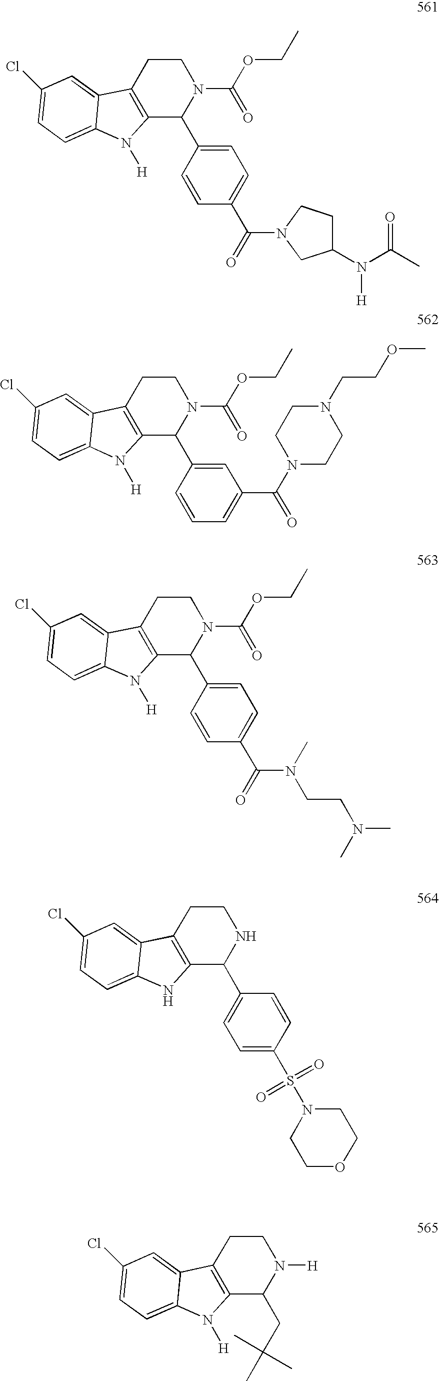

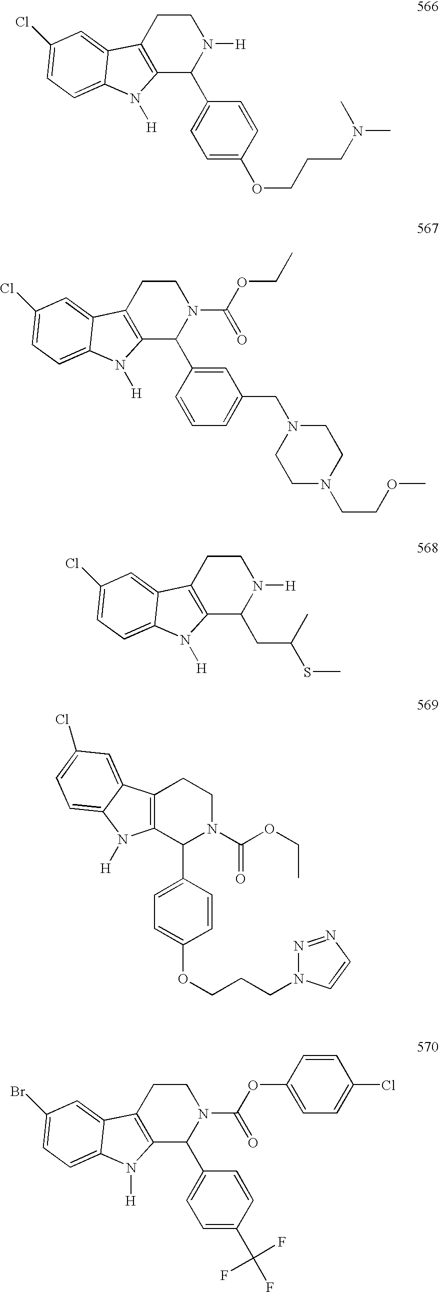

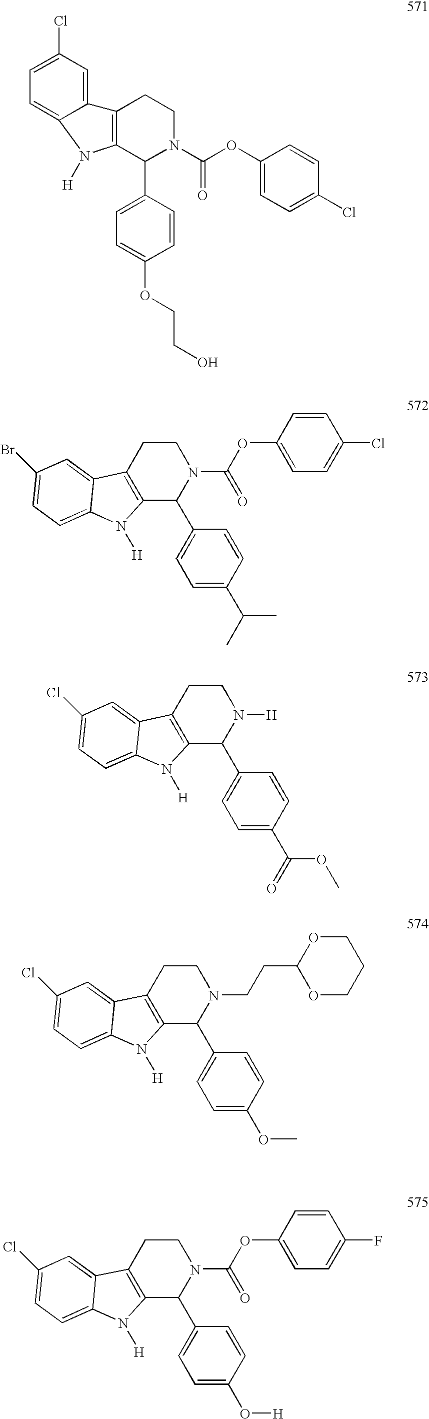

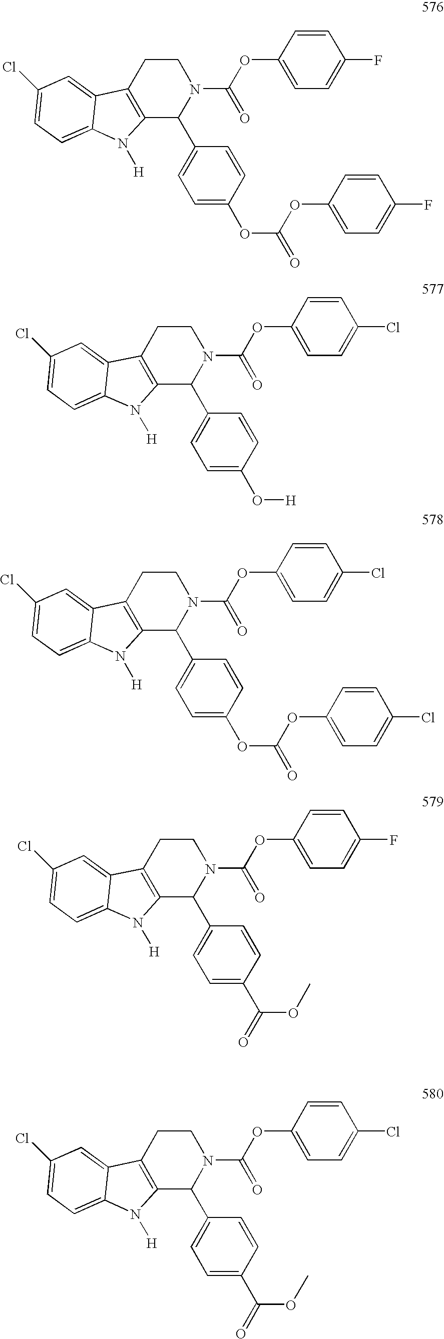

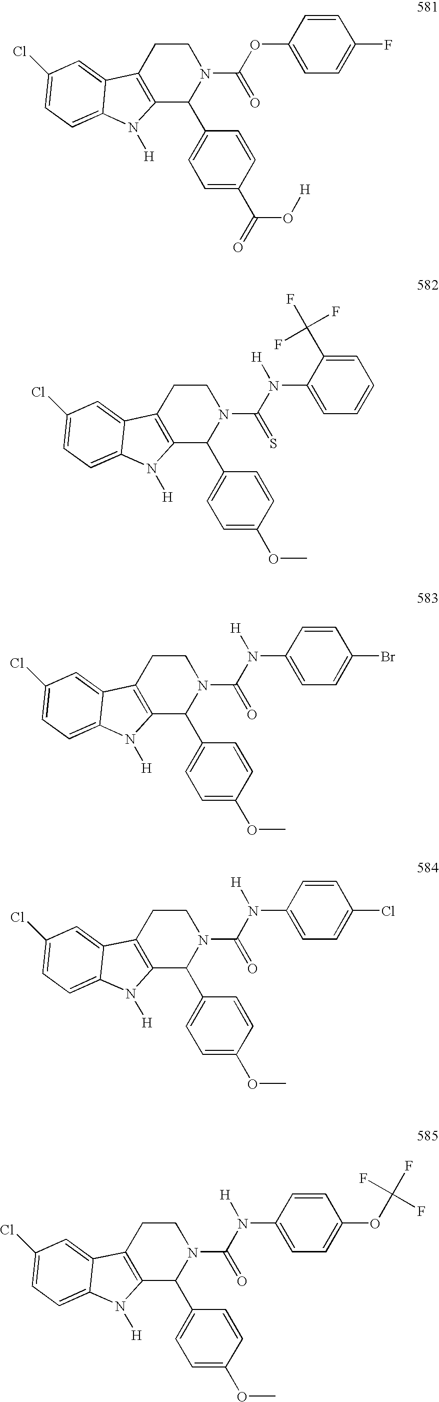

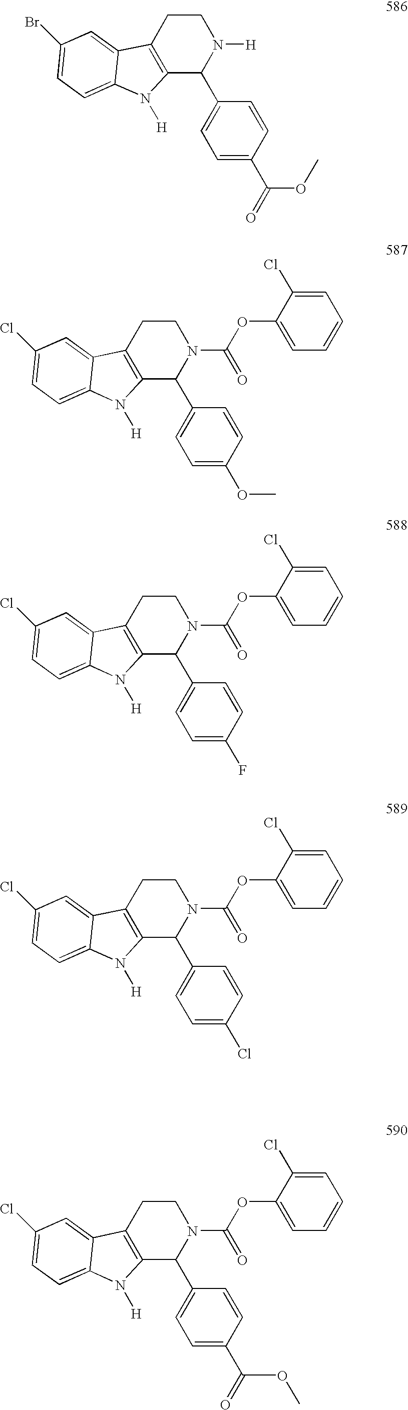

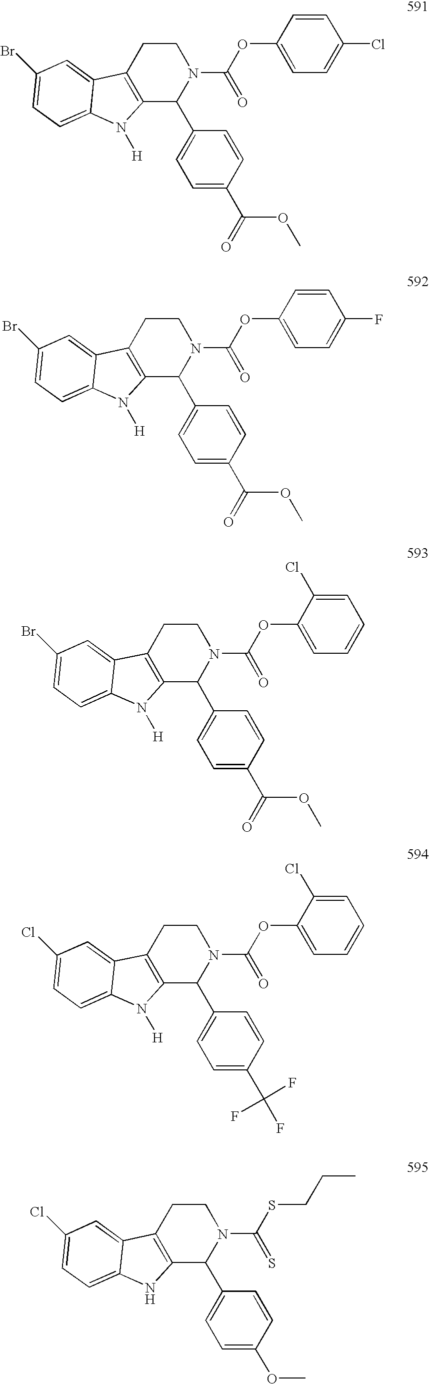

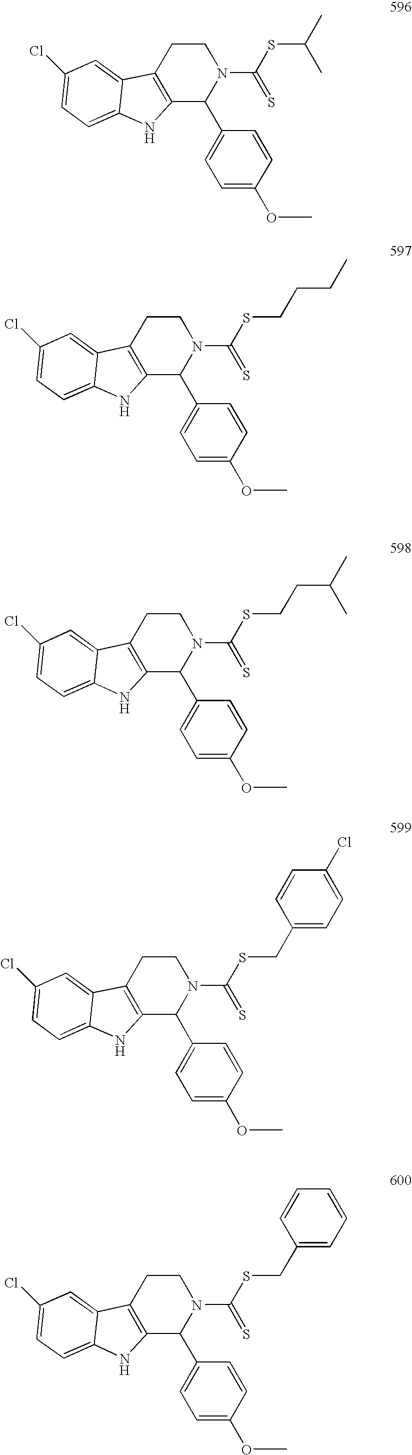

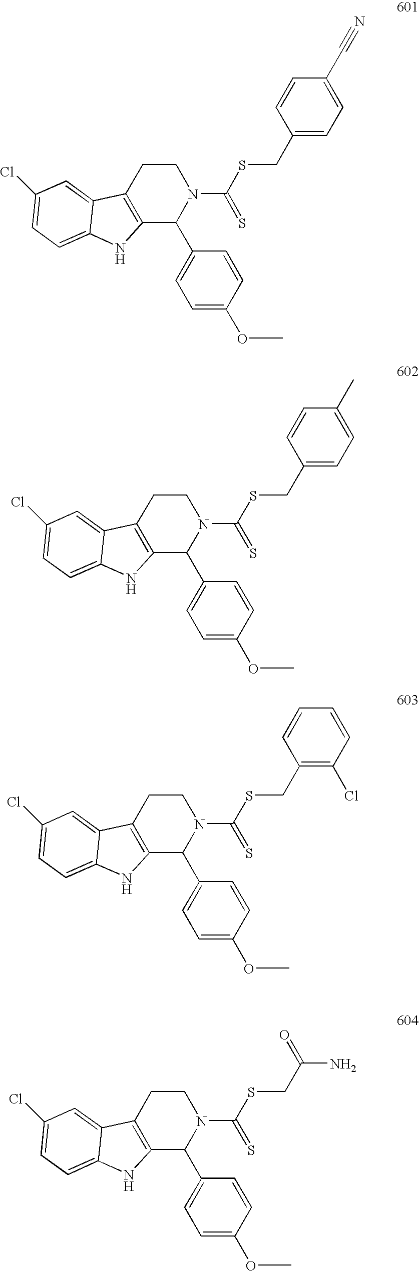

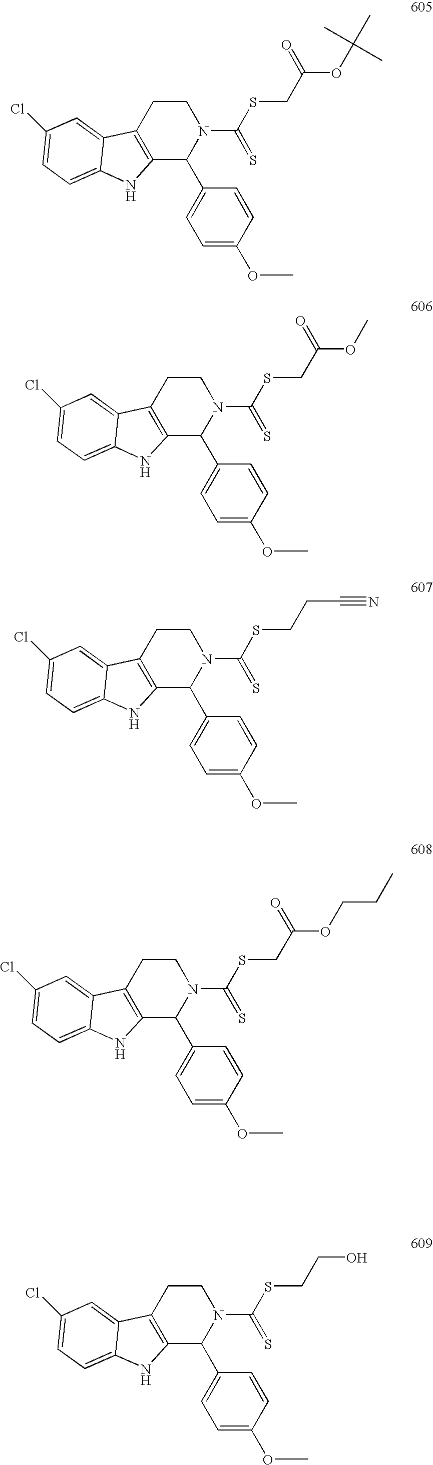

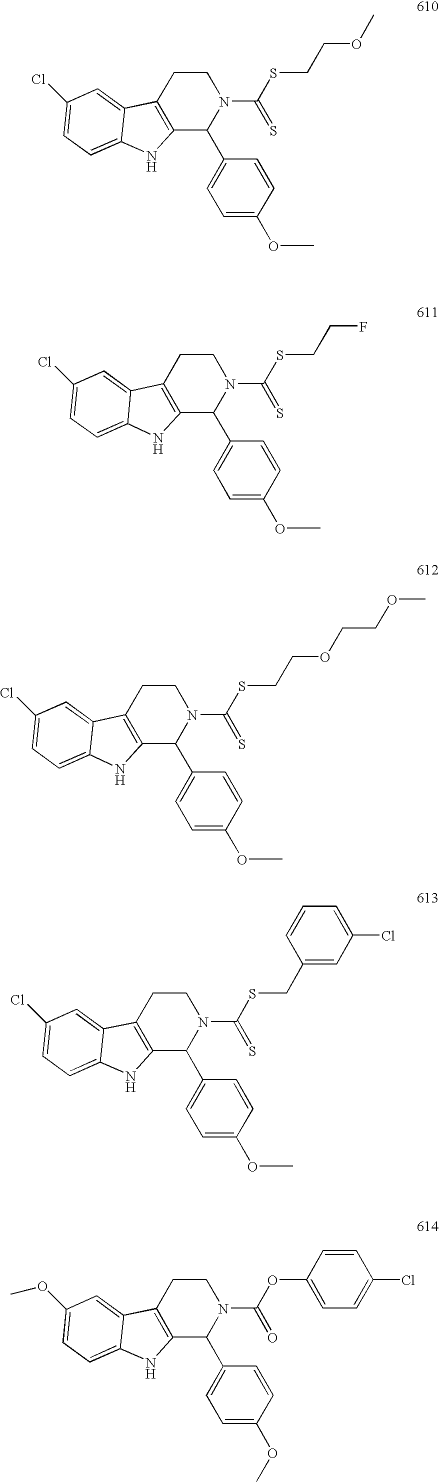

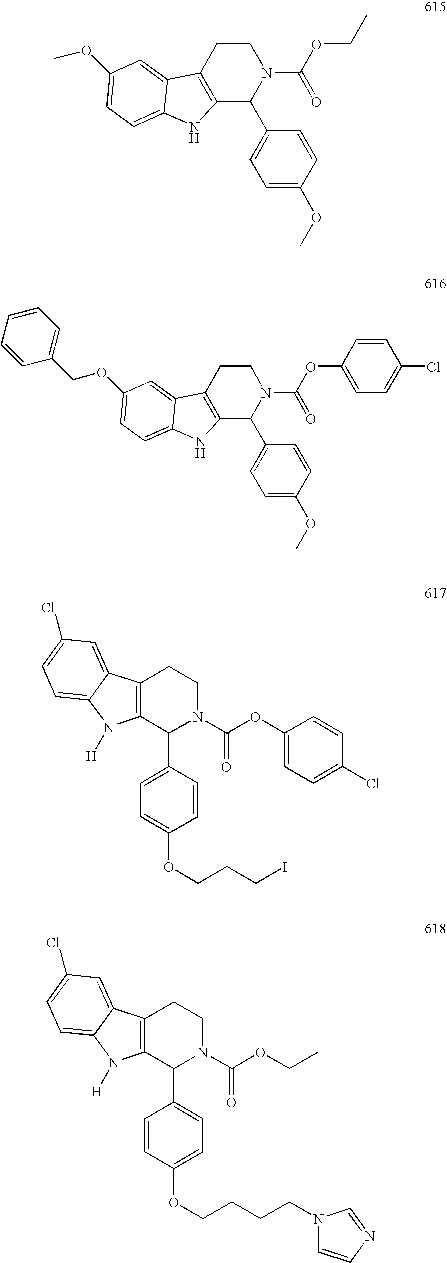

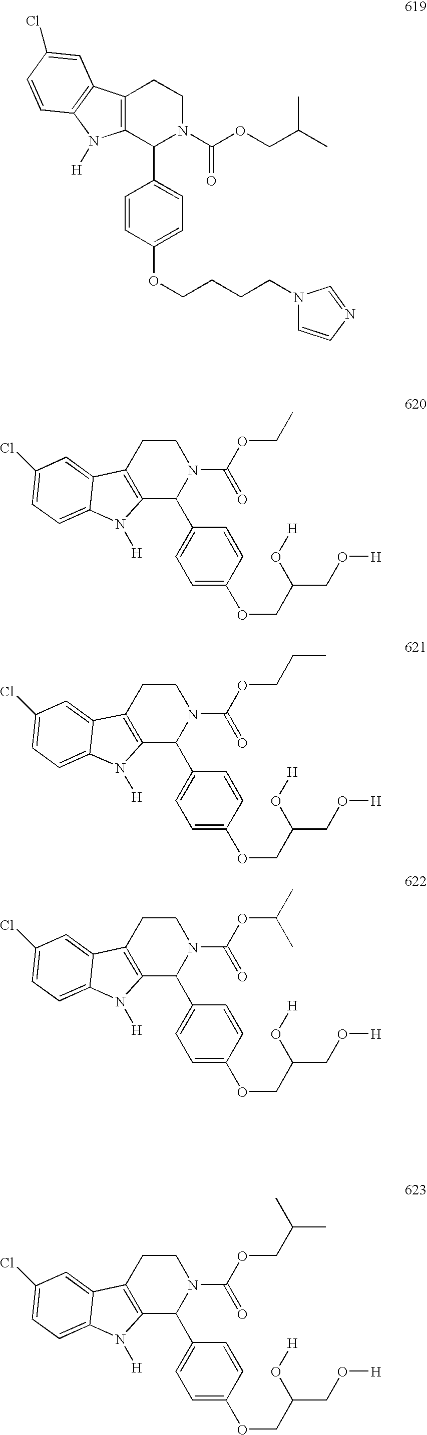

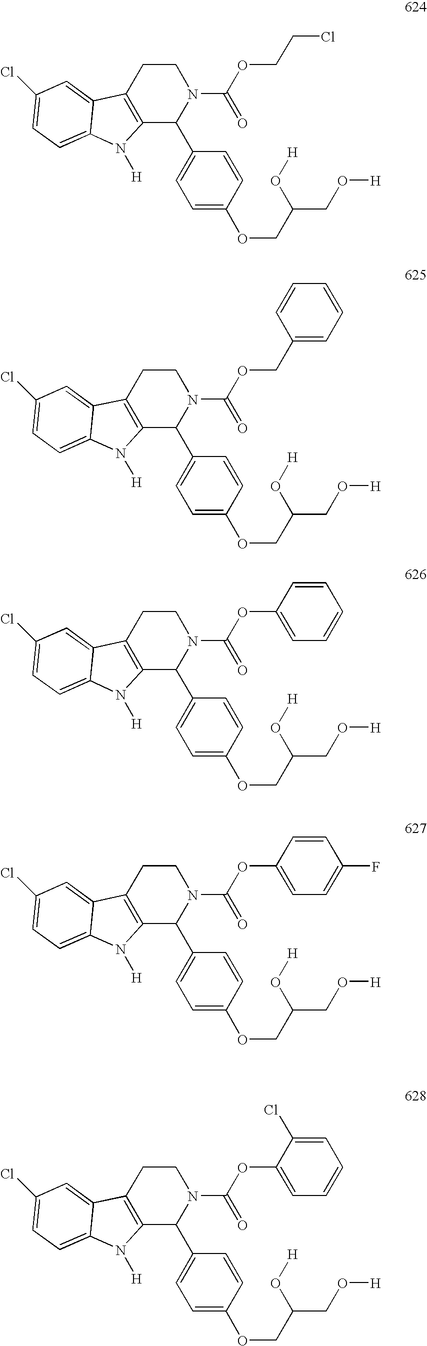

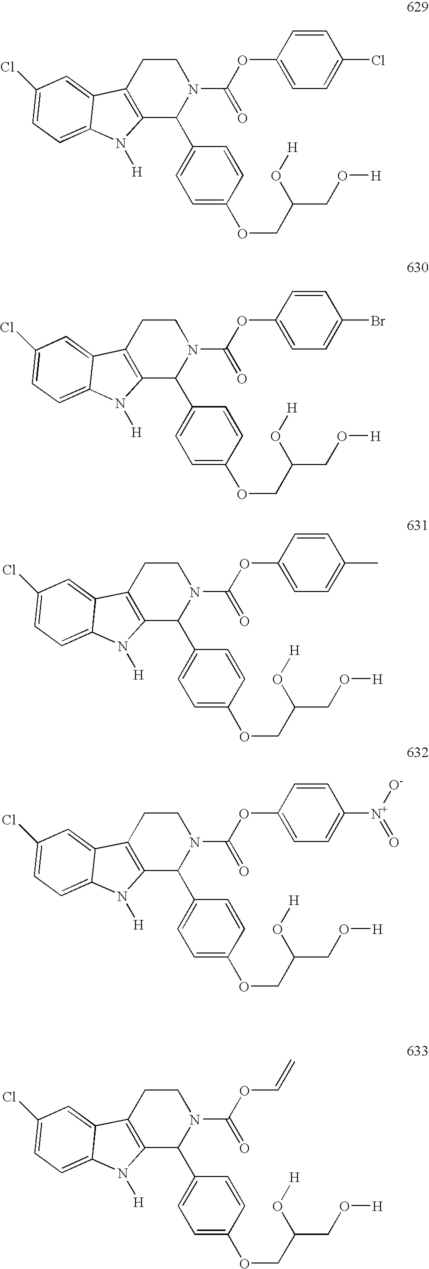

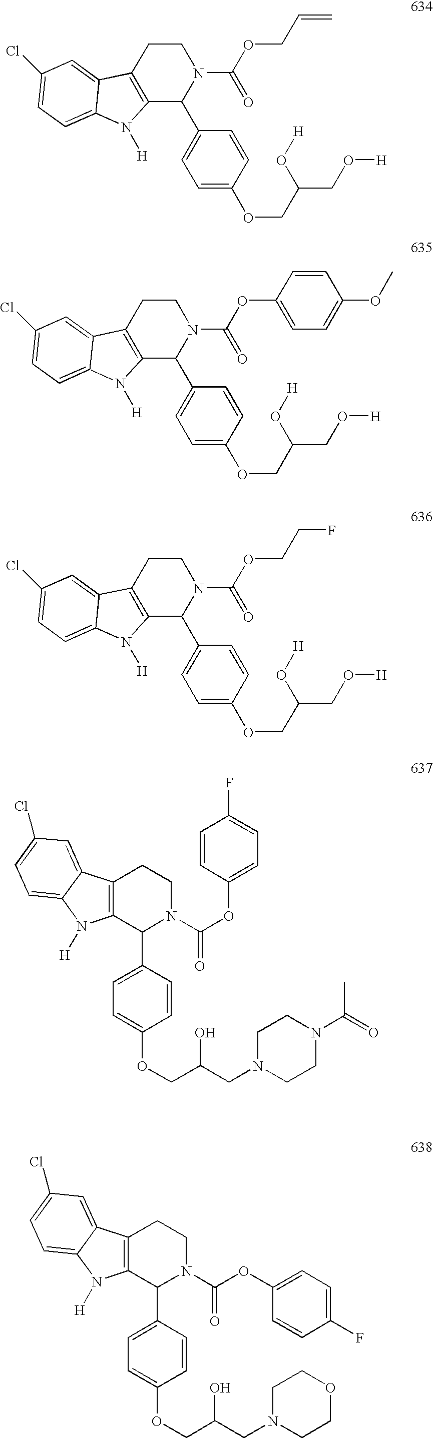

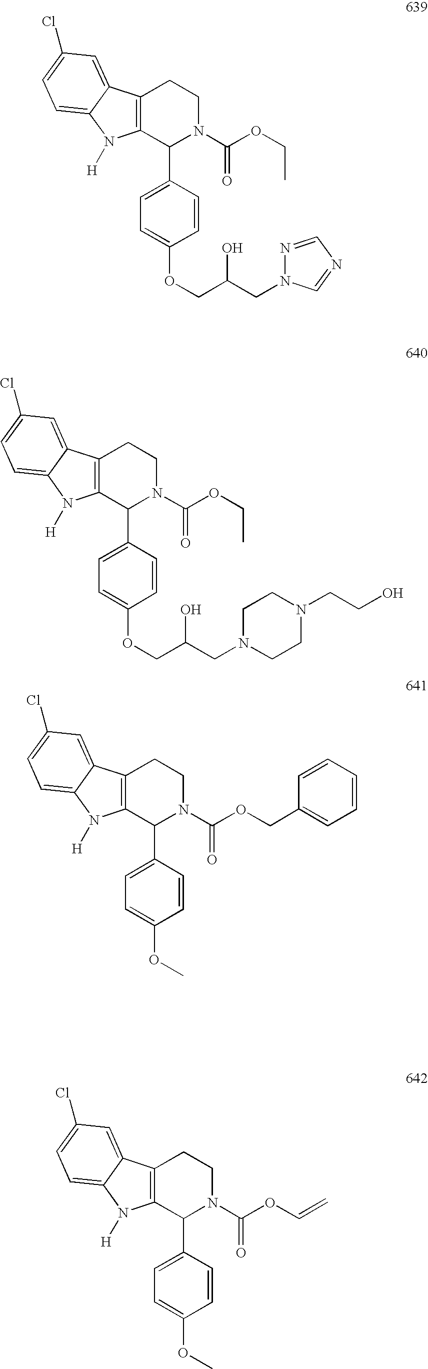

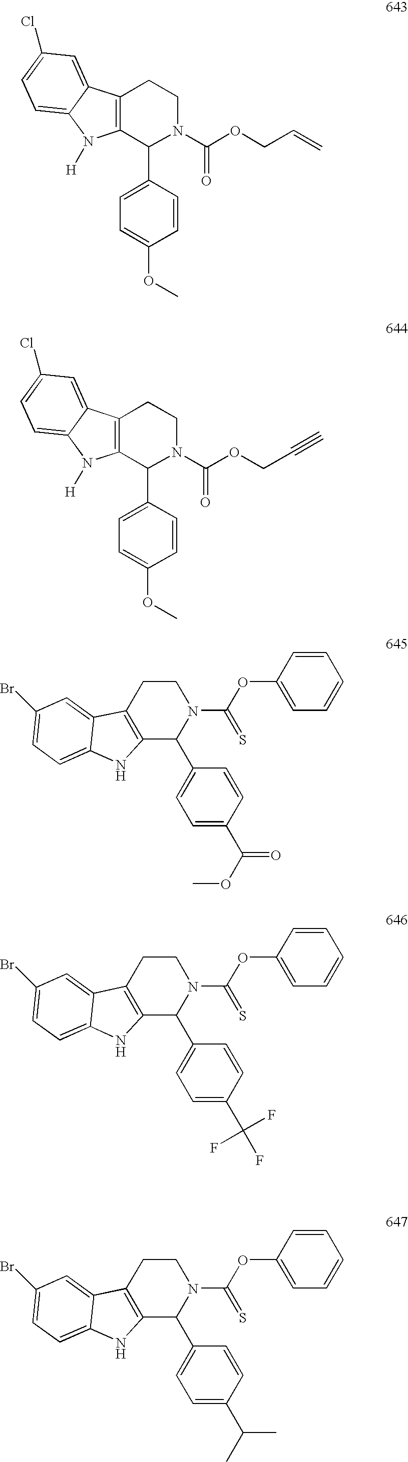

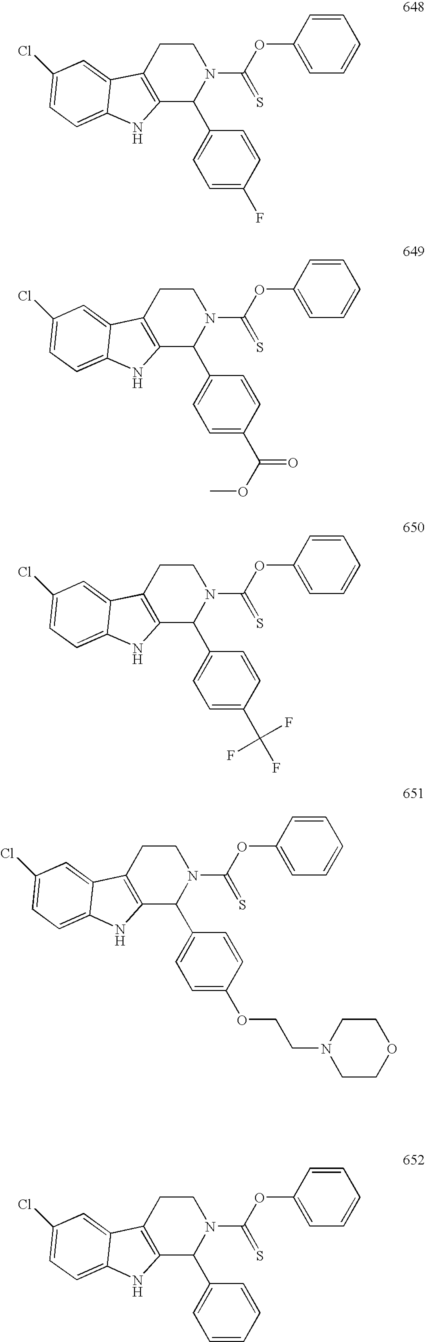

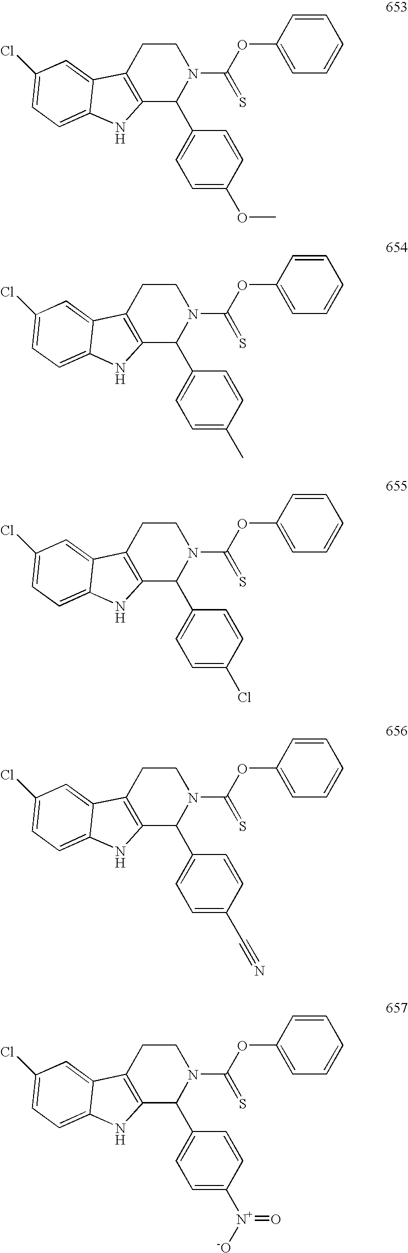

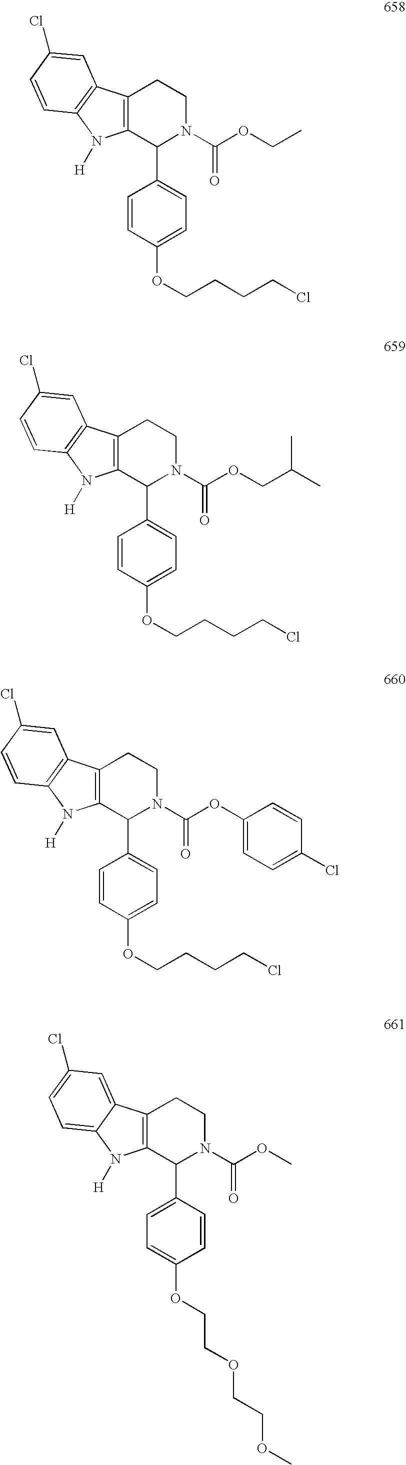

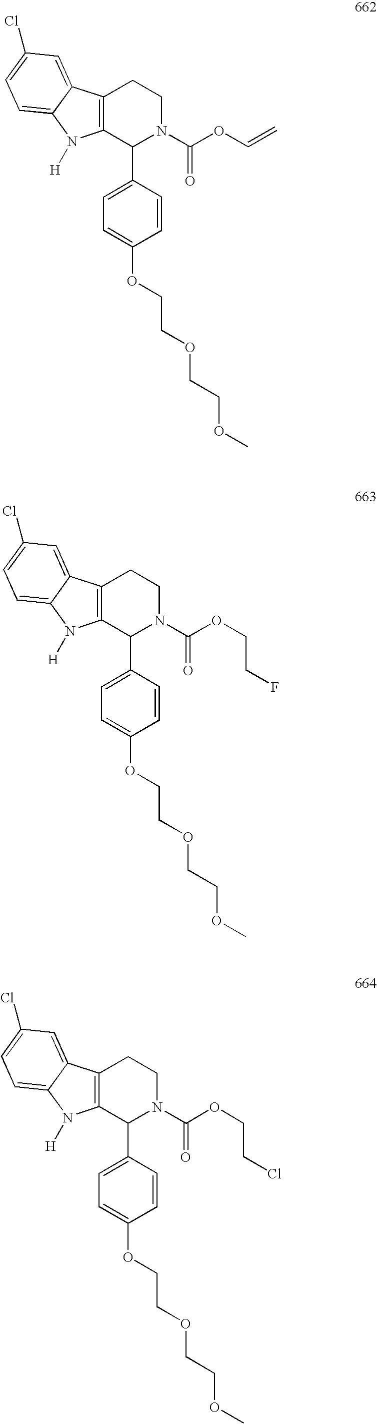

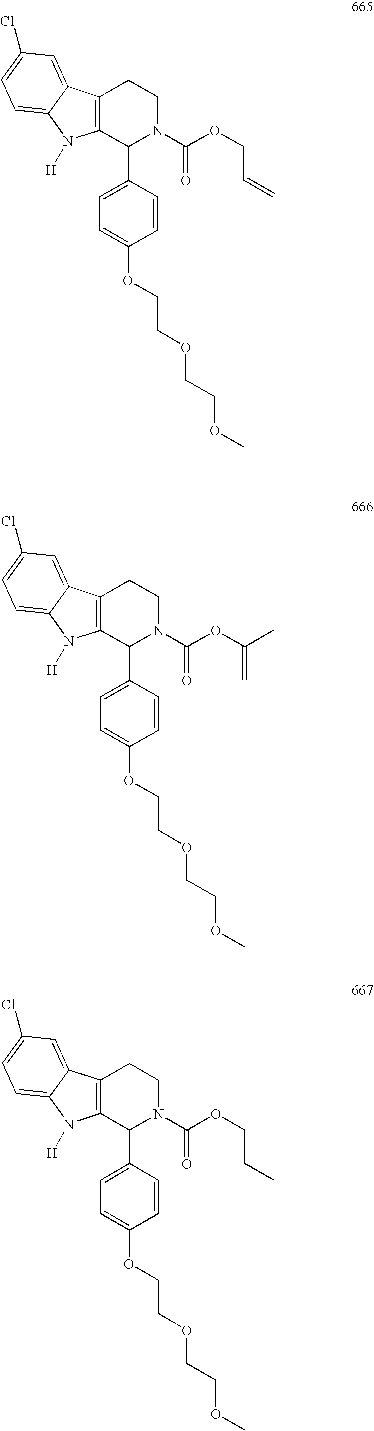

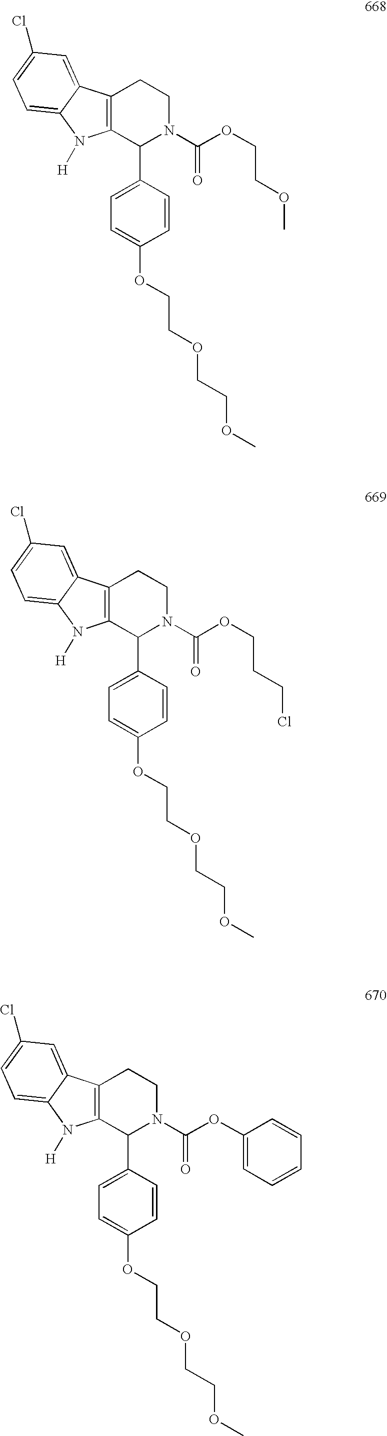

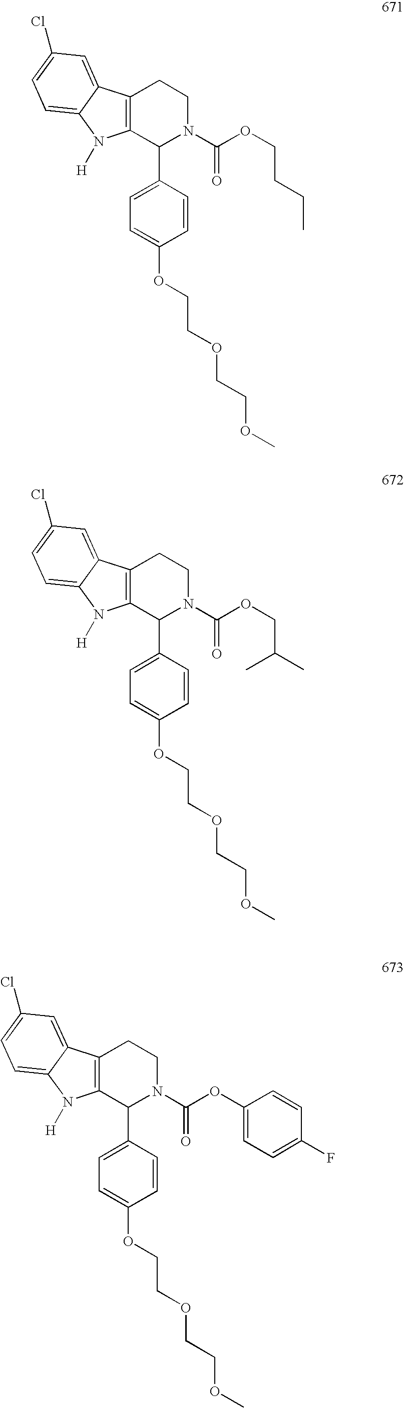

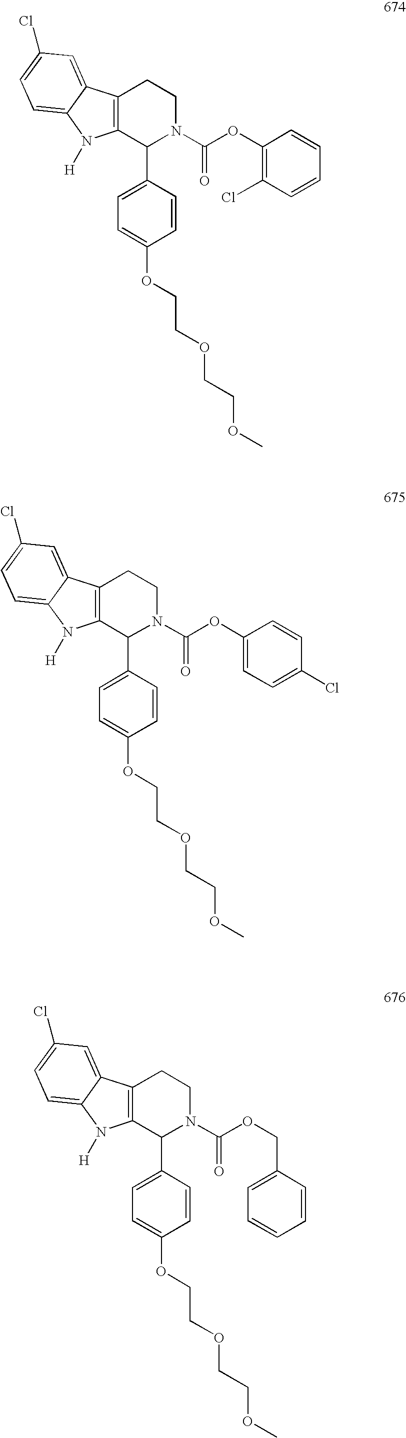

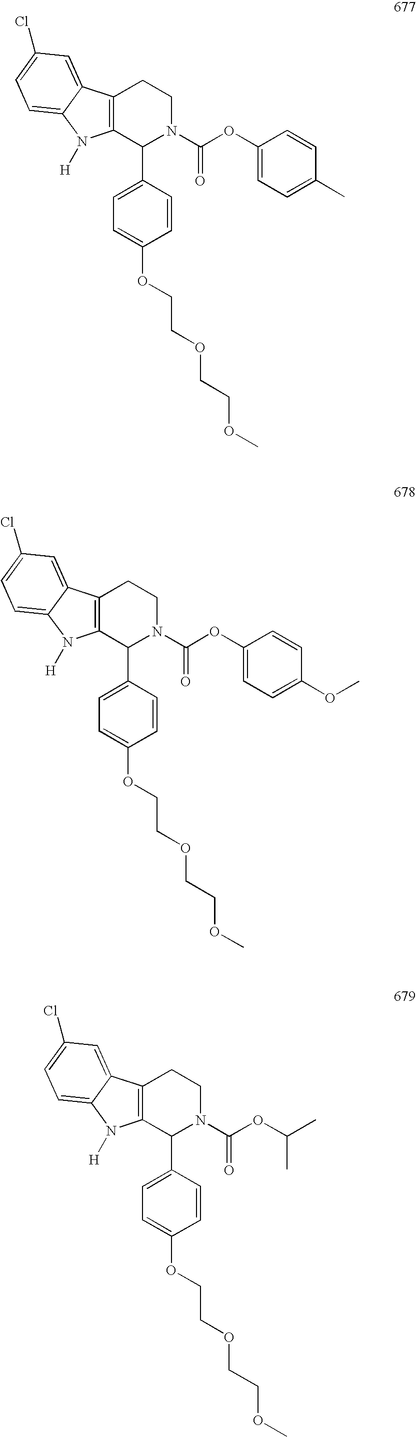

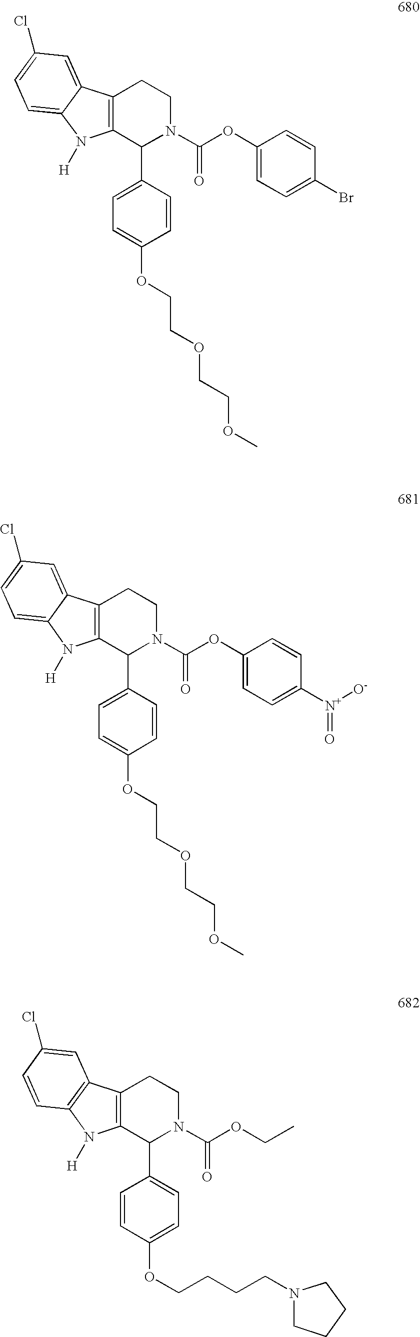

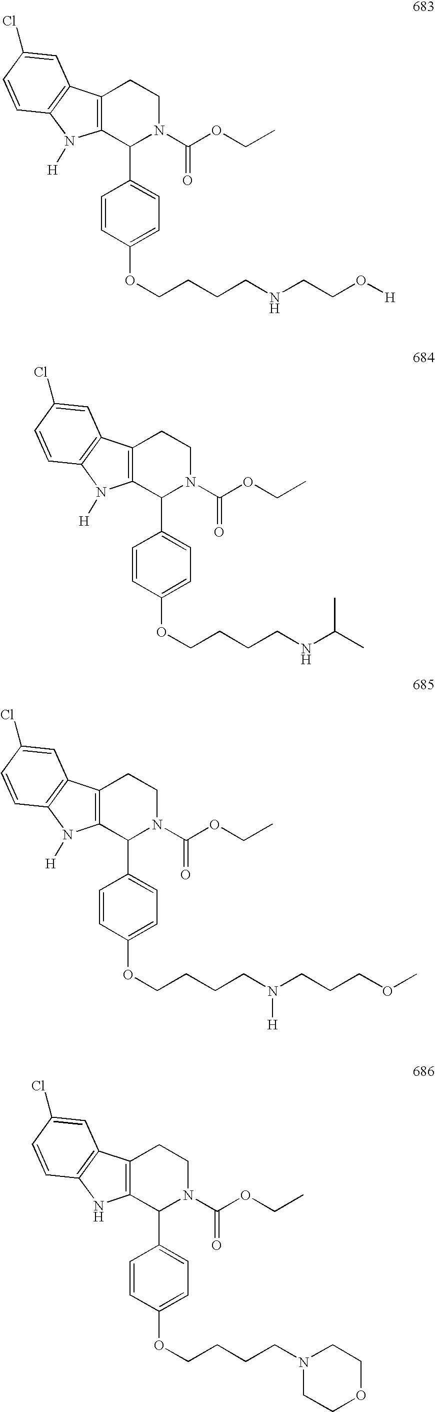

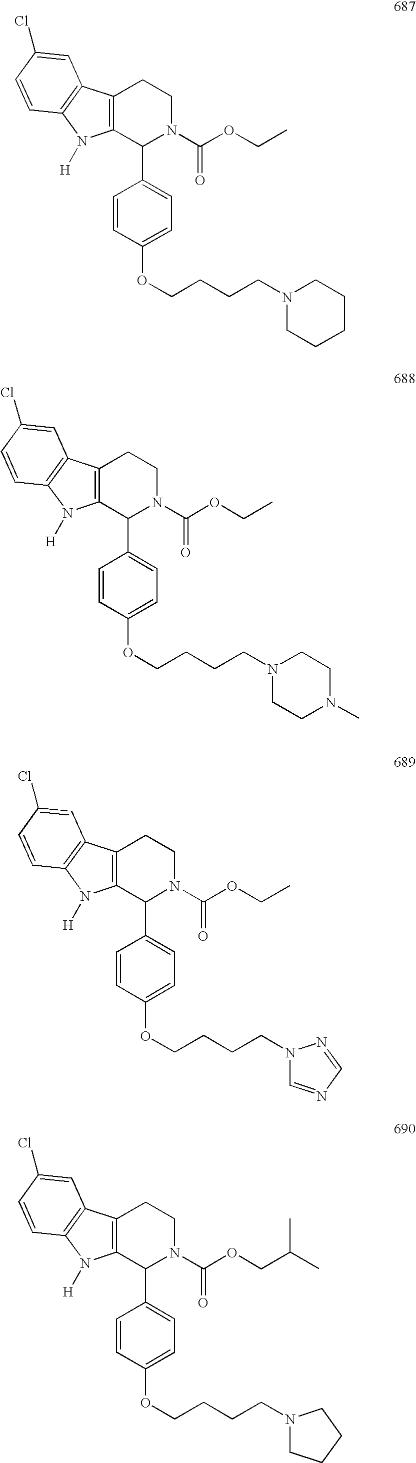

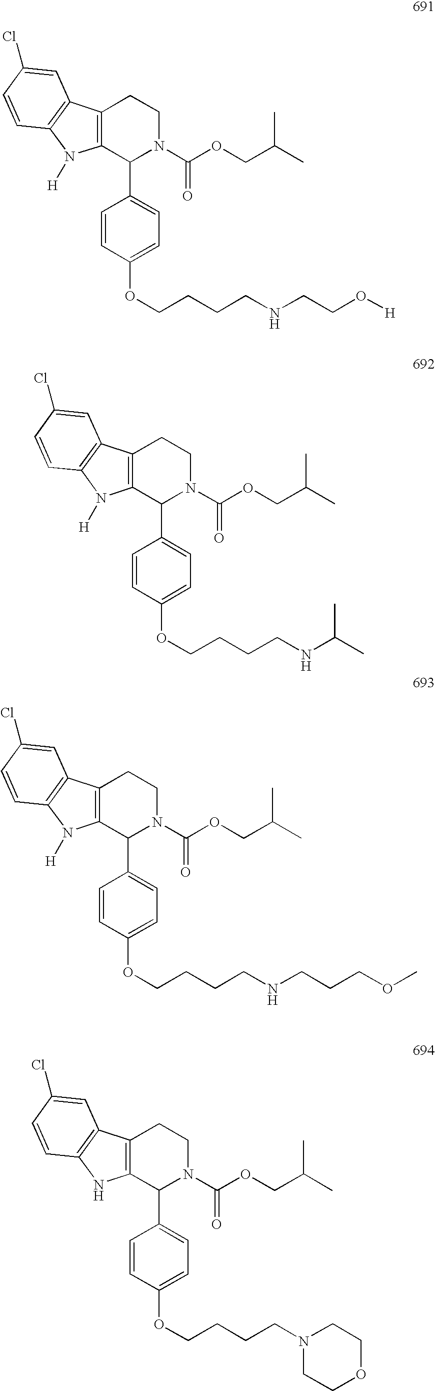

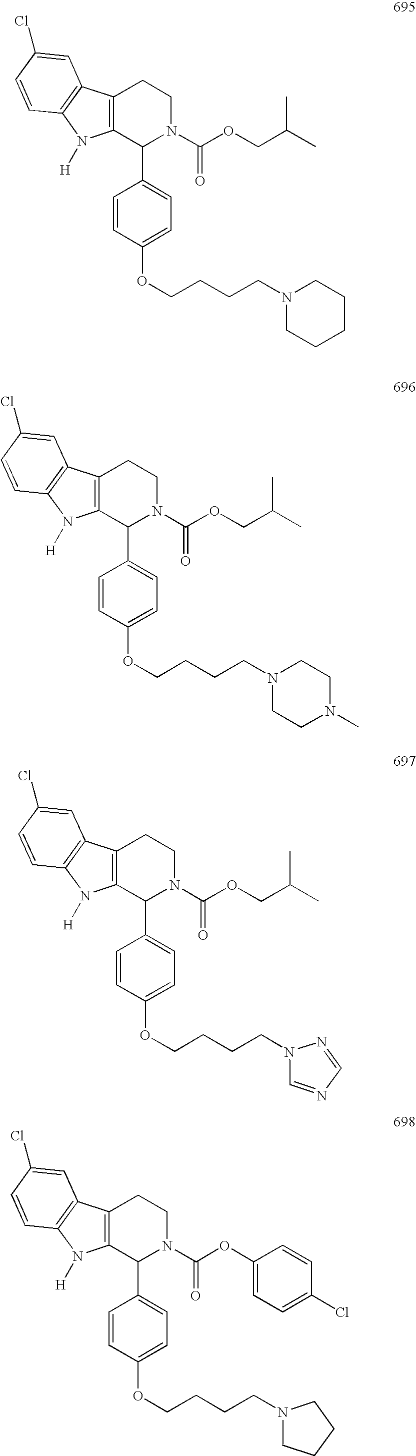

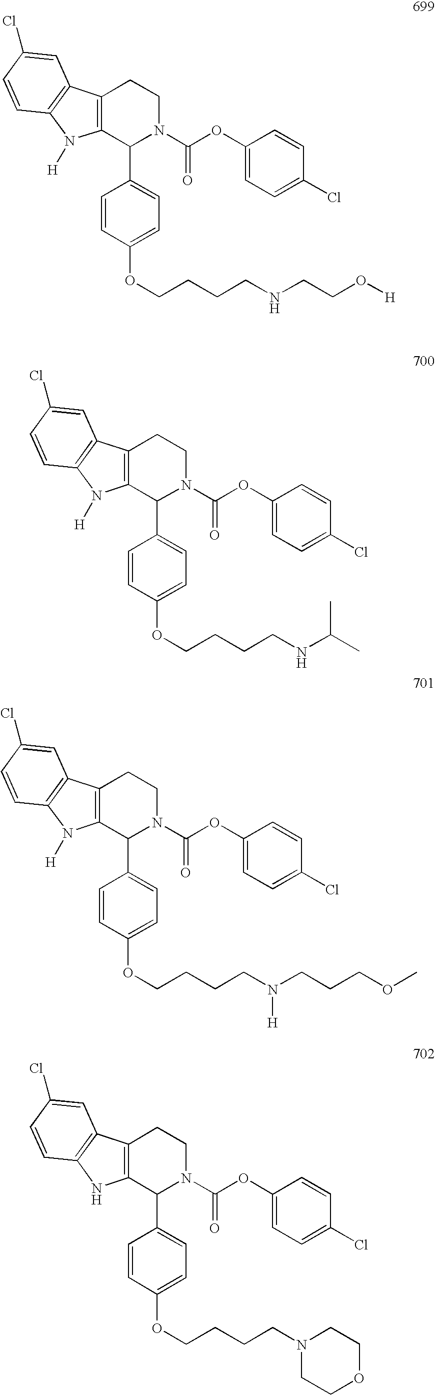

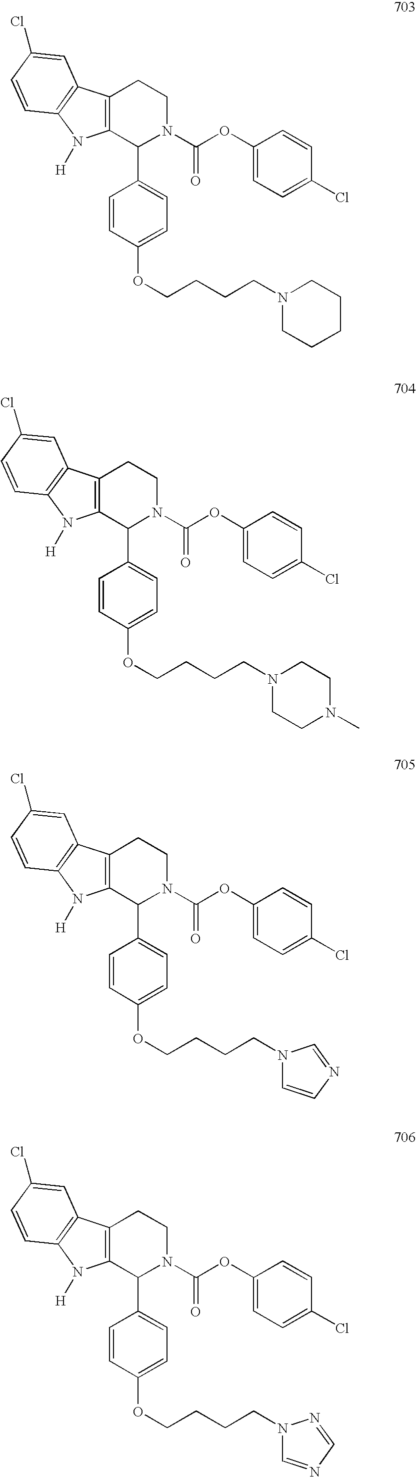

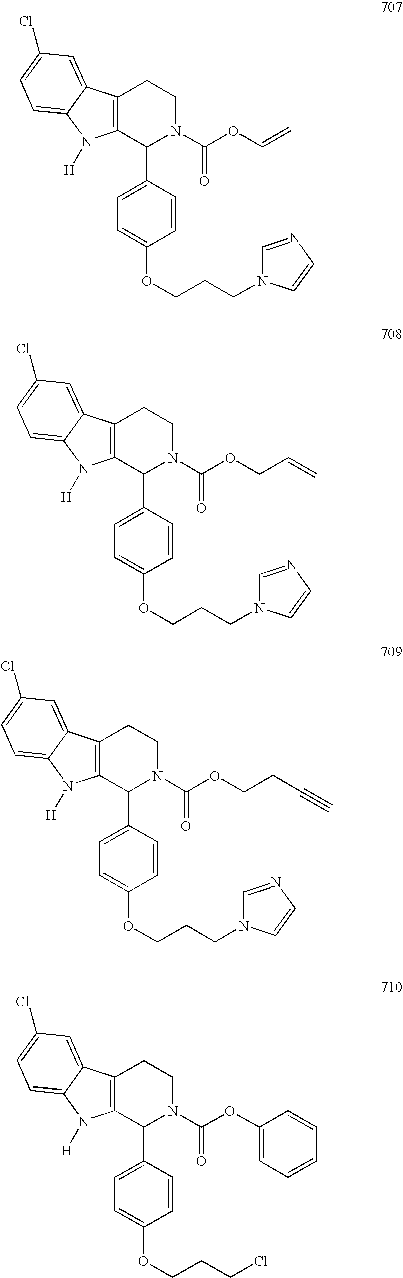

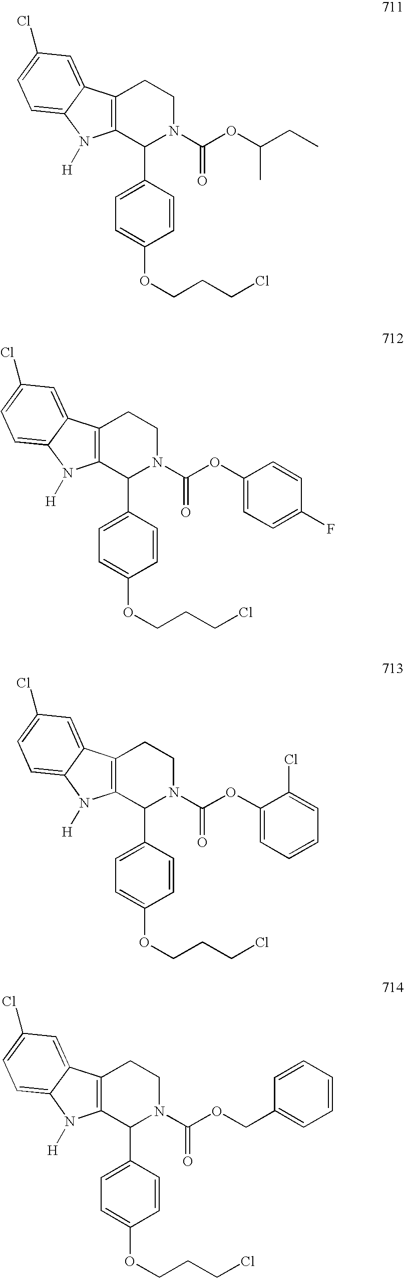

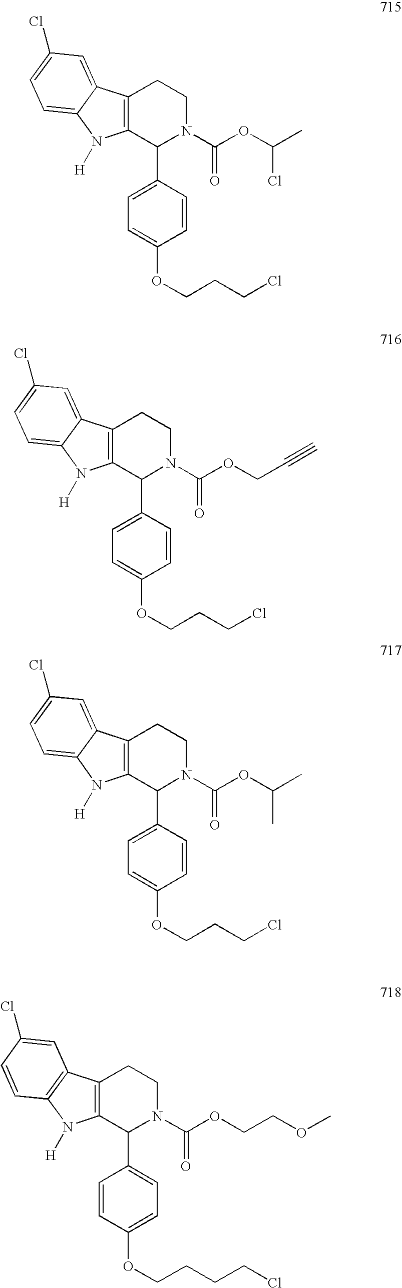

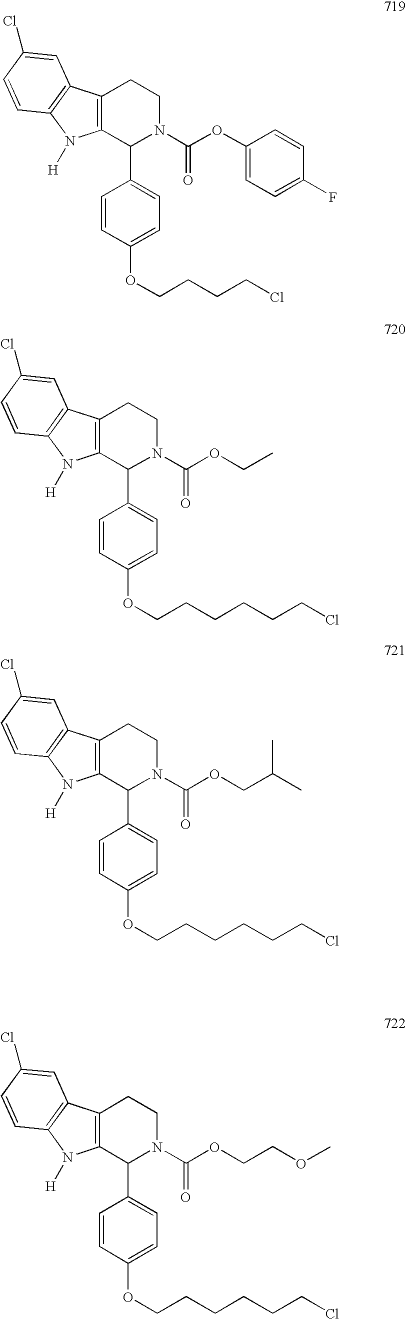

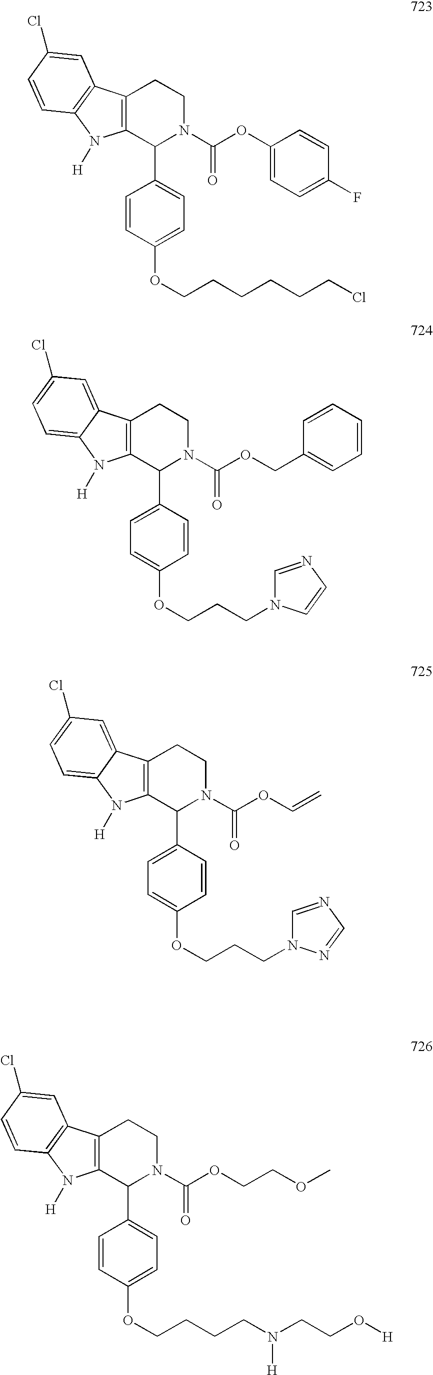

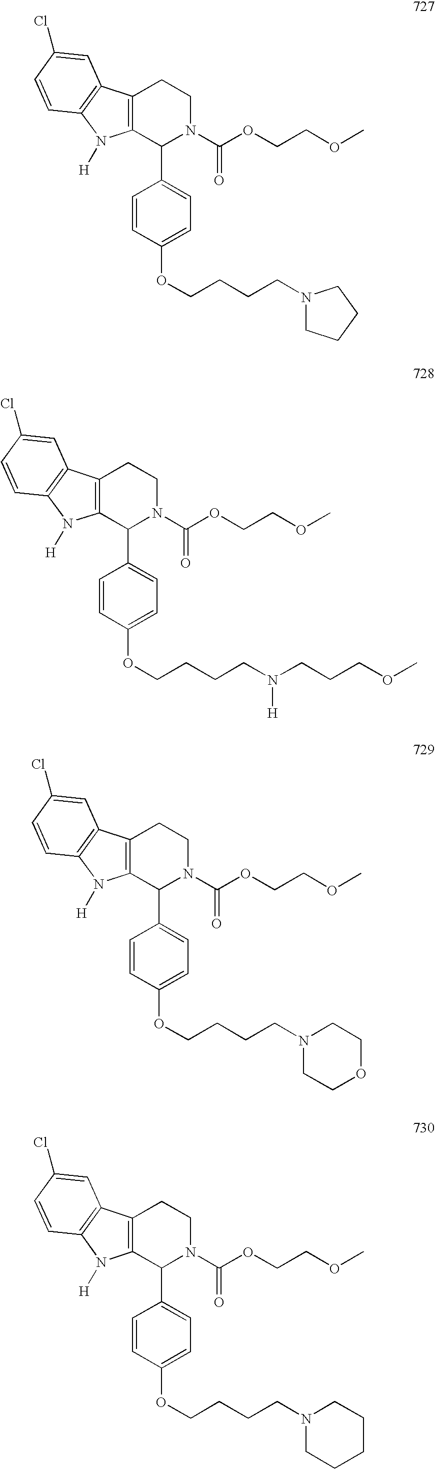

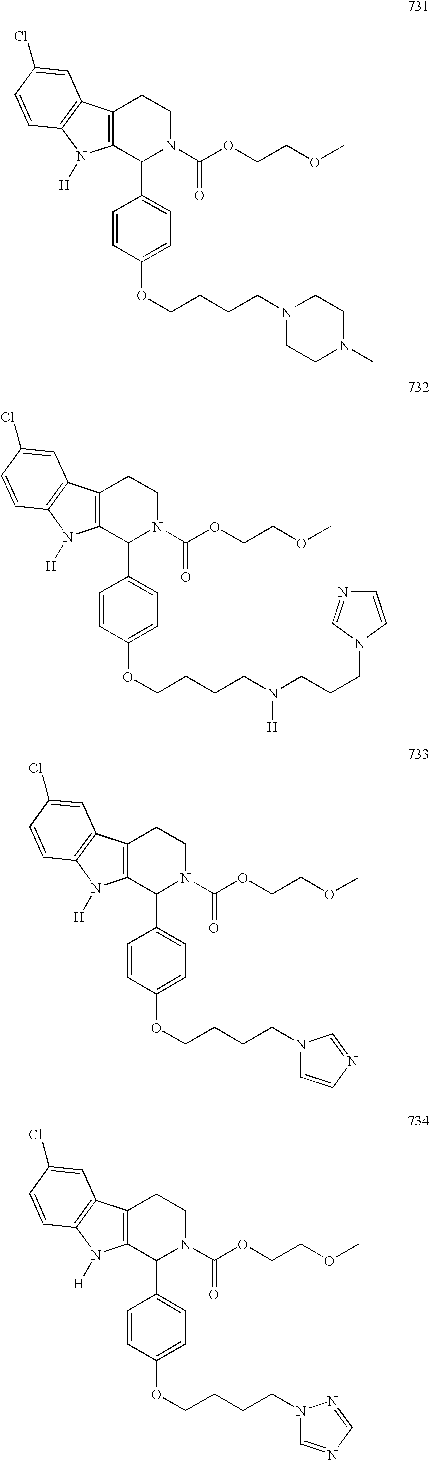

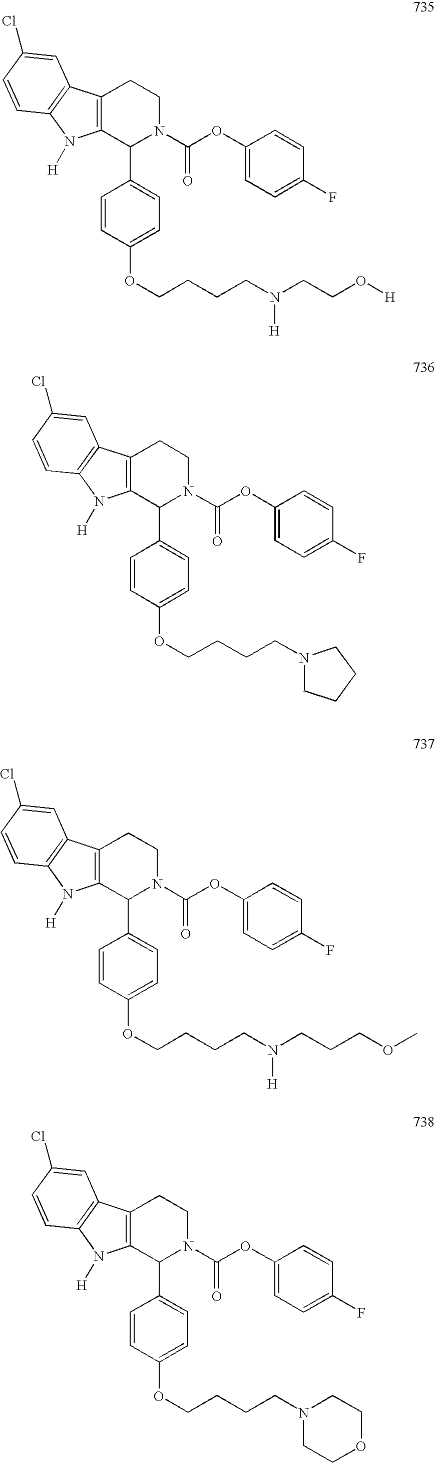

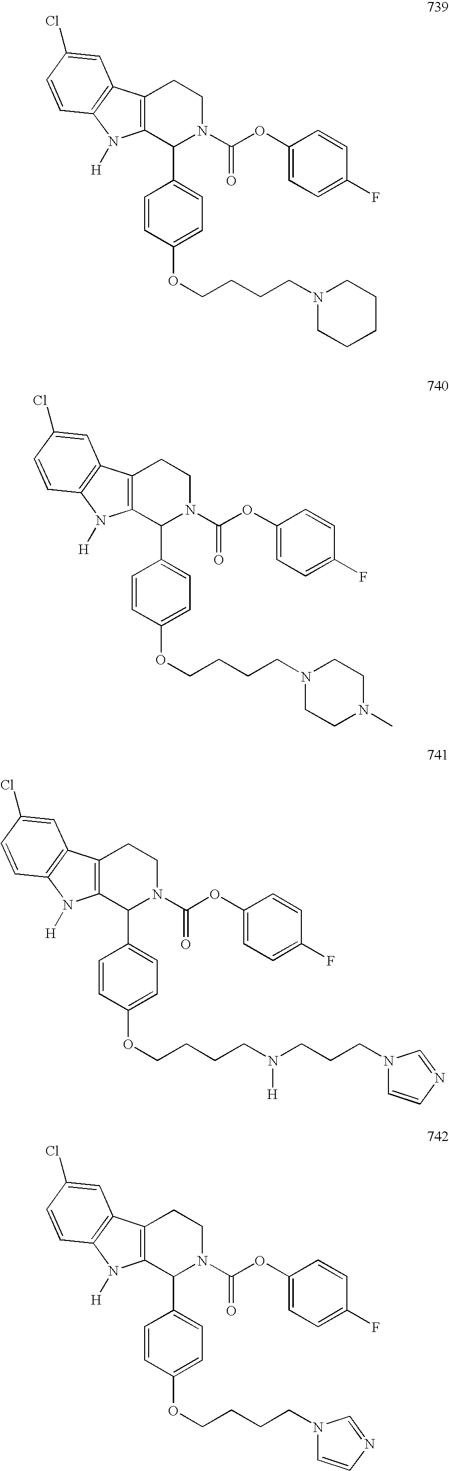

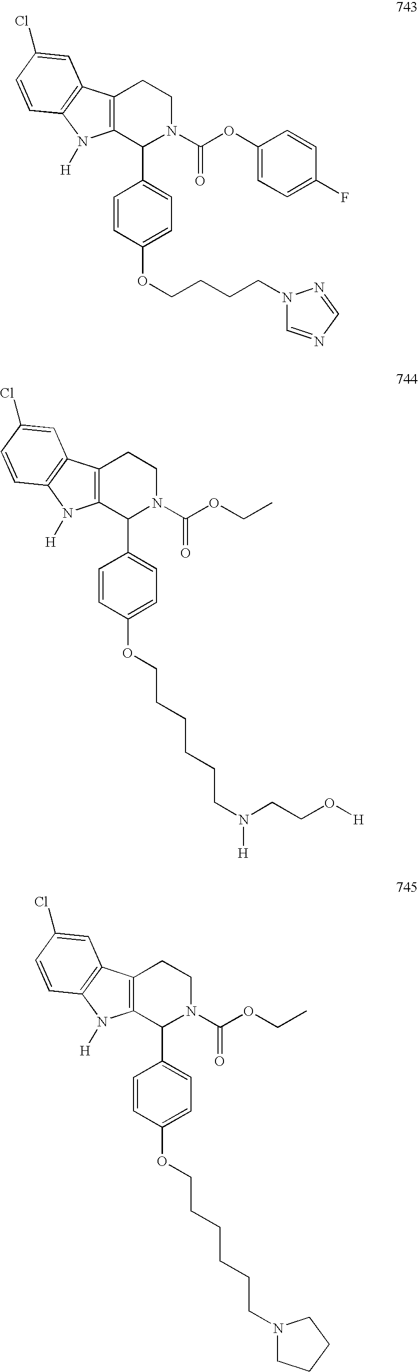

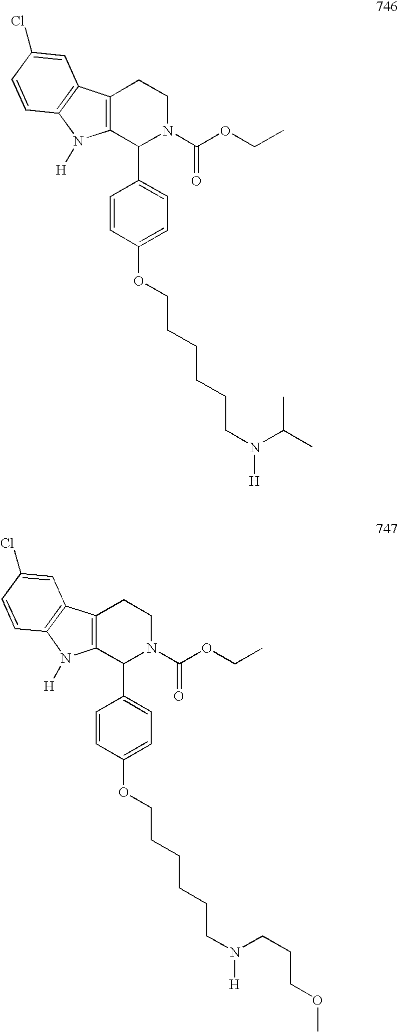

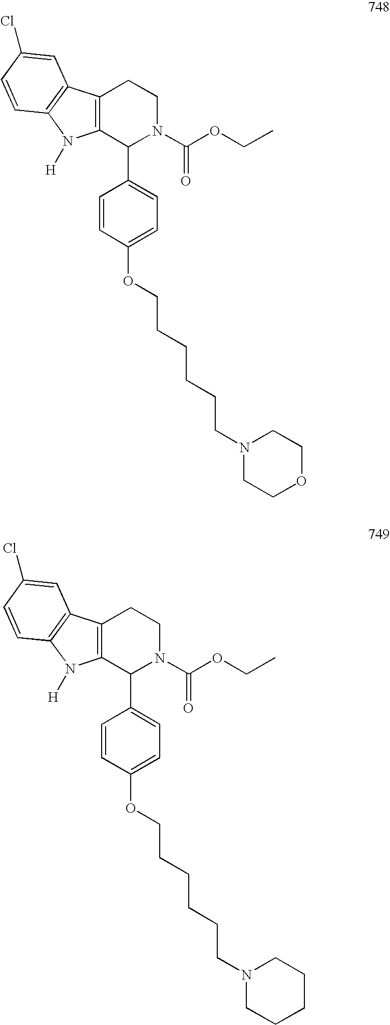

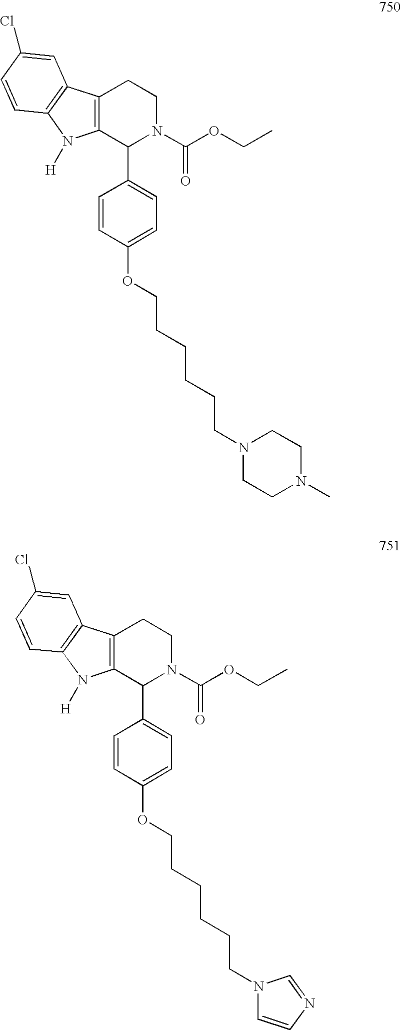

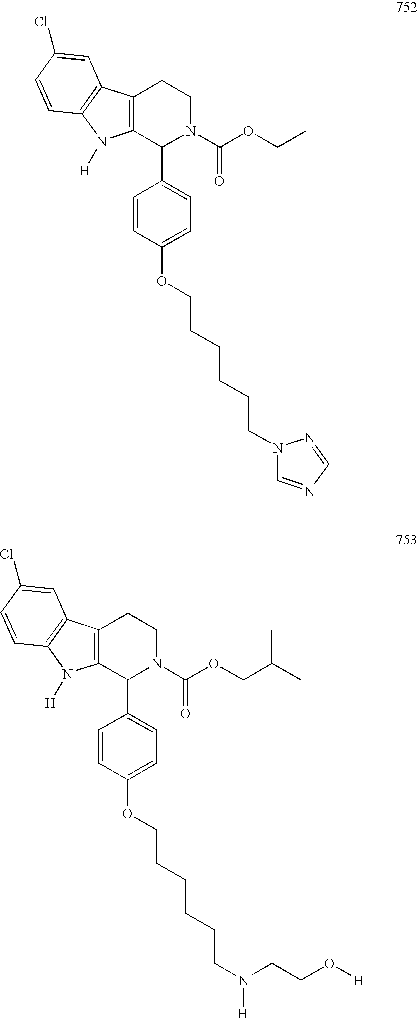

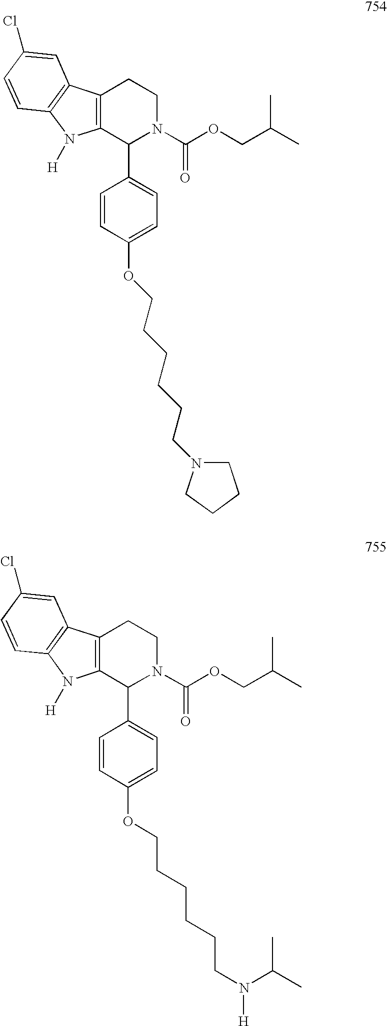

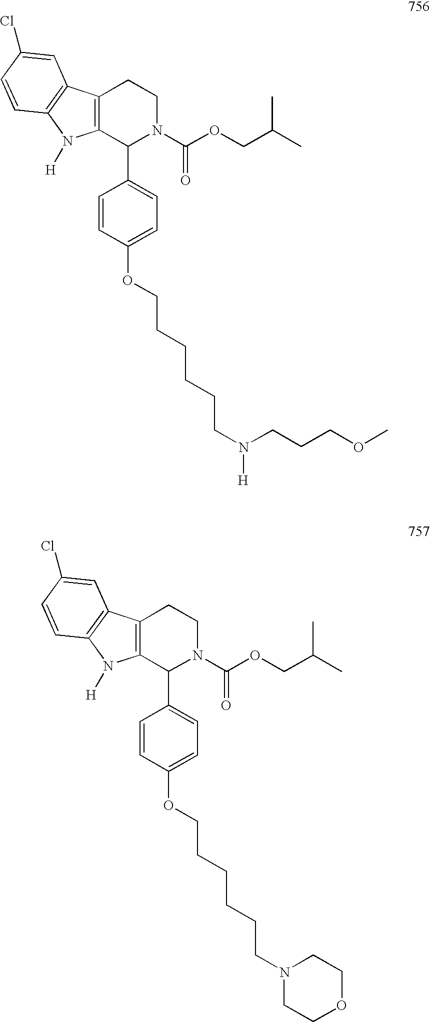

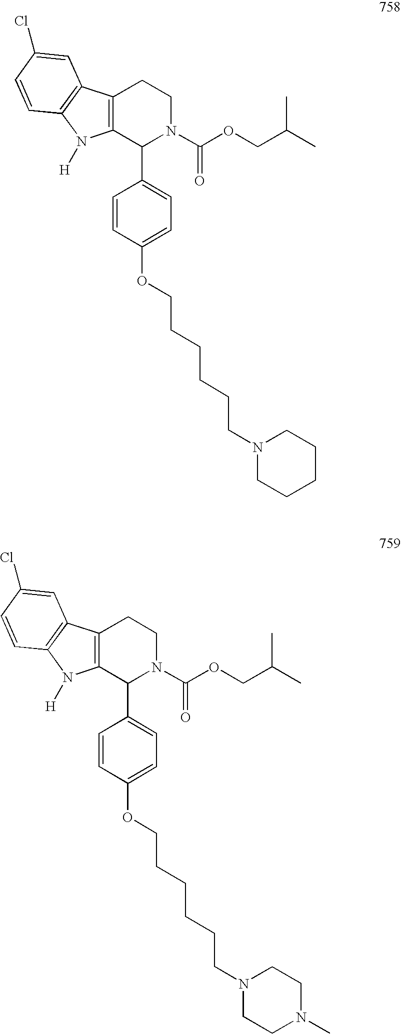

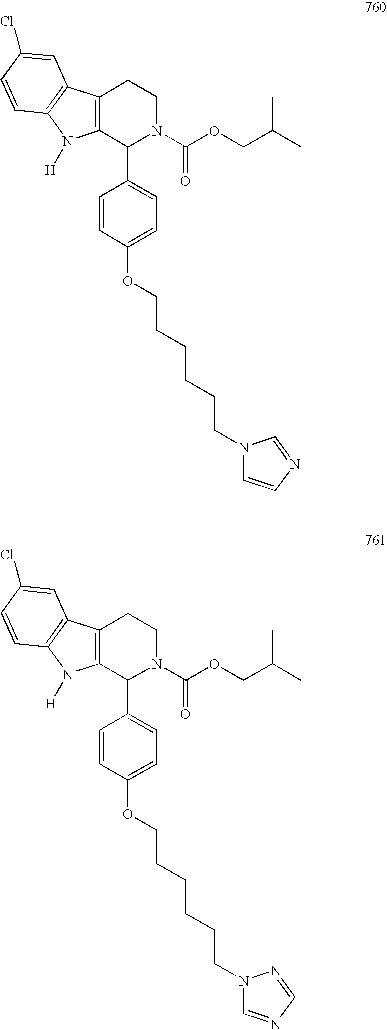

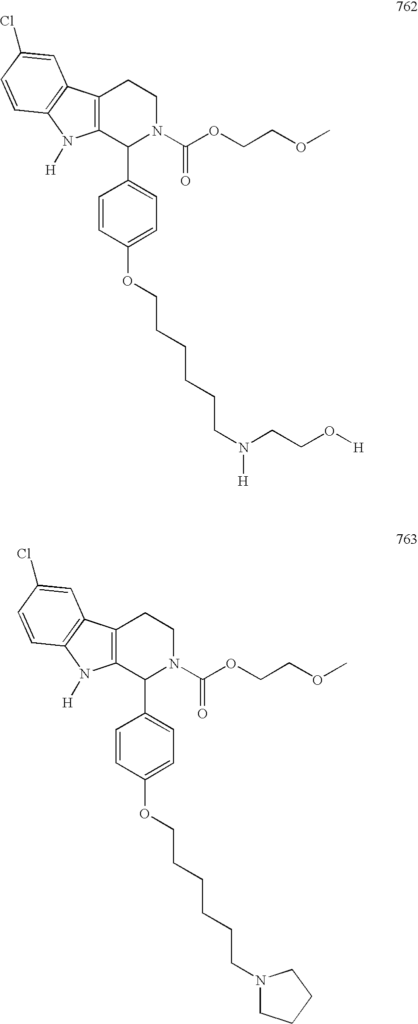

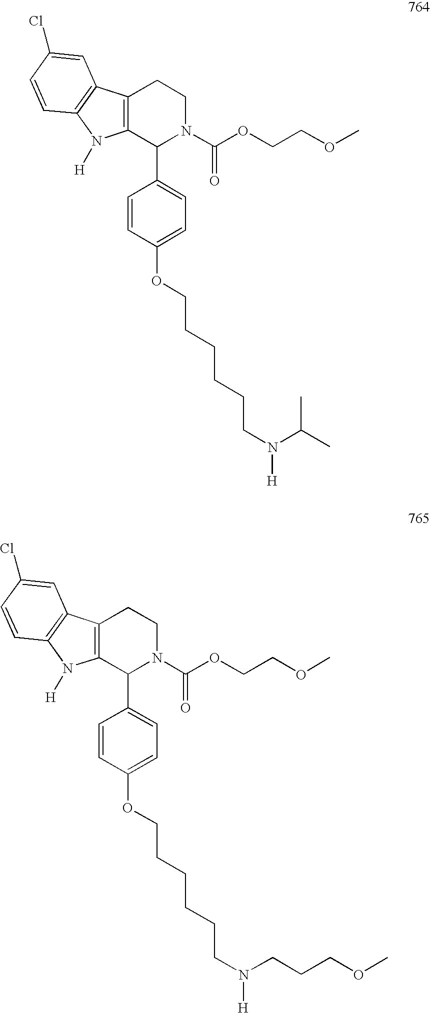

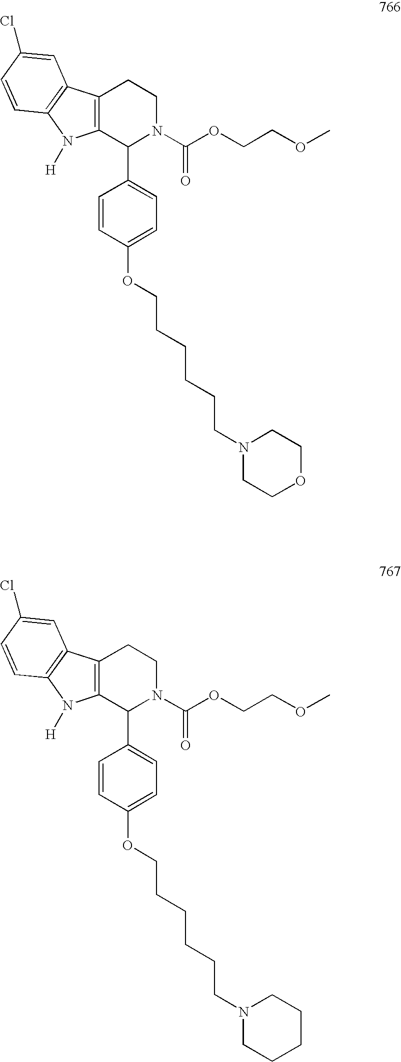

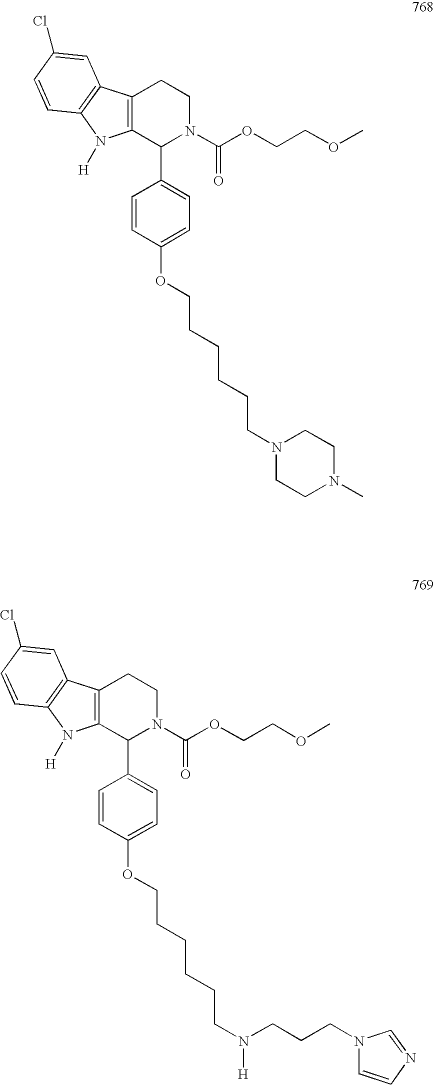

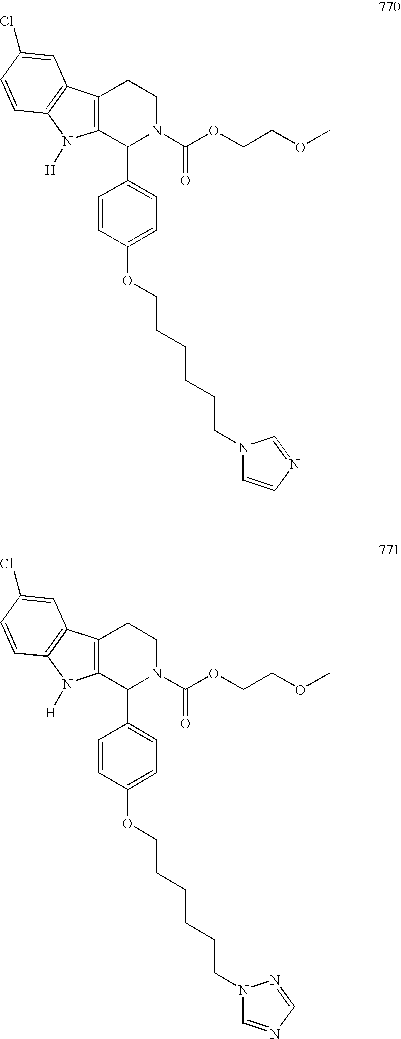

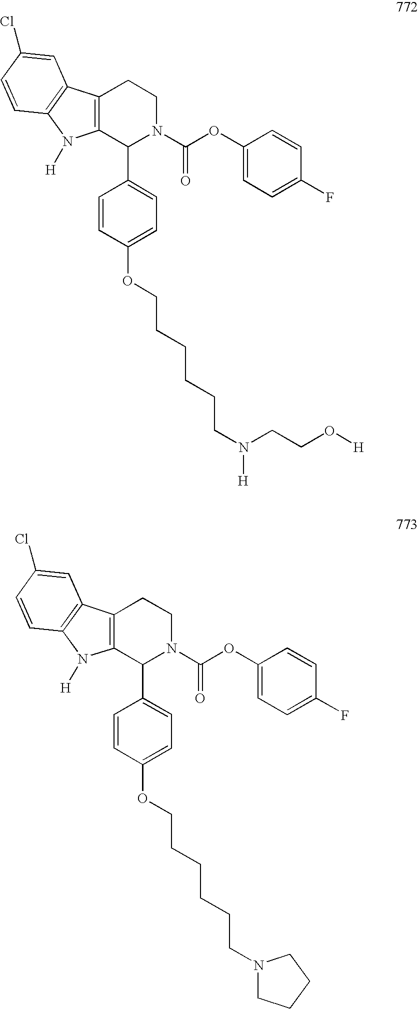

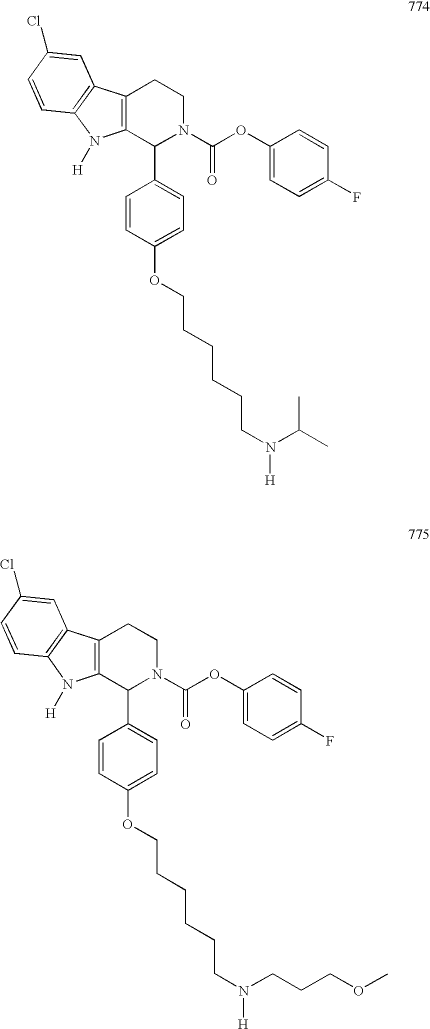

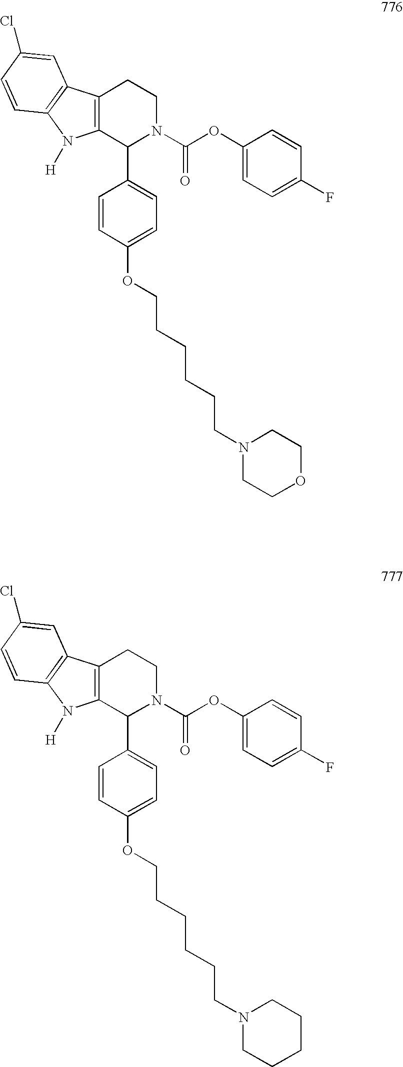

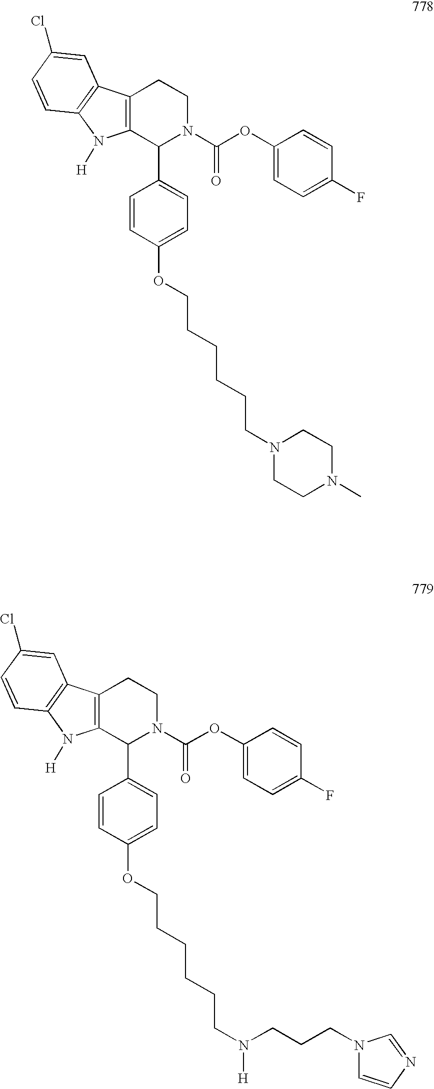

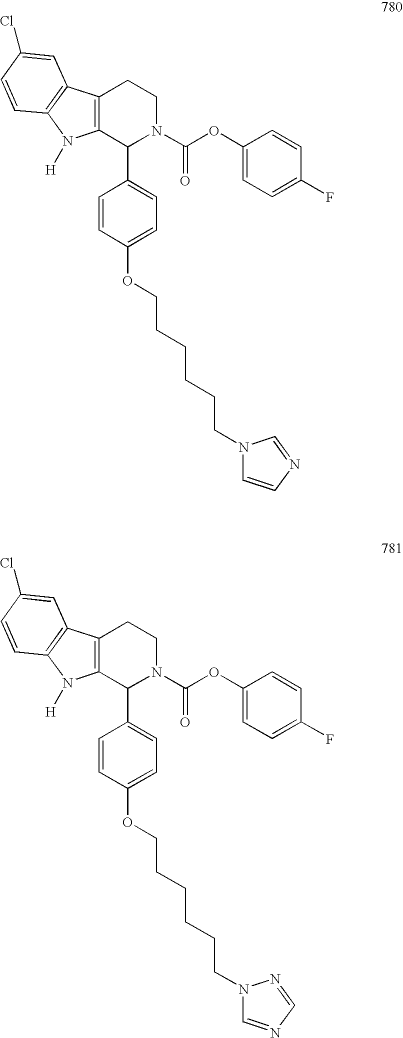

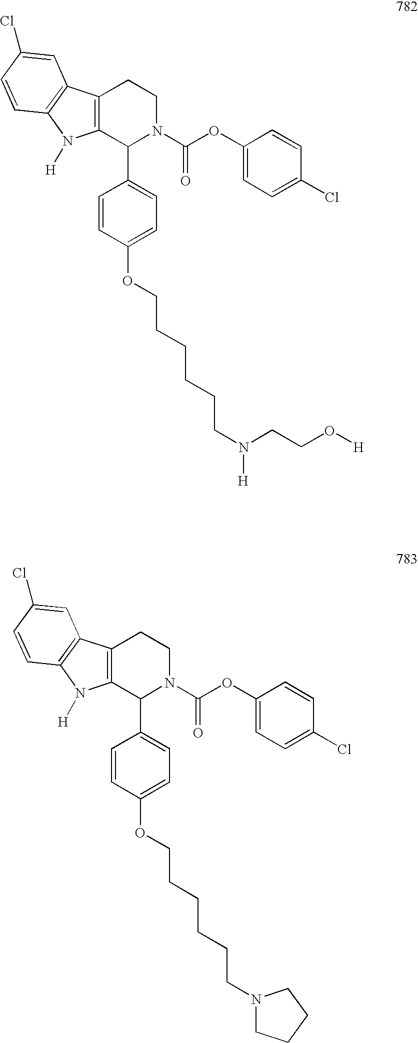

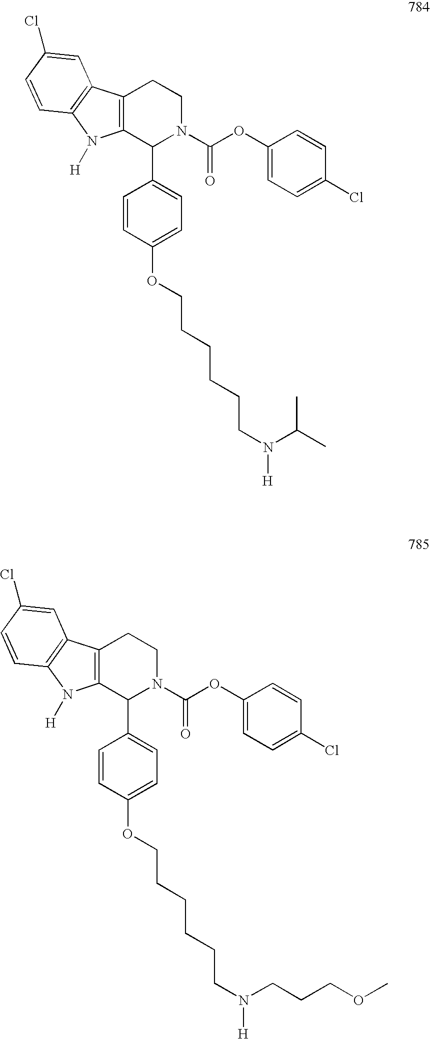

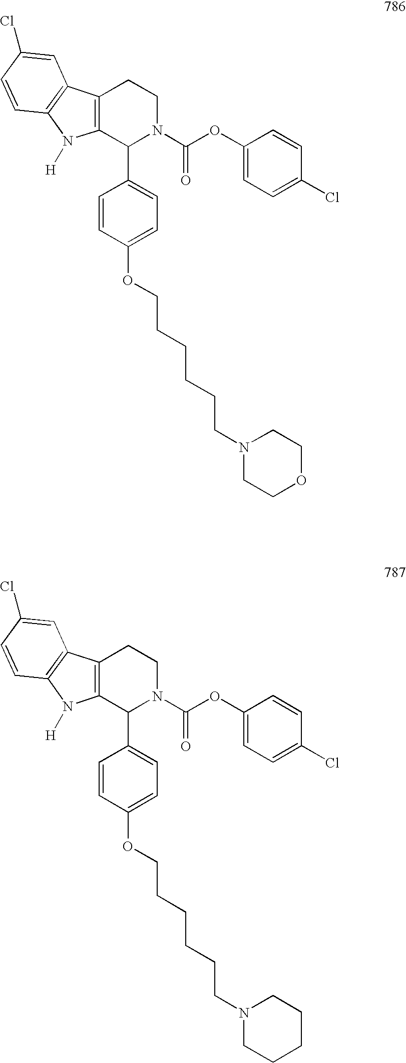

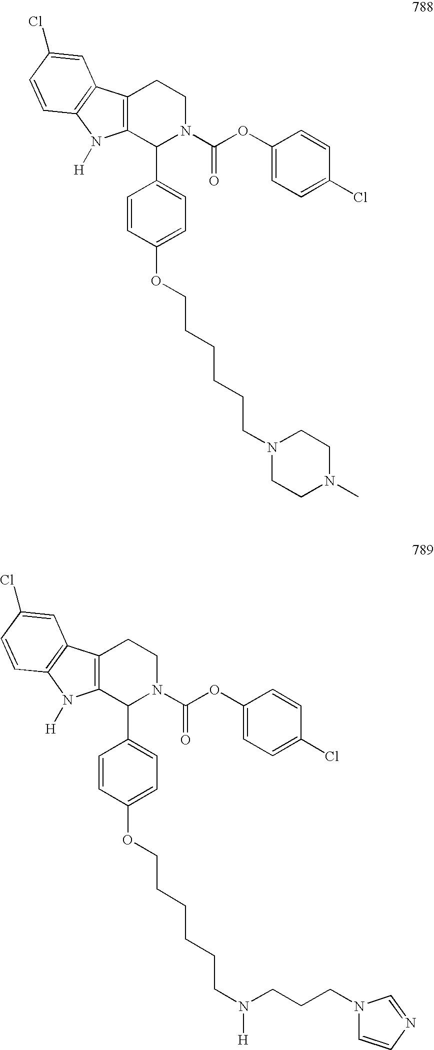

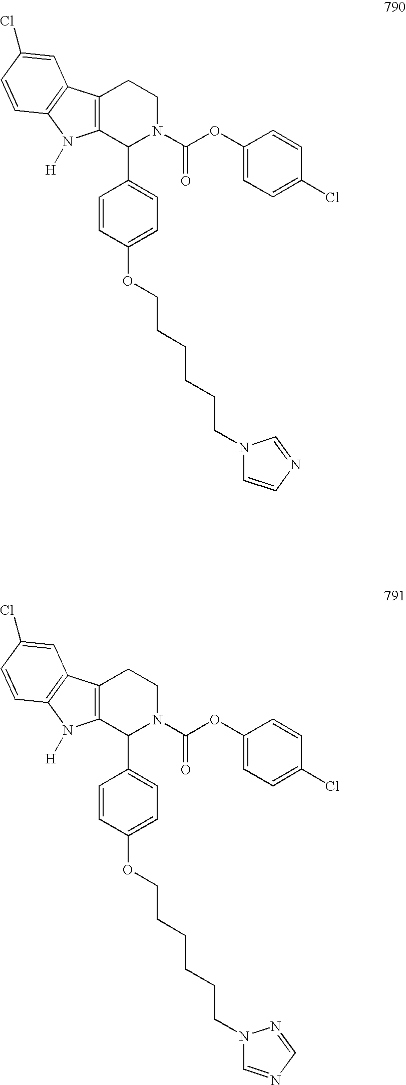

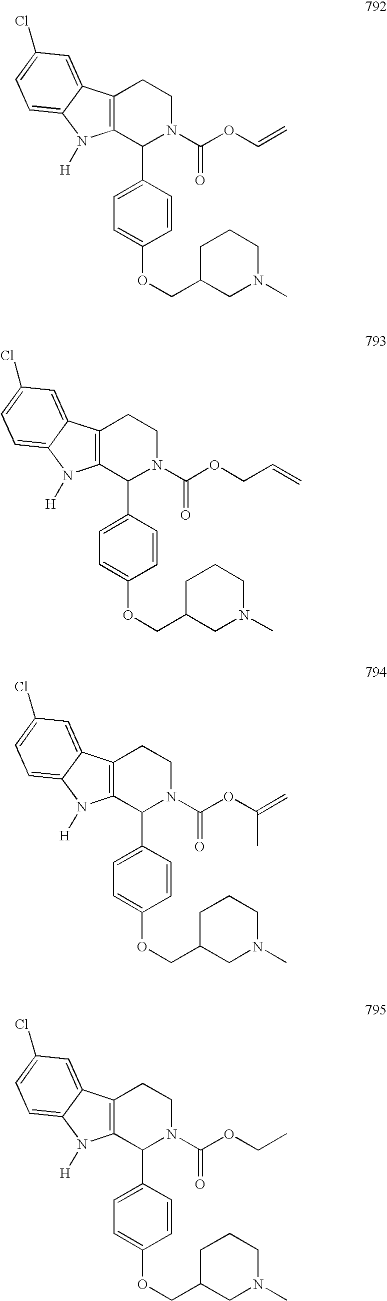

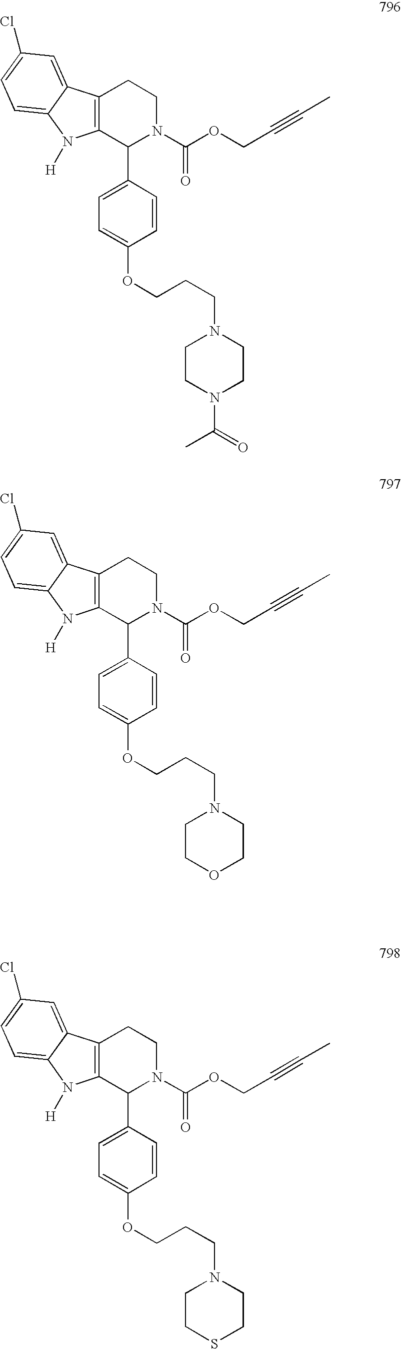

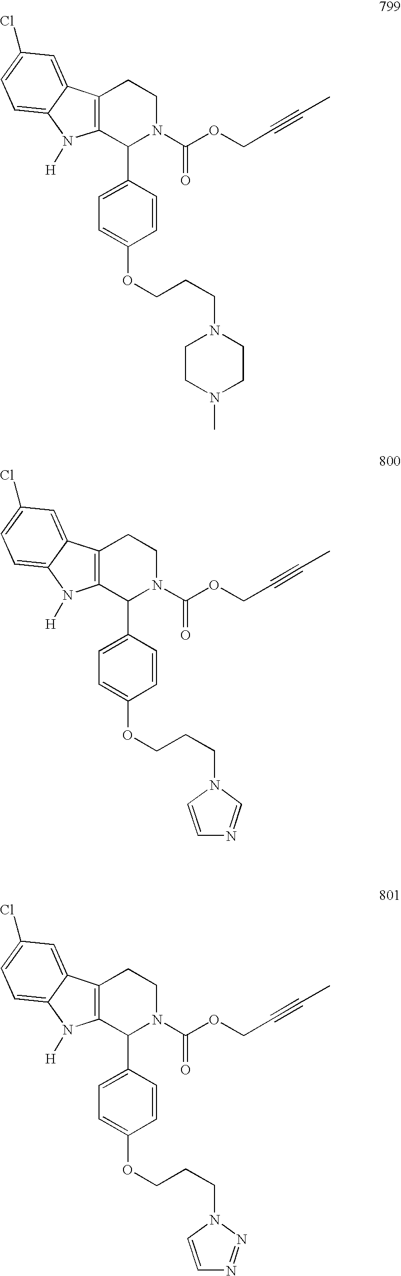

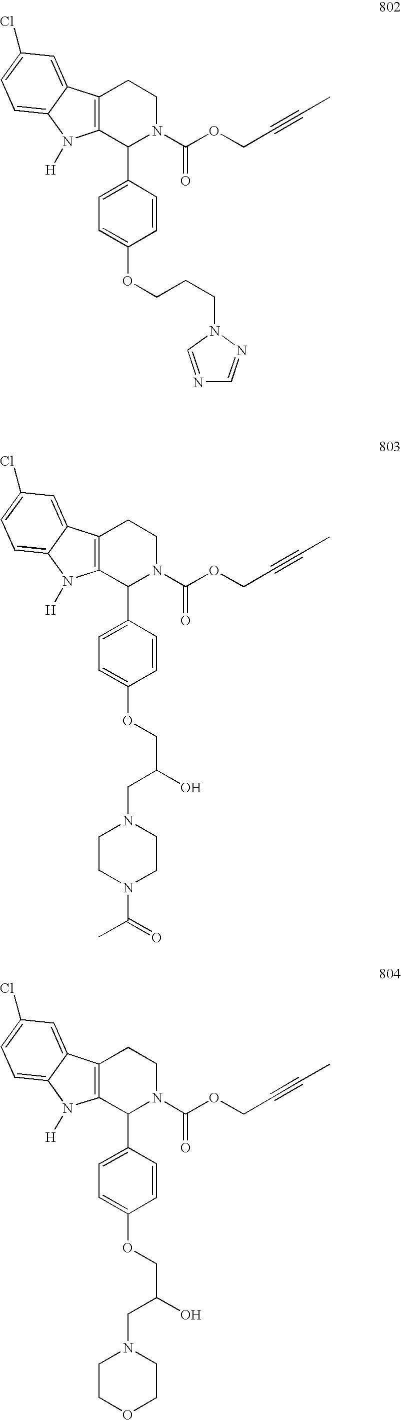

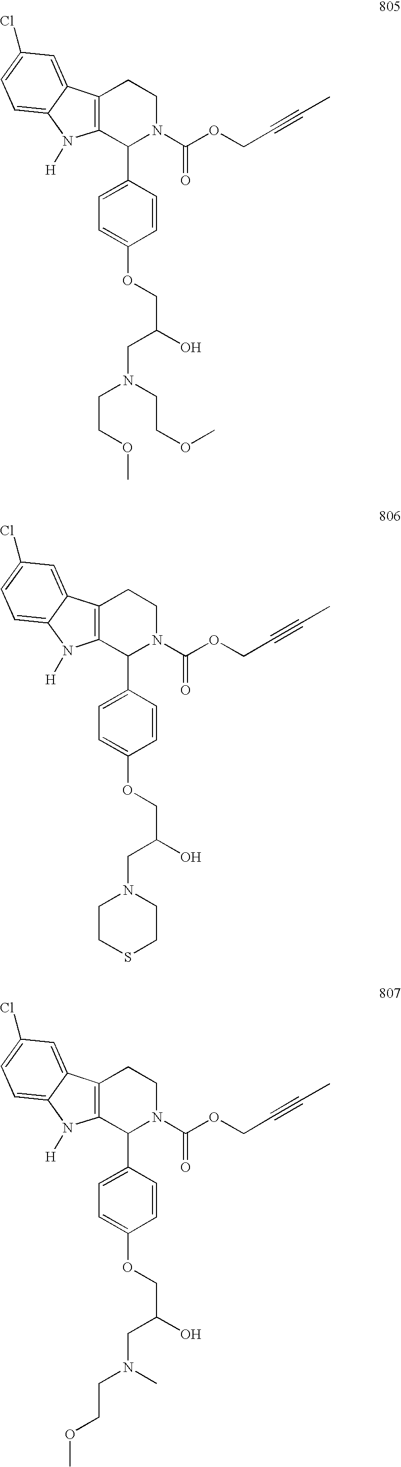

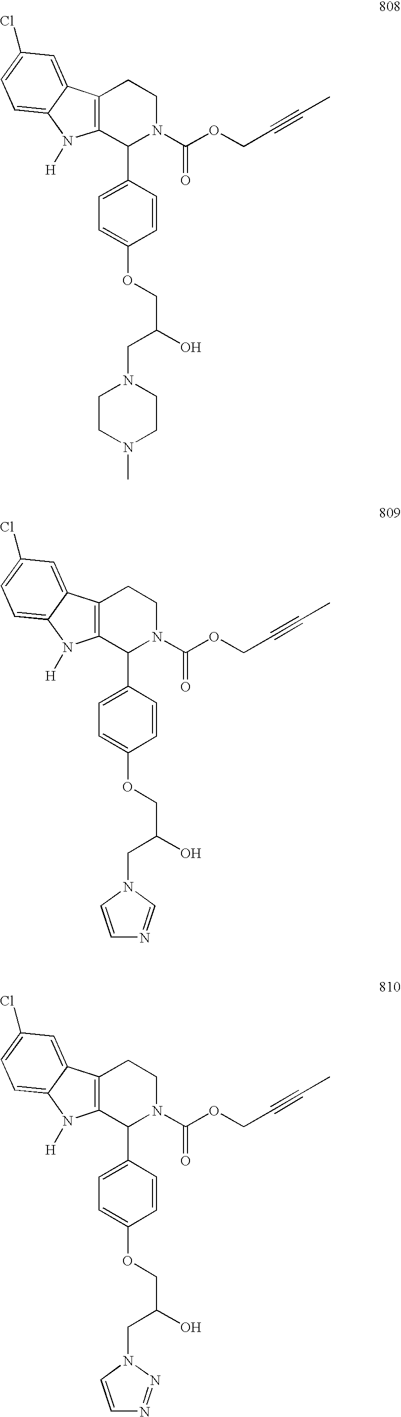

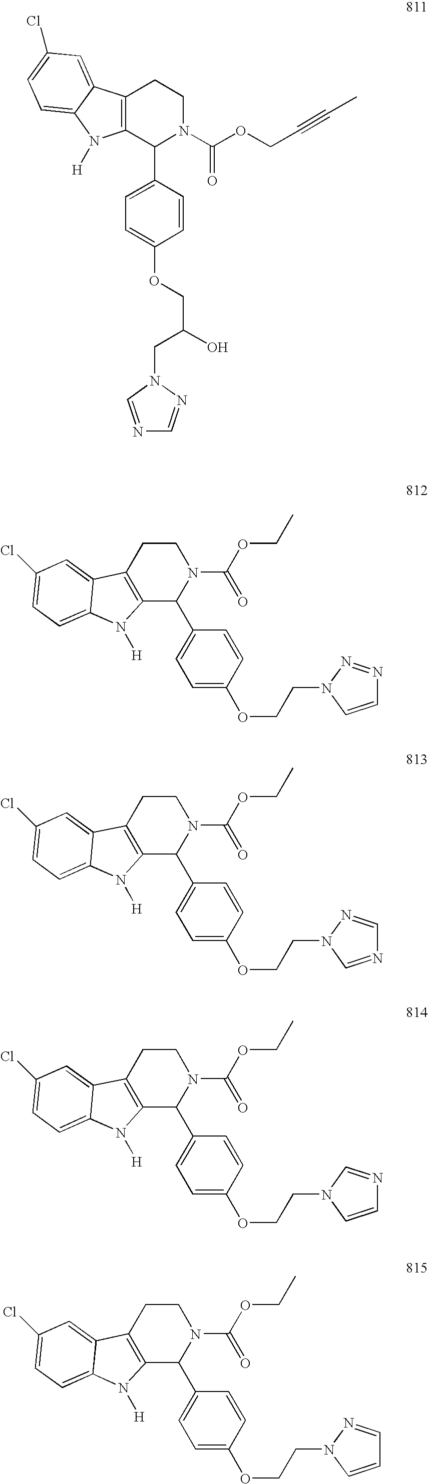

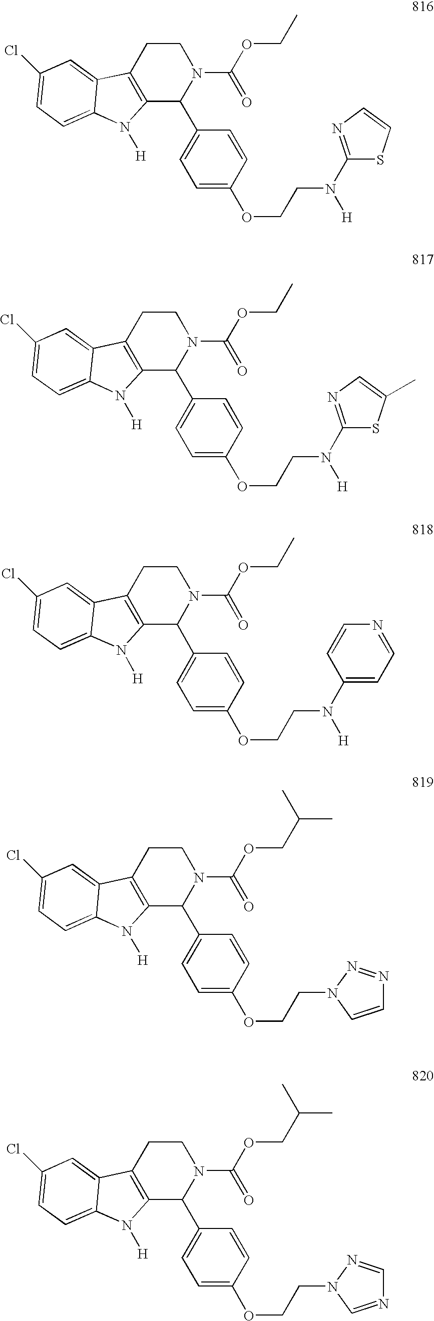

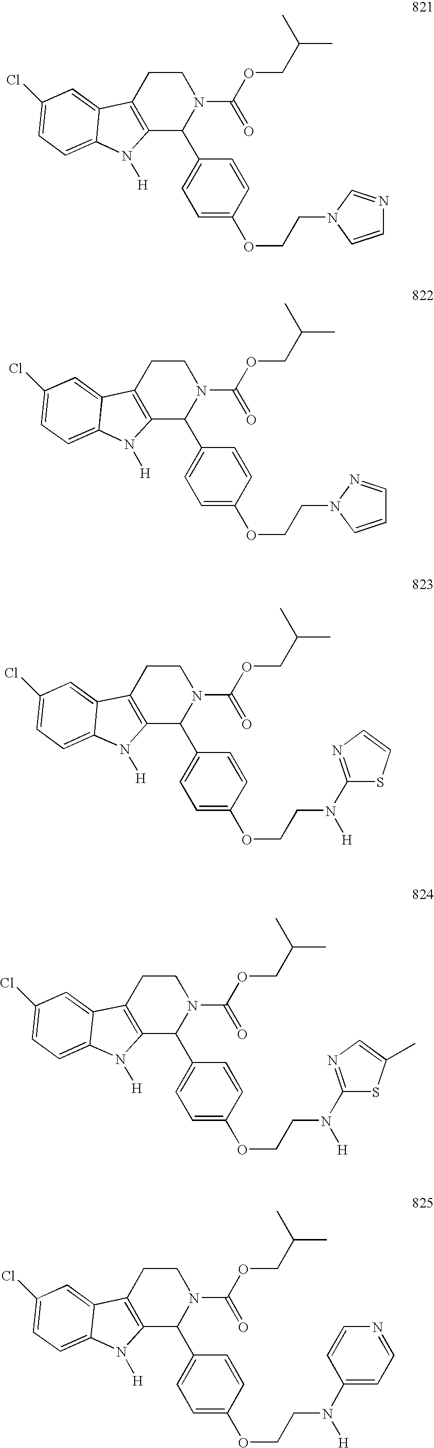

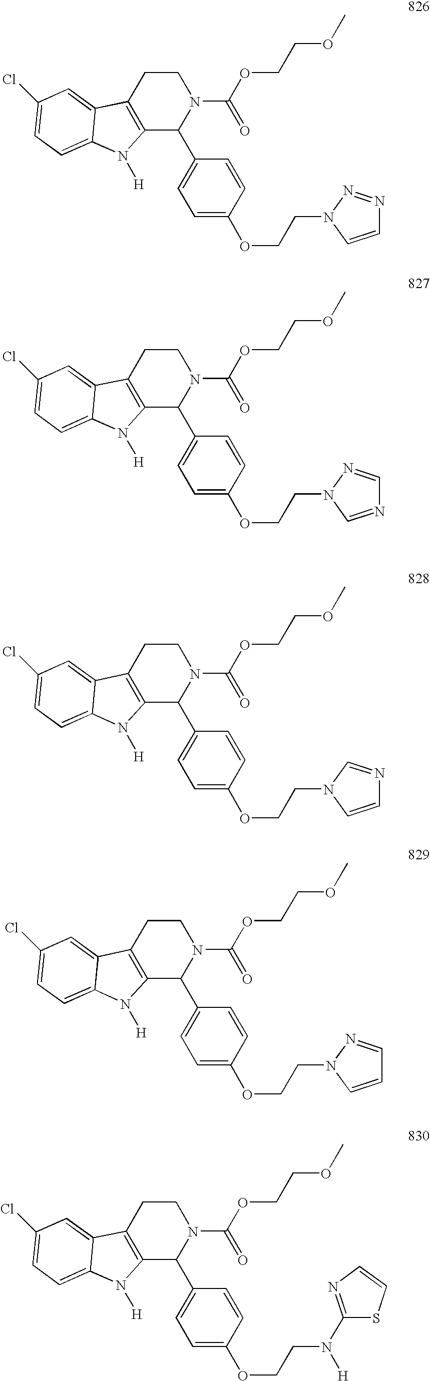

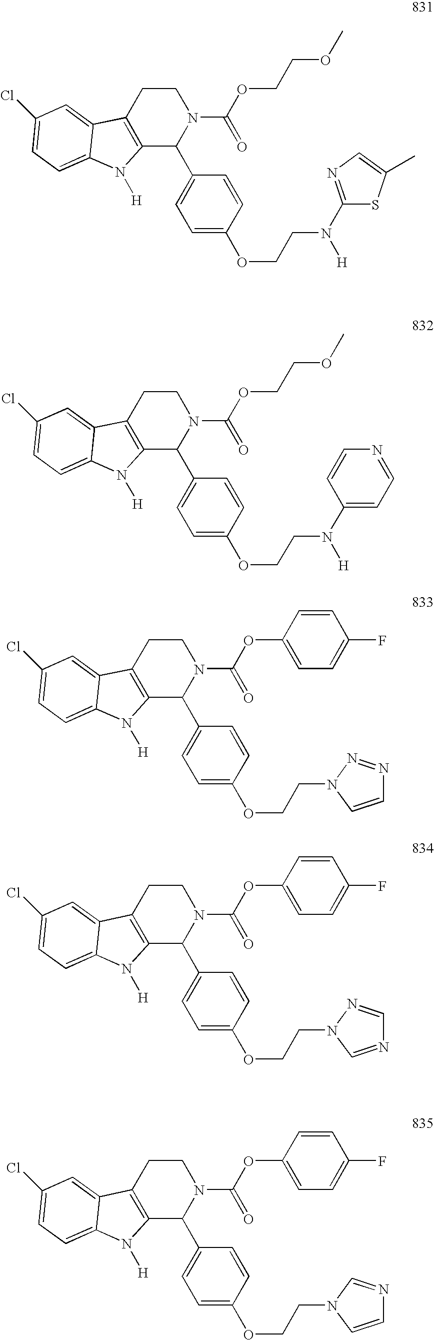

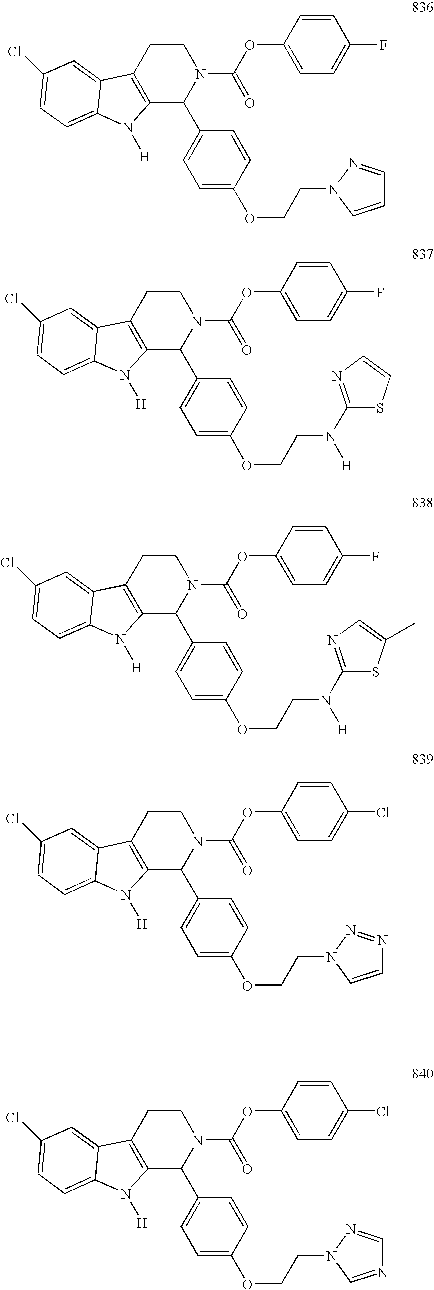

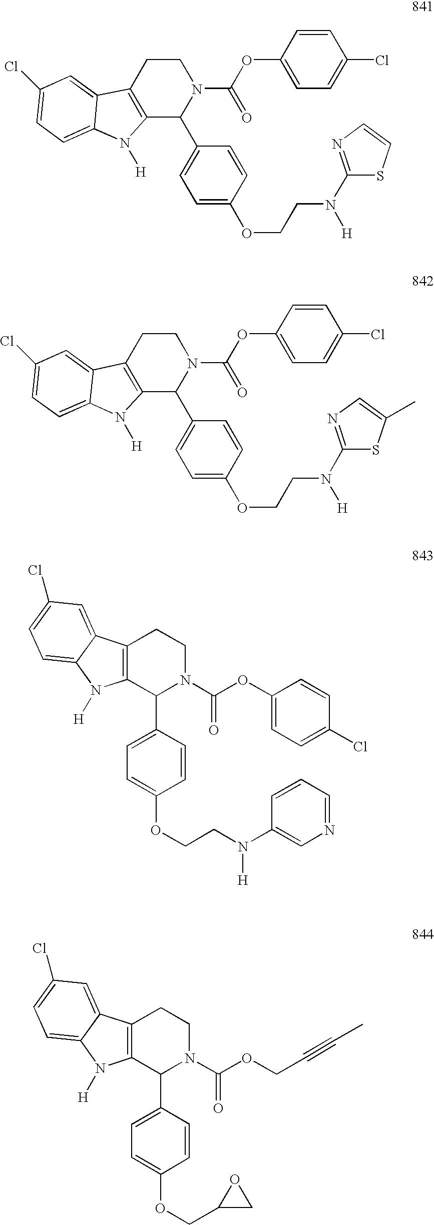

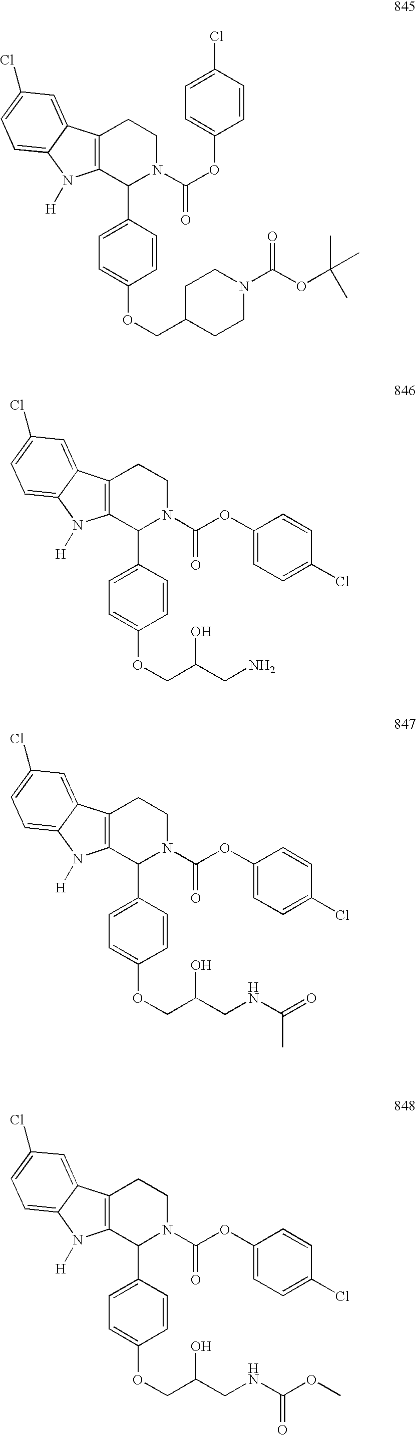

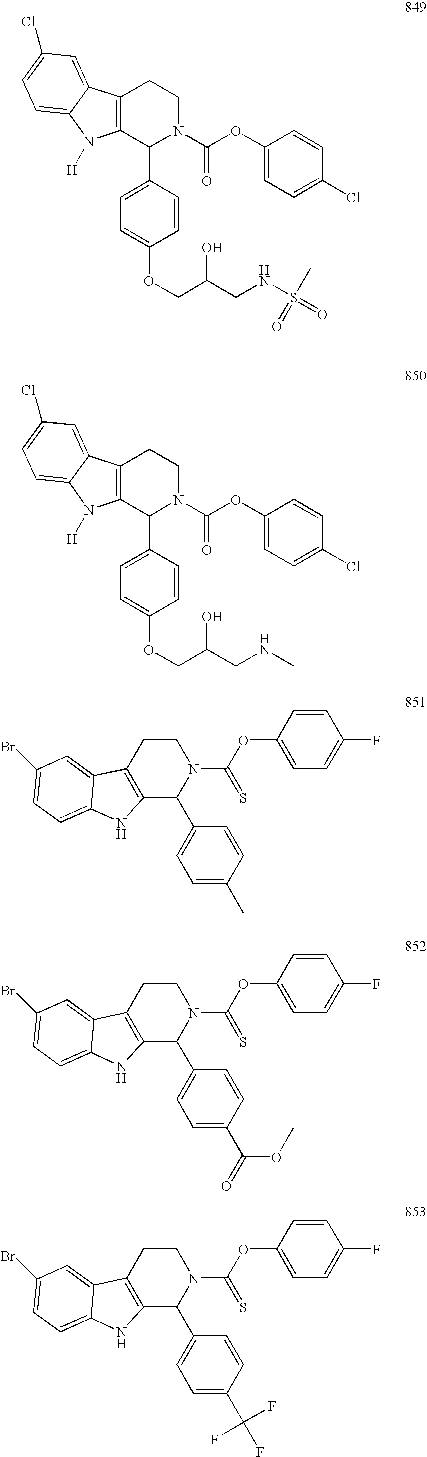

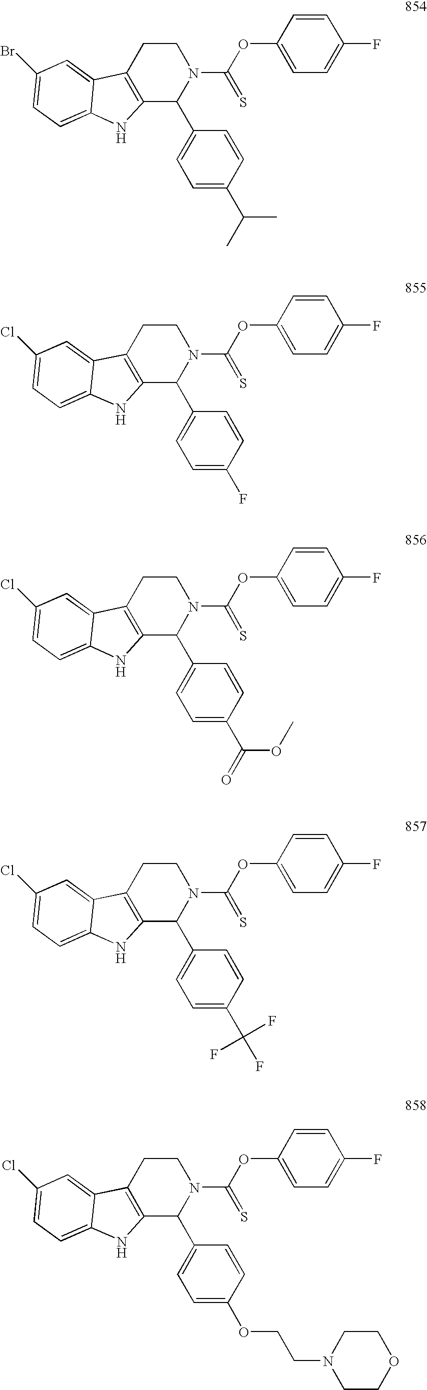

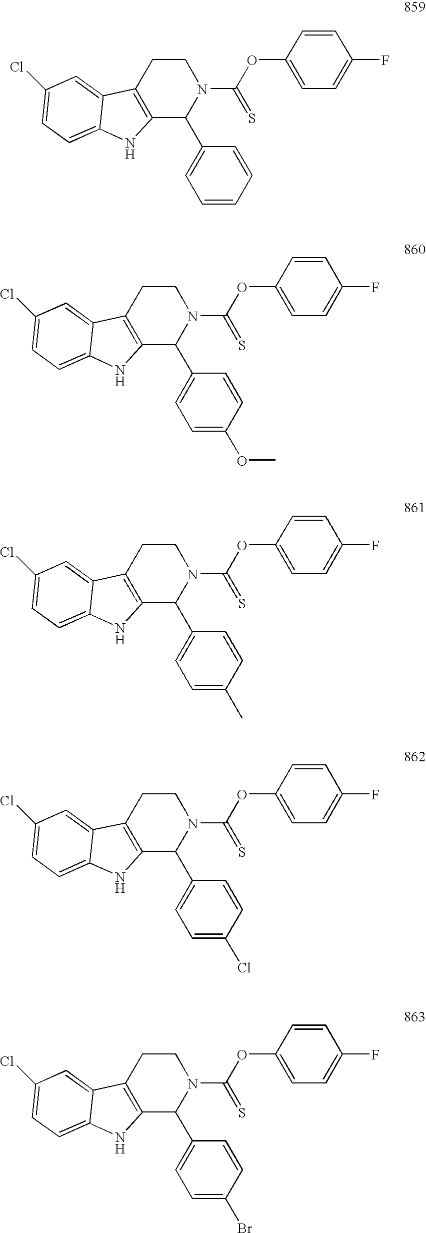

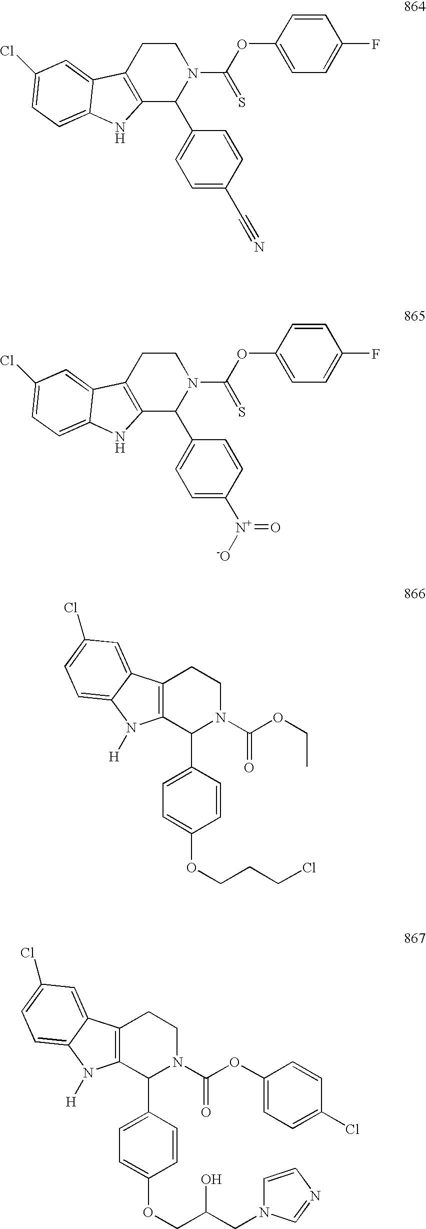

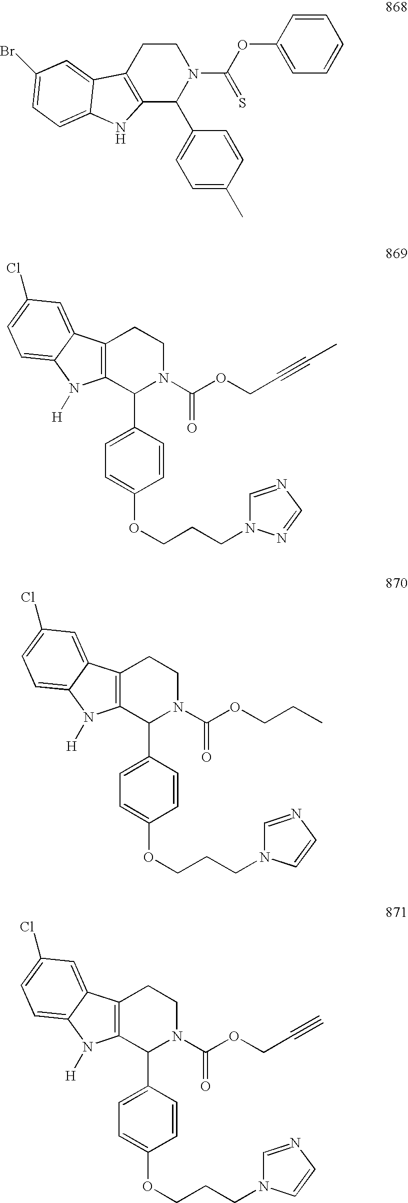

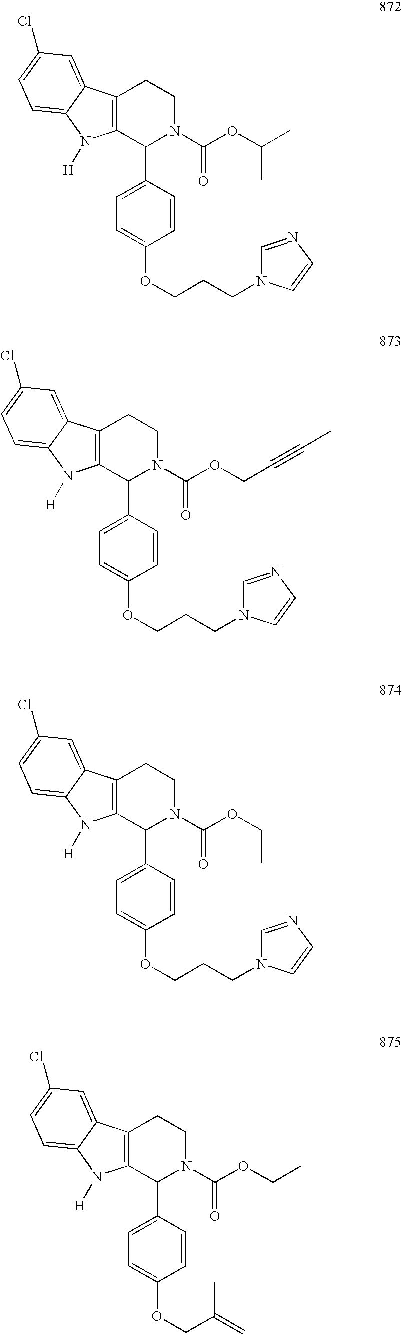

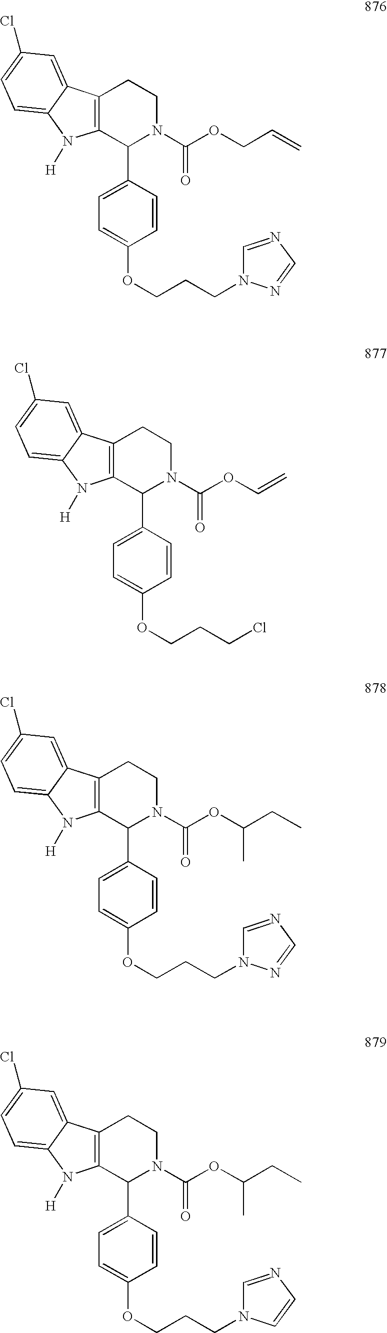

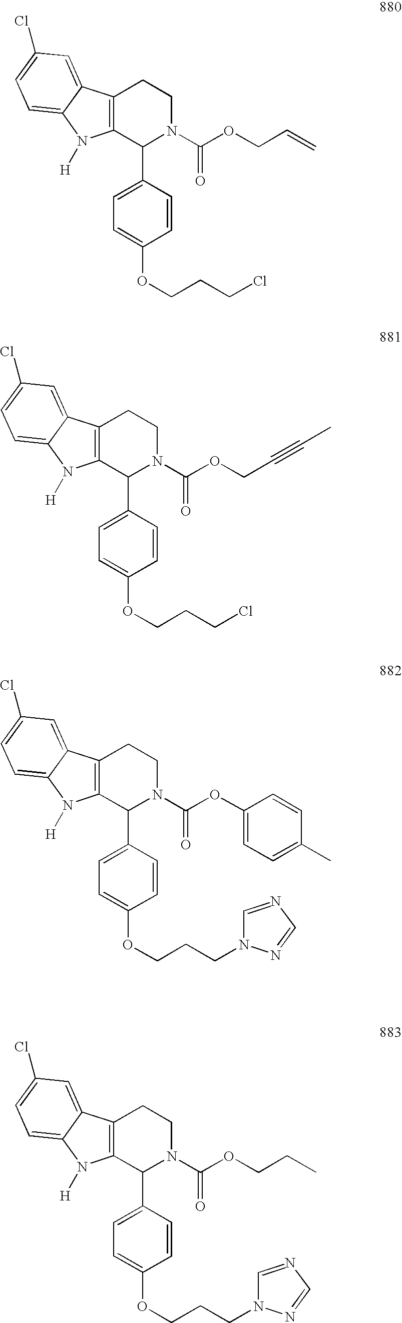

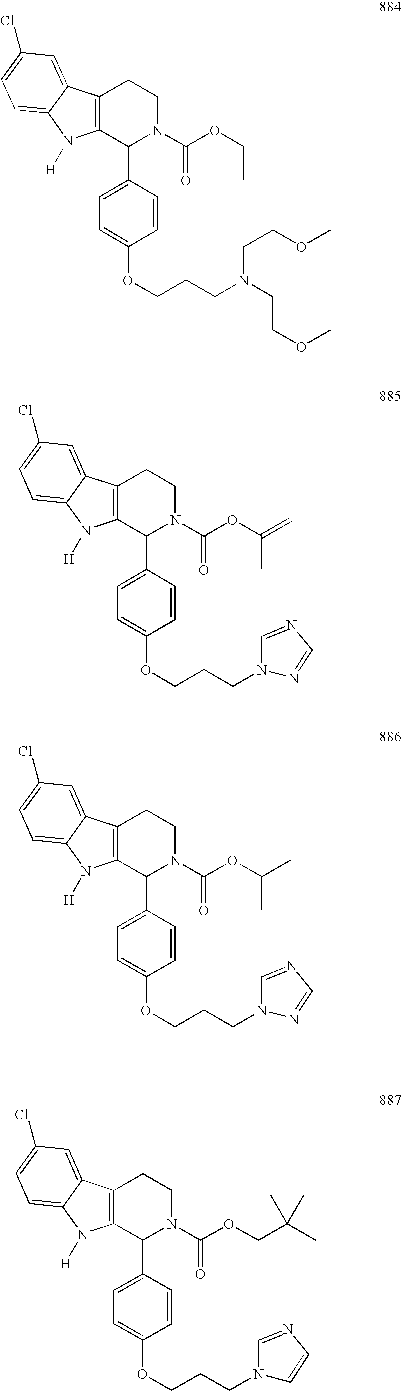

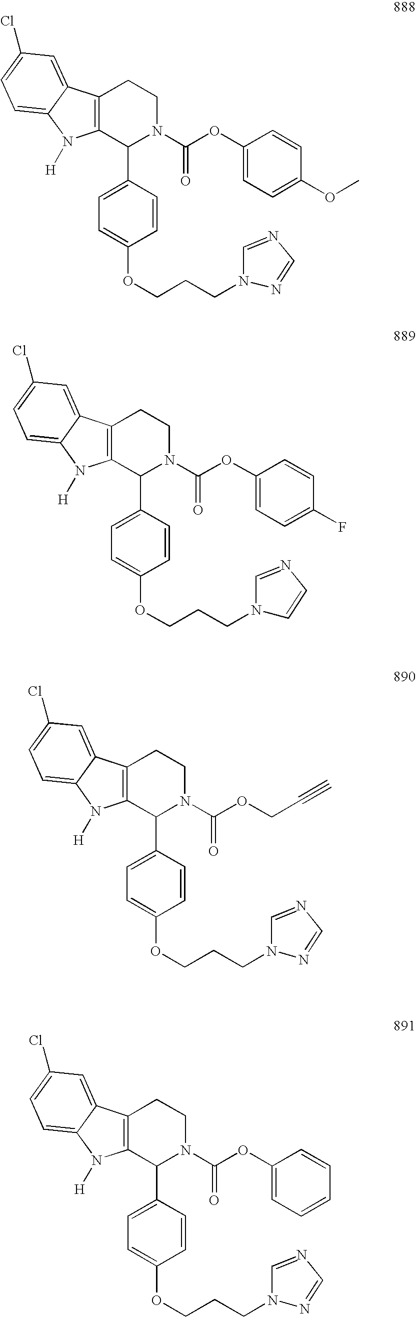

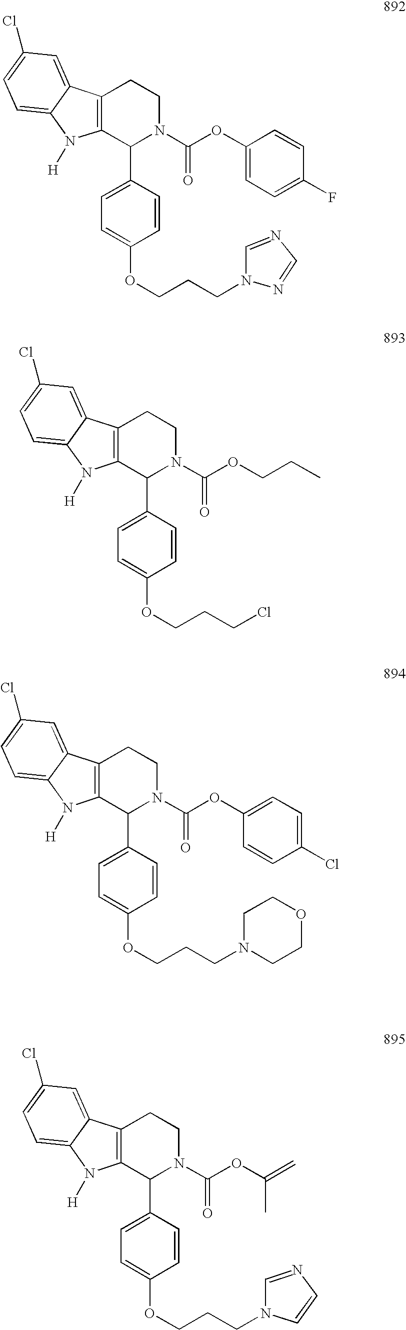

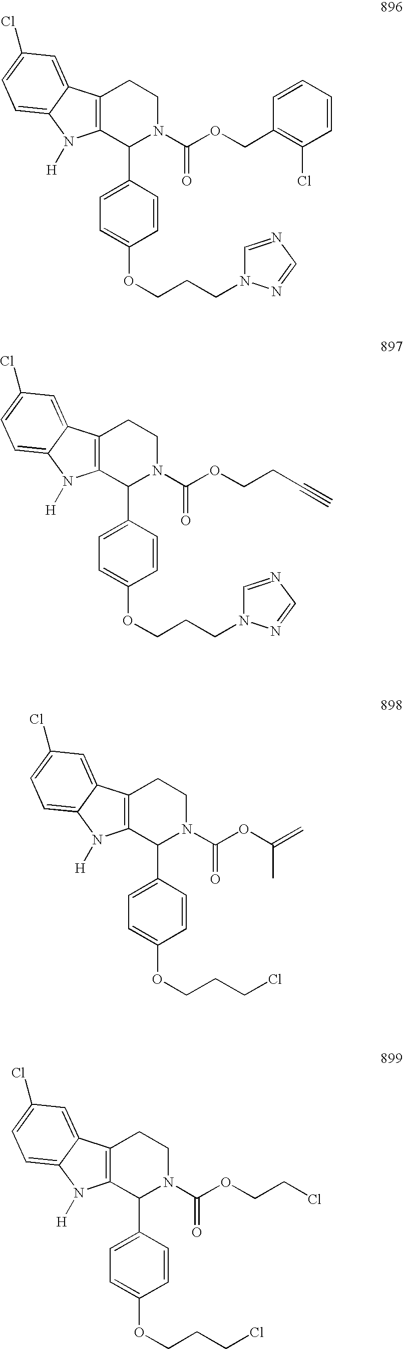

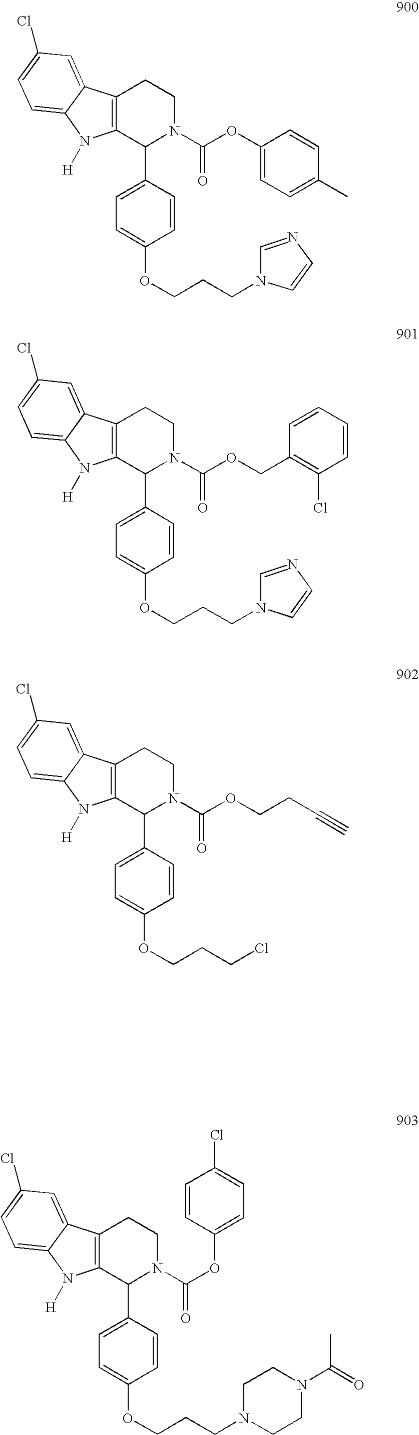

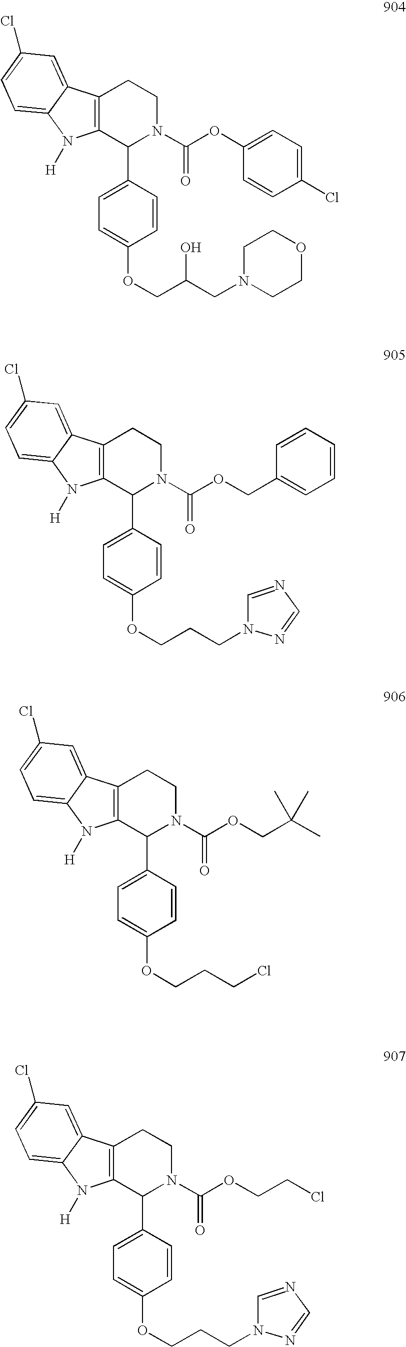

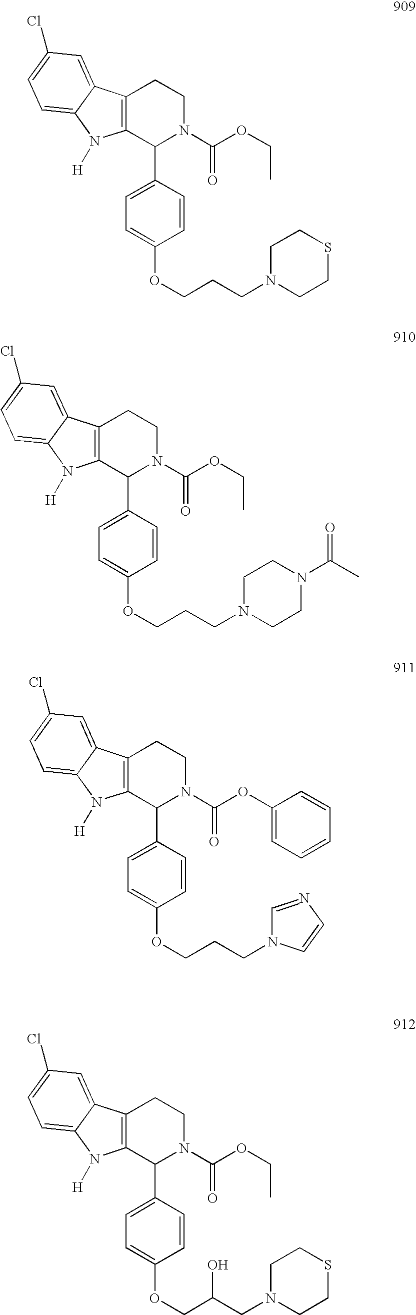

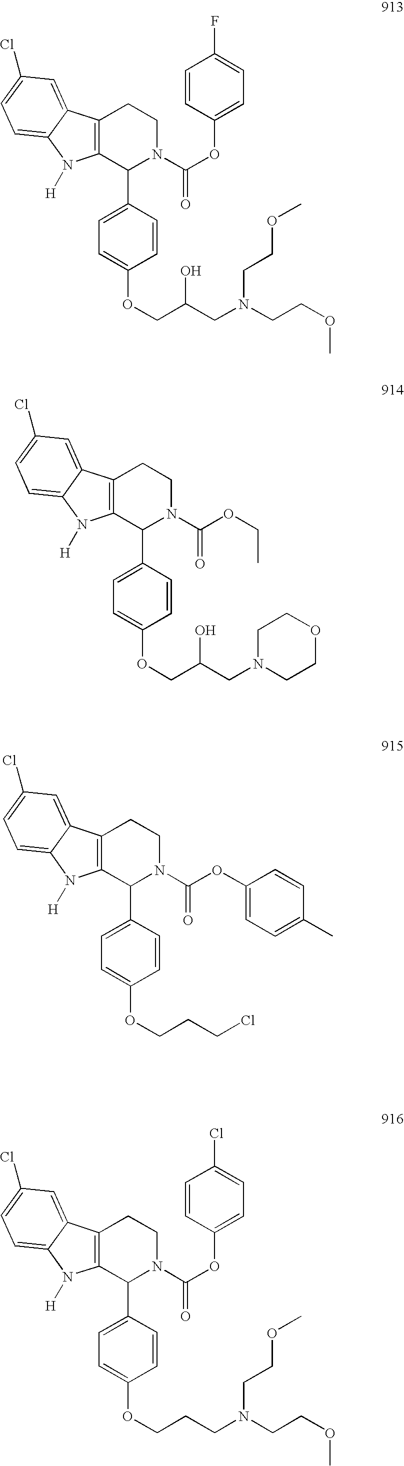

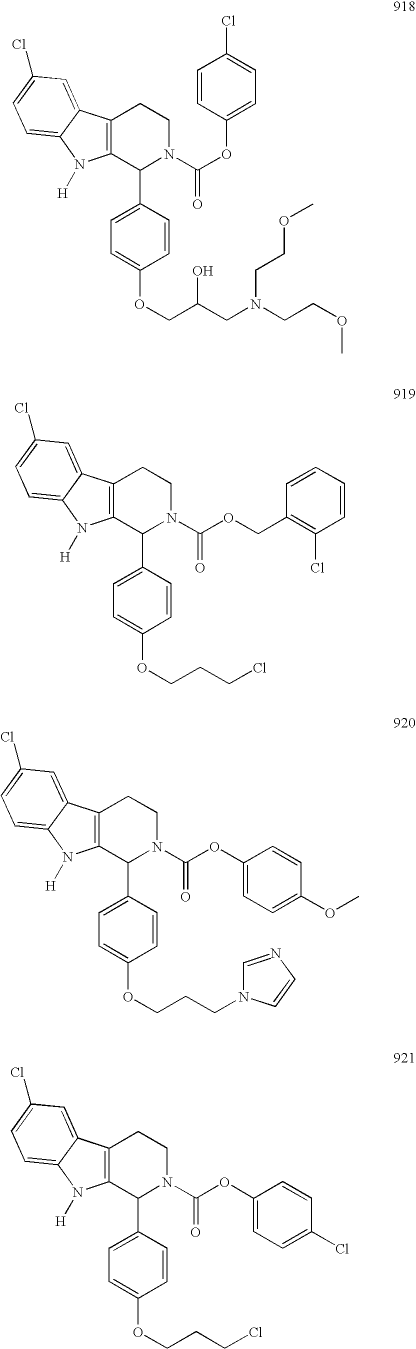

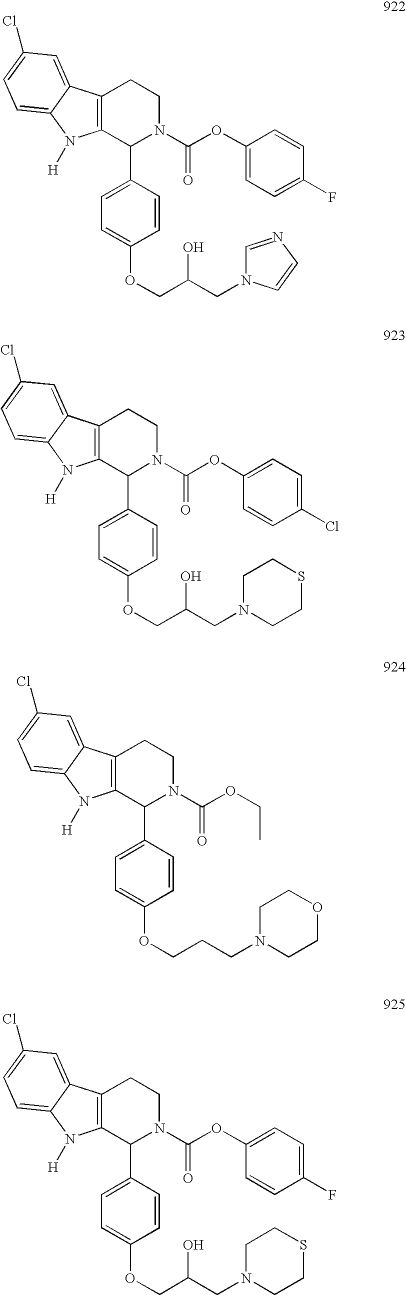

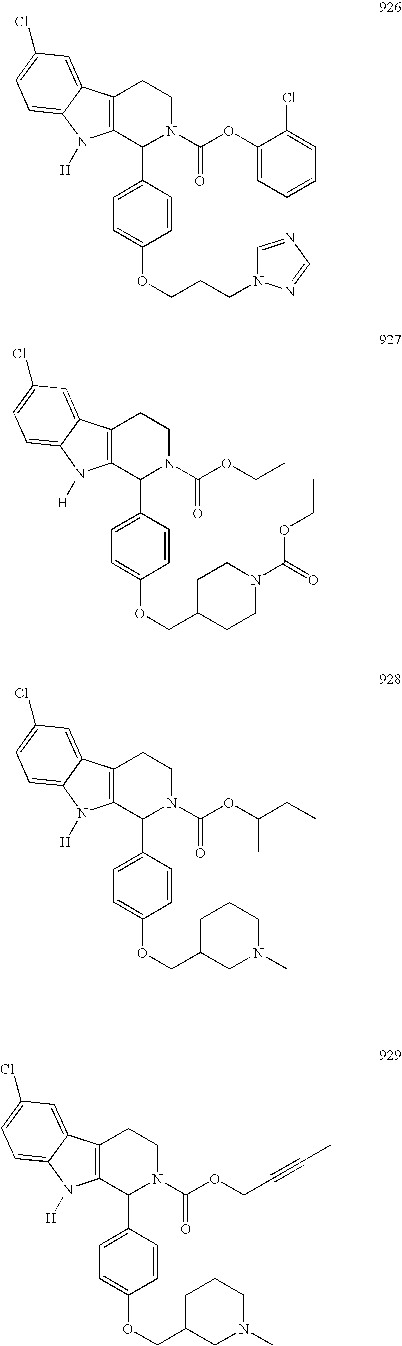

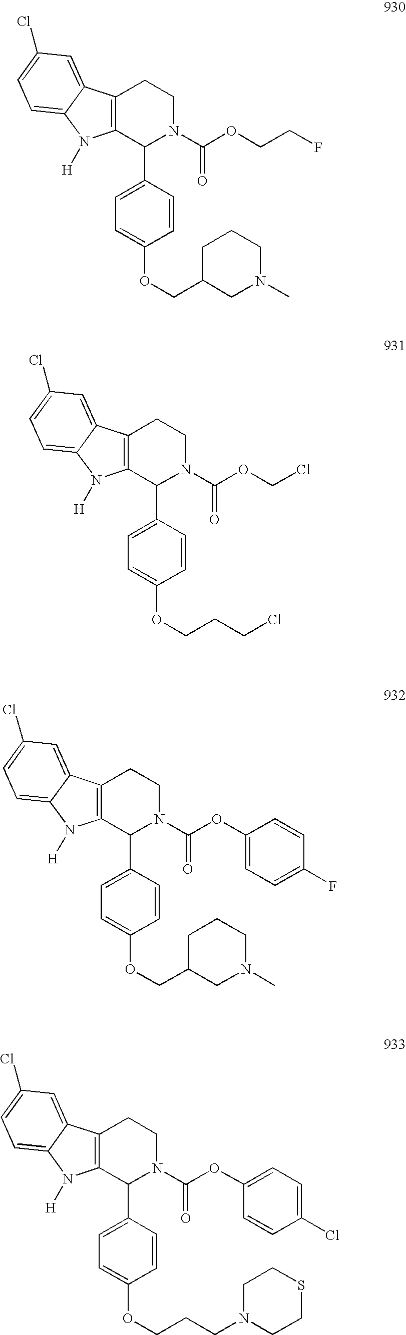

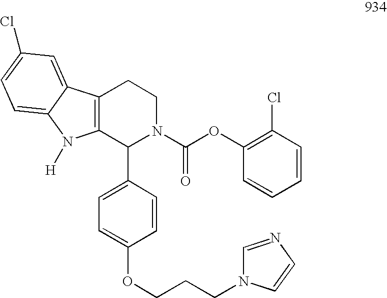

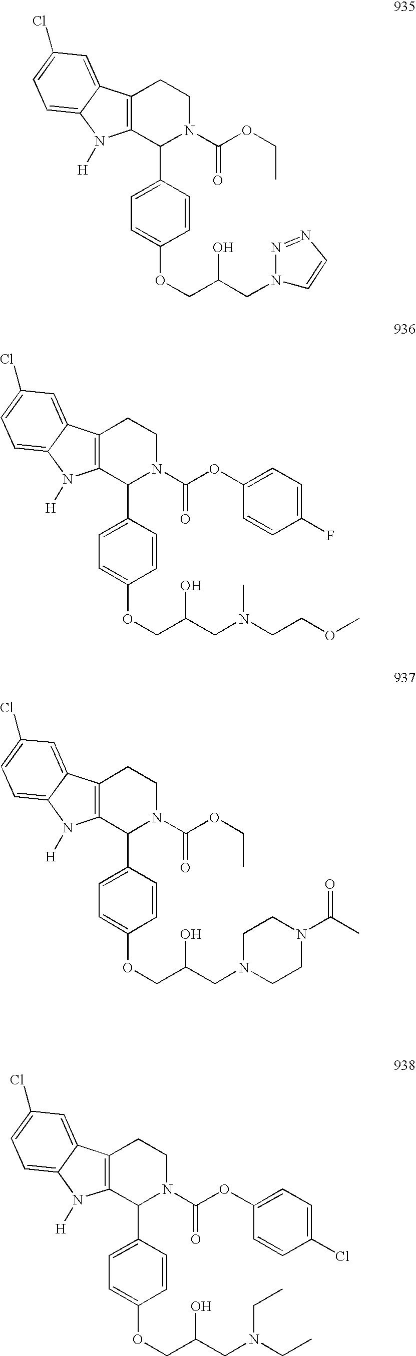

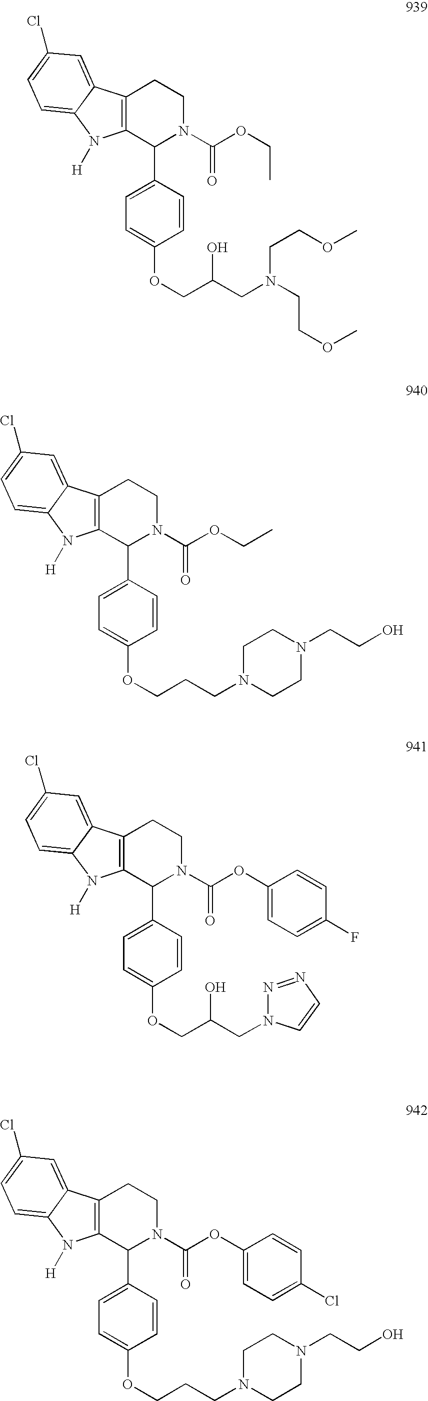

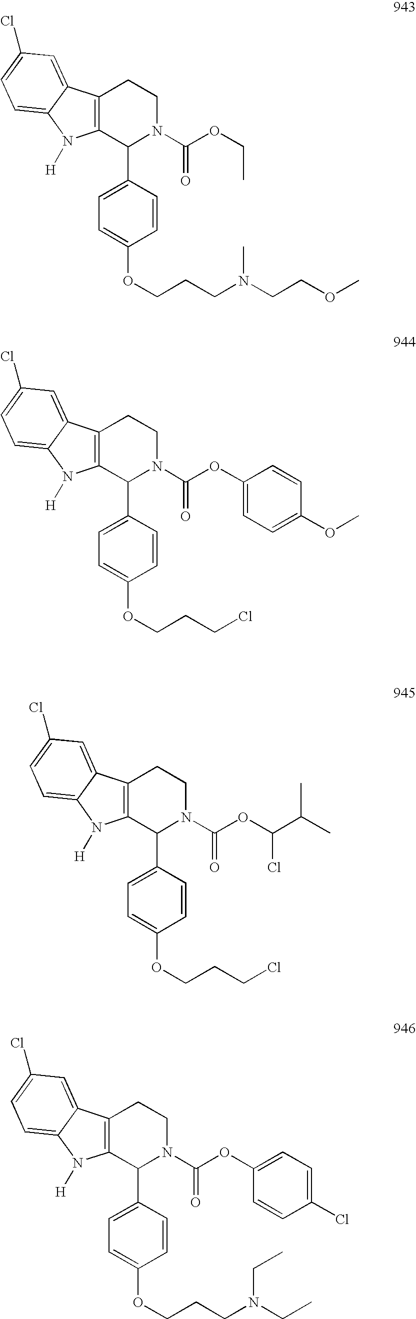

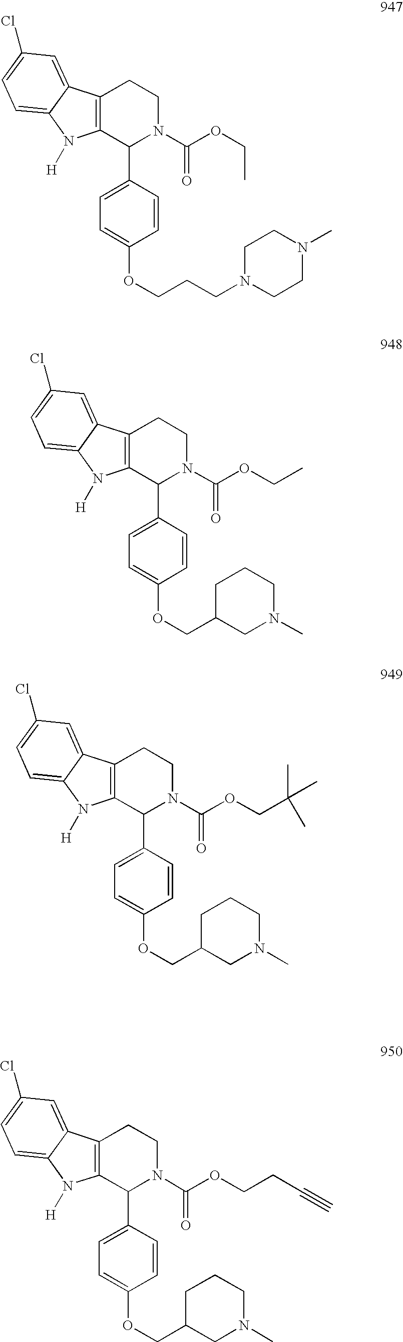

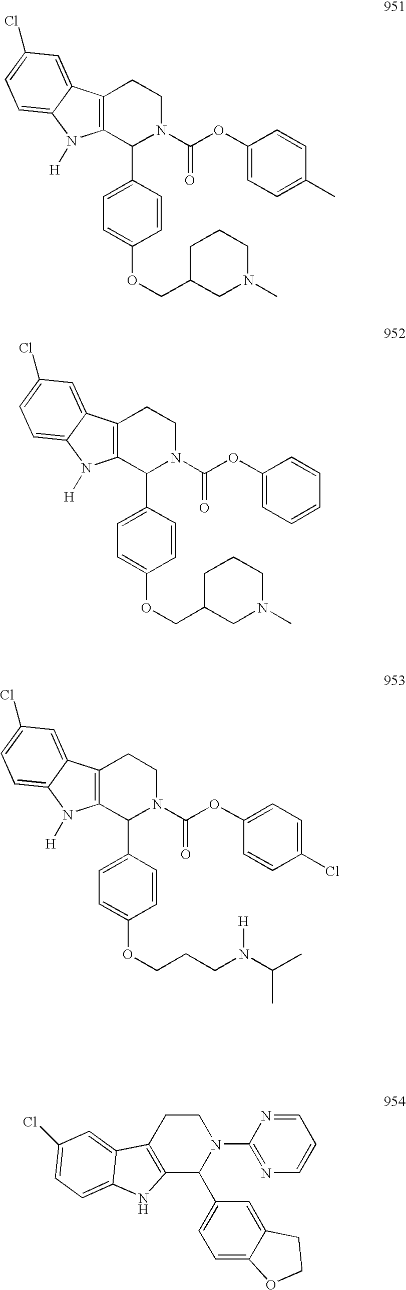

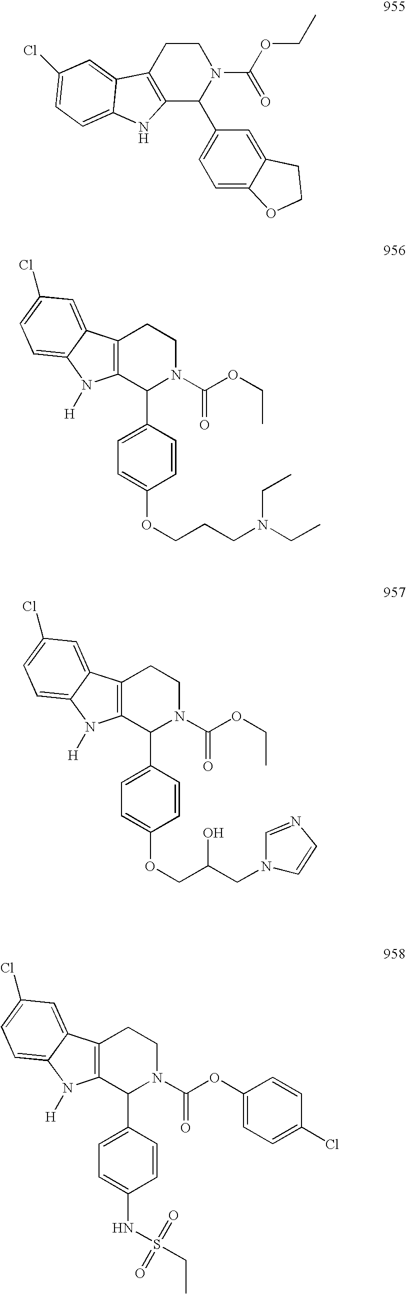

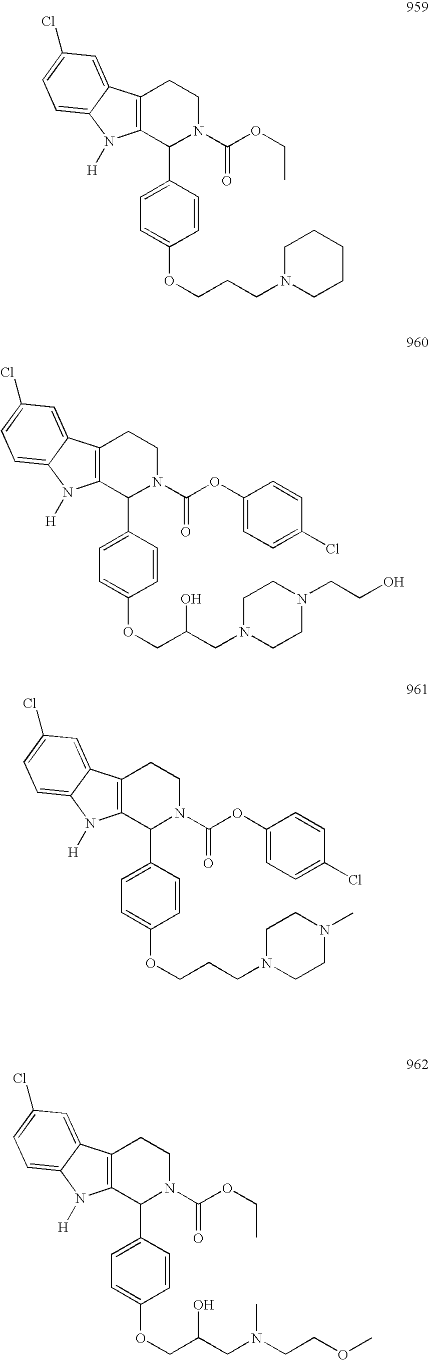

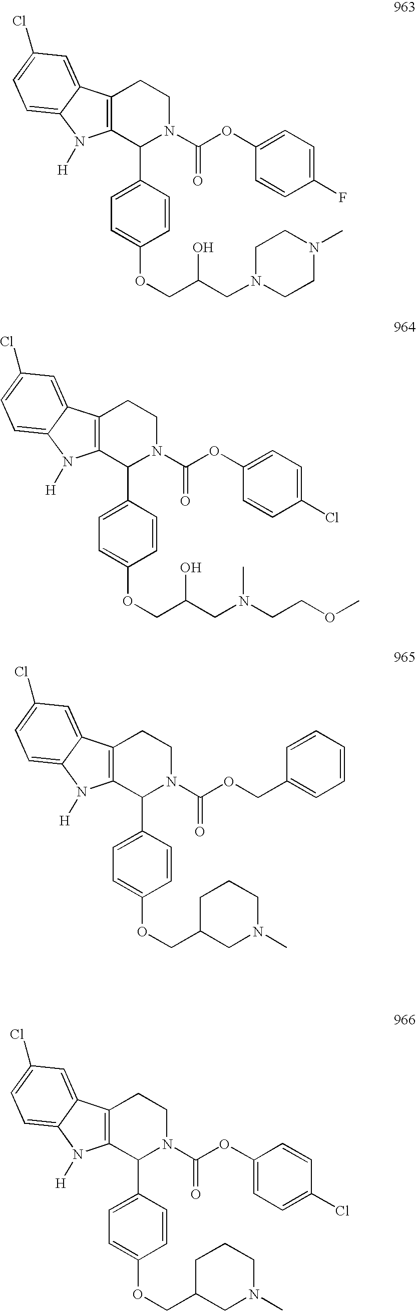

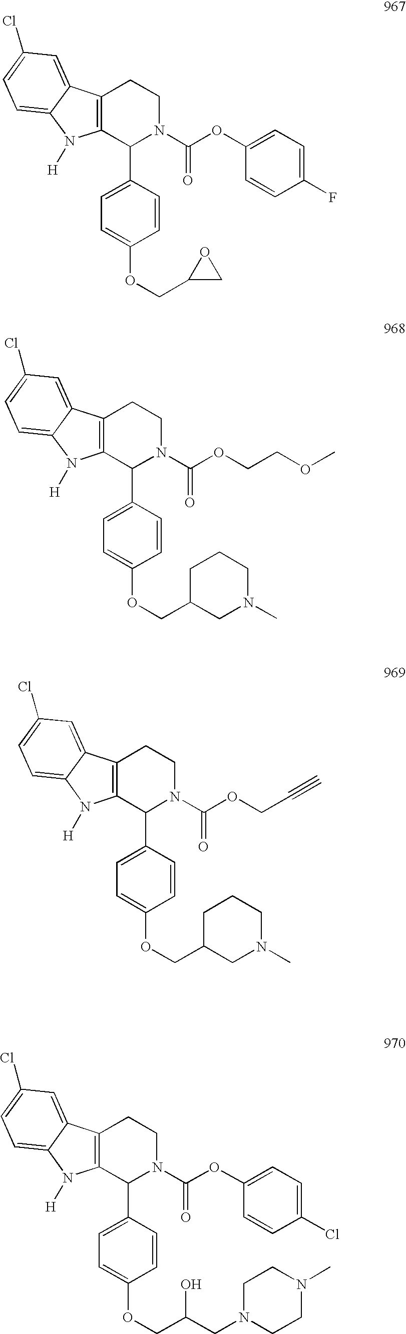

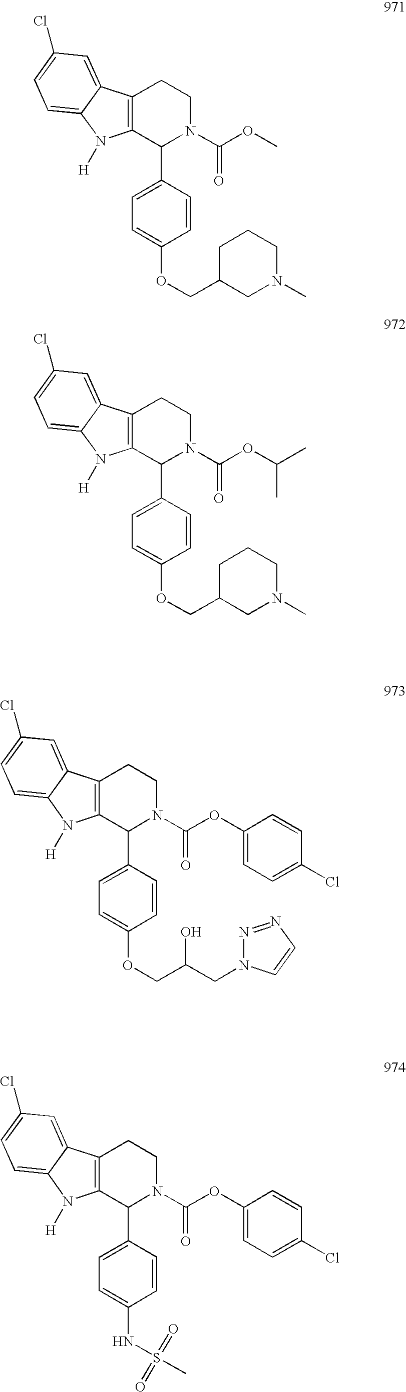

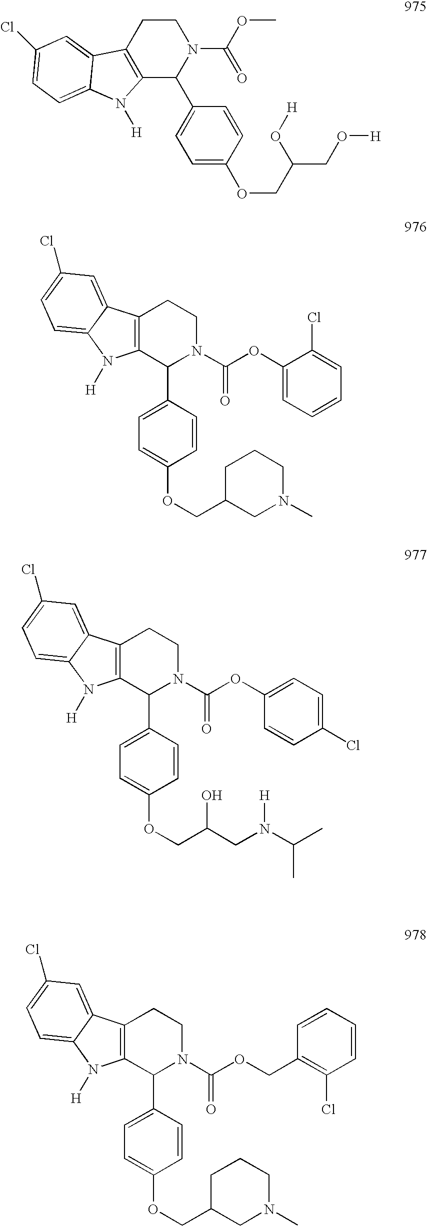

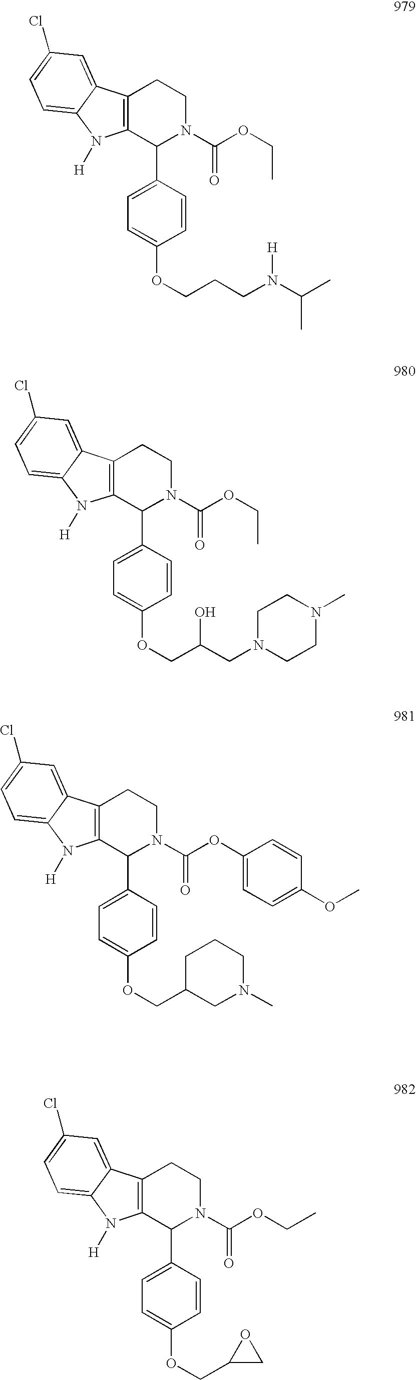

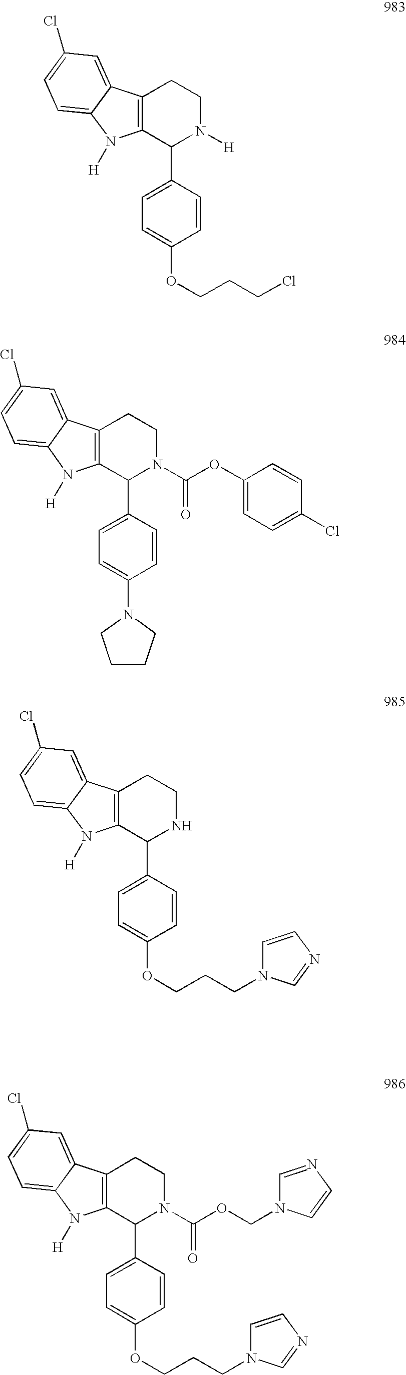

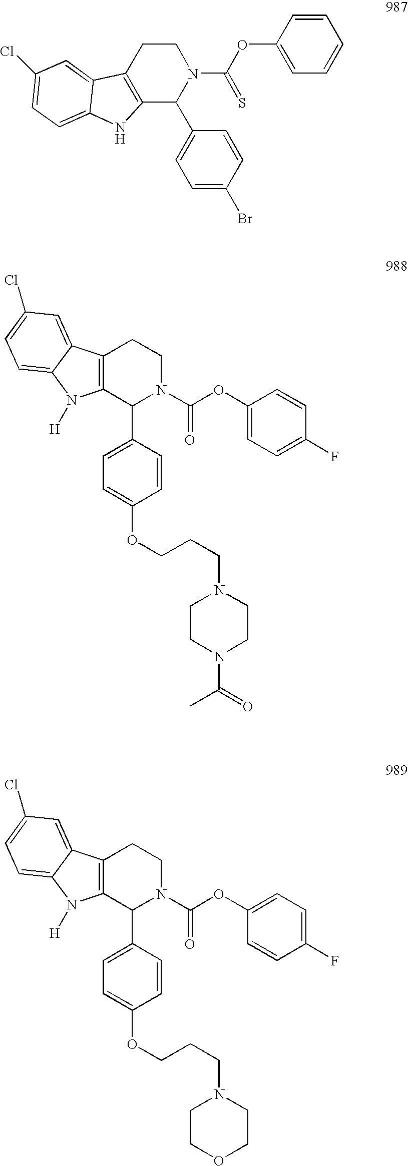

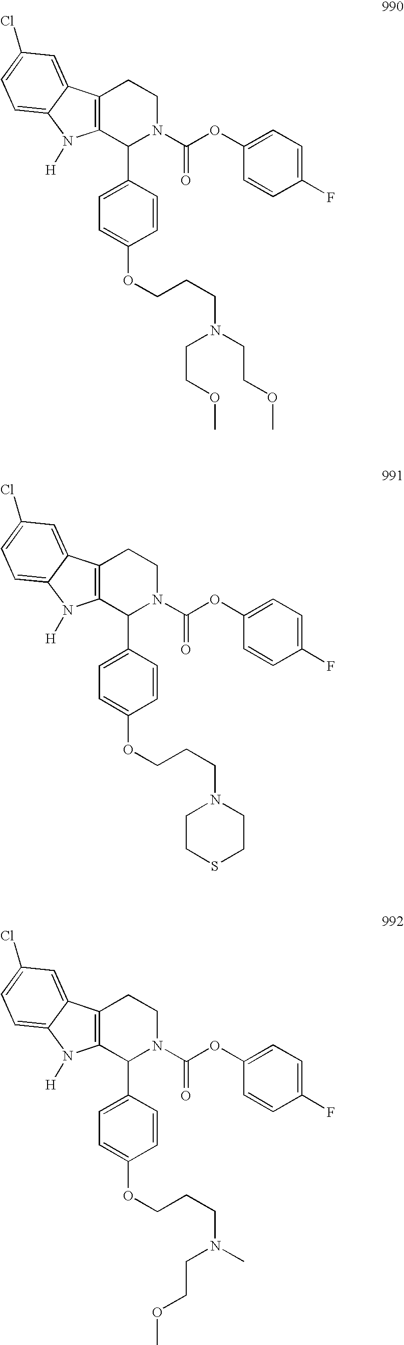

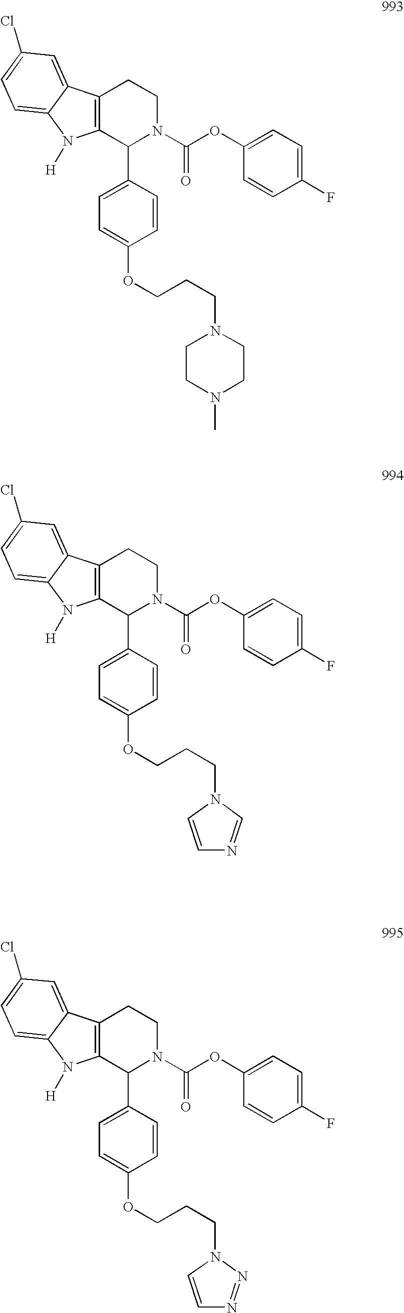

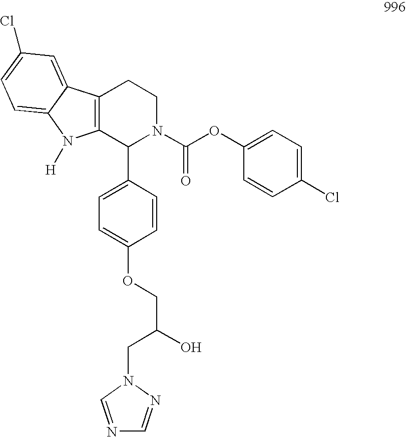

Particularly preferred compounds are selected from the group consisting of Compound Nos: 2, 4, 5, 7, 8, 10, 11, 12, 17, 23, 25, 81, 102, 112, 140, 328, 329, 330, 331, 332, 355, 816, 817, 818, 823, 824, 825, 830, 831, 832, 837, 838, 841, 842, 843, and regioisomers thereof:

| Cpd | Name |
|---|---|
| 2 | ethyl 6-chloro-1-(4-methoxyphenyl)-3,4-dihydro-1H-pyrido[3,4-b]indole-2(9H)- |
| | carboxylate, |
| 4 | ethyl 6-bromo-1-(4-chlorophenyl)-3,4-dihydro-1H-pyrido[3,4-b]indole-2(9H)- |
| | carboxylate, |
| 5 | ethyl 6-chloro-1-(2,3-difluorophenyl)-3,4-dihydro-1H-pyrido[3,4-b]indole-2(9H)- |
| | carboxylate, |
| 7 | ethyl 6-bromo-1-(4-isopropylphenyl)-3,4-dihydro-1H-pyrido[3,4-b]indole-2(9H)- |
| | carboxylate, |
| 8 | ethyl 6-bromo-1-p-tolyl-3,4-dihydro-1H-pyrido[3,4-b]indole-2(9H)-carboxylate, |
| 10 | 4-chlorophenyl 6-chloro-1-(4-methoxyphenyl)-3,4-dihydro-1H-pyrido[3,4-b]indole- |
| | 2(9H)-carboxylate, |
| 11 | ethyl 6-chloro-1-(4-chlorophenyl)-3,4-dihydro-1H-pyrido[3,4-b]indole-2(9H)- |
| | carboxylate, |
| 12 | 2-chloroethyl 6-chloro-1-(4-cyanophenyl)-3,4-dihydro-1H-pyrido[3,4-b]indole-2(9H)- |
| | carboxylate, |
| 17 | p-tolyl 6-chloro-1-(4-methoxyphenyl)-3,4-dihydro-1H-pyrido[3,4-b]indole-2(9H)- |
| | carboxylate, |
| 23 | ethyl 6-chloro-1-(4-fluorophenyl)-3,4-dihydro-1H-pyrido[3,4-b]indole-2(9H)- |
| | carboxylate, |
| 25 | 6-bromo-1-(4-isopropylphenyl)-2-(pyrimidin-2-yl)-2,3,4,9-tetrahydro-1H-pyrido[3,4- |
| | b]indole, |
| 81 | 6-bromo-1-(4-chlorophenyl)-3,4-dihydro-1H-pyrido[3,4-b]indol-2(9H)-ol, |
| 102 | 1-(6-bromo-1-(4-isopropylphenyl)-3,4-dihydro-1H-pyrido[3,4-b]indol-2(9H)- |
| | yl)ethanone, |
| 112 | 6-bromo-1-(4-isopropylphenyl)-2,3,4,9-tetrahydro-1H-pyrido[3,4-b]indole, |
| 140 | 6-bromo-1-(3-chlorophenyl)-N-cyclohexyl-3,4-dihydro-1H-pyrido[3,4-b]indole- |
| | 2(9H)-carboxamide, |
| 328 | 1-(benzo[d][1,3]dioxol-5-yl)-6-chloro-2-(pyrimidin-2-yl)-2,3,4,9-tetrahydro-1H- |
| | pyrido[3,4-b]indole, |
| 329 | 6-bromo-1-(4-methoxyphenyl)-2-(pyrimidin-2-yl)-2,3,4,9-tetrahydro-1H-pyrido[3,4- |
| | b]indole, |
| 330 | 2-fluoroethyl 6-chloro-1-(4-isopropylphenyl)-3,4-dihydro-1H-pyrido[3,4-b]indole- |
| | 2(9H)-carboxylate, |
| 331 | 4-chlorophenyl 6-chloro-1-(4-(2-morpholinoethoxy)phenyl)-3,4-dihydro-1H- |
| | pyrido[3,4-b]indole-2(9H)-carboxylate, |
| 332 | (S)-4-methoxyphenyl 6-bromo-1-(4-chlorophenyl)-3,4-dihydro-1H-pyrido[3,4- |
| | b]indole-2(9H)-carboxylate, |
| 355 | 4-chlorophenyl 6-bromo-1-(4-methoxyphenyl)-3,4-dihydro-1H-pyrido[3,4-b]indole- |
| | 2(9H)-carboxylate, |
| 816 | ethyl 6-chloro-1-(4-(2-(thiazol-2-ylamino)ethoxy)phenyl)-3,4-dihydro-1H-pyrido[3,4- |
| | b]indole-2(9H)-carboxylate, |
| 817 | ethyl 6-chloro-1-(4-(2-(5-methylthiazol-2-ylamino)ethoxy)phenyl)-3,4-dihydro-1H- |
| | pyrido[3,4-b]indole-2(9H)-carboxylate, |
| 818 | ethyl 6-chloro-1-(4-(2-(pyridin-4-ylamino)ethoxy)phenyl)-3,4-dihydro-1H-pyrido[3,4- |
| | b]indole-2(9H)-carboxylate, |
| 823 | isobutyl 6-chloro-1-(4-(2-(thiazol-2-ylamino)ethoxy)phenyl)-3,4-dihydro-1H- |
| | pyrido[3,4-b]indole-2(9H)-carboxylate, |
| 824 | isobutyl 6-chloro-1-(4-(2-(5-methylthiazol-2-ylamino)ethoxy)phenyl)-3,4-dihydro-1H- |
| | pyrido[3,4-b]indole-2(9H)-carboxylate, |
| 825 | isobutyl 6-chloro-1-(4-(2-(pyridin-4-ylamino)ethoxy)phenyl)-3,4-dihydro-1H- |
| | pyrido[3,4-b]indole-2(9H)-carboxylate, |
| 830 | 2-methoxyethyl 6-chloro-1-(4-(2-(thiazol-2-ylamino)ethoxy)phenyl)-3,4-dihydro-1H- |
| | pyrido[3,4-b]indole-2(9H)-carboxylate, |
| 831 | 2-methoxyethyl 6-chloro-1-(4-(2-(5-methylthiazol-2-ylamino)ethoxy)phenyl)-3,4- |
| | dihydro-1H-pyrido[3,4-b]indole-2(9H)-carboxylate, |
| 832 | 2-methoxyethyl 6-chloro-1-(4-(2-(pyridin-4-ylamino)ethoxy)phenyl)-3,4-dihydro-1H- |
| | pyrido[3,4-b]indole-2(9H)-carboxylate, |
| 837 | 4-fluorophenyl 6-chloro-1-(4-(2-(thiazol-2-ylamino)ethoxy)phenyl)-3,4-dihydro-1H- |
| | pyrido[3,4-b]indole-2(9H)-carboxylate, |
| 838 | 4-fluorophenyl 6-chloro-1-(4-(2-(5-methylthiazol-2-ylamino)ethoxy)phenyl)-3,4- |
| | dihydro-1H-pyrido[3,4-b]indole-2(9H)-carboxylate, |
| 841 | 4-chlorophenyl 6-chloro-1-(4-(2-(thiazol-2-ylamino)ethoxy)phenyl)-3,4-dihydro-1H- |
| | pyrido[3,4-b]indole-2(9H)-carboxylate, |
| 842 | 4-chlorophenyl 6-chloro-1-(4-(2-(5-methylthiazol-2-ylamino)ethoxy)phenyl)-3,4- |
| | dihydro-1H-pyrido[3,4-b]indole-2(9H)-carboxylate, and |
| 843 | 4-chlorophenyl 6-chloro-1-(4-(2-(pyridin-3-ylamino)ethoxy)phenyl)-3,4-dihydro-1H- |
| | pyrido[3,4-b]indole-2(9H)-carboxylate. |

In one embodiment, the compounds of the invention are present as a racemic mixture. In another embodiment, the compounds of the invention are present as substantially pure (R), (S), (R,R), (S,S), (R,S), or (S,R) enantiomers. In another embodiment, the compounds of the invention are present as the substantially pure (S) enantiomer at the chiral carbon on position 1 of the compound of Formula (V).

The above compounds are listed only to provide examples that may be used in the methods of the invention. Based upon the instant disclosure, the skilled artisan would recognize other compounds intended to be included within the scope of the presently claimed invention that would be useful in the methods recited herein.

### Preparation of Compounds of the Invention

Compounds within the scope of the invention may be produced in any manner known in the art. Embodiments of said compounds were prepared as previously described in U.S. patent application Ser. No. 11/735,069, filed Apr. 13, 2007, U.S. patent application Ser. No. 11/107,783, filed Apr. 18, 2005 (having corresponding International Application No. PCT/US2006/014547, filed on Apr. 17, 2006) and U.S. patent application Ser. No. 11/079,420, filed Mar. 15, 2005 (having corresponding International Application No. PCT/US2005/008481, filed Mar. 15, 2005), each of which are incorporated herein by reference in their entirety and for all purposes.

The reaction methodologies disclosed therein are useful in preparing the compounds of the invention, as recognized by one of skill in the art. Various modifications to the schemes and procedures described therein will be apparent to one of skill in the art, and the methods and use of such compounds as disclosed in the present invention is not limited specifically thereby.

### Methods of the Invention

In one aspect of the invention, compounds of Formula (V) are provided which are useful in a method for post-transcriptionally inhibiting the expression of VEGF in a subject in need thereof comprising inhibiting VEGF mRNA translation by orally administering once, twice or thrice daily to the subject either (i) a therapeutically effective amount of one or more compounds of Formula (V) or one or more pharmaceutically acceptable salts, hydrates, solvates, clathrates, polymorphs, racemates or stereoisomers thereof, or (ii) a pharmaceutical composition comprising one or more pharmaceutically acceptable excipients and a therapeutically effective amount of one or more compounds of Formula (V) or one or more pharmaceutically acceptable salts, hydrates, solvates, clathrates, polymorphs, racemates or stereoisomers thereof.

In this aspect of the invention, inhibiting VEGF mRNA translation treats a VEGF mediated disorder or a solid tumor cancer by reducing plasma and solid tumor VEGF levels, reducing perivascularly sequestered VEGF, reducing aberrant vascular permeability, or inhibiting angiogenesis.

In one embodiment, the invention is directed to methods for inhibiting mRNA translation comprising administering a therapeutically effective amount of at least one compound of the invention to a subject in need thereof.

In another embodiment, the invention is directed to the use of a therapeutically effective amount of at least one compound of Formulas (I), (II), (III), (IV) and (V), including Formulas (I-a) to (I-m) in the manufacture of a medicament for inhibiting VEGF mRNA translation in a subject in need thereof.

In one embodiment, methods for inhibiting angiogenesis are provided comprising administering a therapeutically effective amount of at least one compound of the invention to a subject in need thereof.

In another embodiment, the invention is directed to the use of a therapeutically effective amount of at least one compound of Formulas (I), (II), (III), (IV) and (V), including Formulas (I-a) to (I-m) in the manufacture of a medicament for inhibiting angiogenesis in a subject in need thereof.

In one embodiment, methods for treating cancer, diabetic retinopathy, rheumatoid arthritis, psoriasis, atherosclerosis, chronic inflammation, other chronic inflammation-related diseases and disorders, obesity, or exudative macular degeneration are provided comprising administering a therapeutically effective amount of at least one compound of the invention to a subject in need thereof.

In another embodiment, the invention is directed to the use of a therapeutically effective amount of at least one compound of Formulas (I), (II), (III), (IV) and (V), including Formulas (I-a) to (I-m) in the manufacture of a medicament for treating cancer, diabetic retinopathy, rheumatoid arthritis, psoriasis, atherosclerosis, chronic inflammation, other chronic inflammation-related diseases and disorders, obesity, or exudative macular degeneration in a subject in need thereof.

In yet a further embodiment, the cancers which can be treated by administering a therapeutically effective amount of at least one compound of the invention to a subject in need thereof include solid tumor cancers. Solid tumor cancers that can be treated by the present invention include solid tumor carcinomas and solid tumor sarcomas. Solid tumor cancers include, but are not limited to, pediatric solid tumors, such as Ewing's sarcoma or Wilms tumor and neuroblastoma, and carcinomas of the epidermis, such as malignant melanomas, as well as lung cancers, cervical cancers, colon cancers and renal cancers. Solid tumor sarcomas include, but are not limited to, fibrosarcomas and neurofibromas, including Class 1 and Class 2 neurofibromas. The methods of treating cancer can further include the optional administration of one or more additional agents useful for treating cancer.

In yet another embodiment of the invention, methods for treating a solid tumor cancer by slowing tumorigenesis of a solid tumor are provided, comprising administering a therapeutically effective amount of at least one compound of the invention to a subject in need thereof, either alone or together with one or more additional agents useful for treating cancer.

In another embodiment of the invention, methods for treating a solid tumor cancer by inhibiting VEGF mRNA translation are provided, comprising administering a therapeutically effective amount of at least one compound of the invention to a subject in need thereof, either alone or together with one or more additional agents useful for treating cancer.

In yet another embodiment of the invention methods for treating a solid tumor cancer by reducing solid tumor VEGF levels are provided, comprising administering a therapeutically effective amount of at least one compound of the invention to a subject in need thereof, either alone or together with one or more additional agents useful for treating cancer.

In yet a further embodiment of the invention, methods for treating a solid tumor cancer by reducing perivascularly sequestered or intratumoral VEGF are provided, comprising administering a therapeutically effective amount of at least one compound of the invention to a subject in need thereof, either alone or together with one or more additional agents useful for treating cancer.

In this aspect, reduced perivascularly sequestered VEGF is an in situ comparison of perivascular VEGF in tumors treated with the compound of the invention and tumors not treated with the compound of the invention. In a preferred aspect, reduced perivascularly sequestered VEGF is compared with levels of perivascular VEGF in tumors treated with antibodies to VEGF.

Without intending to be limited by theory, it is believed that the methods of the present invention act through a combination of mechanisms that modulate the activity of VEGF. In this embodiment of the invention, methods for inhibiting VEGF mRNA translation are provided, comprising administering a therapeutically effective amount of at least one compound of the invention to a subject in need thereof. In one aspect, VEGF mRNA translation is inhibited by greater than 10%, 25%, 50%, 75%, 80%, or 90% compared with an untreated tumor or cell. In another aspect, VEGF mRNA translation is inhibited by greater than 50% compared with an untreated tumor or cell.

In another embodiment of the invention, methods for slowing tumorigenesis at a pre-vascular stage are provided, comprising administering a therapeutically effective amount of at least one compound of the invention to a subject in need thereof, either alone or together with one or more additional cancer agents. The pre-vascular stage of tumorigenesis is clinically known as “carcinoma in situ” and tumors at this stage are characterized by their reliance on nearby blood vessels for oxygen and diffusion of nutrients, due to the tumors absence of its own vascular infrastructure. So, by slowing tumorigenesis at a pre-vascular stage, one is preventing or slowing the development of a vascular infrastructure in the tumor. In this embodiment of the invention, whether tumorigenesis has been slowed at the pre-vascular stage is determined by identifying to what extent the tumor has developed a vascular infrastructure. In a preferred aspect, treated tumor growth is prevented or slowed, as compared to the untreated tumors, by 90%, 80%, 70%, 60%, 50%, 40%, 30%, 20% or 10%.

In yet a further embodiment of the invention, methods for reducing perivascularly sequestered or intratumoral VEGF are provided, comprising administering a therapeutically effective amount of at least one compound of the invention to a subject in need thereof.

In yet an additional embodiment of the invention, methods of diagnosing cancer by measuring tumor plasma, and/or serum levels of VEGF are provided. Tumor levels of VEGF can be measured using biopsy tissue, where plasma or serum VEGF levels can be measured by taking blood. In humans, different tumors secrete different levels of VEGF. Standard ELISA procedures can be used to measure the amount of VEGF in the tumor, serum or plasma. See, for example, Verheul, H. M. W. et al. (2000) Platelet and coagulation activation with vascular endothelial growth factor generation in soft tissue sarcomas. Clin. Cancer Res. 6:166. For tumors that do not secrete large amounts of VEGF into the plasma, the tumor VEGF concentration can be determined to diagnose the tumor progression. For tumors that do secrete large amount of VEGF into the plasma, plasma VEGF concentration can be determined to diagnose the tumor progression. After most known cancer treatments, VEGF levels are not affected, and therefore the plasma or tumor levels of VEGF do not predict efficacy of the treatment (i.e., progression of the cancer). Compounds of the present invention are useful for inhibiting the production of VEGF protein, both in the plasma and tumor, and therefore measuring VEGF protein level may be an accurate way to monitor and/or predict the progression of the cancer (i.e., the efficacy of the treatment) when the methods of the present invention are used for treating cancer.

In yet another embodiment of the invention, methods for reducing solid tumor or plasma VEGF levels are provided, comprising administering a therapeutically effective amount of at least one compound of the invention to a subject in need thereof. In this embodiment, VEGF levels can be measured in a tumor not treated with the compounds of the present invention and the VEGF levels compared to the VEGF levels measured in a tumor treated with the compounds of the present invention, thereby showing that by treatment of tumors with the compounds of the present invention VEGF levels are reduced.

In yet another embodiment of the invention, methods for treating a solid tumor cancer are provided, comprising (a) measuring one or more of serum VEGF levels, plasma VEGF levels, or tumor VEGF levels, and administering a therapeutically effective amount of at least one compound of the invention to a subject in need thereof. In an embodiment, VEGF concentration is measured to determine whether treatment with a compound of the present invention should be undertaken. In this aspect, treatment with a compound of the present invention is preferred and more effective as the VEGF levels increase.

In yet a further embodiment of the invention, methods for treating a solid tumor cancer are provided, comprising administering a therapeutically effective amount of at least one compound of the invention to a subject in need thereof, together with one or more additional cancer agents.

In preferred embodiments, the methods of the invention comprise administering a therapeutically effective amount of at least one compound of the invention, wherein the compound is an (S) isomer.

According to the methods of the invention, the compound(s) may be administered to the subject via any drug delivery route known in the art. Specific exemplary administration routes include oral, ocular, rectal, buccal, topical, nasal, ophthalmic, subcutaneous, intramuscular, intravenous (bolus and infusion), intracerebral, transdermal, and pulmonary.

The term “therapeutically effective amount”, as used herein, refers to an amount of a pharmaceutical agent to treat or ameliorate the identified disease or condition, or to exhibit a detectable therapeutic or inhibitory affect. The term “prophylactically effective amount” as used herein, refers to an amount of a pharmaceutical agent to prevent the identified disease or condition, or to exhibit a detectable prophylactic or inhibitory affect.

The affect can be detected by, for example, the assays disclosed in the following examples. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

Within the scope of the present invention, the therapeutically effective amount of said one or more compounds or of a pharmaceutical composition thereof is a plasma concentration in a range selected from greater than about 0.01 μg/mL, from greater than about 0.05 μg/mL, greater than about 0.10 μg/mL, greater than about 0.15 μg/mL, greater than about 0.20 μg/mL, greater than about 0.25 μg/mL, or greater than about 0.30 μg/mL for a time period of from about 3 to about 24 hours following administration once daily.

Within the scope of the present invention, the therapeutically effective amount of said one or more compounds or of a pharmaceutical composition thereof is a plasma concentration in a range selected from greater than about 0.01 μg/mL, from greater than about 0.05 μg/mL, greater than about 0.10 μg/mL, greater than about 0.15 μg/mL, greater than about 0.20 μg/mL, greater than about 0.25 μg/mL, or greater than about 0.30 μg/mL for a time period of from about 3 to about 12 hours following administration twice daily.

Within the scope of the present invention, the therapeutically effective amount of said one or more compounds or of a pharmaceutical composition thereof is a plasma concentration in a range selected from greater than about 0.01 μg/mL, from greater than about 0.05 μg/mL, greater than about 0.10 μg/mL, greater than about 0.15 μg/mL, greater than about 0.20 μg/mL, greater than about 0.25 μg/mL, or greater than about 0.30 μg/mL for a time period of from about 3 to about 8 hours following administration thrice daily.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

More specifically, the concentration-biological effect relationships observed with regard to the compound(s) of the present invention indicate an initial target plasma concentration ranging from approximately 0.01 μg/mL to approximately 100 μg/mL, from approximately 0.05 μg/mL to approximately 50 μg/mL, or from approximately 0.05 μg/mL to approximately 10 μg/mL following administration once, twice or thrice daily. To achieve such plasma concentrations, the compounds of the invention may be administered at doses that vary from 0.1 μg to 100,000 mg/day, depending upon the route of administration. Guidance as to particular dosages and methods of delivery is provided in the literature and is generally available to practitioners in the art.

In general, the dose will be in the range of about 0.001 mg/day to about 500 mg/day, or about 0.01 mg/day to about 500 mg/day, or about 0.1 mg to about 500 mg/day, or about 1.0 mg/day to about 500 mg/day, in single, divided, or continuous doses for a patient or subject weighing between about 40 to about 100 kg (which dose may be adjusted for patients or subjects above or below this weight range, particularly children under 40 kg).

The dose administered to achieve an effective target plasma concentration may also be administered based upon the weight of the subject or patient. Doses administered on a weight basis may be in the range of about 0.01 mg/kg/day to about 20 mg/kg/day, or about 0.015 mg/kg/day to about 10 mg/kg/day, or about 0.02 mg/kg/day to about 10 mg/kg/day, or about 0.025 mg/kg/day to about 10 mg/kg/day, or about 0.03 mg/kg/day to about 10 mg/kg/day, wherein said amount is orally administered once, twice or thrice daily according to subject weight. In another embodiment, where daily doses are adjusted based upon the weight of the subject or patient, compounds of the invention may be formulated for delivery at about 0.02, 0.025, 0.03, 0.05, 0.06, 0.075, 0.08, 0.09. 0.10, 0.20, 0.25, 0.30, 0.50, 0.60, 0.75, 0.80, 0.90, 1.0, 1.10, 1.20, 1.25, 1.50, 1.75, 2.0, 5.0 or 10 mg/kg/day. Daily doses adjusted based upon the weight of the subject or patient may be administered as a single, divided, or continuous dose. In embodiments where a dose of compound is given more than once per day, it may be administered twice, thrice, or more per day.

Compounds of the invention may be administered to the subject once, twice or thrice daily when the subject is either fasted or fed. For the purposes of administering one or more compounds of the invention, alone or in combination with any other therapeutic agent or treatment, the term “fed” may comprise any meal, such as a meal high in carbohydrates, a meal high in protein, or a meal high in fat. Administration with food, such as a high-fat high-calorie meal immediately prior to administration of a compound of the invention, may increase the absorption of the compound as reflected in the maximum plasma concentration (Cₘₐₓ) achieved relative to fasted subjects.

Increases in the Cₘₐₓ observed for individuals fed a high-fat high-calorie meal may be greater than about 5% to about 10% above the Cₘₐₓ of fasting individuals. In other embodiments, the increase in Cₘₐₓ of fed subjects may be greater than about 5% to about 20%, greater than about 10% to about 20%, greater than about 15% to about 20%, greater than about 15% to about 30%, greater than about 20% to about 40%, or greater than about 20% to about 50% of the Cₘₐₓ of fasting subjects.

In one embodiment, the compounds of the invention are administered twice daily. In another embodiment the compounds are administered twice daily with food. In yet another embodiment, the compounds of the invention can be administered twice daily with a high-fat, high-calorie meal just prior to or at the time of administration. In some embodiments, compounds of the invention can be formulated for administration twice daily accompanying a high-fat high-calorie meal.

In one embodiment, compounds of the invention may be formulated for oral administration from about 0.01 to about 10 mg/kg (mg of compound/kg of subject body weight) twice daily with or without food. In other embodiments, compounds of the invention may be formulated for oral administration over the range of about 0.015 mg/kg to about 10 mg/kg, or about 0.02 mg/kg to about 10 mg/kg, or about 0.025 mg/kg to about 10 mg/kg, or about 0.03 mg/kg to about 10 mg/kg, where the compound is administered twice per day with or without food. In another embodiment, compounds of the invention may be formulated for oral delivery at about 0.02, 0.025, 0.03, 0.05, 0.06, 0.075, 0.08, 0.09. 0.10, 0.20, 0.25, 0.30, 0.50, 0.60, 0.75, 0.80, 0.90, 1.0, 1.10, 1.20, 1.25, 1.50, 1.75, 2.0, 5.0 or 10 mg/kg twice daily with or without food.

In one embodiment, pharmaceutical compositions are formulated for oral administration and comprise from about 1 mg to about 1 g of one or more compounds of the invention. In other embodiments the pharmaceutical compositions are formulated for oral administration and comprise from about 5 mg to about 500 mg, or from about 10 mg to about 400 mg, or from 20 mg to about 300 mg, or from 30 mg to about 200 mg, or from 30 mg to about 300 mg, of one or more compounds of the invention. Such compositions for oral administration may be formulated as a tablet, capsule, solution, or suspension comprising one or more compounds of the invention, or a pharmaceutically acceptable salt, hydrate, solvate, clathrate polymorph, raemate, or stereoisomer there of.

In one embodiment of the invention, the pharmaceutical compositions of the invention are formulated to provide a plasma concentration of a compound of the invention greater than about 0.05 μg/mL for a time period of from about 3 to about 24 hours following oral administration once, twice or thrice daily. In other embodiments, the pharmaceutical compositions of the invention are formulated to provide a plasma concentration of a compound of the invention greater than about 0.1 μg/mL, greater than about 0.15 μg/mL, greater than about 0.20 μg/mL, greater than about 0.25 μg/mL, or greater than about 0.30 μg/mL for a time period of from about 3 to about 24 hours following oral administration once, twice or thrice daily.

In one aspect, compounds of the invention are generally well tolerated by subjects. For example, the compounds can be administered by the oral route with no occurrences, or only mild to moderate occurrences, of nausea, productive cough, constipation, diarrhea, eye pruritus, headache, back pain, or insomnia in one or more subjects. For the purposes of this disclosure, mild or moderate occurrences may be described as those occurrences that would not prevent individual subjects or groups of subjects from continuing to receive the compounds.

Compounds of the invention may be advantageously administered over one or more days without raising safety concerns based upon hERG assays, Novascreen® assays, phosphatase and kinase panel assessments Similarly, no meaningful cytotoxicity is observed in lung fibroblast, skin fibroblast or bone marrow progenitor cell cytotoxicity testing. In some embodiments, compounds of the invention do not display mutagenicity in the Ames assay, chromosomal aberrations, polyploidy or endoreduplication in CHO cell assays, or clastogenic effects in rat micronucleus assays.

In yet other embodiments, no negative cardiopulmonary effects are observed with single doses up to 140 mg/kg, no negative neuorological effects are observed with single doses up to 200 mg/kg, and no significant toxicity is observed at dose of 120 mg/kg QD or at doses of 60 mg/kg BID for 7 days.

Compounds of the invention may be advantageously administered without one or more negative effects observed with other compounds having VEGF inhibitory activity, such as anti VEGF antibodies (e.g., bevacizumab) and other compounds inhibiting VEGFR tyrosine kinase activity. Recognized side effects of bevacizumab include hypertension (see, e.g., Gordon et al., J. Clin. Oncol. 19(3) 843-850 (2001)), proteinuria (id.), and thromboembolism. Other inhibitors of VEGFR, (e.g., PTK787, sunitinib, and ZD6474) also induce a number of additional off-target effects including: light-headedness, ataxia, headache, nausea, vomiting, diarrhea, rash, subungual hemorrhage, myelosuppression, fatigue, hypothyroidism, QT interval prolongation or heart failure. Such effects appear to be due to nonspecific inhibition of tyrosine kinase receptors other than the VEGFR.

In some embodiments, the compounds of the invention may advantageously be administered without causing a substantial incidence of either proteinuria or hypertension. In other embodiments, the compounds of the invention may advantageously be administered without causing a substantial incidence of an increase in the grade of proteinuria. In other embodiments, the compounds of the invention may advantageously be administered without causing a substantial increase in blood pressure.

In some embodiments, a substantial incidence of proteinuria, an increase in the grade of proteinuria, or hypertension is the occurrence of those side effects in greater than about 20% of the subjects or patients treated. In other embodiments, a substantial incidence of either proteinuria or hypertension is the occurrence of either of those side effects in greater than 15% of the subjects or patients treated. In still other embodiments, a substantial incidence of either proteinuria or hypertension is the occurrence of those side effects in greater than 10%, or 5% or 2% or 1% of the subjects or patients treated. Compounds of the invention may cause minor transient changes in heart rate, blood pressure, respiratory rate, and core body temperature. Such changes may remain within normal limits and may be observed at dosages of 30 mg/kg or greater.

For the purposes of this invention, a patient or subject is considered to have Category 1 hypertension with an asymptomatic, transient increase in blood pressure greater than 20 mm Hg (diastolic) above a normal level or greater than 150 mm Hg/100 mm Hg (systolic/diastolic) within a 24 hour period. Category 2 hypertension refers to a recurrent, persistent or symptomatic increase in blood pressure above a normal level for more than 24 hours. For the purposes of this invention, hypertension is defined as a recurrent, persistent or symptomatic elevated blood pressure greater than 20 mm Hg (diastolic) above a normal level or greater than 150 mm Hg/100 mm Hg (systolic/diastolic), during which time one or more compounds of the invention are present at or above a therapeutically effective concentration in the plasma of a subject.

For the purposes of this invention, proteinuria is defined as an elevation in the amount of protein found in the urine of a patient or subject outside of the normal range during the period a compound of the invention is present at or above a therapeutically effective concentration in the plasma of a subject. For example, proteinuria may be found when there is more than 0.15 grams of protein present in a 24 hour urine sample. Grade 1 proteinuria may be found when the amount of protein in a 24 hour urine sample is from 0.15 g to 1 g of protein in a 24 hour urine sample, Grade 2 proteinuria may be found when the amount of protein in a 24 hour urine sample is greater 1 g but less than 3.5 grams of protein in a 24 hour urine sample. Grade 3 proteinuria may be found when the amount of protein in a 24 hour urine sample is greater than 3.5 g of protein in a 24 hour urine sample. Grade 4 proteinuria equates to nephrotic syndrome. An elevation in the amount of protein found in the urine of a patient or subject outside of the normal range may also be found based upon dipstick measurements. A dipstick measurement of “1+” equates to grade 1 proteinuria, a measure of 2+ or 3+ equates to grade 2 proteinuria, and a dipstick measure of 4+ equates to grade 3 proteinuria.

For the purposes of this invention a patient or subject may be considered to have a risk of having a stroke when they have hypertension, and particularly when they have hypertension and a prior medical history of one or more strokes.

In some embodiments, the compounds of the invention may be administered without causing a substantial incidence of grade 1 proteinuria as measured by 24 hour urine analysis or by dipstick analysis of a urine sample. In other embodiments, compounds of the invention may be administered without causing a substantial incidence of grade 2 proteinuria as measured by 24 hour urine analysis or by dipstick analysis of a urine sample. In yet other embodiments, compounds of the invention may be administered without causing an increase in the proteinuria status (e.g., the grade of proteinuria) of a patient from grade 1 proteinuria to grade 2 proteinuria, or from grade 2 proteinuria to grade 3 proteinuria, as measured by 24 hour urine analysis or by dipstick analysis of a urine sample.

In one embodiment, the compounds of the invention are administered to patients having solid tumors that have one or more of high blood pressure (hypertension), proteinuria, or are at risk of having a stroke. Other embodiments of the invention include a method of treatment where a subject having a solid tumor is assessed for high blood pressure (hypertension), proteinuria or for their risk of having a stroke; wherein hypertension, proteinuria, or risk of having a stroke, suggest treatment with a compound of the invention alone or in combination with one or more additional agents useful in the treatment of cancer that do not increase blood pressure, the level of protein in the urine or the risk of stroke.

In other embodiments, where a patient or subject having a solid tumor is assessed and determined to have hypertension or risk of having a stroke, the patient or subject may be treated with one or more compounds of the invention alone or in combination with one or more additional agents useful in the treatment of cancer that increase blood pressure and a one or more agents that reduce blood pressure. Agents that reduce blood pressure include, for example, beta blockers such as TENORMIN®, diuretics such as hydrocholorthiazide, calcium channel blockers such diltiazem, acetylcholine esterase inhibitors (ACE inhibitors) such as lisinopril (ZESTRIL®, PRINIVIL®), and Angiotensin II Receptor Blockers (ARB) such LOSARTAN®.

In still other embodiments, where a patient or subject having a solid tumor is assessed and determined to have proteinuria, the patient or subject may be treated with one or more compounds of the invention alone or in combination with one or more additional agents useful in the treatment of cancer that increase protein in urine and a one or more agents that reduce protein in urine. Agents that reduce protein in urine include, for example, acetylcholine esterase inhibitors (ACE inhibitors) such as lisinopril (ZESTRIL®), and Angiotensin II Receptor Blockers (ARB) such LOSARTAN®.

Another aspect of the invention is a diagnostic assay to assess a subjects suitability for treatment with a compound of the invention along with one or more therapeutics that increase blood pressure, protein levels in the urine or the risk of having a stroke. Unacceptable hypertension, proteinuria, or risk of having a stroke, suggest treatment with a compound of the invention alone or in combination with one or more other therapeutics that do not increase blood pressure or protein in urine, as opposed to combination treatment with a compound of the invention and other therapeutics that increase blood pressure or protein in urine.

The exact dosage amount to achieve a therapeutic effect will be determined by the practitioner, and may be balanced in light of factors related to the subject in need of treatment. Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

### Metabolites of the Compounds of the Invention

Also falling within the scope of the present invention are the in vivo metabolic products of the compounds described herein. For example, such products may result from the oxidation, reduction, hydrolysis, amidation, esterification and the like of the administered compound, primarily due to enzymatic processes. Accordingly, the invention includes compounds produced by a process comprising contacting a compound of this invention with a mammalian tissue or a mammal for a period of time sufficient to yield a metabolic product thereof. Such products typically are identified by preparing a radio-labeled (e.g. C¹⁴ or H³) compound of the invention, administering it in a detectable dose (e.g., greater than about 0.5 mg/kg) to a mammal such as rat, mouse, guinea pig, monkey, or to man, allowing sufficient time for metabolism to occur (typically about 30 seconds to 30 hours), and isolating its conversion products from urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds of the invention even if they possess no biological activity of their own.

### Pharmaceutical Compositions of the Invention

While it is possible for the compounds of the present invention to be administered neat, it may be preferable to formulate the compounds as pharmaceutical compositions. As such, in yet another aspect of the invention, pharmaceutical compositions useful in the methods of the invention are provided. The pharmaceutical compositions of the invention may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8.0.

More particularly, the pharmaceutical compositions of the invention comprise a therapeutically or prophylactically effective amount of at least one compound of the present invention, together with one or more pharmaceutically acceptable excipients. Optionally, the pharmaceutical compositions of the invention may comprise a combination of compounds of the present invention, or may include a second active ingredient useful in the treatment of cancer, diabetic retinopathy, or exudative macular degeneration.

Formulations of the present invention, e.g., for parenteral or oral administration, are most typically solids, liquid solutions, emulsions or suspensions, while inhalable formulations for pulmonary administration are generally liquids or powders, with powder formulations being generally preferred. A preferred pharmaceutical composition of the invention may also be formulated as a lyophilized solid that is reconstituted with a physiologically compatible solvent prior to administration. Alternative pharmaceutical compositions of the invention may be formulated as syrups, creams, ointments, tablets, and the like.

The term “pharmaceutically acceptable excipient” refers to an excipient for administration of a pharmaceutical agent, such as the compounds of the present invention. The term refers to any pharmaceutical excipient that may be administered without undue toxicity. Pharmaceutically acceptable excipients are determined in part by the particular composition being administered, as well as by the particular method used to administer the composition. Accordingly, there exists a wide variety of suitable formulations of pharmaceutical compositions of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions of the invention may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions of the invention may be formulated as suspensions comprising a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions of the invention may be in the form of a sterile injectable preparation, such as a sterile injectable aqueous emulsion or oleaginous suspension. This emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propane-diol. The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids such as oleic acid may likewise be used in the preparation of injectables.

Generally, the compounds of the present invention useful in the methods of the present invention are substantially insoluble in water and are sparingly soluble in most pharmaceutically acceptable protic solvents and in vegetable oils. However, the compounds are generally soluble in medium chain fatty acids (e.g., caprylic and capric acids) or triglycerides and have high solubility in propylene glycol esters of medium chain fatty acids. Also contemplated in the invention are compounds which have been modified by substitutions or additions of chemical or biochemical moieties which make them more suitable for delivery (e.g., increase solubility, bioactivity, palatability, decrease adverse reactions, etc.), for example by esterification, glycosylation, pegylation, etc.

In a preferred embodiment, the compounds of the present invention may be formulated for oral administration in a lipid-based formulation suitable for low solubility compounds. Lipid-based formulations can generally enhance the oral bioavailability of such compounds. As such, a preferred pharmaceutical composition of the invention comprises a therapeutically or prophylactically effective amount of a compound of the present invention, together with at least one pharmaceutically acceptable excipient selected from the group consisting of: medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil.

Lipid-based formulations may comprise a surface active excipient (a bioavailability enhancer/microemulsion component), and an ester of steric acid (a surfactant/solubilizer/microemulsion component) in addition to one or more compounds of the invention. In one embodiment the a surface active excipient is GELUCIRE 44/14® (Gattefosse, Paramus, N.J.). In one embodiment the ester of steric acid is a poly-oxyethylene ester of 12-hydroxystearic acid, preferably SOLUTOL HS 15® (BASF, Roxbury, N.J.). In another embodiment, the lipid-based formulations comprise one or more compounds of the invention and an excipient, where the excipient comprises approximately equal portions by weight of GELUCIRE 44/41®, and SOLUTOL HS 15®. The lipid-based formulations comprising a surface active excipient and an ester of steric acid in addition to one or more compounds of the invention may be packaged for oral delivery, (e.g., packaged into hard gelatin capsules). Any of the aforementioned compositions may contain additional components such as BHT which acts as an anti-oxidant.

In one embodiment, the lipid-based formulations comprise a surface active excipient, a triglyceride, and an ester of steric acid in addition to one or more compounds of the invention. In one embodiment, a surface active excipient is GELUCIRE 44/14® (Gattefosse, Paramus, N.J.). In one embodiment, a triglyceride is a medium chain triglyceride, preferably LABRAFAC CC®(Gattefosse, Paramus, N.J.). In another embodiment, an ester of steric acid is a poly-oxyethylene ester of 12-hydroxystearic acid, preferably SOLUTOL HS 15® (BASF, Roxbury, N.J.). In another embodiment, the lipid-based formulations comprise one or more compounds of the invention and an excipient, where the excipient comprises approximately equal portions by weight of GELUCIRE 44/41®, LABRAFAC CC®, and SOLUTOL HS 15®. In another embodiment, the lipid-based formulations comprise one or more compounds of the invention and an excipient, wherein the excipient comprises about 35% GELUCIRE®, about 35% LABRAFAC CC®, and about 30% SOLUTOL® by weight. The lipid-based formulations comprising a surface active excipient, a triglyceride, and an ester of steric acid in addition to one or more compounds of the invention may be packaged for oral delivery, (e.g., packaged into hard gelatin capsules). Any of the aforementioned compositions may contain additional components such as BHT which acts as an anti-oxidant.

In an alternative preferred embodiment, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-β-cyclodextrin (HPBC), which may be added to any of the above-described compositions to further improve the aqueous solubility characteristics of the compounds of the present invention. In one embodiment, the composition comprises 0.1% to 20% hydroxypropyl-β-cyclodextrin, more preferably 1% to 15% hydroxypropyl-β-cyclodextrin, and even more preferably from 2.5% to 10% hydroxypropyl-β-cyclodextrin. The amount of solubility enhancer employed will depend on the amount of the compound of the present invention in the composition.

### Combination Therapy

It is also possible to combine a compound of the present invention with one or more other active ingredients or agents useful in the treatment of cancer, including compounds, in a unitary dosage form, or in separate dosage forms intended for simultaneous or sequential administration to a patient in need of treatment. When administered sequentially, the combination may be administered in two or more administrations. In an alternative embodiment, it is possible to administer one or more compounds of the present invention and one or more additional active ingredients by different routes.

The skilled artisan will recognize that a variety of active ingredients may be administered in combination with the compounds of the present invention that may act to augment or synergistically enhance the VEGF-inhibiting and/or anti-angiogenesis activity of the compounds of the invention.

More specifically, for methods involving the treatment of cancer, agents known in the art to be useful for treating cancer are provided. Such agents include, but are not limited to, radiation therapy, agents that cause DNA damage, agents that reduce the concentration or effect of a growth factor, agents that inhibit angiogenesis, paclitaxel, fluorouracil, tamoxifen, doxorubicin, aromasin, exemistane, taxol, 5-fluororuracil, letrozole, CPT-11, a tyrosine kinase inhibitor, a COX-2 inhibitor, thalidomide, gemcitabine, squalamine, endostatin, angiostatin, AE-941, lenalidomide, medi-522, 2-methoxyestradiol, carboxyamidotriazole, combretastatin A4 phosphate, SU6668, SU11248, BMS-275291, COL-3, cilengitide, IMC-1121B, vatalanib, LY317615, VEGF Trap, ZD6474, halofuginone, hydrobromide, celecoxib, interferon alpha, interleukin-12, and antibodies capable of binding VEGF or a VEGF receptor, such as bevacizumab. VEGF receptors include VEGF receptor 1, VEGF receptor 2, and VEGF receptor 3, and the neuropilins (e.g., neurophilin-1 (np-1) and neurorphilin-2 (np-2)). In another embodiment, the compounds of the present invention are used in combination with an agent that blocks the activity of a VEGF receptor. In yet another embodiment, the compounds of the present invention can be used in combination with agents that can block the VEGF signaling pathway. Treatment only with a factor that can block VEGF signaling may cause an increase in VEGF concentration. In such a case, including a compound of the present invention in the treatment protocol can prevent the subsequent increase in VEGF levels. Similarly, use of the compounds of the present invention in combination with an antibody is highly preferred. Antibodies are relatively large and may not cross tight barriers, allowing secreted VEGF to remain in areas such as the perivascular space. Post-transcriptional control of VEGF expression can prevent the tumor from retaining as much VEGF in the perivascular space, in the extracellular matrix, or in other spaces and vessels that have a physical barrier to antibodies.

According to the methods of the invention, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof. The examples illustrate the testing of the compounds of the present invention in vitro and/or in vivo. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

### Plasma Concentrations of Orally Administered Compounds and the Effect of Food

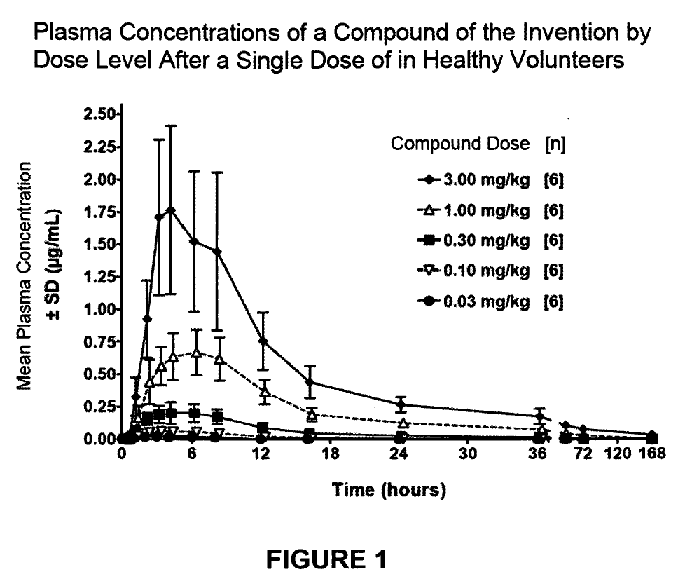

Groups of normal healthy human volunteers (six per group) are administered a single oral dose (0.03, 0.10, 0.30, 1.00, or 3.00 mg/kg) of a compound of the invention. At the indicated times after administration samples of venous blood are withdrawn from the volunteers and the plasma concentration of the compound in each sample is determined using liquid chromatography and tandem mass spectroscopy (LC-MS/MS). Mean plasma concentrations are plotted versus time along with the standard deviation of the values. See FIG. 1. Pharmacokinetic parameters, including the maximum concentration (Cₘₐₓ); the time of maximum concentration (Tₘₐₓ) and the area under the curve (AUC) for a given time period (e.g., the AUC over the first 24 hours AUC₀₋₂₄) are calculated from the data. Error ranges presented are given as ±the standard deviation.

### Example 2

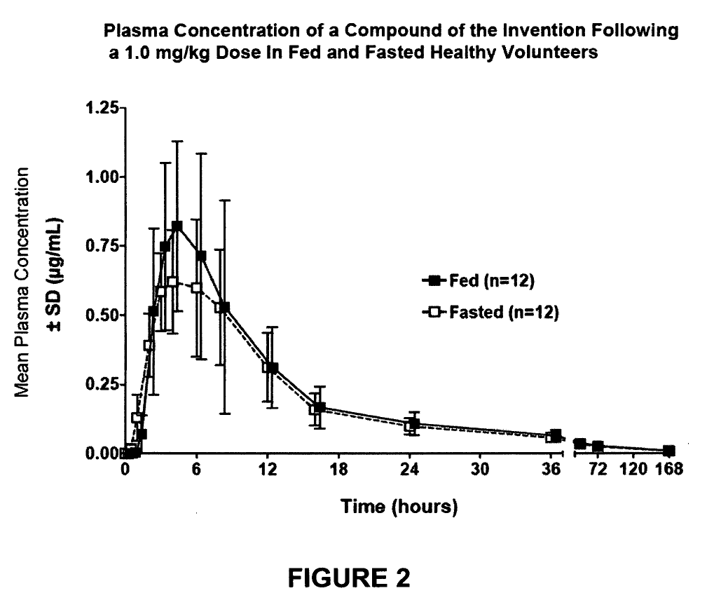

In order to evaluate the effect of food on the plasma concentration of orally administered compounds of the invention a group of twelve healthy human volunteers, six male and six female, are employed. The volunteers are randomly assigned to one of two groups, a fed group and a fasted group for the first week of the evaluation. Immediately prior to compound administration (1 mg/kg) the fed group is given a high-fat high-calorie meal, whereas the fasted group did not receive a meal prior to administration of the compound. At multiple times after compound administration, samples of venous blood are withdrawn from the volunteers and the plasma concentration of the compound in each sample is determined using liquid chromatography and tandem mass spectroscopy (LC-MS/MS). In the second week of the experiment, the groups are crossed over with the fed group becoming the fasted group an the fasted group becomes the fed group. The data are shown in FIG. 2, mean plasma concentrations are plotted versus time along with the standard deviation of the values.

### Example 3

### Plasma Concentrations Over Multiple Days with Dosing Twice Daily

### Part A—Studies in Mice

Compounds of the invention are tested in a mouse fibrosarcoma model. Briefly, nude mice bearing HT1080 fibrosarcoma xenografts are treated with a compound of the invention the range of 1 to 10 mg/kg (mg of compound/kg of body weight) given once per day or twice per day for 18 days. After 18 days of treatment tumor and plasma VEGF levels are measured by ELISA. Treatment either once or twice per day with a compound of the present invention over the range of 1-10 mg/kg reduces mean tumor VEGF concentrations greater than 85% and plasma VEGF levels 95% or greater relative to control values.

### Part B—Canine Studies

Four male beagles are given escalating doses of a compound of the invention. On days 1, 4, 8, and 12 of the study, each animal is given a single dose of vehicle (n=1) and test compound at 30 mg/kg, 60 mg/kg or 120 mg/kg (n=1 each), respectively. Compounds are administered orally in a lipid-based formulation comprising a surface active excipient, a triglyceride, and an ester of steric acid in addition to one or more compounds of the invention in an orally administered capsule once daily.

Clinical parameters including body weight and hemodynamic parameters (diastolic, systolic and mean arterial blood pressure) are assessed daily. Heart rate, core body temperature, and ECG's are collected for 24 hours before and after each dose of vehicle or compound. Arterial blood samples are obtained at 3, 4, 5, 6, 7, and 8 hours post dose to measure blood pH, partial pressure of CO₂, (pCO₂), partial pressure of oxygen (pO₂), saturated oxygen, and blood bicarbonate. All clinical parameters remain in the normal range over the duration of the study.

Part C—Human Studies Plasma Concentrations Over Multiple Days with Dosing Twice Daily

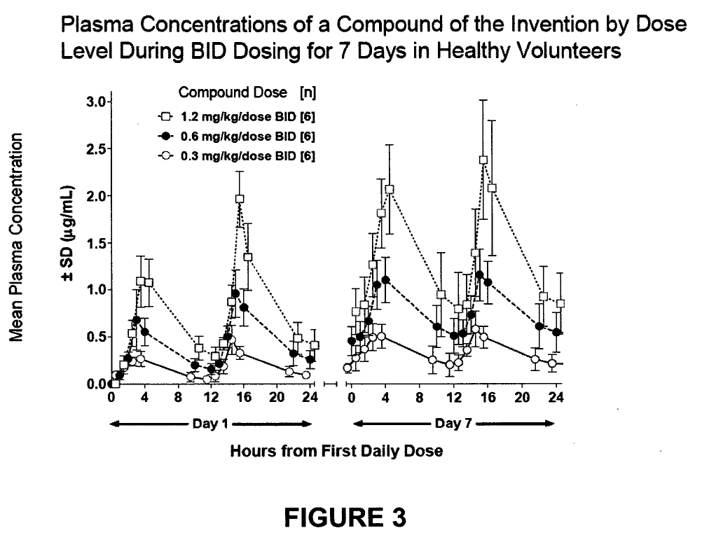

Groups of normal healthy human volunteers (8 per group, 3 male and 3 female receiving drug, 1 placebo per gender at each dose level, 24 individuals total) are administered oral doses (0.03, 0.60, or 1.20 mg/kg) of a compound of the invention twice daily for seven days. Vital signs (including pulse and blood pressure) and samples for urinalysis are collected at baseline and repeatedly during the study. Samples of venous blood are withdrawn from the volunteers prior to administration of the first dose. Samples of venous blood are also withdrawn at multiple times on the first and seventh day, before the morning and evening doses on days two through six of the study, then at 6, 12, 24 and 36 hours after administration of the last dose of the compound, and once on days 14 and 21 of the study. The plasma concentration of the compound in each sample is determined using liquid chromatography and tandem mass spectroscopy (LC-MS/MS). While side effects including headache, dizziness, nausea, vomiting and stomach discomfort are observed, no serious, dose limiting, or definitive drug-related events, such as the development of hypertension or proteinuria are observed. All side effects are reversible and there is no correlation of any side effect with dose. Mean plasma concentrations of compound are plotted versus time along with the standard deviation of the values for study days 1 and 7. See FIG. 3. Pharmacokinetic parameters, including C_{max, T}ₘₐₓ, and the AUC are calculated from the data and presented as in Example 2.

### Example 4

### Plasma Concentrations Over Multiple Days with Dosing Thrice Daily

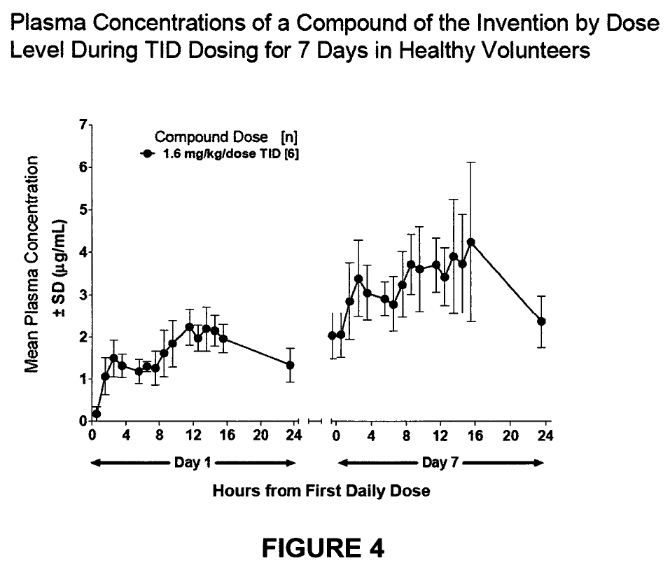

Groups of normal healthy human volunteers (eight per group) are administered oral doses of 1.6 mg/kg of a compound of the invention three times daily for seven days. Samples of venous blood are withdrawn from the volunteers prior to administration of the first dose. Samples of venous blood are withdrawn at multiple times on the first and seventh day, before the morning and evening doses on days two through six of the study, at 6, 12, 24, and 36 hours after administration of the last dose of the compound and once on days 14 and 21 of the study. The plasma and/or serum concentration of the compound in each sample is determined using liquid chromatography and tandem mass spectroscopy (LC-MS/MS). While side effects including headache, dizziness, nausea, vomiting and stomach discomfort are observed, no serious, dose limiting, or definitive drug-related events, such as the development of hypertension or proteinuria are observed. In addition, no bleeding, clotting, hypertension or proteinuria are observed. All side effects are reversible and there is no correlation of any side effect with dose. Mean plasma concentrations are plotted versus time along with the standard deviation of the values. See FIG. 4.

### Example 5

### Xenograft Model Utilizing MCF-7 Cells Transfected with Human Aromatase Combination Treatment with FEMARA® (Brand of Letrozole)

The relative effects of the aromatase inhibitor agent letrozole and a test compound of the present invention were evaluated, when administered alone or in combination, on the in vivo growth of MCF-7 breast cancer cells transfected with the human aromatase gene.

In this study, cells (7.0×10⁶ cells/mouse) were implanted subcutaneously in female ovariectomized athymic nude mice (n=10). Androstenedione was administered (via slow-release pellets) as a source of androgens. After 28 days, when the tumors had become established (i.e., the mean tumor size had reached ˜200 mm³), mice were divided into 6 treatment groups, and treatment was administered orally once per day (as shown in Table 1) until a mean tumor size of ˜1500 mm was reached.

TABLE 1

| | | | Dose |
|---|---|---|---|
| Treatment | | Dose Volume | Concentration |
| Group | Dose/mouse | (mL/kg)^{b} | (mg/mL) |
| Vehicle^{c} | 0 | 3.7 | 0 |
| letrazole only | 10 μg/mouse | 3.7 | 0.1 |
| letrazole only | 30 μg/mouse | 3.7 | 0.3 |
| Cpd only | 10 mg/kg | 3.7 | 2.5 |
| Combination | 10 μg/mouse | 3.7 | 0.1 |
| | 10 mg/kg | 3.7 | 2.7 |
| Combination | 30 μg/mouse | 3.7 | 0.3 |
| | 10 mg/kg | 3.7 | 2.7 |

The daily dose was administered in a target volume of 0.1 mL, which was adjusted according to subject weight. The oral vehicle was L21 (a mixture of 35% Labrasol, 35% Labrafac, and 30% Solutol). Individual vehicle-treated mice were removed from study when the tumor reached 1500 mm³. After 100 days of treatment, treatment was stopped.

As shown in Table 2, tumors were approximately 200 mm³ at the start of the study. By Day 42, the mean tumor size was ˜800 mm³ and the mice with the largest tumors were removed from study (escape tumor size of greater than 500 mm³). Table 2 shows that only four of the ten mice in the group reached 1500 mm³. One vehicle-treated mouse was found dead, possibly due to a dosing mishap. However, six of nine mice had tumors that reached 500 mm³.

TABLE 2

| | | Mean % | | |
|---|---|---|---|---|
| | | Inhibition of | | |
| | | Tumor Size | | |
| Treatment | | vs Vehicle at | Mean Tumor | Percent of mice with cures |
| Group | Dose/mouse | Day 42^{c} | Size at Day 98 | and Escape to 500 mm^{3d, e} |
| Vehicle^{c} | 0 | NA | 189 mm³ | 20% cured |
| | | | | 1 of 5 cured, 6 escaped, 1 dead |
| letrazole only | 10 μg/mouse | 72% | 225 mm³ | 0% cured |
| | | | | 0 of 9 cured, 2 escaped, 1 dead |
| letrazole only | 30 μg/mouse | 91%** | 146 mm³ | 22% cured |
| | | | | 2 of 9 cured, 0 escaped, 1 dead |
| Cpd only | 10 mg/kg | 92%** | 22 mm³ | 56% cured |
| | | | | 5 of 9 cured, 0 escaped, 1 dead |
| Combination | 10 μg/mouse | 79% | 43 mm³ | 40% cured |
| | 10 mg/kg | | | 4 of 10 cured, 0 escaped |
| Combination | 30 μg/mouse | 91%** | 34 mm³ | 30% cured |
| | 10 mg/kg | | | 3 of 10 cured, 0 escaped |

### Discussion and Results

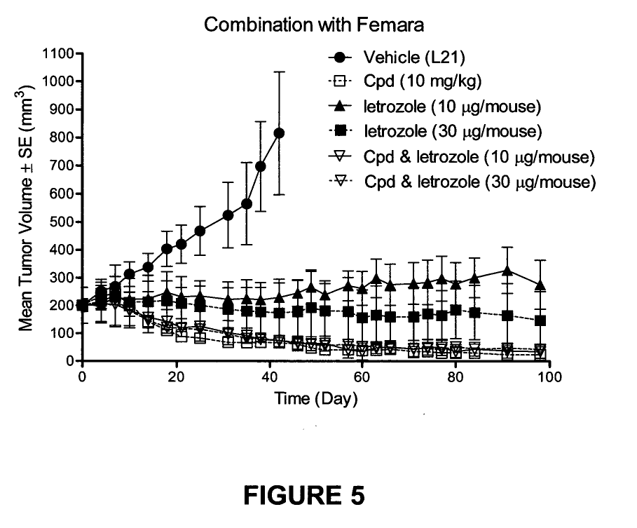

As shown in FIG. 5, in mice treated with test compound alone, tumors regressed and growth was completely prevented. At Day 100, there were five mice with cures (no measurable tumors, with tumor size less than 50 mm³ or less) and four mice with tumors that were less than 60 mm³. One test compound-treated mouse was found dead at Day 31, possibly due to a dosing mishap.

In mice treated with letrozole (10 μg/mouse) alone, although the tumors did not regress, tumor growth was prevented. In mice treated with letrozole (30 μg/mouse) alone, the tumors regressed, with the decrease in tumor size reaching statistical significance (p<0.05, Student's t-test, Dunn's method) compared to the initial tumor size at Day 18. At Day 100, two of the nine mice in the group had cures (no measurable tumor, with tumor size less than 50 mm³ or less) and the remaining mice had tumors that ranged from 90 mm³ to 260 mm³.

Throughout the study, the decrease in tumor size with test compound treatment was better than that observed with letrozole (each compared to vehicle-treated subjects), with differences in tumor size between mice treated with test compound and letrozole at 10 or at 30 μg/mouse reaching statistical significance (p<0.05, Student's t-test, Dunn's method) by Day 14 and remaining significant throughout the study.

Based on the effectiveness of the test compound, no further effect could be noted with the combination of letrozole and the test compound at either dose. In observing the animals, there was no evidence of toxicity associated with any of the treatments.

The discussion above provides illustrative features and embodiments of the present invention, but the invention is not limited to the particular features and embodiments disclosed. Those skilled in the relevant arts will readily appreciate that variations to the disclosed features and embodiments may be made without departing from the spirit and scope of the present invention. For example, one or more of the disclosed features or embodiments may be combined with one or more other features or embodiments.

All publications and patent applications cited herein are incorporated by reference to the same extent as if each individual publication or patent application was specifically and individually indicated to be incorporated by reference.

What is claimed:

1. A use of one or more compounds of Formula (V): 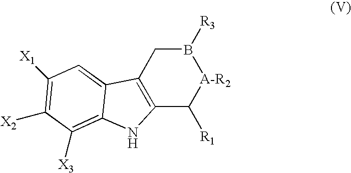
or a pharmaceutically acceptable salt, hydrate, solvate, clathrate, polymorph, racemate or stereoisomer thereof wherein,
X₁ is hydrogen; C₁ to C₆ alkyl optionally substituted with one or more halogen substituents; hydroxyl; halogen; or, C₁ to C₅ alkoxy optionally substituted with aryl;
X₂ is hydrogen or C₁ to C₆ alkoxy;
X₃ is hydrogen or C₁ to C₆ alkyl;
A is CH or N;
B is CH or N, with the proviso that at least one of A or B is N, and the other is CH;
R₁ is one substituent selected from hydroxyl; C₁ to C₈ alkyl, wherein C₁ to C₈ alkyl is optionally substituted with C₁ to C₄ alkylthio, 5 to 10 membered heteroaryl, or aryl, wherein aryl is optionally substituted with one or more substituents independently selected from Rₒ; C₂ to C₈ alkenyl; C₂ to C₈ alkynyl; C₃₋₁₄cycloalkyl; 3 to 12 membered heterocycle, wherein heterocycle is optionally substituted with one or more substituents independently selected from halogen, oxo, amino, C₁ to C₄ alkylamino, acetamino, thio, or C₁ to C₄ alkylthio; 5 to 12 membered heteroaryl, wherein heteroaryl is optionally substituted with one or more substituents independently selected from halogen, oxo, amino, C₁ to C₄ alkylamino, acetamino or C₁ to C₄ alkylthio; or aryl, wherein aryl is optionally substituted with one or more substituents independently selected from Rₒ;
Rₒ is one, two, three, four or five substituents selected from halogen; cyano; nitro; sulfonyl substituted with C₁ to C₆ alkyl or 3 to 10 membered heterocycle; amino, wherein amino is optionally mono- or disubstituted with C₁ to C₆ alkyl, —C(O)—R_{b}, —C(O)O—R_{b}, C₁ to C₄ alkylsulfonyl, or 3 to 10 membered heterocycle, wherein heterocycle is optionally substituted with oxo or —C(O)O—R_{f}; 5 to 6 membered heterocycle; 5 to 6 membered heteroaryl; C₁ to C₆ alkyl, wherein C₁ to C₆ alkyl is optionally substituted with one or more substituents independently selected from hydroxyl, halogen, amino or 3 to 12 membered heterocycle, wherein amino and heterocycle are optionally substituted with one or more substituents independently selected from C₁ to C₄ alkyl or C₁ to C₄ acetyl, wherein C₁ to C₄ alkyl is optionally substituted with one or more substituents independently selected from C₁ to C₄ alkoxy, amino, C₁ to C₄ alkylamino, or 5 to 10 membered heterocycle; —C(O)—R_{b}; —C(O)O—Rₑ; or —ORₐ;
Rₐ is hydrogen; C₂ to C₈ alkenyl; —C(O)—R_{b}; —C(O)O—R_{b}; —C(O)—NH—R_{b}; or C₁ to C₈ alkyl, wherein C₁ to C₈ alkyl is optionally substituted with one or more substituents independently selected from hydroxyl, halogen, C₁ to C₄ alkoxy, amino, C₁ to C₄ alkylamino, acetamino, —OC(O)—R_{b}, —C(O)—R_{b}, —C(O)O—R_{b}, aryl, 3 to 12 membered heterocycle, or 5 to 12 heteroaryl; further wherein C₁ to C₄ alkoxy is optionally substituted with —C(O)—R_{b}, —C(O)O—R_{b} or is optionally further substituted with C₁ to C₄ alkoxy; further wherein amino is optionally substituted with —C(O)—R_{b}, —C(O)O—R_{b}, C₁ to C₄ alkylsulfonyl or 5 to 12 membered heteroaryl, wherein heteroaryl is optionally substituted with C₁ to C₄ alkyl; further wherein C₁ to C₄ alkylamino is optionally substituted on C₁ to C₄ alkyl with hydroxyl, C₁ to C₄ alkoxy, or 5 to 12 membered heteroaryl, wherein heteroaryl is optionally substituted with C₁ to C₄ alkyl; further wherein acetamide is optionally substituted with C₁ to C₄ alkoxy or C₁ to C₄ alkylsulfonyl; further wherein aryl is optionally substituted with 5 to 12 membered heteroaryl optionally substituted with C₁ to C₄ alkyl; and, further wherein heterocycle is optionally substituted with oxo or C₁ to C₄ alkyl optionally substituted with hydroxyl, amino, C₁ to C₄ alkylamino, —C(O)—R_{f}, —C(O)O—R_{f}, or oxo;
R_{b} is hydroxyl; amino optionally substituted with 3 to 12 membered heterocycle optionally substituted with one or more substituents selected from C₁ to C₆ alkyl, C₁ to C₄ alkoxy, oxo or —C(O)O—R_{f}; C₁ to C₄ alkylamino, wherein C₁ to C₄ alkylamino is optionally substituted on C₁ to C₄ alkyl with hydroxyl, amino, C₁ to C₄ alkylamino, C₁ to C₄ alkoxy, 5 to 12 membered heteroaryl, 3 to 12 membered heterocycle optionally substituted with one or more substituents independently selected from C₁ to C₆ alkyl, C₁ to C₄ alkoxy, oxo, —C(O)O—Rₙ, or 5 to 12 membered heteroaryl optionally substituted with a C₁ to C₄ alkyl; C₂ to C₈ alkenyl; C₂ to C₈ alkynyl; aryl, wherein the aryl is optionally substituted with one or more substituents selected from halogen or C₁ to C₄ alkoxy; 5 to 12 membered heteroaryl; 3 to 12 membered heterocycle, wherein heterocycle is optionally substituted with one or more substituents independently selected from acetamino, —C(O)O—Rₙ, 5 to 6 membered heterocycle, C₃₋₁₄cycloalkyl or C₁ to C₆ alkyl, wherein C₁ to C₆ alkyl is optionally further substituted with one or more substituents independently selected from hydroxyl, C₁ to C₄ alkoxy, amino or C₁ to C₄ alkylamino; or C₁ to C₈ alkyl, wherein C₁ to C₈ alkyl is optionally substituted with one or more substituents independently selected from C₁ to C₄ alkoxy, aryl, amino, C₁ to C₄ alkylamino, —C(O)O—Rₙ, —NH—C(O)O—R_{f}, or 3 to 12 membered heterocycle, wherein heterocycle is optionally substituted with one or more substituents independently selected from C₁ to C₆ alkyl, oxo, or —C(O)O—Rₙ;
R₂ is hydrogen, hydroxyl, 5 to 10 membered heteroaryl, C₁ to C₈ alkyl, wherein C₁ to C₈ alkyl is optionally substituted with hydroxyl, C₁ to C₄ alkoxy, 3 to 10 membered heterocycle, 5 to 10 membered heteroaryl or aryl, —C(O)—R_{c}, —C(O)O—R_{d}, —C(O)—N(R_{d}R_{d}), —C(S)—N(R_{d}R_{d}), —C(S)—O—Rₑ, —SO₂—Rₑ, —C(NRₑ)—S—Rₑ, —C(S)—S—R_{f}, or —C(O)—C(O)O—R_{f};
R_{c} is hydrogen; aryl, wherein aryl is optionally substituted with one or more substituents independently selected from halogen, haloalkyl, hydroxyl, C₁ to C₄ alkoxy, C₁ to C₆ alkyl, aryl or —C(O)—Rₙ; 5 to 6 membered heterocycle, wherein heterocycle is optionally substituted with —C(O)—Rₙ; 5 to 6 membered heteroaryl; thiazole-amino; C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)—Rₙ, —C(O)O—Rₙ, —OC(O)—Rₙ, hydroxyl or amino, wherein C₁ to C₄ alkoxy is optionally further substituted with C₁ to C₄ alkoxy, and wherein amino is optionally further substituted with —C(O)O—Rₙ;
R_{d} is independently hydrogen; C₂ to C₈ alkenyl; C₂ to C₈ alkynyl; aryl, wherein aryl is optionally substituted with one or more substituents independently selected from halogen, nitro, C₁ to C₆ alkyl, haloalkyl, —C(O)O—Rₑ or —ORₑ; 5 to 6 membered heteroaryl, wherein heteroaryl is optionally substituted with C₁ to C₆ alkyl or haloalkyl; C₃₋₁₄cycloalkyl, wherein C₃₋₁₄cycloalkyl is optionally substituted with one or more substituents independently selected from halogen, C₁ to C₄ alkyl or C₁ to C₄ alkoxy; or, C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)—Rₙ, —O—C(O)—Rₙ, or hydroxyl, wherein aryl is optionally substituted with one or more substituents independently selected from halogen or haloalkyl;
Rₑ is hydrogen; C₁ to C₆ alkyl, C₃₋₁₄cycloalkyl or aryl, wherein C₁ to C₆ alkyl is optionally substituted with one or more substituents independently selected from halogen, C₁ to C₄ alkoxy or aryl, wherein each instance of aryl is optionally substituted with one or more substituents independently selected from halogen or C₁ to C₄ alkoxy;
R_{f} is C₁ to C₆ alkyl optionally substituted with one or more substituents independently selected from halogen, hydroxyl, C₁ to C₄ alkoxy, cyano, aryl or —C(O)—Rₙ, wherein C₁ to C₄ alkoxy may be optionally substituted with C₁ to C₄ alkoxy and wherein aryl may be optionally substituted with one or more substituents independently selected from halogen, hydroxyl, C₁ to C₄ alkoxy, cyano, or C₁ to C₆ alkyl;
Rₙ is hydroxyl, C₁ to C₄ alkoxy, amino, or C₁ to C₆ alkyl optionally substituted with C₁ to C₄ alkoxy optionally further substituted with C₁ to C₄ alkoxy which is optionally further substituted with C₁ to C₄ alkoxy;
R₃ is hydrogen; C₁ to C₆ alkyl optionally substituted with hydroxy; aryl optionally substituted with C₁ to C₄ alkoxy; or —C(O)—R_{g}; and
R_{g} is hydroxyl or amino, wherein amino is optionally substituted with C₃₋₁₄cycloalkyl or 5 to 10 membered heteroaryl, wherein heteroaryl is optionally substituted with C₁ to C₄ alkyl; or 5 to 10 membered heterocycle, wherein heterocycle is optionally substituted with —C(O)—Rₙ;
in the manufacture of a medicament for post-transcriptionally inhibiting the expression of VEGF in a subject in need thereof comprising inhibiting VEGF mRNA translation by orally administering said medicament once, twice or thrice daily to the subject.

2. The use of claim 1, wherein the compound of Formula (V) includes a compound wherein,
X₁ is hydrogen; C₁ to C₆ alkyl optionally substituted with one or more halogen substituents; hydroxyl; halogen; or, C₁ to C₅ alkoxy optionally substituted with aryl,
with the proviso that, when X₁ is C₁ to C₅ alkoxy and R₂ is —C(O)O—R_{d}, wherein R_{d} is C₁ to C₄ alkyl, then R₁ is other than unsubstituted C₁ to C₈ alkyl;
X₂ is hydrogen or C₁ to C₆ alkoxy;
X₃ is hydrogen or C₁ to C₆ alkyl;
A is CH or N;
B is CH or N, with the proviso that at least one of A or B is N, and the other is CH;
R₁ is one substituent selected from hydroxyl; C₁ to C₈ alkyl, wherein C₁ to C₈ alkyl is optionally substituted with C₁ to C₄ alkylthio, 5 to 10 membered heteroaryl, or aryl, wherein aryl is optionally substituted with one or more substituents independently selected from Rₒ; C₂ to C₈ alkenyl; C₂ to C₈ alkynyl; C₃₋₁₄cycloalkyl; 3 to 12 membered heterocycle, wherein heterocycle is optionally substituted with one or more substituents independently selected from halogen, oxo, amino, C₁ to C₄ alkylamino, acetamino, thio, or C₁ to C₄ alkylthio; 5 to 12 membered heteroaryl, wherein heteroaryl is optionally substituted with one or more substituents independently selected from halogen, oxo, amino, C₁ to C₄ alkylamino, acetamino or C₁ to C₄ alkylthio; or aryl, wherein aryl is optionally substituted with one or more substituents independently selected from Rₒ,
with the proviso that, when R₁ is unsubstituted phenyl, then X₁ is other than hydrogen;
Rₒ is one, two, three, four or five substituents selected from halogen; cyano; nitro; sulfonyl substituted with C₁ to C₆ alkyl or 3 to 10 membered heterocycle; amino, wherein amino is optionally mono- or disubstituted with C₁ to C₆ alkyl, —C(O)—R_{b}, —C(O)O—R_{b}, C₁ to C₄ alkylsulfonyl, or 3 to 10 membered heterocycle, wherein heterocycle is optionally substituted with oxo or —C(O)O—R_{f}; 5 to 6 membered heterocycle; 5 to 6 membered heteroaryl; C₁ to C₆ alkyl, wherein C₁ to C₆ alkyl is optionally substituted with one or more substituents independently selected from hydroxyl, halogen, amino or 3 to 12 membered heterocycle, wherein amino and heterocycle are optionally substituted with one or more substituents independently selected from C₁ to C₄ alkyl or C₁ to C₄ acetyl, wherein C₁ to C₄ alkyl is optionally substituted with one or more substituents independently selected from C₁ to C₄ alkoxy, amino, C₁ to C₄ alkylamino, or 5 to 10 membered heterocycle; —C(O)—R_{b}; —C(O)O—Rₑ; or —ORₐ;
Rₐ is hydrogen; C₂ to C₈ alkenyl; —C(O)—R_{b}; —C(O)O—R_{b}; —C(O)—NH—R_{b}; or C₁ to C₈ alkyl, wherein C₁ to C₈ alkyl is optionally substituted with one or more substituents independently selected from hydroxyl, halogen, C₁ to C₄ alkoxy, amino, C₁ to C₄ alkylamino, acetamino, —OC(O)—R_{b}, —C(O)—R_{b}, —C(O)O—R_{b}, aryl, 3 to 12 membered heterocycle, or 5 to 12 heteroaryl; further wherein C₁ to C₄ alkoxy is optionally substituted with —C(O)—R_{b}, —C(O)O—R_{b} or is optionally further substituted with C₁ to C₄ alkoxy; further wherein amino is optionally substituted with —C(O)—R_{b}, —C(O)O—R_{b}, C₁ to C₄ alkylsulfonyl or 5 to 12 membered heteroaryl, wherein heteroaryl is optionally substituted with C₁ to C₄ alkyl; further wherein C₁ to C₄ alkylamino is optionally substituted on C₁ to C₄ alkyl with hydroxyl, C₁ to C₄ alkoxy, or 5 to 12 membered heteroaryl, wherein heteroaryl is optionally substituted with C₁ to C₄ alkyl; further wherein acetamide is optionally substituted with C₁ to C₄ alkoxy or C₁ to C₄ alkylsulfonyl; further wherein aryl is optionally substituted with 5 to 12 membered heteroaryl optionally substituted with C₁ to C₄ alkyl; and, further wherein heterocycle is optionally substituted with oxo or C₁ to C₄ alkyl optionally substituted with hydroxyl, amino, C₁ to C₄ alkylamino, —C(O)—R_{f}, —C(O)O—R_{f}, or oxo;
R_{b} is hydroxyl; amino optionally substituted with 3 to 12 membered heterocycle optionally substituted with one or more substituents selected from C₁ to C₆ alkyl, C₁ to C₄ alkoxy, oxo or —C(O)O—R_{f}; C₁ to C₄ alkylamino, wherein C₁ to C₄ alkylamino is optionally substituted on C₁ to C₄ alkyl with hydroxyl, amino, C₁ to C₄ alkylamino, C₁ to C₄ alkoxy, 5 to 12 membered heteroaryl, 3 to 12 membered heterocycle optionally substituted with one or more substituents independently selected from C₁ to C₆ alkyl, C₁ to C₄ alkoxy, oxo, —C(O)O—Rₙ, or 5 to 12 membered heteroaryl optionally substituted with a C₁ to C₄ alkyl; C₂ to C₈ alkenyl; C₂ to C₈ alkynyl; aryl, wherein the aryl is optionally substituted with one or more substituents selected from halogen or C₁ to C₄ alkoxy; 5 to 12 membered heteroaryl; 3 to 12 membered heterocycle, wherein heterocycle is optionally substituted with one or more substituents independently selected from acetamino, —C(O)O—Rₙ, 5 to 6 membered heterocycle, C₃₋₁₄cycloalkyl or C₁ to C₆ alkyl, wherein C₁ to C₆ alkyl is optionally further substituted with one or more substituents independently selected from hydroxyl, C₁ to C₄ alkoxy, amino or C₁ to C₄ alkylamino; or C₁ to C₈ alkyl, wherein C₁ to C₈ alkyl is optionally substituted with one or more substituents independently selected from C₁ to C₄ alkoxy, aryl, amino, C₁ to C₄ alkylamino, —C(O)O—Rₙ, —NH—C(O)O—R_{f}, or 3 to 12 membered heterocycle, wherein heterocycle is optionally substituted with one or more substituents independently selected from C₁ to C₆ alkyl, oxo, or —C(O)O—Rₙ;
R₂ is hydrogen, hydroxyl, 5 to 10 membered heteroaryl, C₁ to C₈ alkyl, wherein C₁ to C₈ alkyl is optionally substituted with hydroxyl, C₁ to C₄ alkoxy, 3 to 10 membered heterocycle, 5 to 10 membered heteroaryl or aryl, —C(O)—R_{c}, —C(O)O—R_{d}, —C(O)—N(R_{d}R_{d}), —C(S)—N(R_{d}R_{d}), —C(S)—O—Rₑ, —SO₂—Rₑ, —C(NRₑ)—S—Rₑ, —C(S)—S—R_{f}, or —C(O)—C(O)O—R_{f},
with the proviso that, when R₂, R₃, X₁, X₂ and X₃ are hydrogen, then R₁ is other than fluorenyl, substituted carbazolyl or phenyl, wherein phenyl is optionally monosubstituted with halogen, nitro or substituted amino, or di- and tri-substituted with C₁ to C₄ alkoxy;
with the proviso that, when R₂ is —C(O)—R_{c}, —C(O)O—R_{d}, —C(O)—NH(R_{d}) or —C(S)—NH(R_{d}), wherein R_{c} is C₁ to C₈ alkyl substituted with optionally substituted phenyl, wherein R_{d} is optionally substituted phenyl, cyclohexyl or C₁ to C₈ alkyl optionally substituted with optionally substituted phenyl or —C(O)O—Rₙ, and R₃, X₁, X₂ and X₃ are hydrogen, then R₁ is other than unsubstituted benzo[1,3]dioxolyl or optionally substituted phenyl, wherein phenyl is optionally disubstituted with chloro and methoxy;
R_{c} is hydrogen; aryl, wherein aryl is optionally substituted with one or more substituents independently selected from halogen, haloalkyl, hydroxyl, C₁ to C₄ alkoxy, C₁ to C₆ alkyl, aryl or —C(O)—Rₙ; 5 to 6 membered heterocycle, wherein heterocycle is optionally substituted with —C(O)—Rₙ; 5 to 6 membered heteroaryl; thiazole-amino; C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)—Rₙ, —C(O)O—Rₙ, —OC(O)—Rₙ, hydroxyl or amino, wherein C₁ to C₄ alkoxy is optionally further substituted with C₁ to C₄ alkoxy, and wherein amino is optionally further substituted with —C(O)O—Rₙ;
R_{d} is independently hydrogen; C₂ to C₈ alkenyl; C₂ to C₈ alkynyl; aryl, wherein aryl is optionally substituted with one or more substituents independently selected from halogen, nitro, C₁ to C₆ alkyl, haloalkyl, —C(O)O—Rₑ or —ORₑ; 5 to 6 membered heteroaryl, wherein heteroaryl is optionally substituted with C₁ to C₆ alkyl or haloalkyl; C₃₋₁₄cycloalkyl, wherein C₃₋₁₄cycloalkyl is optionally substituted with one or more substituents independently selected from halogen, C₁ to C₄ alkyl or C₁ to C₄ alkoxy; or, C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)—Rₙ, —O—C(O)—Rₙ, or hydroxyl, wherein aryl is optionally substituted with one or more substituents independently selected from halogen or haloalkyl;
Rₑ is hydrogen; C₁ to C₆ alkyl, C₃₋₁₄cycloalkyl or aryl, wherein C₁ to C₆ alkyl is optionally substituted with one or more substituents independently selected from halogen, C₁ to C₄ alkoxy or aryl, wherein each instance of aryl is optionally substituted with one or more substituents independently selected from halogen or C₁ to C₄ alkoxy;
R_{f} is C₁ to C₆ alkyl optionally substituted with one or more substituents independently selected from halogen, hydroxyl, C₁ to C₄ alkoxy, cyano, aryl or —C(O)—Rₙ, wherein C₁ to C₄ alkoxy may be optionally substituted with C₁ to C₄ alkoxy and wherein aryl may be optionally substituted with one or more substituents independently selected from halogen, hydroxyl, C₁ to C₄ alkoxy, cyano, or C₁ to C₆ alkyl;
Rₙ is hydroxyl, C₁ to C₄ alkoxy, amino, or C₁ to C₆ alkyl optionally substituted with C₁ to C₄ alkoxy optionally further substituted with C₁ to C₄ alkoxy which is optionally further substituted with C₁ to C₄ alkoxy;
R₃ is hydrogen; C₁ to C₆ alkyl optionally substituted with hydroxy; aryl optionally substituted with C₁ to C₄ alkoxy; or —C(O)—R_{g}; and
R_{g} is hydroxyl or amino, wherein amino is optionally substituted with C₃₋₁₄cycloalkyl or 5 to 10 membered heteroaryl, wherein heteroaryl is optionally substituted with C₁ to C₄ alkyl; or 5 to 10 membered heterocycle, wherein heterocycle is optionally substituted with —C(O)—Rₙ,
with the proviso that, when R₃ is —C(O)—R_{g} and R_{g} is hydroxyl and R₂, X₁, X₂ and X₃ are hydrogen, then R₁ is other than unsubstituted C₁ to C₈ alkyl, unsubstituted phenyl or (4-methoxy)phenyl,
with the proviso that, when R₃ is —C(O)—R_{g} and R_{g} is hydroxyl and R₂ is tert-butoxycarbonyl, then R₁ is other than indole optionally substituted with C₁ to C₈ alkyl or benzyl, and
with the proviso that, when R₃ is —C(O)—R_{g} and R_{g} is amino substituted with benzothiazolyl and R₂ is hydrogen or tert-butoxycarbonyl, then R₁ is other than cyclohexyl.

3. The use of claim 1, wherein the compound of Formula (V) includes a compound wherein,
X is hydrogen; C₁ to C₆ alkyl; hydroxyl; halogen; or, C₁ to C₅ alkoxy optionally substituted with aryl,
with the proviso that, when X is C₁ to C₅ alkoxy and R₂ is —C(O)O—R_{d}, wherein R_{d} is C₁ to C₄ alkyl, then R₁ is other than unsubstituted C₁ to C₈ alkyl;
R₁ is one substituent selected from hydroxyl; C₁ to C₈ alkyl, wherein C₁ to C₈ alkyl is optionally substituted with C₁ to C₄ alkylthio or aryl, wherein aryl is optionally substituted with one or more substituents independently selected from Rₒ; C₂ to C₈ alkenyl; C₃₋₁₄cycloalkyl; 3 to 12 membered heterocycle, wherein heterocycle is optionally substituted with one or more substituents independently selected from halogen or oxo; 5 to 12 membered heteroaryl, wherein heteroaryl is optionally substituted with one or more substituents independently selected from halogen, oxo, C₁ to C₄ alkylamino, acetamino or C₁ to C₄ alkylthio; or, aryl, wherein aryl is optionally substituted with one or more substituents independently selected from Rₒ,
with the proviso that, when R₁ is unsubstituted phenyl, then X is other than hydrogen;
Rₒ is one, two or three substituents selected from halogen; cyano; nitro; sulfonyl substituted with C₁ to C₆ alkyl or 3 to 10 membered heterocycle; amino, wherein amino is optionally mono- or disubstituted with C₁ to C₆ alkyl, —C(O)—R_{b}, —C(O)O—R_{b} or 3 to 10 membered heterocycle, wherein heterocycle is optionally substituted with —C(O)O—R_{f}; C₁ to C₆ alkyl, wherein C₁ to C₆ alkyl is optionally substituted with one or more substituents independently selected from hydroxyl, halogen, amino or 3 to 12 membered heterocycle, wherein amino and heterocycle are optionally substituted with C₁ to C₄ alkyl, wherein C₁ to C₄ alkyl is optionally substituted with C₁ to C₄ alkoxy or 5 to 10 membered heterocycle; —C(O)—R_{b}; —C(O)O—Rₑ; or —ORₐ;
Rₐ is hydrogen; C₂ to C₈ alkenyl; —C(O)—R_{b}; —C(O)O—R_{b} or C₁ to C₈ alkyl, wherein C₁ to C₈ alkyl is optionally substituted with one or more substituents independently selected from hydroxyl, halogen, C₁ to C₄ alkoxy, amino, C₁ to C₄ alkylamino, —OC(O)—R_{b}, aryl, 3 to 12 membered heterocycle, or 5 to 12 heteroaryl; further wherein C₁ to C₄ alkoxy is optionally further substituted with C₁ to C₄ alkoxy; further wherein amino is optionally substituted with —C(O)—R_{b}, —C(O)O—R_{b}, C₁ to C₄ alkylsulfonyl or 5 to 12 membered heteroaryl, wherein heteroaryl is optionally substituted with C₁ to C₄ alkyl; further wherein C₁ to C₄ alkylamino is optionally substituted on C₁ to C₄ alkyl with hydroxyl,
C₁ to C₄ alkoxy, or 5 to 12 membered heteroaryl, further wherein heterocycle is optionally substituted with oxo or C₁ to C₄ alkyl optionally substituted with hydroxyl, C₁ to C₄ alkylamino, —C(O)—R_{f} or —C(O)O—R_{f};
R_{b} is amino optionally substituted with 3 to 12 membered heterocycle, optionally substituted on heterocycle with —C(O)O—R_{f}; C₁ to C₄ alkylamino, wherein C₁ to C₄ alkylamino is optionally substituted on C₁ to C₄ alkyl with hydroxyl, C₁ to C₄ alkylamino, C₁ to C₄ alkoxy, 5 to 12 membered heteroaryl, 3 to 12 membered heterocycle optionally substituted with one or more substituents independently selected from C₁ to C₆ alkyl or oxo; C₂ to C₈ alkenyl; aryl, wherein the aryl is optionally substituted with one or more substituents selected from halogen or C₁ to C₄ alkoxy; 5 to 12 membered heteroaryl; 3 to 12 membered heterocycle, wherein heterocycle is optionally substituted with one or more substituents independently selected from acetamino, —C(O)O—Rₙ, 5 to 6 membered heterocycle, C₃₋₁₄cycloalkyl or C₁ to C₆ alkyl, wherein C₁ to C₆ alkyl is optionally further substituted with one or more substituents independently selected from hydroxyl, C₁ to C₄ alkoxy, amino or C₁ to C₄ alkylamino; or C₁ to C₈ alkyl, wherein C₁ to C₈ alkyl is optionally substituted with one or more substituents independently selected from C₁ to C₄ alkoxy, aryl, amino, C₁ to C₄ alkylamino, —C(O)O—Rₙ, —NH—C(O)O—R_{f}, or 3 to 12 membered heterocycle, wherein heterocycle is optionally substituted with one or more oxo substituents;
R₂ is hydrogen, hydroxyl, 5 to 10 membered heteroaryl, C₁ to C₈ alkyl, wherein C₁ to C₈ alkyl is optionally substituted with 3 to 10 membered heterocycle, 5 to 10 membered heteroaryl or aryl, —C(O)—R_{c}, —C(O)O—R_{d}, —C(O)—N(R_{d}R_{d}), —C(S)—N(R_{d}R_{d}), —C(S)—O—Rₑ, —SO₂—Rₑ, —C(NRₑ)—S—Rₑ, —C(S)—S—R_{f}, or —C(O)—C(O)O—R_{f},
with the proviso that, when R₂, R₃, X₁, X₂ and X₃ are hydrogen, then R₁ is other than fluorenyl, substituted carbazolyl or phenyl, wherein phenyl is optionally monosubstituted with halogen, nitro or substituted amino, or di- and tri-substituted with C₁ to C₄ alkoxy;
with the proviso that, when R₂ is —C(O)—R_{c}, —C(O)O—R_{d}, —C(O)—NH(R_{d}) or —C(S)—NH(R_{d}), wherein Rₑ is C₁ to C₈ alkyl substituted with optionally substituted phenyl, wherein R_{d} is optionally substituted phenyl, cyclohexyl or C₁ to C₈ alkyl optionally substituted with optionally substituted phenyl or —C(O)O—Rₙ, and R₃, X₁, X₂ and X₃ are hydrogen, then R₁ is other than unsubstituted benzo[1,3]dioxolyl or optionally substituted phenyl, wherein phenyl is optionally disubstituted with chloro and methoxy;
R_{c} is aryl, wherein aryl is optionally substituted with one or more substituents independently selected from halogen or aryl; 5 to 6 membered heterocycle, wherein heterocycle is optionally substituted with —C(O)—Rₙ; 5 to 6 membered heteroaryl; C₁ to C₈ alkyl, wherein C₁ to C₈ alkyl is optionally substituted with one or more substituents independently selected from halogen, C₁ to C₄ alkoxy, phenyloxy, aryl, 5 to 6 membered heteroaryl, —C(O)O—Rₙ, —OC(O)—Rₙ or amino, wherein C₁ to C₄ alkoxy is optionally further substituted with C₁ to C₄ alkoxy, and wherein amino is optionally further substituted with —C(O)O—Rₙ;
R_{d} is independently hydrogen; C₂ to C₈ alkenyl; C₂ to C₈ alkynyl; aryl, wherein aryl is optionally substituted with one or more substituents independently selected from halogen, nitro, C₁ to C₆ alkyl, haloalkyl, —C(O)O—Rₑ or —ORₑ; 5 to 6 membered heteroaryl, wherein heteroaryl is optionally substituted with C₁ to C₆ alkyl; C₃₋₁₄cycloalkyl, wherein C₃₋₁₄cycloalkyl is optionally substituted with one or more C₁ to C₄ alkyl substituents; or, C₁ to C₈ alkyl, wherein C₁ to C₈ alkyl is optionally substituted with one or more substituents independently selected from halogen, C₁ to C₄ alkoxy, aryl or 5 to 6 membered heteroaryl;
Rₑ is hydrogen; C₁ to C₆ alkyl, C₃₋₁₄cycloalkyl or aryl, wherein C₁ to C₆ alkyl is optionally substituted with aryl, wherein each instance of aryl is optionally substituted with one or more halogen substituents;
R_{f} is C₁ to C₆ alkyl optionally substituted with one or more substituents independently selected from halogen, hydroxyl, C₁ to C₄ alkoxy, cyano, aryl or —C(O)—Rₙ, wherein C₁ to C₄ alkoxy may be optionally substituted with C₁ to C₄ alkoxy and wherein aryl may be optionally substituted with one or more substituents independently selected from halogen, cyano, or C₁ to C₆ alkyl;
Rₙ is C₁ to C₄ alkoxy, amino, or C₁ to C₆ alkyl;
R₃ is hydrogen; C₁ to C₆ alkyl optionally substituted with hydroxy; aryl optionally substituted with C₁ to C₄ alkoxy; or —C(O)—R_{g}; and
R_{g} is hydroxyl or amino, wherein amino is optionally substituted with C₃₋₁₄cycloalkyl or 5 to 10 membered heteroaryl, wherein heteroaryl is optionally substituted with C₁ to C₄ alkyl; or 5 to 10 membered heterocycle, wherein heterocycle is optionally substituted with —C(O)—Rₙ,
with the proviso that, when R₃ is —C(O)—R_{g} and R_{g} is hydroxyl and R₂, X₁, X₂ and X₃ are hydrogen, then R₁ is other than unsubstituted C₁ to C₈ alkyl, unsubstituted phenyl or (4-methoxy)phenyl,
with the proviso that, when R₃ is —C(O)—R_{g} and R_{g} is hydroxyl and R₂ is tert-butoxycarbonyl, then R₁ is other than indole optionally substituted with C₁ to C₈ alkyl or benzyl, and
with the proviso that, when R₃ is —C(O)—R_{g} and R_{g} is amino substituted with benzothiazolyl and R₂ is hydrogen or tert-butoxycarbonyl, then R₁ is other than cyclohexyl.

4. The use of claim 1, wherein the compound of Formula (V) includes a compound wherein,
X is hydrogen; C₁ to C₆ alkyl; hydroxyl; halogen; or, C₁ to C₅ alkoxy optionally substituted with phenyl,
with the proviso that, when X is C₁ to C₅ alkoxy and R₂ is —C(O)O—R_{d}, wherein R_{d} is C₁ to C₄ alkyl, then R₁ is other than unsubstituted C₁ to C₈ alkyl;
R₁ is one substituent selected from hydroxyl; C₁ to C₈ alkyl, wherein C₁ to C₈ alkyl is optionally substituted with C₁ to C₄ alkylthio or aryl, wherein aryl is optionally substituted with one or more substituents independently selected from Rₒ; C₂ to C₈ alkenyl; cyclohex-3-enyl; benzo[1,3]dioxolyl optionally substituted with halogen; 4H-chromenyl optionally substituted with oxo; dihydro-benzofuranyl, tetrahydrofuranyl, furanyl, thiazolyl, pyrimidinyl, indolyl, wherein each of furanyl, thiazolyl, pyrimidinyl and indolyl are optionally substituted with one or more substituents independently selected from halogen, oxo, C₁ to C₄ alkylamino, acetamino or C₁ to C₄ alkylthio; or, phenyl, wherein phenyl is optionally substituted with one or more substituents independently selected from Rₒ, with the proviso that, when R₁ is unsubstituted phenyl, then X is other than hydrogen;
Rₒ is one, two or three substituents selected from halogen; cyano; nitro; sulfonyl substituted with C₁ to C₆ alkyl or morpholinyl; amino, wherein amino is optionally mono- or disubstituted with C₁ to C₆ alkyl, —C(O)—R_{b}, —C(O)O—R_{b}, piperidinyl or tetrahydro-2H-pyranyl, wherein piperidinyl is optionally substituted with —C(O)O—R_{f}; C₁ to C₆ alkyl, wherein C₁ to C₆ alkyl is optionally substituted with one or more substituents independently selected from hydroxyl, halogen, amino or piperazinyl, wherein amino and piperazinyl are optionally substituted with C₁ to C₄ alkyl, wherein C₁ to C₄ alkyl is optionally substituted with C₁ to C₄ alkoxy or morpholinyl; —C(O)—R_{b}; —C(O)O—Rₑ; or —ORₐ;
Rₐ is hydrogen; C₂ to C₈ alkenyl; —C(O)—R_{b}; —C(O)O—R_{b} or C₁ to C₈ alkyl, wherein C₁ to C₈ alkyl is optionally substituted with one or more substituents independently selected from hydroxyl, halogen, C₁ to C₄ alkoxy, amino, C₁ to C₄ alkylamino, —OC(O)—R_{b}, phenyl, oxiranyl, pyrrolidinyl, morpholinyl, thiomorpholinyl, piperidinyl, piperazinyl, dioxolidinyl, imidazolyl, pyrazolyl or triazolyl; further wherein C₁ to C₄ alkoxy is optionally further substituted with C₁ to C₄ alkoxy; further wherein amino is optionally substituted with —C(O)—R_{b}, —C(O)O—R_{b}, C₁ to C₄ alkylsulfonyl, thiazolyl or pyridinyl, wherein thiazolyl is optionally substituted with C₁ to C₄ alkyl; further wherein C₁ to C₄ alkylamino is optionally substituted on C₁ to C₄ alkyl with hydroxyl, C₁ to C₄ alkoxy or imidazolyl, wherein imidazolyl is optionally substituted with C₁ to C₄ alkyl; wherein dioxolidinyl is optionally substituted with oxo; and, wherein each of pyrrolidinyl, piperidinyl and piperazinyl are optionally substituted with C₁ to C₄ alkyl, wherein C₁ to C₄ alkyl is optionally substituted with hydroxyl, C₁ to C₄ alkylamino, —C(O)—R_{f} or —C(O)O—R_{f};
R_{b} is amino optionally substituted with piperidinyl, wherein piperidinyl is optionally substituted with —C(O)O—R_{f}; C₁ to C₄ alkylamino, wherein C₁ to C₄ alkylamino is optionally substituted on C₁ to C₄ alkyl with hydroxyl, C₁ to C₄ alkylamino, C₁ to C₄ alkoxy, imidazolyl; pyridinyl, tetrahydrofuranyl, pyrrolidinyl, dioxolidinyl or morpholinyl, wherein each of pyrrolidinyl and dioxolidinyl are optionally substituted with one or more substituents independently selected from C₁ to C₆ alkyl or oxo; C₂ to C₈ alkenyl; phenyl, wherein phenyl is optionally substituted with one or more halogen substituents; furanyl, pyrrolidinyl, piperidinyl, piperazinyl, oxazolidinyl, 1,4-diazepanyl, wherein each of pyrrolidinyl, piperidinyl, piperazinyl and 1,4-diazepanyl are optionally substituted with one or more substituents independently selected from acetamino, —C(O)O—Rₙ, pyrrolidinyl, piperidinyl, cyclohexyl or C₁ to C₆ alkyl, wherein C₁ to C₆ alkyl is optionally further substituted with one or more substituents independently selected from hydroxyl, C₁ to C₄ alkoxy, amino or C₁ to C₄ alkylamino; or C₁ to C₈ alkyl, wherein C₁ to C₈ alkyl is optionally substituted with one or more substituents independently selected from C₁ to C₄ alkoxy, aryl, amino, C₁ to C₄ alkylamino, —C(O)O—R_{d}, —NH—C(O)O—R_{f}, morpholinyl or hexahydro-1H-thieno[3,4-d]imidazolyl substituted on the imidazolyl portion with oxo;
R₂ is hydrogen, hydroxyl, pyrazinyl, pyrimidinyl, C₁ to C₈ alkyl, wherein C₁ to C₈ alkyl is optionally substituted with 1,3-dioxanyl, furanyl or phenyl, —C(O)—R_{c}, —C(O)O—R_{d}, —C(O)—N(R_{d}R_{d}), —C(S)—N(R_{d}R_{d}), —C(S)—O—Rₑ, —SO₂—Rₑ, —C(NRₑ)—S—Rₑ, —C(S)—S—R_{f}, or —C(O)—C(O)O—R_{f},
with the proviso that, when R₂, R₃ and X are hydrogen, then R₁ is other than fluorenyl, substituted carbazolyl or phenyl, wherein phenyl is optionally monosubstituted with halogen, nitro or substituted amino, or di- and tri-substituted with C₁ to C₄ alkoxy;
with the proviso that, when R₂ is —C(O)—R_{c}, —C(O)O—R_{d}, —C(O)—NH(R_{d}) or —C(S)—NH(R_{d}), wherein R_{c} is C₁ to C₈ alkyl substituted with optionally substituted phenyl, wherein R_{d} is optionally substituted phenyl, cyclohexyl or C₁ to C₈ alkyl optionally substituted with optionally substituted phenyl or —C(O)O—Rₙ, and R₃ and X are hydrogen, then R₁ is other than unsubstituted benzo[1,3]dioxolyl or optionally substituted phenyl, wherein phenyl is optionally disubstituted with chloro and methoxy;
R_{c} is phenyl, wherein phenyl is optionally substituted with one or more substituents independently selected from halogen or phenyl; morpholinyl, pyrrolidinyl or piperazinyl, wherein each of pyrrolidinyl and piperazinyl are optionally substituted with —C(O)—Rₙ; 5 to 6 membered heteroaryl; C₁ to C₈ alkyl, wherein C₁ to C₈ alkyl is optionally substituted with one or more substituents independently selected from halogen, C₁ to C₄ alkoxy, phenyloxy, phenyl, thienyl, —C(O)O—Rₙ, —OC(O)—Rₙ or amino, wherein C₁ to C₄ alkoxy is optionally further substituted with C₁ to C₄ alkoxy, and wherein amino is optionally further substituted with —C(O)O—Rₙ;
R_{d} is independently hydrogen; C₂ to C₈ alkenyl; C₂ to C₈ alkynyl; phenyl, wherein phenyl is optionally substituted with one or more substituents independently selected from halogen, nitro, C₁ to C₆ alkyl, haloalkyl, —C(O)O—Rₑ or —ORₑ; imidazolyl or thiazolyl, wherein thiazolyl is optionally substituted with C₁ to C₆ alkyl; cyclohexyl, wherein cyclohexyl is optionally substituted with one or more C₁ to C₄ alkyl substituents; or, C₁ to C₈ alkyl, wherein C₁ to C₈ alkyl is optionally substituted with one or more substituents independently selected from halogen, C₁ to C₄ alkoxy, phenyl or imidazolyl;
Rₑ is hydrogen; C₁ to C₆ alkyl, cyclohexyl or phenyl, wherein C₁ to C₆ alkyl is optionally substituted with phenyl, wherein each instance of phenyl is optionally substituted with one or more halogen substituents;
R_{f} is C₁ to C₆ alkyl optionally substituted with one or more substituents independently selected from halogen, hydroxyl, C₁ to C₄ alkoxy, cyano, phenyl or —C(O)—Rₙ, wherein C₁ to C₄ alkoxy may be optionally substituted with C₁ to C₄ alkoxy and wherein phenyl may be optionally substituted with one or more substituents independently selected from halogen, cyano, or C₁ to C₆ alkyl;
R₃ is hydrogen; C₁ to C₆ alkyl optionally substituted with hydroxy; phenyl optionally substituted with C₁ to C₄ alkoxy; or —C(O)—R_{g}; and
R_{g} is hydroxyl or amino, wherein amino is optionally substituted with cyclohexyl or thiazolyl, wherein thiazolyl is optionally substituted with C₁ to C₄ alkyl; or piperazinyl, wherein piperazinyl is optionally substituted with —C(O)—Rₙ,
with the proviso that, when R₃ is —C(O)—R_{g} and R_{g} is hydroxyl and R₂ and X are hydrogen, then R₁ is other than unsubstituted C₁ to C₈ alkyl, unsubstituted phenyl or (4-methoxy)phenyl,
with the proviso that, when R₃ is —C(O)—R_{g} and R_{g} is hydroxyl and R₂ is tert-butoxycarbonyl, then R₁ is other than indole optionally substituted with C₁ to C₈ alkyl or benzyl, and
with the proviso that, when R₃ is —C(O)—R_{g} and R_{g} is amino substituted with benzothiazolyl and R₂ is hydrogen or tert-butoxycarbonyl, then R₁ is other than cyclohexyl; and, all other variables are as previously described.

5. The use of claim 1, wherein inhibiting VEGF mRNA translation treats a VEGF mediated disorder or a solid tumor cancer by reducing plasma and solid tumor VEGF levels, reducing perivascularly sequestered VEGF, reducing aberrant vascular permeability, or inhibiting angiogenesis.

6. The use of claim 5, wherein the VEGF mediated disorder is selected from cancer, diabetic retinopathy, exudative macular degeneration, rheumatoid arthritis, psoriasis, atherosclerosis, chronic inflammation, other chronic inflammation-related diseases and disorders or obesity.

7. The use of claim 6, wherein the cancer is a solid tumor cancer selected from a pediatric solid tumor, an Ewing's sarcoma, a Wilms tumor, a neuroblastoma, a neurofibroma, a carcinoma of the epidermis, a malignant melanoma, a cervical carcinoma, a colon carcinoma, a lung carcinoma, a renal carcinoma, a breast cancinoma or a breast sarcoma.

8. The use of claim 1, wherein the therapeutically effective amount is in a range of from about 0.01 mg/kg/day to about 20 mg/kg/day, or from about 0.015 mg/kg/day to about 10 mg/kg/day, or from about 0.02 mg/kg/day to about 10 mg/kg/day, or from about 0.025 mg/kg/day to about 10 mg/kg/day, or from about 0.03 mg/kg/day to about 10 mg/kg/day, wherein said amount is orally administered once, twice or thrice daily according to subject weight.

9. The use of claim 8, wherein the therapeutically effective amount provides a plasma concentration selected from greater than about 0.01 μg/mL, greater than about 0.05 μg/mL, greater than about 0.10 μg/mL, greater than about 0.15 μg/mL, greater than about 0.20 μg/mL, greater than about 0.25 μg/mL, or greater than about 0.30 μg/mL for a time period of from about 3 to about 24 hours following administration once daily.

10. The use of claim 9, wherein the time period is from about 3 to about 12 hours following administration twice daily.

11. The use of claim 9, wherein the time period is from about 3 to about 8 hours following administration thrice daily.

12. The use of any of claim 9, 10 or 11, wherein the plasma concentration is in a range of from about 0.01 μg/mL to about 100 μg/mL, from about 0.05 μg/mL to about 50 μg/mL, or from about 0.05 μg/mL to about 10 μg/mL following administration once, twice or thrice daily.

13. The use of claim 1, wherein administration once, twice or thrice daily to the subject occurs when the subject is either fasted or fed.

14. The use of claim 13, wherein the Cₘₐₓ for a fed subject may be above the Cₘₐₓ for a fasted subject in a range of greater than about 5% to about 10%, greater than about 5% to about 20%, greater than about 10% to about 20%, greater than about 15% to about 20%, greater than about 15% to about 30%, greater than about 20% to about 40%, or greater than about 20% to about 50%.

15. The use of claim 1, wherein said subject has hypertension or proteinuria, or is at risk of having a stroke and said administration of a therapeutically effective amount of said compound once, twice or thrice daily does not result in a substantial incidence of either proteinuria or hypertension.

16. The use of claim 15, wherein said compound is optionally administered in combination with one or more additional agents useful in the treatment of cancer.

17. The use of claim 16, wherein said agents are selected from the group consisting of paclitaxel, fluorouracil, tamoxifen, doxorubicin, aromasin, exemistane, taxol, 5-fluororuracil, letrozole, CPT-11, a tyrosine kinase inhibitor, a COX-2 inhibitor, thalidomide, gemcitabine, squalamine, endostatin, angiostatin, AE-941, lenalidomide, medi-522, 2-methoxyestradiol, carboxyamidotriazole, combretastatin A4 phosphate, SU6668, SU11248, BMS-275291, COL-3, cilengitide, IMC-1121B, vatalanib, LY317615, VEGF Trap, ZD6474, halofuginone, hydrobromide, celecoxib, interferon alpha, interleukin-12, and bevacizumab.

18. The use of claim 17, wherein said agents that cause a recurrent, persistent or symptomatic elevated blood pressure greater than 20 mm Hg (diastolic) above a normal level or greater than 150 mm Hg/100 mm Hg (systolic/diastolic) are coadministered with at least one agent that reduces blood pressure.

19. The use of claim 15, wherein said agents that cause an increase in protein in said subject's urine or that cause an increase in the grade of proteinuria from grade 1 proteinuria to grade 2 proteinuria are coadministered with at least one agent that reduces protein in urine.

20. The use of claim 1, wherein said one or more compounds of Formula (V) or said pharmaceutical composition thereof, is one or more compounds of Formula (I), Formula (II), Formula (III) or Formula (IV), or one or more compounds of any of Formulas (I-a) through (I-m), or pharmaceutically acceptable salts, hydrates, solvates, clathrates, polymorphs, racemates or stereoisomers thereof: 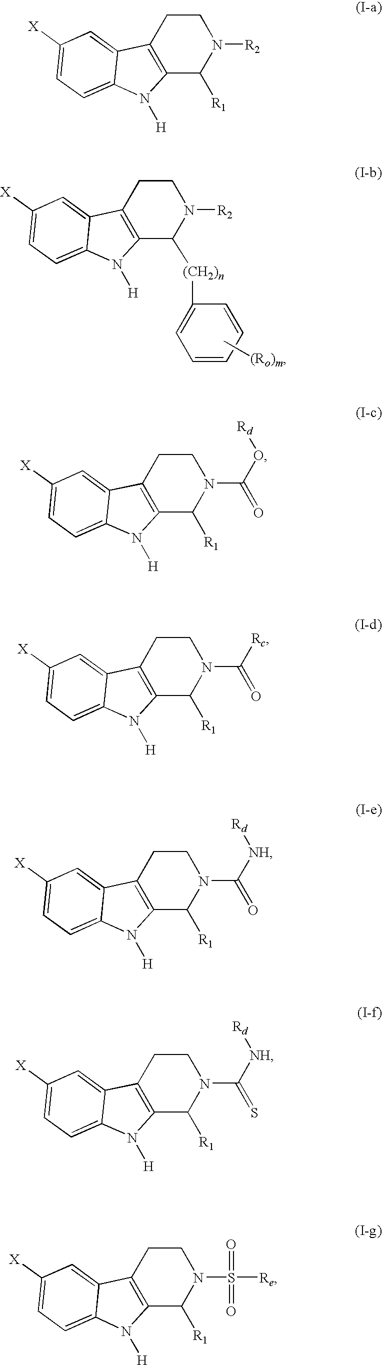 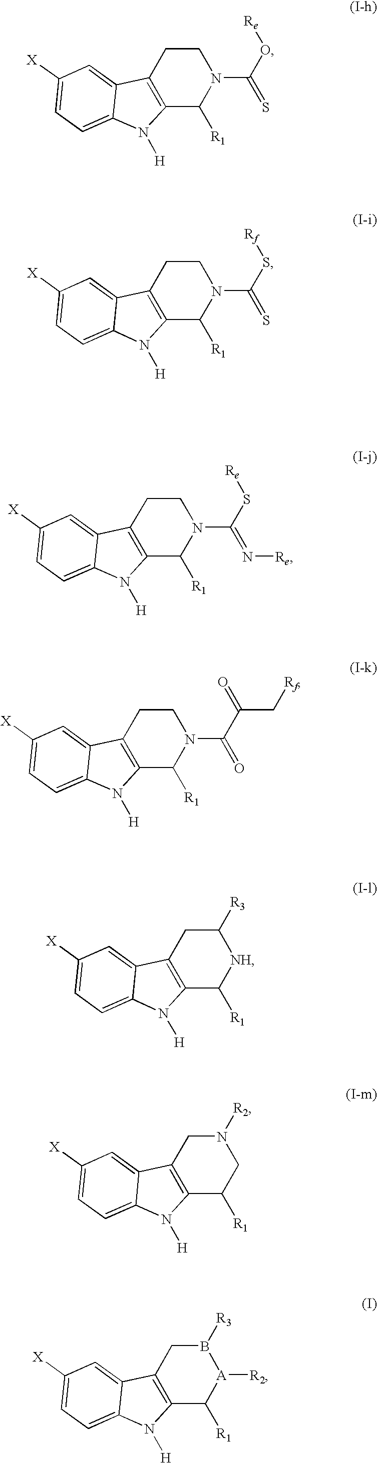 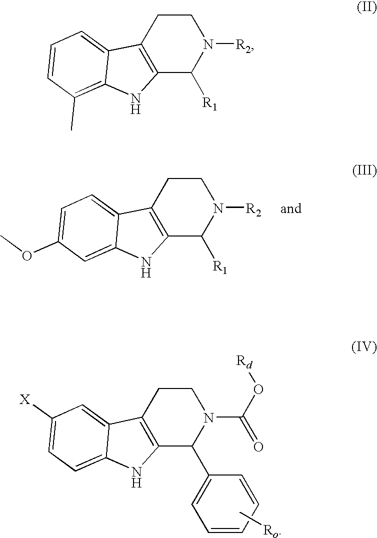
wherein all variables are as defined previously.

21. The use of claim 1, wherein said one or more compounds of Formula (V) or pharmaceutically acceptable salts, hydrates, solvates, clathrates, polymorphs, racemates or stereoisomers thereof is selected from the group consisting of:
ethyl 6-chloro-1-(4-methoxyphenyl)-3,4-dihydro-1H-pyrido[3,4-b]indole-2(9H)-carboxylate,
ethyl 6-bromo-1-(4-chlorophenyl)-3,4-dihydro-1H-pyrido[3,4-b]indole-2(9H)-carboxylate,
ethyl 6-chloro-1-(2,3-difluorophenyl)-3,4-dihydro-1H-pyrido[3,4-b]indole-2(9H)-carboxylate,
ethyl 6-bromo-1-(4-isopropylphenyl)-3,4-dihydro-1H-pyrido[3,4-b]indole-2(9H)-carboxylate,
ethyl 6-bromo-1-p-tolyl-3,4-dihydro-1H-pyrido[3,4-b]indole-2(9H)-carboxylate,
4-chlorophenyl 6-chloro-1-(4-methoxyphenyl)-3,4-dihydro-1H-pyrido[3,4-b]indole-2(9H)-carboxylate,
ethyl 6-chloro-1-(4-chlorophenyl)-3,4-dihydro-1H-pyrido[3,4-b]indole-2(9H)-carboxylate,
2-chloroethyl 6-chloro-1-(4-cyanophenyl)-3,4-dihydro-1H-pyrido[3,4-b]indole-2(9H)-carboxylate,
p-tolyl 6-chloro-1-(4-methoxyphenyl)-3,4-dihydro-1H-pyrido[3,4-b]indole-2(9H)-carboxylate,
ethyl 6-chloro-1-(4-fluorophenyl)-3,4-dihydro-1H-pyrido[3,4-b]indole-2(9H)-carboxylate,
6-bromo-1-(4-isopropylphenyl)-2-(pyrimidin-2-yl)-2,3,4,9-tetrahydro-1H-pyrido[3,4-b]indole,
6-bromo-1-(4-chlorophenyl)-3,4-dihydro-1H-pyrido[3,4-b]indol-2(9H)-ol,
1-(6-bromo-1-(4-isopropylphenyl)-3,4-dihydro-1H-pyrido[3,4-b]indol-2(9H)-yl)ethanone,
6-bromo-1-(4-isopropylphenyl)-2,3,4,9-tetrahydro-1H-pyrido[3,4-b]indole,
6-bromo-1-(3-chlorophenyl)-N-cyclohexyl-3,4-dihydro-1H-pyrido[3,4-b]indole-2(9H)-carboxamide,
1-(benzo[d][1,3]dioxol-5-yl)-6-chloro-2-(pyrimidin-2-yl)-2,3,4,9-tetrahydro-1H-pyrido[3,4-b]indole,
6-bromo-1-(4-methoxyphenyl)-2-(pyrimidin-2-yl)-2,3,4,9-tetrahydro-1H-pyrido[3,4-b]indole,
2-fluoroethyl 6-chloro-1-(4-isopropylphenyl)-3,4-dihydro-1H-pyrido[3,4-b]indole-2(9H)-carboxylate,
4-chlorophenyl 6-chloro-1-(4-(2-morpholinoethoxy)phenyl)-3,4-dihydro-1H-pyrido[3,4-b]indole-2(9H)-carboxylate,
(S)-4-methoxyphenyl 6-bromo-1-(4-chlorophenyl)-3,4-dihydro-1H-pyrido[3,4-b]indole-2(9H)-carboxylate,
4-chlorophenyl 6-bromo-1-(4-methoxyphenyl)-3,4-dihydro-1H-pyrido[3,4-b]indole-2(9H)-carboxylate,
ethyl 6-chloro-1-(4-(2-(thiazol-2-ylamino)ethoxy)phenyl)-3,4-dihydro-1H-pyrido[3,4-b]indole-2(9H)-carboxylate,
ethyl 6-chloro-1-(4-(2-(5-methylthiazol-2-ylamino)ethoxy)phenyl)-3,4-dihydro-1H-pyrido[3,4-b]indole-2(9H)-carboxylate,
ethyl 6-chloro-1-(4-(2-(pyridin-4-ylamino)ethoxy)phenyl)-3,4-dihydro-1H-pyrido[3,4-b]indole-2(9H)-carboxylate,
isobutyl 6-chloro-1-(4-(2-(thiazol-2-ylamino)ethoxy)phenyl)-3,4-dihydro-1H-pyrido[3,4-b]indole-2(9H)-carboxylate,
isobutyl 6-chloro-1-(4-(2-(5-methylthiazol-2-ylamino)ethoxy)phenyl)-3,4-dihydro-1H-pyrido[3,4-b]indole-2(9H)-carboxylate,
isobutyl 6-chloro-1-(4-(2-(pyridin-4-ylamino)ethoxy)phenyl)-3,4-dihydro-1H-pyrido[3,4-b]indole-2(9H)-carboxylate,
2-methoxyethyl 6-chloro-1-(4-(2-(thiazol-2-ylamino)ethoxy)phenyl)-3,4-dihydro-1H-pyrido[3,4-b]indole-2(9H)-carboxylate,
2-methoxyethyl 6-chloro-1-(4-(2-(5-methylthiazol-2-ylamino)ethoxy)phenyl)-3,4-dihydro-1H-pyrido[3,4-b]indole-2(9H)-carboxylate,
2-methoxyethyl 6-chloro-1-(4-(2-(pyridin-4-ylamino)ethoxy)phenyl)-3,4-dihydro-1H-pyrido[3,4-b]indole-2(9H)-carboxylate,
4-fluorophenyl 6-chloro-1-(4-(2-(thiazol-2-ylamino)ethoxy)phenyl)-3,4-dihydro-1H-pyrido[3,4-b]indole-2(9H)-carboxylate,
4-fluorophenyl 6-chloro-1-(4-(2-(5-methylthiazol-2-ylamino)ethoxy)phenyl)-3,4-dihydro-1H-pyrido[3,4-b]indole-2(9H)-carboxylate,
4-chlorophenyl 6-chloro-1-(4-(2-(thiazol-2-ylamino)ethoxy)phenyl)-3,4-dihydro-1H-pyrido[3,4-b]indole-2(9H)-carboxylate,
4-chlorophenyl 6-chloro-1-(4-(2-(5-methylthiazol-2-ylamino)ethoxy)phenyl)-3,4-dihydro-1H-pyrido[3,4-b]indole-2(9H)-carboxylate, and
4-chlorophenyl 6-chloro-1-(4-(2-(pyridin-3-ylamino)ethoxy)phenyl)-3,4-dihydro-1H-pyrido[3,4-b]indole-2(9H)-carboxylate.

22. The use of any one of claims 1 through 21, wherein said compounds are present as a substantially pure enantiomer.

23. The use of claim 22, wherein said substantially pure enantiomer is present as the (S) enantiomer at the chiral carbon on position 1 of the compound.

24. The use of any one of claim 22 or 23, wherein said substantially pure enantiomer is present in an amount greater than or equal to 90%, in an amount greater than or equal to 92%, in an amount greater than or equal to 95%, in an amount greater than or equal to 98%, in an amount greater than or equal to 99%, or in an amount equal to 100%.

25. The use of claim 1, further comprising a kit having instructions for orally administering a therapeutically effective amount of said compounds or medicament thereof once, twice or thrice daily to a subject in need thereof.

26. The use of claim 1, wherein said medicament further comprises a pharmaceutical composition having a therapeutically effective amount of said compounds and one or more pharmaceutically acceptable excipients.

27. The use of claim 26, wherein said pharmaceutical composition is a lipid-based, orally administered formulation comprising a therapeutically effective amount of said compounds and a surface active excipient, a triglyceride and an ester of steric acid.
